(19) United States
(12) Patent Application Publication
(10) Pub. No.: US 2023/0126375 A1
(43) Pub. Date: Apr. 27, 2023

(54) ENGINEERED BACTERIA AND METHODS OF PRODUCING SUSTAINABLE BIOMOLECULES

(71) Applicant: PRESIDENT AND FELLOWS OF HARVARD COLLEGE, Cambridge, MA (US)

(72) Inventors: Shannon Noel NANGLE, Cambridge, MA (US); Marika ZIESACK, Cambridge, MA (US); Pamela A. SILVER, Cambridge, MA (US); Sarabeth BUCKLEY, Cambridge, MA (US)

(73) Assignee: PRESIDENT AND FELLOWS OF HARVARD COLLEGE, Cambridge, MA (US)

(21) Appl. No.: 17/797,301

(22) PCT Filed: Feb. 3, 2021
(86) PCT No.: PCT/US2021/016406

§ 371 Date: Aug. 3, 2022

Related U.S. Application Data
(60) Provisional application No. 62/969,796, filed on Feb. 4, 2020.

(51) Int. Cl. C12N 1/20; C12N 9/10; C12N 9/16

(52) U.S. Cl. CPC C12N 1/205; C12N 9/1029; C12Y 203/01; C12N 9/16; C12Y 301/00

(57) ABSTRACT

The technology described herein is directed to engineered chemoautotrophic bacteria and methods of producing sustainable biomolecules. In several aspects, described herein are engineered bacteria and corresponding methods, compositions, and systems for the production of products such as polyhydroxyalkanoates (PHA), sugar feedstocks, and lipochitooligosaccharide (LCO) fertilizers.

### CROSS-REFERENCE TO RELATED APPLICATIONS

This application claims benefit under 35 U.S.C. § 119(e) of U.S. Provisional Application No. 62/969,796 filed Feb. 4, 2020, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Feb. 2, 2021, is named 002806-095240WOPT_SL.txt and is 270,322 bytes in size.

### TECHNICAL FIELD

The technology described herein relates to engineered bacteria and methods of producing sustainable biomolecules.

### BACKGROUND

A sustainable future relies, in part, on minimizing the usage of petrochemicals and reducing greenhouse gas (GHG) emissions. One way to accomplish this goal is through increasing the usage of sustainable fuel and bioproducts from engineered microorganisms, i.e., microbial bioproduction. Traditional microbial bioproduction utilizes carbohydrate-based feedstocks, but some of the cheapest and most sustainable feedstocks are gases (e.g., CO, CO₂, H₂, CH₄) from various point sources (e.g., steel mills, ethanol production plants, steam reforming plants, biogas). Compared to traditional bioproduction, gas fermentation represents a more cost-effective method that uses land more efficiently and has a smaller carbon footprint.

C. necator H16 (formerly known as Ralstonia eutropha H₁₆) is an attractive species for industrial gas fermentation. It is a facultative chemolithotrophic bacterium that derives its energy from H₂ and carbon from CO₂, is genetically tractable, can be cultured with inexpensive minimal media components, is non-pathogenic, has a high-flux carbon storage pathway, and fixes the majority of fed CO₂ into biomass. However, many previous C. necator bioproduction methods have relied upon carbohydrate-based feedstocks (see e.g., U.S. Pat. No. 7,622,277; EP Patent 2,935,599; Green et al. Biomacromolecules. 2002 Jan.-Feb., 3(1):208-13; Brigham et al. Deletion of Glyoxylate Shunt Pathway Genes Results in a 3-Hydroxybutyrate Overproducing Strain of Ralstonia eutropha. 2015 Synthetic Biology: Engineering, Evolution & Design. Poster Abstract 17: p. 32; the content of each of which is incorporated by reference in its entirety). There is a need to expand from this work by engineering C. necator to produce a large diversity of products using gas fermentation in order to promote the sustainable development of industrial bioproduction.

### SUMMARY

The technology described herein is directed to engineered chemoautotrophic bacteria and methods of using them to produce sustainable biomolecules. In one aspect, described herein are engineered bacteria and corresponding methods, compositions, and systems for the production of bioplastics such as polyhydroxyalkanoates (PHA). In another aspect, described herein are engineered bacteria and corresponding methods, compositions, and systems for the production of feedstocks such as sucrose feedstocks. In another aspect, described herein are engineered heterotrophs and corresponding methods, compositions, and systems for the production of secondary products from said feedstocks. In another aspect, described herein are engineered bacteria and corresponding methods, compositions, and systems for the production of fertilizers such as lipochitooligosaccharide (LCO).

Herein, C. necator is shown to bridge the gap between cheap gaseous feedstocks and versatile bioproduction. The methods and compositions described herein permit the production of tailored polymers using C. necator, something not achieved by prior gas fermentation applications. Three avenues are addressed for bioproduction that were selected for their ability to reduce greenhouse gas (GHG) emissions, e.g., when industrially scaled. First, for bioproduction to play a major role in replacing unsustainable industries, the existing infrastructure can be provided for by producing feedstocks for heterotrophs from CO₂ rather than from plant material. Second, to demonstrate the versatility of commodity products C. necator is well-positioned to address, described herein are engineered bacteria to diversify the types of PHA co-polymers that can be made lithotrophically—beyond polyhydroxybutyrate (PHB). Third, C. necator was used to produce a plant growth enhancer to promote crop yields and offset fertilizer use. Implementation of these three avenues can reduce the demands set on agriculture to generate bioproducts while increasing land-use efficiency for food.

Accordingly, in one aspect described herein is an engineered Cupriavidus necator bacterium, comprising: at least one exogenous copy of at least one functional polyhydroxyalkanoate (PHA) synthase gene; and at least one exogenous copy of at least one functional thioesterase gene.

In some embodiments of any of the aspects, the engineered bacterium further comprises: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product.

In some embodiments of any of the aspects, the engineered bacterium further comprises: (i) at least one endogenous beta-oxidation gene comprising at least one engineered inactivating modification; or (ii) at least one exogenous inhibitor of an endogenous beta-oxidation gene or gene product.

In some embodiments of any of the aspects, said engineered bacteria is a chemoautotroph.

In some embodiments of any of the aspects, wherein said engineered bacteria uses CO₂ as its sole carbon source, and/or said engineered bacteria uses H₂ as its sole energy source.

In some embodiments of any of the aspects, the endogenous PHA synthase comprises phaC.

In some embodiments of any of the aspects, the functional PHA synthase gene is heterologous.

In some embodiments of any of the aspects, the functional heterologous PHA synthase gene comprises a Pseudomonas aeruginosa phaC1, a Pseudomonas aeruginosa phaC2 gene, and/or Pseudomonas spp. 61-3 phaC1.

In some embodiments of any of the aspects, the functional thioesterase gene is heterologous.

In some embodiments of any of the aspects, the functional heterologous thioesterase gene comprises a Umbellularia californica FatB2 gene, a Cuphea palustris FatB1 gene, a Cuphea palustris FatB2 gene, or a Cuphea palustris FatB2-FatB1 hybrid gene.

In some embodiments of any of the aspects, the endogenous beta-oxidation gene is 3-hydroxyacyl-CoA dehydrogenase (fadB) or acyl-CoA ligase.

In some embodiments of any of the aspects, an engineered inactivating modification of a gene comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

In some embodiments of any of the aspects, the inhibitor of an endogenous beta-oxidation enzyme is acrylic acid.

In some embodiments of any of the aspects, said engineered bacteria produces medium chain length PHA.

In another aspect described herein is a method of producing medium-chain-length polyhydroxyalkanoate (MCL-PHA), comprising: (a) culturing the engineered bacterium as described herein in a culture medium comprising CO₂ and/or H₂; and (b) isolating, collecting, or concentrating MCL-PHA from said engineered bacterium or from the culture medium of said engineered bacterium.

In some embodiments of any of the aspects, the isolated MCL-PHA comprises an R group fatty acid which is 6 to 14 carbons long (C6-C14).

In some embodiments of any of the aspects, the total PHA isolated comprises at least 50% MCL-PHA.

In some embodiments of any of the aspects, the total PHA isolated comprises at least 80% MCL-PHA.

In some embodiments of any of the aspects, the total PHA isolated comprises at least 95% MCL-PHA.

In some embodiments of any of the aspects, the total PHA isolated comprises at least 98% MCL-PHA.

In some embodiments of any of the aspects, the total PHA isolated comprises at least 95% MCL-PHA with an R group fatty acid of C10-C14.

In some embodiments of any of the aspects, the total PHA isolated comprises at least 80% MCL-PHA with an R group fatty acid of C12-C14.

In some embodiments of any of the aspects, the culture medium comprises CO₂ as the sole carbon source, and/or the culture medium comprises H₂ as the sole energy source.

In another aspect described herein is an engineered C. necator bacterium, comprising one or more of the following: (a) at least one exogenous copy of at least one functional sugar synthesis gene; and/or (b) at least one exogenous copy of at least one functional sugar porin gene.

In some embodiments of any of the aspects, said engineered bacteria is a chemoautotroph.

In some embodiments of any of the aspects, said engineered bacteria uses CO₂ as its sole carbon source, and/or said engineered bacteria uses H₂ as its sole energy source.

In some embodiments of any of the aspects, the at least one functional sugar synthesis gene is heterologous.

In some embodiments of any of the aspects, the at least one functional sugar synthesis gene comprises at least one functional sucrose synthesis gene.

In some embodiments of any of the aspects, the at least one functional heterologous sucrose synthesis gene comprises Synechocystis sp. PCC 6803 sucrose phosphate synthase (SPS) and/or Synechocystis sp. PCC 6803 sucrose phosphate phosphatase (SPP).

In some embodiments of any of the aspects, the functional sugar porin gene is heterologous.

In some embodiments of any of the aspects, the functional sugar porin gene is a functional sucrose porin gene.

In some embodiments of any of the aspects, the functional heterologous sucrose porin gene comprises E. coli sucrose porin (scrY).

In some embodiments of any of the aspects, said engineered bacteria produces a feedstock solution.

In some embodiments of any of the aspects, said bacterium is co-cultured with a second microbe that consumes the feedstock solution

In another aspect described herein is an engineered heterotroph, comprising one or more of the following: (a) at least one overexpressed functional sucrose catabolism gene; (b)(i) at least one endogenous sucrose catabolism repressor gene comprising at least one engineered inactivating modification; or (b)(ii) at least one exogenous inhibitor of an endogenous sucrose catabolism repressor gene or gene product; (c)(i) at least one endogenous arabinose utilization gene comprising at least one engineered inactivating modification; or (c)(ii) at least one exogenous inhibitor of an endogenous arabinose utilization gene or gene product; and/or (d) at least one exogenous copy of at least one functional secondary product synthesis gene.

In some embodiments of any of the aspects, the engineered heterotroph is E. coli.

In some embodiments of any of the aspects, the at least overexpressed functional sucrose catabolism gene is endogenous.

In some embodiments of any of the aspects, the at least overexpressed functional sucrose catabolism gene comprises an invertase (CscA), a sucrose permease (CscB), and/or a fructokinase (CscK).

In some embodiments of any of the aspects, the endogenous sucrose catabolism repressor gene comprises the repressor (CscR).

In some embodiments of any of the aspects, the endogenous arabinose utilization gene comprises araB, araA, araD, and/or araC.

In some embodiments of any of the aspects, the at least one functional secondary product synthesis gene is heterologous.

In some embodiments of any of the aspects, the at least one functional secondary product synthesis gene comprises a violacein synthesis gene.

In some embodiments of any of the aspects, the at least one functional violacein synthesis gene comprises VioA, VioB, VioC, VioD, and/or VioE.

In some embodiments of any of the aspects, the at least one functional secondary product synthesis gene comprises a β-carotene synthesis gene.

In some embodiments of any of the aspects, the at least one functional β-carotene synthesis gene comprises CrtE, CrtB, CrtI, and/or CrtY.

In some embodiments of any of the aspects, the engineered heterotroph has enhanced sucrose utilization as compared to the same heterotroph lacking the engineered sucrose catabolism gene(s), sucrose catabolism repressor(s), arabinose utilization gene(s), and/or secondary product synthesis gene(s).

In another aspect described herein is a method of producing a feedstock solution, comprising: (a) culturing the engineered bacterium as described herein in a culture medium comprising CO₂ and/or H₂; and (b) isolating, collecting, or concentrating a feedstock solution from said engineered bacterium or from the culture medium of said engineered bacterium.

In some embodiments of any of the aspects, the culture medium comprises CO₂ as the sole carbon source, and/or the culture medium comprises H₂ as the sole energy source.

In some embodiments of any of the aspects, the culture medium further comprises arabinose.

In some embodiments of any of the aspects, the feedstock solution comprises a sucrose concentration of at least 100 mg/mL.

In some embodiments of any of the aspects, the feedstock solution comprises a sucrose concentration of at least 150 mg/mL.

In some embodiments of any of the aspects, the feedstock solution comprises a sucrose feedstock for at least one heterotroph.

In some embodiments of any of the aspects, the at least one heterotroph comprises an organism with enhanced sucrose utilization.

In some embodiments of any of the aspects, the at least one heterotroph comprises E. coli and/or S. cerevisiae.

In some embodiments of any of the aspects, the at least one heterotroph comprises an engineered bacterium as described herein.

In another aspect described herein is an engineered C. necator bacterium comprising at least one exogenous copy of at least one functional lipochitooligosaccharide synthesis gene.

In some embodiments of any of the aspects, said engineered bacteria is a chemoautotroph.

In some embodiments of any of the aspects, said engineered bacteria uses CO₂ as its sole carbon source, and/or said engineered bacteria uses H₂ as its sole energy source.

In some embodiments of any of the aspects, the at least one functional lipochitooligosaccharide synthesis gene comprises an N-acetylglucosaminyltransferase gene, a deacetylase gene, and/or an acetyltransferase gene.

In some embodiments of any of the aspects, the at least one functional lipochitooligosaccharide synthesis gene is heterologous.

In some embodiments of any of the aspects, the at least one functional heterologous lipochitooligosaccharide synthesis gene comprises B. japonicum NodC, B. japonicum NodB, and/or B. japonicum NodA.

In some embodiments of any of the aspects, said engineered bacteria produces lipochitooligosaccharide.

In another aspect described herein is a method of producing a fertilizer solution, comprising: (a) culturing the engineered bacterium as described herein in a culture medium comprising CO₂ and/or H₂; and (b) isolating, collecting, or concentrating a fertilizer solution from said engineered bacterium or from the culture medium of said engineered bacterium.

In some embodiments of any of the aspects, the culture medium comprises CO₂ as the sole carbon source, and/or the culture medium comprises H₂ as the sole energy source.

In some embodiments of any of the aspects, the fertilizer comprises lipochitooligosaccharides.

In some embodiments of any of the aspects, the fertilizer solution comprises a lipochitooligosaccharide concentration of at least 1 mg/L.

In another aspect described herein is a system comprising: (a) a reactor chamber with a solution contained therein, wherein the solution comprises hydrogen (H₂) and carbon dioxide (CO₂); and (b) at least one of the following engineered bacteria in the solution: (i) the engineered bioplastics bacterium as described herein; (ii) the engineered sugar feedstock bacterium as described herein; (iii) the engineered heterotroph as described herein; or (iv) the engineered fertilizer solution bacterium as described herein.

In some embodiments of any of the aspects, the system further comprises a pair of electrodes in contact with the solution that split water to form the hydrogen.

In some embodiments of any of the aspects, the system further comprises an isolated gas volume above a surface of the solution within a head space of a reactor chamber.

In some embodiments of any of the aspects, the isolated gas volume comprises primarily carbon dioxide.

In some embodiments of any of the aspects, the system further comprises a power source comprising a renewable source of energy.

In some embodiments of any of the aspects, the renewable source of energy comprises a solar cell, wind turbine, generator, battery, or grid power.

### BRIEF DESCRIPTION OF THE DRAWINGS

FIG. 1A-1C is a series of schematics showing metabolic pathways that were modified in C. necator. FIG. 1A is a schematic showing the sucrose synthesis pathway. Enzymes from Synechocystis sp. PCC 6803: SPS, sucrose phosphate synthase that binds UDP-glucose and fructose-6-phosphate; SPP, sucrose phosphate phosphatase that removes the phosphate on the fructose to release sucrose. Enzymes from E. coli: scrY, sucrose porin that exports sucrose through active diffusion. FIG. 1B is a schematic showing the PHA synthesis pathway. Thioesterase (TE) enzymes from: U. californica FatB2 a 12:0 acyl-ACP TE and an engineered chimera of C. palustris FatB1(aa 1-218) and FatB2 (aa 219-316)—Chimera 4 (chim4) that produce free fatty acids of specific lengths. PHA synthase (phaC) enzymes: Native C. necator C4 phaC_{Cn}; a C12 P. aeruginosa PAO1 phaC2_{Pa}; and a Pseudomonas spp 61-3 phaC1_{Ps}, each of which performs the final step in PHA polymerization and grows the chain. FIG. 1C is a schematic showing the nodulation factor synthesis pathway for Nod Cn-V (C_{18:1}). Enzymes from B. japonicum: NodC protein, an N-acetylglucosaminyltransferase that builds the backbone, NodB, a deacetylase that acts on the non-reducing end, and NodA, an acetyltransferase that attaches a fatty acid.

FIG. 2A-2F is a series of graphs showing sucrose-based C. necator-E. coli co-culture fueled by CO₂/H₂. Depicted are average values with error bars indicating standard deviation. FIG. 2A is a line graph showing sucrose production in supernatant from C. necator without porin (dark grey diamonds as indicated) or with porin (light grey diamonds as indicated) without arabinose induction (empty symbols) or with 0.3% arabinose after 3 days (filled symbols). FIGS. 2B, 2D, and 2F are a series of line graphs showing C. necator-E. coli co-culture. FIG. 2B shows PAS842 growth in co-culture with WT C. necator H16 and induction (dark grey circles), with PAS837 without induction (empty light grey circles) or with induction (filled light grey circles). FIG. 2D shows sucrose concentrations in supernatant of conditions as described above (dark grey diamonds, light grey empty diamonds, light grey filled diamonds respectively). FIG. 2F shows C. necator growth in conditions as described above (dark grey circles, empty light grey circles, filled light grey circles respectively). FIG. 2C is a line graph showing a comparison of PAS842 growth in supernatant derived from PAS837 with co-culture. PAS842 was grown in increasing sucrose concentrations to generate a standard curve. PAS842 growth grown in PAS837 supernatant in relation to measured sucrose in supernatant is indicated in grey circles. PAS842 growth in co-culture with uninduced PAS837 (empty black circles) and induced PAS837 (filled black circles) are shown. FIG. 2E is a schematic and pair of bar graphs showing violacein and carotene production in co-culture. PAS845 and PAS846 were grown in co-culture with PAS837 with induction. Samples were harvested, violacein and carotene were extracted and quantified as described in the Methods.

FIG. 3A-3E is a series of bar graphs showing lithotrophic production of tailored PHA. 3-hydroxyalkanoate mol/mol ratio from methanolyzed methyl esters of the purified PHA. Values are based on area under the curve (AUC) for each peak at m/z=103 via GCMS (n=3 for each condition). Left panels are strains with native phaC_{Cn} background; right shaded panels are in the knock-out ΔphaC_{Cn} background. Bottom panels are with the administration of 240 μg/mL acrylic acid with induction. FIG. 3A is a series of bar graphs. Panel one (upper left), wild-type phaC_{Cn} produces 100% 3HB. Panel two (upper right), knock-out ΔphaC_{Cn} does not produce detectable amounts of PHAs. Third (lower left) and fourth (lower right) panels, the composition of the polymer is not strongly affected by acrylic acid in either strain. FIG. 3B. PAS828 (phaC_{Cn}, pBAD UcFatB2, phaC1_{Pa}); PAS829 (ΔphaC_{Cn}, pBAD UcFatB2, phaC1_{Pa}) FIG. 3C. PAS830 (phaC_{Cn}, pBAD chim4, phaC1_{Ps}); PAS831 (ΔphaC_{Cn}, pBAD chim4, phaC1_{Ps}) FIG. 3D. PAS832 (phaC_{Cn}, pBAD UcFatB2, phaC2_{Pa}); PAS833 (ΔphaC_{Cn}, pBAD UcFatB2, phaC2_{Pa}) FIG. 3E is a series of bar graphs showing a side-by-side comparison of representative co-polymers in each condition to demonstrate the predictable trends of those conditions. Fatty acids represented from bottom to top of bar stacks: C4; C6; C8; C10; C12; C14.

FIG. 4A-4H is a series of graphs showing lithotrophic production of Nod Cn-V (C18:1) in C. necator. FIG. 4A is a bar graph showing yields of wild type B. japonicum strain 6 (dark grey) and LCO-producing C. necator (light grey)+/− inducer (genistein and arabinose, respectively). FIG. 4B shows a LC-MS mass spectrum of eluted peak at 77.65 min containing Nod Cn-V (C_{18:1}). FIG. 4C is a line graph showing germination rates in seeds in response to LCO application for spinach, soybean, and corn. Seeds were treated with: water (black circles), extract from C. necator vector control (grey squares), a standard LCO (Nod Bj-V (C18:1 MeFuc)) control from B. japonicum (dark grey up-triangles), and extracted Nod Cn-V (C18:1) from C. necator (light grey down-triangles). FIG. 4D is a dot plot showing that spinach germination weight increased in Nod Cn-V (C18:1) compared to Bj-V (C_{18:1}MeFuc) (p=0.0072); not all seeds germinated which is reflected in the different number of samples in each group. Two-tailed Mann-Whitney test was used for FIG. 4D. FIG. 4E is a dot plot showing that corn germination weight increased significantly in Nod Cn-V (C18:1) compared to all conditions: water (p<0.0001), vector control (p<0.0001), and Nod Bj-V (C18:1 MeFuc) (p=0.0003). FIG. 4F is a dot plot showing growth characteristics of greenhouse corn. Nod Cn-V (C18:1) extract samples were derived from 1-butanol extraction. Nod Cn-V (C18:1) increased corn wet weight compared to Nod Bj-V (C_{18:1}MeFuc) (p=0.0023) and Nod Cn-V (C18:1). FIG. 4G is a dot plot showing that Nod Cn-V (C18:1) significantly increased leaf number compared to fertilizer (p=0.036), Nod Bj-V (C_{18:1}MeFuc) (p=0.007). FIG. 4H is a dot plot showing that Nod Cn-V (C18:1) significantly increased the corn height as compared to water (p=0.04). Multiple comparisons one-way ANOVA were used for FIG. 4E-4H). Asterisks indicate significance: *=<0.05, **=<0.01 and ***=<0.001.

FIG. 5A-5D is a series of schematics and graphs showing sustainability comparisons of existing strategies. FIG. 5A is a schematic showing a comparison between sugarcane, cyanobacteria, and C. necator. Solar-to-biomass conversion efficiency of plants is approx. 1% annually, cyanobacteria is 3% in open ponds and 5-7% in photobioreactors. Photovoltaics (PVs) with an average solar-to-energy conversion of 22% can generate H₂ at 14% efficiency. This converts to 30-70 ton ha^{-l}yr⁻¹ of sugarcane biomass with 20% biomass-to-sucrose efficiency of 20% to 6-14 ton ha^{-l}yr⁻¹ sucrose. For cyanobacteria, with an 80% biomass-to-sucrose efficiency, 5-40 ton ha^{-l}yr⁻¹ sucrose. For C. necator, using PV area as the land use equivalent and using the biomass-to-sucrose efficiency demonstrated herein of 11%, 4,510 ton ha^{-l}yr^{-l} biomass can produce 510 ton ha^{-l}yr⁻¹ of sucrose. FIG. 5B is a bar graph showing GHG emissions for main classes of plastics (PET, polyethylene terephthalate; PP, polypropylene; PLA, polylactic acid; PHA, polyhydroxyalkanoates) with current energy mix. Based on the conversion efficiency demonstrated herein of PHA 50% DCW and the CO₂ drawdown rate of 0.61-0.65 g of biomass per g CO₂ FIG. 5C. is a series of graphs comparing carbon footprints and biodegradability. Top panel: the carbon footprint of the end-of-life of plastics. Bottom panel: the relative biodegradability of main classes of plastics. Petrochemical plastics are not processed in industrial composter or anaerobic digestion. Depending on conditions of the composters/digesters, PLA will not degrade. FIG. 5D is a bar graph showing fertilizer (NPK, nitrogen-phosphorus-potassium, NPK) offset from LCO supplementation. Based on average corn production of 11.1 ton ha^{-l}yr⁻¹ and 40% yield increase conferred by fertilizer, which served at 100% of possible growth increase. Intercropping uses different crops to increase soil quality. CO₂e values are based only on NPK offset to produce equivalent yields. LCO current are values based on field study yields. LCO optimized are values based on optimized greenhouse growth conditions.

FIG. 6A-6C are a series of schematics showing the experimental setup. All strains were initially inoculated in rich broth, washed, and inoculated in minimal Schuster media, placed in a vacuum jar, and supplied CO₂ and H₂ as the sole carbon and energy source, respectively. These cultures were then grown until they reach an OD 600=2-3, approximately 6 days. Once the cultures had switched to lithoautotrophic metabolism they were back-diluted into fresh media at OD 600=0.2 (for PHAs and LCOs) or OD 600=0.5 (for sucrose). After induction, the cultures were resupplied with fresh CO₂ and H₂ daily or every other day. FIG. 6A show the experimental set-up for engineered sucrose feedstock bacteria. Three days after back-dilution lithotrophic sucrose-producing C. necator were induced and inoculated with E. coli at OD 600=0.01. The co-culture was grown for an additional 7 days, plated and assayed for sucrose concentration every other day. FIG. 6B show the experimental set-up for engineered bioplastics bacteria. PHA-producing strains were induced at OD 600=1 or approximately 2 days in nitrogen-limiting media (if needed, acrylic acid was also added at this time). Strains were then grown for an additional 4 days to accumulate PHAs. Cells were then washed, lyophilized and lysed by NaClO—. The PHA pellets were lyophilized, then subjected to methanolysis. 3-hydroxy acids were solubilized in chloroform and then analyzed by GC-MS. FIG. 6C show the experimental set-up for engineered LCO bacteria. LCO-producing strains were induced at OD 600=1 or approximately 2 days after back-dilution. After an additional 4 days of growth they were harvested, washed, subjected to butanol extraction, then concentrated by rotary evaporator. Samples to be analyzed by HPLC and LC-MS were solubilized in 20% acetonitrile. Samples to be applied for germination experiments were solubilized in water and applied to seeds. The seeds were grown in a growth chamber for 9 days and then analyzed. Samples that were applied to greenhouse experiments were purified from rich media due to volume limitations of the lithotrophic conditions (50 mL). Purified LCOs were applied to corn seeds, which were then planted. LCOs were applied a second time when planted. After 2 weeks the corn plant growth was analyzed.

FIG. 7 is a bar graph showing a comparison of sucrose producing enzymes from different cyanobacterial species expressed in C. necator. Sucrose phosphate synthetase and sucrose phosphate phosphatase from cyanobacterial species were expressed in C. necator and sucrose production in supernatant was determined after 7 days. Shown are three biological replicates with mean and standard deviation.

FIG. 8 is a series of bar graphs showing sucrose titration. E. coli W and S. cerevisiae W303 strains were grown in Schuster media supplemented with varying concentrations of sucrose. OD 600 was recorded after 2 days of anaerobic growth. Reported are mean values of three biological replicates with error bars indicating standard deviation.

FIG. 9 is a series of dot plots showing heterotroph growth in C. necator supernatant. E. coli PAS842 and S. cerevisiae PAS844 were grown for 2 days anaerobically at 30° C. in supernatant from C. necator PAS837 that was grown lithotrophically for 7 days with and without induction. Colony count (cfu/mL) was assessed by plating at the beginning of the experiment and after 48 h and doublings were calculated. As a control, heterotrophs were grown in Schuster media with and without sucrose. In all conditions except for induced C. necator supernatant, 0.3% arabinose were added.

FIG. 10 is a line graph showing octanoate production by C. necator. Concentration as determined by GC-MS analysis. Known concentrations of C6, C8, C10, and C12 fatty acids were used to generate a standard curve and to quantify the production of single fatty acid species. Time points indicate days post-induction. Wildtype samples were below detection.

FIG. 11 shows PHA content relative to dry cell weight (DCW). Reported are mean values and standard deviation of three biological replicates for each strain.

FIG. 12A-12C shows 3HA ratios in tailored PHAs. Values represent data in FIG. 3. FIG. 12A: PAS828 (phaC Cn, pBAD Uc FatB2, phaC1_{Pa}); PAS829 (ΔphaC Cn, pBAD Uc FatB2, phaC1 Pa). FIG. 12B: PAS830 (phaC Cn, pBAD chim4, phaC1_{Ps}) PAS831 (ΔphaC Cn, pBAD chim4, phaC1_{Ps}). FIG. 12C: PAS832 (phaC Cn, pBAD Uc FatB2, phaC2_{Pa}) PAS833 (ΔphaC Cn, pBAD Uc FatB2, phaC2_{Pa}). Reported are mean values and standard deviation of three biological replicates for each strain. Fatty acids represented by: C4; C6; C8; C10; C12; C14.

FIG. 13A-13B is a series of line graphs showing representative LCO HPLC elution profiles. FIG. 13A shows representative spectra from HPLC analysis of Nod Cn-V (C 18:1) (e.g., light grey). Purified extracts from induced and uninduced, vector control and engineered C. necator. Extracts from Standard (black), vector control C. necator (dark grey), or the engineered C. necator (PAS838) (light grey). Induced cultures are shown by solid lines and uninduced by dashed lines. FIG. 13B shows induced B. japonicum 100 compared to an LCO standard from B. japonicum 523C; both indicate a characteristic double elution peak, which is seen in the engineered C. necator (PAS838) strain (see e.g., FIG. 13A). Extracts from Standard (black) or B. japonicum (grey). Induced cultures are shown by solid lines and uninduced by dashed lines.

FIG. 14A-14B shows representative LCO LC-MS spectra. FIG. 14A is a spectrum showing that Nod Cn-V (C 18:1) contains the characteristic peaks for the N-acetylglucosamine backbone with the largest peak at m/z=1256 rather than m/z=1416, indicating the lack of the fucose group found in B. japonicum 100. FIG. 14B is a spectrum showing Nod Bj-V (C18:1 MeFuc). Relevant peaks in FIG. 14A-14B are bolded and underlined.

FIG. 15 is a series of images showing representative germinated spinach seeds. The 10 longest seeds are shown in the water condition (left) and Nod Cn-V (C 18:1) condition (right).

FIG. 16A-16B is a series of graphs showing corn germination experiments. FIG. 16A is a line graph showing germination rates in seeds in response to LCO application for corn. Seeds were treated with: water (black circles), a vector control (grey squares), a standard LCO (Nod Bj-V (C18:1 MeFuc)) control from B. japonicum (dark grey up-triangles) and the extract from C. necator vector control (light grey down triangle). FIG. 16B is a dot plot showing that corn shoot length showed increased length in the Nod Cn-V (C 18:1) compared to water (p=0.0003) and the vector control (p=0.0003). Asterisks indicate significance: ***<0.0001 as analyzed by multiple comparison one-way ANOVA.

FIG. 17 is an image showing 160 corn plants that were grown in a greenhouse for two weeks (10 replicates in each condition). Plants were grown and harvested in a blinded experimental setup.

FIG. 18A is a schematic representation of a reactor. FIG. 18B is a schematic representation of the production of one or more products within the reactor of FIG. 18A. Adapted from US 2018/0265898 A1.

### DETAILED DESCRIPTION

Embodiments of the technology described herein are directed to engineered bacteria and methods of producing sustainable biomolecules. The methods and compositions described herein permit the production of tailored polymers using C. necator, something not achieved by prior gas fermentation applications. In one aspect, described herein are engineered bacteria and corresponding methods, compositions, and systems for the production of bioplastics such as polyhydroxyalkanoates (PHA). In another aspect, described herein are engineered bacteria and corresponding methods, compositions, and systems for the production of feedstocks such as sucrose feedstocks. In another aspect, described herein are engineered heterotrophs and corresponding methods, compositions, and systems for the production of secondary products from said feedstocks. In another aspect, described herein are engineered bacteria and corresponding methods, compositions, and systems for the production of fertilizers such as lipochitooligosaccharide (LCO).

As shown herein, coupling recent advancements in genetic engineering of microbes and gas-driven fermentation provides a path towards sustainable commodity chemical production. C. necator H16 is a suitable species primarily because it effectively utilizes H₂ and CO₂ and is genetically tractable. Demonstrated herein is the versatility of this organism in lithotrophic conditions, for example the production of sucrose, polyhydroxyalkanoates (PHAs), and lipochitooligosaccharides (LCOs). Sucrose production was engineered in a co-culture system, demonstrating heterotrophic growth 30 times that of unengineered wildtype C. necator. Because C. necator is known to produce polyhydroxyalkanoates (PHAs), its composition can be tailored by combining different thioesterases and phaCs to produce co-polymers directly from CO₂. Tailored PHA accumulated to ˜50% DCW (20-60% DCW) across all strains. Next, bacteria were engineered to produce a molecule—lipochitooligosaccharide (LCOs)—that has yet to be produced outside its native organism (Bradyrhizobium) and can address unsustainable practices in agriculture. C. necator was engineered to convert CO₂ into a LCO, a plant growth enhancer with titers of ˜1.4 mg/L—equivalent to yields in the native source, Bradyrhizobium. The LCOs were applied to germinating seeds as well as corn plants and significant increases were observed in a variety of growth parameters. Each of these results are examples of how a gas-utilizing bacteria can promote sustainable production.

Described herein are engineered bacteria that can be used to sustainably produce biomolecules. In some embodiments of any of the aspects, the engineered bacterium is a chemoautotroph. In some embodiments of any of the aspects, the engineered bacterium can grow under chemoautotrophic (i.e., lithotrophic) conditions. As used herein, the term “chemoautotroph” refers to an organism that uses inorganic energy sources to synthesize organic compounds from carbon dioxide. The term “chemolithotroph” can be used interchangeably with chemoautotroph. Chemoautotrophs stand in contrast to heterotrophs. As used herein, the term “heterotroph” refers to an organism that derives its nutritional requirements from complex organic substances (e.g., sugars).

In some embodiments of any of the aspects, the engineered bacterium is a chemolithotroph. As used herein, the term “chemolithotroph” refers to an organism that is able to use inorganic reduced compounds (e.g., hydrogen, nitrite, iron, sulfur) as a source of energy (e.g., as electron donors). The chemolithotrophy process is accomplished through oxidation of inorganic compounds and ATP synthesis. The majority of chemolithotrophs are able to fix carbon dioxide (CO₂) through the Calvin cycle, a metabolic pathway in which carbon enters as CO₂ and leaves as glucose (see e.g., Kuenen, G. (2009). “Oxidation of Inorganic Compounds by Chemolithotrophs”. In Lengeler, J.; Drews, G.; Schlegel, H. (eds.). Biology of the Prokaryotes. John Wiley & Sons. p. 242. ISBN 9781444313307). The chemolithotroph group of organisms includes sulfur oxidizers, nitrifying bacteria, iron oxidizers, and hydrogen oxidizers. The term “chemolithotrophy” refers to a cell's acquisition of energy from the oxidation of inorganic compounds, also known as electron donors. This form of metabolism is known to occur only in prokaryotes. See e.g., Table 1 for non-limiting examples of chemolithotrophic bacteria and archaea.

TABLE 1
Chemolithotrophic bacteria and archaea

| | Non-Limiting | Source of | Respiration |
|---|---|---|---|
| | Examples of | energy and | electron |
| Bacteria | Chemolithotrophs | electrons | acceptor |
| Iron bacteria | Acidithiobacillus | Fe²⁺ (ferrous iron) → | O₂ (oxygen) → |
| | ferrooxidans | Fe³⁺ (ferric iron) + e⁻ | H₂O (water) |
| Nitrosifying bacteria | Nitrosomonas | NH₃ (ammonia) → | O₂ (oxygen) → |
| | | NO₂⁻ (nitrite) + e⁻ | H₂O (water) |
| Nitrifying bacteria | Nitrobacter | NO₂_{^{−}} (nitrite) → | O₂ (oxygen) → |
| | | NO₃_{^{−}} (nitrate) + e⁻ | H₂O (water) |
| Chemotrophic | Halothiobacillaceae | S²⁻ (sulfide) → | O₂ (oxygen) → |
| purple | | S⁰ (sulfur) + e⁻ | H₂O (water) |
| sulfur bacteria | | | |
| Sulfur-oxidizing | Chemotrophic | S⁰ (sulfur) → | O₂ (oxygen) → |
| bacteria | Rhodobacteraceae | SO₄²⁻ (sulfate) + e⁻ | H₂O (water) |
| | and Thiotrichaceae | | |
| Aerobic hydrogen | Cupriavidus necator, | H₂ (hydrogen) → | O₂ (oxygen) → |
| bacteria | Cupriavidus | H₂O (water) + e⁻ | H₂O (water) |
| | metallidurans | | |
| Anammox bacteria | Planctomycetes | NH₄⁺ (ammonium) → | N₂ (nitrogen) + |
| | | NO₂_{^{−}} (nitrite) | H₂O (water) |
| Thiobacillus | Thiobacillus | S⁰ (sulfur) → | NO₃_{^{−}} (nitrate) |
| denitrificans | denitrificans | SO₄²⁻ (sulfate) + e⁻ | |
| Sulfate-reducing | Desulfovibrio paquesii | H₂ (hydrogen) → | Sulfate |
| bacteria: Hydrogen | | H₂O (water) + e⁻ | (SO₄²⁻) |
| bacteria | | | |
| Sulfate-reducing | Desulfotignum | PO₃³⁻ (phosphite) → | Sulfate |
| bacteria: Phosphite | phosphitoxidans | PO₄³⁻ (phosphate) + e⁻ | (SO₄²⁻) |
| bacteria | | | |
| Methanogens | Archaea | H₂ (hydrogen) → | CO₂ (carbon |
| | | H₂O (water) + e⁻ | dioxide) |
| Carboxydotrophic | Carboxydothermus | carbon monoxide | H₂O (water) → |
| bacteria | hydrogenoformans | (CO) → carbon | H₂ (hydrogen) |
| | | dioxide (CO₂) + e⁻ | |

In some embodiments of any of the aspects, the engineered bacteria is a chemolithotroph belonging to a classification selected from the group consisting of Acidithiobacillus, Alcaligenes, Carboxydothermus, Cupriavidus, Desulfotignum, Desulfovibrio, Halothiobacillaceae, Hydrogenomonas, Nitrobacter, Nitrosomonas, Planctomycetes, Ralstonia, Rhodobacteraceae, Thiobacillus, Thiotrichaceae, and Wautersia. In some embodiments of any of the aspects, the engineered organism is a methanogenic archaea (e.g., belonging to the genera Methanosarcina or Methanothrix). In some embodiments of any of the aspects, the engineered bacteria is selected from the group consisting of Acidithiobacillus ferrooxidans, Carboxydothermus hydrogenoformans, Cupriavidus metallidurans, Cupriavidus necator, Desulfotignum phosphitoxidans, Desulfovibrio paquesii, Thiobacillus denitrificans. In some embodiments of any of the aspects, the engineered bacteria is further engineered to be chemolithotrophic. In some embodiments of any of the aspects, the engineered bacterium is aerobic and uses O₂ as its respiration electron acceptor. In some embodiments of any of the aspects, the engineered bacteria can be a heterotroph or a chemolithotroph, e.g., depending on environmental conditions.

In some embodiments of any of the aspects, the engineered bacteria uses CO₂ as its sole carbon source or H₂ as its sole energy source. In some embodiments of any of the aspects, the engineered bacteria uses CO₂ as its sole carbon source and H₂ as its sole energy source. In some embodiments of any of the aspects, the engineered bacteria uses H₂ as its sole energy source. In some embodiments of any of the aspects, the engineered bacteria uses CO₂ as its sole carbon source.

In some embodiments of any of the aspects, the engineered bacteria is engineered from a bacteria that uses CO₂ as its sole carbon source or H₂ as its sole energy source. In some embodiments of any of the aspects, the engineered bacteria is engineered from a bacteria that uses CO₂ as its sole carbon source and H₂ as its sole energy source. In some embodiments of any of the aspects, the engineered bacteria is engineered from a bacteria that uses H₂ as its sole energy source. In some embodiments of any of the aspects, the engineered bacteria is engineered from a bacteria that uses CO₂ as its sole carbon source.

In some embodiments of any of the aspects, the engineered bacteria obtains at least 90%, at least 95%, at least 98%, at least 99% or more of its carbon from CO₂. In some embodiments of any of the aspects, the engineered bacteria obtains at least 90%, at least 95%, at least 98%, at least 99% or more of its energy from H₂. In some embodiments of any of the aspects, the engineered bacteria obtains at least 90%, at least 95%, at least 98%, at least 99% or more of its carbon from CO₂ and at least 90%, at least 95%, at least 98%, at least 99% or more of its energy from H₂.

As used herein, the term “carbon source” refers to the molecules used by an organism as the source of carbon for building its biomass; a carbon source can be an organic compound or an inorganic compound. “Source” denotes an environmental source. In some embodiments of any of the aspects, the engineered bacteria fixes carbon dioxide (CO₂) through the Calvin cycle, a metabolic pathway in which carbon enters as CO₂ and leaves as glucose. As used herein, the term “sole carbon source” denotes that the engineered bacteria uses only the indicated carbon source (e.g., CO₂) and no other carbon sources. For example, “sole carbon source” is intended to mean where the suitable conditions comprise a culture media containing a carbon source such that, as a fraction of the total carbon atoms in the media, the specific carbon source (e.g., CO₂), respectively, represent about 100% of the total carbon atoms in the media. In some embodiments, the sole carbon source of the engineered bacteria is inorganic carbon, including but not limited to carbon dioxide (CO₂) and bicarbonate (HCO3⁻). In some embodiments of any of the aspects, the sole carbon source is atmospheric CO₂.

In some embodiments of any of the aspects, the engineered bacteria uses CO₂ as its major carbon source, meaning at least 50% of its carbon atoms are obtained from CO₂. As a non-limiting example, the engineered bacteria obtains at least 50%, at least 60%, at least 70%, at least 80%, at least 90%, at least 95%, or at least 99% of its carbon atoms from CO₂.

In some embodiments of any of the aspects, the engineered bacteria does not use organic carbon as a carbon source. Non-limiting example of organic carbon sources include fatty acids, gluconate, acetate, fructose, decanoate; see e.g., Jiang et al. Int J Mol Sci. 2016 July; 17(7): 1157).

In some embodiments of any of the aspects, the engineered bacteria uses H₂ as its sole energy source. As used herein, the term “energy source” refers to molecules that contribute electrons and contribute to the process of ATP synthesis. As described here, the engineered bacterium can be a chemolithotroph, i.e., an organism that is able to use inorganic reduced compounds (e.g., hydrogen, nitrite, iron, sulfur) as a source of energy (e.g., as electron donors). As used herein, the term “sole energy source” denotes that the engineered bacteria uses only the indicated energy source (e.g., H₂) and no other energy sources. In some embodiments of any of the aspects, the sole energy source is atmospheric H₂.

In some embodiments of any of the aspects, the engineered bacteria uses H₂ as its major energy source, meaning at least 50% of its donated electrons (e.g., used for ATP synthesis) are obtained from H₂. As a non-limiting example, the engineered bacteria obtains at least 50%, at least 60%, at least 70%, at least 80%, at least 90%, at least 95%, or at least 99% of its donated electrons from H₂.

Bacteria used in the systems and methods disclosed herein may be selected so that the bacteria both oxidize hydrogen as well as consume carbon dioxide. Accordingly, in some embodiments, the bacteria may include an enzyme capable of metabolizing hydrogen as an energy source such as with hydrogenase enzymes. Additionally, the bacteria may include one or more enzymes capable of performing carbon fixation such as Ribulose-1,5-bisphosphate carboxylase/oxygenase (RuBisCO). One possible class of bacteria that may be used in the systems and methods described herein to produce a product include, but are not limited to, chemolithoautotrophs. Additionally, appropriate chemolithoautotrophs may include any one or more of Ralstonia eutropha (R. eutropha) as well as Alcaligenes paradoxs I 360 bacteria, Alcaligenes paradoxs 12/X bacteria, Nocardia opaca bacteria, Nocardia autotrophica bacteria, Paracoccus denitrificans bacteria, Pseudomonas facilis bacteria, Arthrobacter species 11X bacteria, Xanthobacter autotrophicus bacteria, Azospirillum lipferum bacteria, Derxia Gummosa bacteria, Rhizobium japonicum bacteria, Microcyclus aquaticus bacteria, Microcyclus ebruneus bacteria, Renobacter vacuolatum bacteria, and any other appropriate bacteria.

In some embodiments of any of the aspects, the engineered bacteria belongs to the Cupriavidus genus. The Cupriavidus genus of bacteria includes the former genus Wautersia. Cupriavidus bacteria are characterized as Gram-negative, motile, rod-shaped organisms with oxidative metabolism. Cupriavidus bacteria possess peritrichous flagella, are obligate aerobic organisms, and are chemoorganotrophic or chemolithotrophic. In some embodiments of any of the aspects, the engineered bacteria is selected from the group consisting of Cupriavidus alkal/philus, Cupriavidus basilensis, Cupriavidus campinensis, Cupriavidus gilardii, Cupriavidus laharis, Cupriavidus metallidurans, Cupriavidus necator, Cupriavidus nantongensis, Cupriavidus numazuensis, Cupriavidus oxalaticus, Cupriavidus pampae, Cupriavidus pauculus, Cupriavidus pinatubonensis, Cupriavidus plantarum, Cupriavidus respiraculi, Cupriavidus taiwanensis, and Cupriavidus yeoncheonensis.

In some embodiments of any of the aspects, the engineered bacterium is Cupriavidus necator. Cupriavidus necator can also be referred to as Ralstonia eutropha, Hydrogenomonas eutrophus, Alcaligenes eutropha, or Wautersia eutropha. In some embodiments of any of the aspects, the engineered bacterium is Cupriavidus necator strain H16. In some embodiments of any of the aspects, the engineered bacterium is Cupriavidus necator strain N-1.

Members of the species and genera described herein can be identified genetically and/or phenotypically. By way of non-limiting example, the engineered bacterium as described herein comprises a 16S rDNA sequence at least 97% identical to a 16S rDNA sequence present in a reference strain operational taxonomic unit for Cupriavidus necator. In some embodiments of any of the aspects, the engineered bacterium as described herein comprises a 16S rDNA that is at least 95% identical (e.g., at least 95%, at least 96%, at least 97%, at least 98%, or at least 99%) identical to the sequence of SEQ ID NO: 79 or SEQ ID NO: 91. In some embodiments of any of the aspects, the bacterium as described herein is engineered from Cupriavidus necator (e.g., strain H16 or strain N-1).

| SEQ ID NO: 79, Cupriavidus necator strain N-1 16S ribosomal RNA, | |
|---|---|
| partial sequence, NCBI Reference Sequence: NR_028766.1, 1356 bp | |
| 1 | ttagattgaa cgctggcggc atgccttaca catgcaagtc gaacggcagc acgggcttcg |
| 61 | gcctggtggc gagtggcgaa cgggtgagta atacatcgga acgtgccctg tagtggggga |
| 121 | taactagtcg aaagattagc taataccgca tacgacctga gggtgaaagc gggggaccgc |
| 181 | aaggcctcgc gctacaggag cggccgatgt ctgattagct agttggtggg gtaaaagcct |
| 241 | accaaggcga cgatcagtag ctggtctgag aggacgatca gccacactgg gactgagaca |
| 301 | cggcccagac tcctacggga ggcagcagtg gggaattttg gacaatgggg gcaaccctga |
| 361 | tccagcaatg ccgcgtgtgt gaagaaggcc ttcgggttgt aaagcacttt tgtccggaaa |
| 421 | gaaatggctc tggttaatac ccggggtcga tgacggtacc ggaagaataa gcaccggcta |
| 481 | actacgtgcc agcagccgcg gtaatacgta gggtgcgagc gttaatcgga attactgggc |
| 541 | gtaaagcgtg cgcaggcggt tttgtaagac aggcgtgaaa tccccgagct caacttggga |
| 601 | atggcgcttg tgactgcaag gctagagtat gtcagagggg ggtagaattc cacgtgtagc |
| 661 | agtgaaatgc gtagagatgt ggaggaatac cgatggcgaa ggcagccccc tgggacgtca |
| 721 | ctgacgctca tgcacgaaag cgtggggagc aaacaggatt agataccctg gtagtccacg |
| 781 | ccctaaacga tgtcaactag ttgttgggga ttcatttctt cagtaacgta gctaacgcgt |
| 841 | gaagttgacc gcctggggag tacggtcgca agattaaaac tcaaaggaat tgacggggac |
| 901 | ccgcacaagc ggtggatgat gtggattaat tcgatgcaac gcgaaaaacc ttacctaccc |
| 961 | ttgacatgcc actaacgaag cagagatgca ttaggtgccc gaaagggaaa gtggacacag |
| 1021 | gtgctgcatg gctgtcgtca gctcgtgtcg tgagatgttg ggttaagtcc cgcaacgagc |
| 1081 | gcaacccttg tctctagttg ctacgaaagg gcactctaga gagactgccg gtgacaaacc |
| 1141 | ggaggaaggt ggggatgacg tcaagtcctc atggccctta tgggtagggc ttcacacgtc |
| 1201 | atacaatggt gcgtacagag ggttgccaac ccgcgagggg gagctaatcc cagaaaacgc |
| 1261 | atcgtagtcc ggatcgtagt ctgcaactcg actacgtgaa gctggaatcg ctagtaatcg |
| 1321 | cggatcagca tgccgcggtg aatacgttcc cggtct |

SEQ ID NO: 91 Cupriavidus necator strain H16 16S ribosomal RNA
(1537 nucleotides (nt))
AGATTGAACTGAAGAGTTTGATCCTGGCTCAGATTGAACGCTGGCGGCATGCCTTACACA
TGCAAGTCGAACGGCAGCACGGGCTTCGGCCTGGTGGCGAGTGGCGAACGGGTGAGTAA
TACATCGGAACGTGCCCTGTAGTGGGGGATAACTAGTCGAAAGATTAGCTAATACCGCA
TACGACCTGAGGGTGAAAGCGGGGGACCGCAAGGCCTCGCGCTACAGGAGCGGCCGAT
GTCTGATTAGCTAGTTGGTGGGGTAAAAGCCTACCAAGGCGACGATCAGTAGCTGGTCT
GAGAGGACGATCAGCCACACTGGGACTGAGACACGGCCCAGACTCCTACGGGAGGCAG
CAGTGGGGAATTTTGGACAATGGGGGCAACCCTGATCCAGCAATGCCGCGTGTGTGAAG
AAGGCCTTCGGGTTGTAAAGCACTTTTGTCCGGAAAGAAATGGCTCTGGTTAATACCCGG
GGTCGATGACGGTACCGGAAGAATAAGCACCGGCTAACTACGTGCCAGCAGCCGCGGTA
ATACGTAGGGTGCGAGCGTTAATCGGAATTACTGGGCGTAAAGCGTGCGCAGGCGGTTT
TGTAAGACAGGCGTGAAATCCCCGAGCTCAACTTGGGAATGGCGCTTGTGACTGCAAGG
CTAGAGTATGTCAGAGGGGGAAGAATTCCACGTGTAGCAGTGAAATGCGTAGAGATGTG
GAGGAATACCGATGGCGAAGGCAGCCCCCTGGGACGTCACTGACGCTCATGCACGAAAG
CGTGGGGAGCAAACAGGATTAGATACCCTGGTAGTCCACGCCCTAAACGATGTCAACTA
GTTGTTGGGGATTCATTTCTTCAGTAACGTAGCTAACGCGTGAAGTTGACCGCCTGGGGA
GTACGGTCGCAAGATTAAAACTCAAAGGAATTGACGGGGACCCGCACAAGCGGTGGATG
ATGTGGATTAATTCGATGCAACGCGAAAAACCTTACCTACCCTTGACATGCCACTAACGA
AGCAGAGATGCATTAGGTGCCCGAAAGGGAAAGTGGACACAGGTGCTGCATGGCTGTCG
TCAGCTCGTGTCGTGAGATGTTGGGTTAAGTCCCGCAACGAGCGCAACCCTTGTCTCTAG
TTGCTACGAAAGGGCACTCTAGAGAGACTGCCGGTGACAAACCGGAGGAAGGTGGGGAT
GACGTCAAGTCCTCATGGCCCTTATGGGTAGGGCTTCACACGTCATACAATGGTGCGTAC
AGAGGGTTGCCAACCCGCGAGGGGGAGCTAATCCCAGAAAACGCATCGTAGTCCGGATC
GTAGTCTGCAACTCGACTACGTGAAGCTGGAATCGCTAGTAATCGCGGATCAGCATGCC
GCGGTGAATACGTTCCCGGGTCTTGTACACACCGCCCGTCACACCATGGGAGTGGGTTTT
GCCAGAAGTAGTTAGCCTAACCGCAAGGAGGGCGATTACCACGGCAGGGTTCATGACTG
GGGTGAAGTCGTAACAAGGTAGCCGTATCGGAAGGTGCGGCTGGATCACCTCCTTTC

In some embodiments of any of the aspects, the engineered bacterium comprises at least one engineered inactivating modification of at least one endogenous gene. In some embodiments of any of the aspects, an engineered inactivating modification of an endogenous gene comprises one or more of: i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion. Non-limiting examples of inactivating modifications include a mutation that decreases gene or polypeptide expression, a mutation that decreases gene or polypeptide transport, a mutation that decreases gene or polypeptide activity, a mutation in the active site of an enzyme that decreases enzymatic activity, or a mutation that decreases the stability of a nucleic acid or polypeptide. Examples of loss-of-function mutations for each gene can be clear to a person of ordinary skill (e.g., a premature stop codon, a frameshift mutation); they can be measurable by an assay of nucleic acid or protein function, activity, expression, transport, and/or stability; or they can be known in the art.

In some embodiments of any of the aspects, an inactivating modification of an endogenous gene can be engineered in a bacterium using an integration vector (e.g., pT18mobsacB). In some embodiments of any of the aspects, the engineering of an inactivating modification of an endogenous gene in a bacterium further comprises conjugation methods and/or counterselection methods (e.g., sucrose counterselection). In some embodiments of any of the aspects, the introduction of an integration vector comprising an endogenous gene comprising an inactivating modification causes the endogenous gene to be replaced with the endogenous gene comprising an inactivating modification.

In some embodiments of any of the aspects, the engineered bacterium comprises at least one overexpressed gene. In some embodiments of any of the aspects, the overexpressed gene is endogenous. In some embodiments of any of the aspects, the overexpressed gene is exogenous. In some embodiments of any of the aspects, the overexpressed gene is heterologous. In some embodiments of any of the aspects, a gene can be overexpressed using an expression vector (e.g., pBAD, pCR2.1).

In some embodiments of any of the aspects, the engineered bacterium comprises at least one exogenous copy of a functional gene. As a non-limiting example, the engineered bacterium can comprise 1, 2, 3, 4, or at least 5 exogenous copies of a functional gene. As used herein, the term “functional” refers to a form of a molecule which possesses either the native biological activity of the naturally existing molecule of its type, or any specific desired activity, for example as judged by its ability to bind to ligand molecules. In some embodiments of any of the aspects, a molecule can comprise at least 50%, at least 60%, at least 70%, at least 75%, at least 80%, at least 85%, at least 90%, at least 95%, at least 96%, at least 97%, at least 98%, at least 99%, or at least 99% of the activity of the wild-type molecule, e.g., in its native organism.

In some embodiments of any of the aspects, a functional gene as described herein is exogenous. In some embodiments of any of the aspects, a functional gene as described herein is ectopic. In some embodiments of any of the aspects, a functional gene as described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism, in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments of any of the aspects, the engineered bacterium comprises at least one functional heterologous gene. As used herein, the term “heterologous” refers to that which is not endogenous to, or naturally occurring in, a referenced sequence, molecule (including e.g., a protein), virus, cell, tissue, or organism. For example, a heterologous sequence of the present disclosure can be derived from a different species, or from the same species but substantially modified from an original form. Also for example, a nucleic acid sequence that is not normally expressed in a virus or a cell is a heterologous nucleic acid sequence. The term “heterologous” can refer to DNA, RNA, or protein that does not occur naturally as part of the organism in which it is present or which is found in a location or locations in the genome that differ from that in which it occurs in nature. It is DNA, RNA, or protein that is not endogenous to the virus or cell and has been artificially introduced into the virus or cell.

In some embodiments of any of the aspects, at least one exogenous copy of a functional gene can be engineered into a bacterium using an expression vector (e.g., pBadT). In some embodiments of any of the aspects, the expression vector (e.g., pBadT) is translocated from a donor bacterium (e.g., MFDpir) into the engineered bacterium under conditions that promote conjugation.

In some embodiments of any of the aspects, at least one exogenous or heterologous gene as described herein can comprise a detectable label, including but not limited to c-Myc, HA, VSV-G, HSV, FLAG, V5, HIS, or biotin. Detectable labels can also include, but are not limited to, radioisotopes, bioluminescent compounds, chromophores, antibodies, chemiluminescent compounds, fluorescent compounds, metal chelates, and enzymes.

In some embodiments of any of the aspects, the engineered bacterium further comprises a selectable marker. Non-limiting examples of selectable markers include a positive selection marker; a negative selection marker; a positive and negative selection marker; resistance to at least one of ampicillin, kanamycin, triclosan, and/or chloramphenicol; or an auxotrophy marker. In some embodiments of any of the aspects, the selectable marker is selected from the group consisting of beta-lactamase, Neo gene (e.g., Kanamycin resistance cassette) from Tn5, mutant FabI gene, and an auxotrophic mutation.

In one aspect, described herein is a combination of any two of the bacteria described herein. Examples of pairwise combinations are provided in Table 4, wherein “X” denotes the presence of the indicated bacterium. Two-way, three-way, four-way, or more complex combinations are specifically contemplated herein. In some embodiments of any of the aspects, a system as described herein can comprise any of the combinations in Table 4.

TABLE 4
Exemplary combinations of engineered bacteria

| | Engineered | | Engineered |
|---|---|---|---|
| Engineered | feedstock | | fertilizer |
| bioplastic | solution | Engineered | solution |
| bacterium | bacterium | heterotroph | bacterium |
| X | | | |
| | X | | |
| X | X | | |
| | | X | |
| X | | X | |
| | X | X | |
| X | X | X | |
| | | | X |
| X | | | X |
| | X | | X |
| X | X | | X |
| | | X | X |
| X | | X | X |
| | X | X | X |
| X | X | X | X |

Described herein are methods of sustainably producing a product (e.g., bioplastic, feedstock solution, fertilizer solution) comprising: (a) culturing an engineered bacterium as described herein in a culture medium comprising CO₂ and/or H₂; and (b) isolating, collecting, or concentrating the product from said engineered bacterium or from the culture medium of said engineered bacterium.

In some embodiments of any of the aspects, the cells can be maintained in culture. As used herein, “maintaining” refers to continuing the viability of a cell or population of cells. A maintained population of cells will have at least a subpopulation of metabolically active cells.

As used herein, the term “sustainable” refers to a method of harvesting or using a resource so that the resource is not depleted or permanently damaged. In some embodiments of any of the aspects, the resource is a product that is produced by an engineered bacterium as described herein. In some embodiments of any of the aspects, the engineered bacterium sustainably produces a product using a minimal culture medium that comprises CO₂ as the sole carbon source and H₂ as the sole energy source.

As used herein the term “culture medium” refers to a solid, liquid or semi-solid designed to support the growth of microorganisms or cells. In some embodiments of any of the aspects, the culture medium is a liquid. In some embodiments of any of the aspects, the culture medium comprises both the liquid medium and the bacterial cells within it.

In some embodiments of any of the aspects, the culture medium is a minimal medium. As used herein, the term “minimal medium” refers to a cell culture medium in which only few and necessary nutrients are supplied, such as a carbon source, a nitrogen source, salts and trace metals dissolved in water with a buffer. Non-limiting examples of components in a minimal medium include Na₂HPO₄ (e.g., 3.5 g/L), KH₂PO₄ (e.g., 1.5 g/L), (NH₄)₂SO₄ (e.g., 1.0 g/L), MgSO₄.7H₂O (e.g., 80 mg/L), CaSO₄.2H₂O (e.g., 1 mg/L), NiSO₄.7H₂O (e.g., 0.56 mg/L), ferric citrate (e.g., 0.4 mg/L), and NaHCO₃ (200 mg/L). In some embodiments of any of the aspects, a minimal medium can be used to promote lithotrophic growth, e.g., of a chemolithotroph.

In some embodiments of any of the aspects, the culture medium is a rich medium. As used herein, the term “rich medium” refers to a cell culture medium in which more than just a few and necessary nutrients are supplied, i.e., a non-minimal medium. In some embodiments of any of the aspects, rich culture medium can comprise nutrient broth (e.g., 17.5 g/L), yeast extract (7.5 g/L), and/or (NH₄)₂SO₄ (e.g., 5 g/L). In some embodiments of any of the aspects, a rich medium does necessarily promote lithotrophic growth.

In some embodiments of any of the aspects, the culture medium, culture vessel, or environment surrounding the culture medium or culture vessel (e.g., an incubator) comprises approximately 30% H₂ and approximately 15% CO₂. In some embodiments of any of the aspects, the culture medium, culture vessel, or environment surrounding the culture medium or culture vessel (e.g., an incubator) comprises at most 10% H₂, at most 20% H₂, at most 30% H₂, at most 40% H₂, or at most 50% H₂. In some embodiments of any of the aspects, the culture medium, culture vessel, or environment surrounding the culture medium or culture vessel (e.g., an incubator) comprises at most 5% CO₂, at most 10% CO₂, at most 15% CO₂ at most 20% CO₂, or at most 25% CO₂.

In some embodiments of any of the aspects, the culture medium comprises CO₂ as the sole carbon source. In some embodiments of any of the aspects, CO₂ is at least 90%, at least 95%, at least 98%, at least 99% or more of the carbon sources present in the culture medium. In some embodiments of any of the aspects, the culture medium comprises CO₂ in the form of bicarbonate (e.g., HCO₃⁻, NaHCO₃) and/or dissolved CO₂ (e.g., atmospheric CO₂; e.g., CO₂ provided by a cell culture incubator). In some embodiments of any of the aspects, the culture medium does not comprise organic carbon as a carbon source. Non-limiting example of organic carbon sources include fatty acids, gluconate, acetate, fructose, decanoate; see e.g., Jiang et al. Int J Mol Sci. 2016 July; 17(7): 1157).

In some embodiments of any of the aspects, the culture medium comprises H₂ as the sole energy source. In some embodiments of any of the aspects, H₂ is at least 90%, at least 95%, at least 98%, at least 99% or more of the energy sources present in the culture medium. In some embodiments of any of the aspects, H₂ is supplied by water-splitting electrodes in the culture medium. Accordingly, in one aspect described herein is a system comprising a reactor chamber with a solution (e.g., culture medium) contained therein. The solution may include hydrogen (H₂), carbon dioxide (CO₂), bioavailable nitrogen (e.g., ammonia, (NH₄)₂SO₄, amino acids), and an engineered bacterium as described herein. Gasses such as one or more of hydrogen (H₂), carbon dioxide (CO₂), nitrogen (N₂), and oxygen (O₂) may also be located within a headspace of the reactor chamber, though embodiments in which a reactor does not include a headspace such as in a flow through reactor are also contemplated. The system may also include a pair of electrodes immersed in the solution (e.g., culture medium). The electrodes are configured to apply a voltage potential to, and pass a current through, the solution to split water contained within the culture medium to form at least hydrogen (H₂) and oxygen (O₂) gasses in the solution. These gases may then become dissolved in the solution. During use, a concentration of the bioavailable nitrogen in the solution may be maintained below a threshold nitrogen concentration that causes the bacteria to produce a desired product (e.g., PHA). This product may either by excreted from the bacteria and/or stored within the bacteria as the disclosure is not so limited (see e.g., US Patent Publication 2018/0265898, the contents of which are incorporated herein by reference in their entirety).

In some embodiments of any of the aspects, the culture medium does not comprise oxygen (O₂) gasses in the solution, i.e., the culture is grown under anaerobic conditions. In some embodiments of any of the aspects, the culture medium comprises low levels of oxygen (O₂) gasses in the solution, i.e., the culture is grown under hypoxic conditions. As a non-limiting example, the culture medium can comprise at most 30%, at most 20%, at most 15%, at most 10%, at most 5%, at most 4%, at most 3%, at most 2%, or at most 1% O₂ gasses in the solution.

In some embodiments of any of the aspects, methods described herein comprise isolating, collecting, or concentrating a product from an engineered bacterium or from the culture medium of an engineered bacterium. As used herein the terms “isolate,” “collect,” “concentrate”, “purify” and “extract” are used interchangeably and refer to a process whereby a target component (e.g., PHA, MCL-PHA) is removed from a source, such as a fluid (e.g., culture medium). In some embodiments of any of the aspects, methods of isolation, collection, concentration, purification, and/or extraction comprise a reduction in the amount of at least one heterogeneous element (e.g., proteins, nucleic acids; i.e., a contaminant). In some embodiments of any of the aspects, methods of isolation, collection, concentration, purification, and/or extraction reduce by 1%, 2%, 3%, 4%, 5%, 10%, 15%, 20%, 25%, 30%, 35%, 40%, 45%, 50%, 55%, 60%, 65%, 70%, 75%, 80%, 85%, 90% or 95%, or more, the amount of heterogeneous elements, for example biological macromolecules such as proteins or DNA, that may be present in a sample comprising a molecule of interest. The presence of heterogeneous proteins can be assayed by any appropriate method including High-performance Liquid Chromatography (HPLC), gel electrophoresis and staining and/or ELISA assay. The presence of DNA and other nucleic acids can be assayed by any appropriate method including gel electrophoresis and staining and/or assays employing polymerase chain reaction.

Described herein are systems comprising at least one of the engineered bacteria as described herein. In one aspect, the system comprises at least one of the engineered bacteria and a support. In some embodiments of any of the aspects, the bacteria is linked to the support using intrinsic mechanisms (e.g., pili, biofilm, etc.) and/or extrinsic mechanisms (e.g., chemical crosslinking, antibiotics, opsonin, etc.). In some embodiments of any of the aspects, the system further comprises a container and a solution, in which the bacteria linked to the support are submerged. In some embodiments of any of the aspects, the system further comprises a pair of electrodes that split water contained within the solution to form hydrogen. In some embodiments of any of the aspects, the solution (e.g., a culture medium) comprises hydrogen (H₂) and carbon dioxide (CO₂).

In some embodiments of any of the aspects, the support comprises a solid substrate. Examples of solid substrate can include, but are not limited to, film, beads or particles (including nanoparticles, microparticles, polymer microbeads, magnetic microbeads, and the like), filters, fibers, screens, mesh, tubes, hollow fibers, scaffolds, plates, channels, gold particles, magnetic materials, medical apparatuses (e.g., needles or catheters) or implants, dipsticks or test strips, filtration devices or membranes, hollow fiber cartridges, microfluidic devices, mixing elements (e.g., spiral mixers), extracorporeal devices, and other substrates commonly utilized in assay formats, and any combinations thereof. In some embodiments of any of the aspects, the solid substrate can be a magnetic particle or bead.

In several aspects, the system comprises a reactor chamber and at least one of the engineered bacteria as described herein. Accordingly, in one aspect, described herein is a system comprising: (a) a reactor chamber with a solution contained therein, wherein the solution comprises hydrogen (H₂) and carbon dioxide (CO₂); and (b) at least one engineered bacterium as described herein in the solution. In some embodiments of any of the aspects, the system further comprises a pair of electrodes in contact with the solution that split water to form the hydrogen. In one aspect, described herein is a system comprising: (a) a reactor chamber; and (b) at least one engineered bacterium. In some embodiments of any of the aspects, the system further comprises a pair of electrodes in contact with reactor chamber.

In one aspect, described herein is a system comprising: (a) a reactor chamber with a solution contained therein, wherein the solution comprises hydrogen (H₂) and carbon dioxide (CO₂); (b) at least one of the following engineered bacteria in the solution: (i) an engineered bioplastics bacterium as described herein; (ii) an engineered sugar feedstock bacterium as described herein; (iii) an engineered heterotroph as described herein; or (iv) an engineered fertilizer solution bacterium as described herein. In some embodiments of any of the aspects, the system further comprises a pair of electrodes in contact with the solution that split water to form the hydrogen.

In some embodiments of any of the aspects, the system (e.g., a system comprising a reactor chamber, a system comprising a support) can comprise any combination of engineered bacteria as described herein. In some embodiments of any of the aspects, the system comprises (i) an engineered bioplastics bacterium as described herein. In some embodiments of any of the aspects, the system comprises (ii) an engineered sugar feedstock bacterium as described herein. In some embodiments of any of the aspects, the system comprises (iii) an engineered heterotroph as described herein. In some embodiments of any of the aspects, the system comprises (iv) an engineered fertilizer solution bacterium as described herein.

In some embodiments of any of the aspects, the system comprises (i) an engineered bioplastics bacterium as described herein; and (ii) an engineered sugar feedstock bacterium as described herein. In some embodiments of any of the aspects, the system comprises (i) an engineered bioplastics bacterium as described herein; and (iii) an engineered heterotroph as described herein. In some embodiments of any of the aspects, the system comprises (i) an engineered bioplastics bacterium as described herein; and (iv) an engineered fertilizer solution bacterium as described herein. In some embodiments of any of the aspects, the system comprises (ii) an engineered sugar feedstock bacterium as described herein; and (iii) an engineered heterotroph as described herein. In some embodiments of any of the aspects, the system comprises (ii) an engineered sugar feedstock bacterium as described herein; and (iv) an engineered fertilizer solution bacterium as described herein. In some embodiments of any of the aspects, the system comprises (iii) an engineered heterotroph as described herein; and (iv) an engineered fertilizer solution bacterium as described herein.

In some embodiments of any of the aspects, the system comprises (i) an engineered bioplastics bacterium as described herein; (ii) an engineered sugar feedstock bacterium as described herein; and (iii) an engineered heterotroph as described herein. In some embodiments of any of the aspects, the system comprises (i) an engineered bioplastics bacterium as described herein; (ii) an engineered sugar feedstock bacterium as described herein; and (iv) an engineered fertilizer solution bacterium as described herein. In some embodiments of any of the aspects, the system comprises (i) an engineered bioplastics bacterium as described herein; (iii) an engineered heterotroph as described herein; and (iv) an engineered fertilizer solution bacterium as described herein. In some embodiments of any of the aspects, the system comprises (ii) an engineered sugar feedstock bacterium as described herein; (iii) an engineered heterotroph as described herein; and (iv) an engineered fertilizer solution bacterium as described herein. In some embodiments of any of the aspects, the system comprises (i) an engineered bioplastics bacterium as described herein; (ii) an engineered sugar feedstock bacterium as described herein; (iii) an engineered heterotroph as described herein; and (iv) an engineered fertilizer solution bacterium as described herein.

In one aspect, described herein is a system comprising: (a) a reactor chamber with a solution contained therein, wherein the solution comprises hydrogen (H₂) and carbon dioxide (CO₂); (b) an engineered bioplastics bacterium as described herein in the solution; and (c) a pair of electrodes in contact with the solution that split water to form the hydrogen.

In one aspect, described herein is a system comprising: (a) a reactor chamber with a solution contained therein, wherein the solution comprises hydrogen (H₂) and carbon dioxide (CO₂); (b) an engineered sugar feedstock bacterium as described herein in the solution; and (c) a pair of electrodes in contact with the solution that split water to form the hydrogen.

In one aspect, described herein is a system comprising: (a) a reactor chamber with a solution contained therein, wherein the solution comprises hydrogen (H₂) and carbon dioxide (CO₂); (b) an engineered sugar feedstock bacterium as described herein in the solution; (c) an engineered heterotroph as described herein in the solution; and (d) a pair of electrodes in contact with the solution that split water to form the hydrogen.

In one aspect, described herein is a system comprising: (a) a reactor chamber with a solution contained therein, wherein the solution comprises hydrogen (H₂) and carbon dioxide (CO₂); (b) an engineered fertilizer solution bacterium as described herein in the solution; and (c) a pair of electrodes in contact with the solution that split water to form the hydrogen.

In some embodiments of any of the aspects, the pair of electrodes comprise a cathode including a cobalt-phosphorus alloy and an anode including cobalt phosphate. In some embodiments of any of the aspects, a concentration of the bioavailable nitrogen in the solution is below a threshold nitrogen concentration to cause the engineered bacteria to produce a product. In some embodiments of any of the aspects, the solution is also referred to as a culture medium and can comprise a minimal medium as described further herein.

In one embodiment, a system includes a reactor chamber containing a solution. The solution may include hydrogen (H₂), carbon dioxide (CO₂), bioavailable nitrogen, and an engineered bacteria. Gasses such as one or more of hydrogen (H₂), carbon dioxide (CO₂), nitrogen (N₂), and oxygen (O₂) may also be located within a headspace of the reactor chamber, though embodiments in which a reactor does not include a headspace such as in a flow through reactor are also contemplated. The system may also include a pair of electrodes immersed in the solution. The electrodes are configured to apply a voltage potential to, and pass a current through, the solution to split water contained within the solution to form at least hydrogen (H₂) and oxygen (O₂) gasses in the solution. These gases may then become dissolved in the solution. During use, a concentration of the bioavailable nitrogen in the solution may be maintained below a threshold nitrogen concentration that causes the bacteria to produce a desired product. This product may either by excreted from the bacteria and/or stored within the bacteria as the disclosure is not so limited.

Concentrations of the above noted gases both dissolved within a solution, and/or within a headspace above the solution, may be controlled in any number of ways including bubbling gases through the solution, generating the dissolved gases within the solution as noted above (e.g. electrolysis/water splitting), periodically refreshing a composition of gases located within a headspace above the solution, or any other appropriate method of controlling the concentration of dissolved gas within the solution. Additionally, the various methods of controlling concentration may either be operated in a steady-state mode with constant operating parameters, and/or a concentration of one or more of the dissolved gases may be monitored to enable a feedback process to actively change the concentrations, generation rates, or other appropriate parameter to change the concentration of dissolved gases to be within the desired ranges noted herein. Monitoring of the gas concentrations may be done in any appropriate manner including pH monitoring, dissolved oxygen meters, gas chromatography, or any other appropriate method.

As noted above, in one embodiment, the composition of a volume of gas located in a headspace of a reactor may include one or more of carbon dioxide, oxygen, hydrogen, and nitrogen. A concentration of the carbon dioxide may be between 10 volume percent (vol %) and 100 vol %. However, carbon dioxide may also be greater than equal to 0.04 vol % and/or any other appropriate concentration. For example, carbon dioxide may be between or equal to 0.04 vol % and 100 vol %. A concentration of the oxygen may be between 1 vol % and 99 vol % and/or any other appropriate concentration. A concentration of the hydrogen may be greater than or equal to 0.05 vol % and 99%. A concentration of the nitrogen may be between 0 vol % and 99 vol %.

As also noted, in one embodiment, a solution within a reactor chamber may include water as well as one or more of carbon dioxide, oxygen, and hydrogen dissolved within the water. A concentration of the carbon dioxide in the solution may be between 0.04 vol % to saturation within the solution. A concentration of the oxygen in the solution may be between 1 vol % to saturation within the solution. A concentration of the hydrogen in the solution may be between 0.05 vol % to saturation within the solution provided that appropriate concentrations of carbon dioxide and/or oxygen are also present.

As noted previously, and as described further below, production of a desired end product by bacteria located within the solution may be controlled by limiting a concentration of bioavailable nitrogen, such as in the form of ammonia, amino acids, or any other appropriate source of nitrogen useable by the bacteria within the solution to below a threshold nitrogen concentration. However, and without wishing to be bound by theory, the concentration threshold may be different for different bacteria and/or for different concentrations of bacteria. For example, a solution containing enough ammonia to support a Ralstonia eutropha (i.e., Cupriavidus necator) population up to an optical density (OD) of 2.3 produces product at molar concentrations less than or equal to 0.03 M while a population with an OD of 0.7 produces product at molar concentrations less than or equal to 0.9 mM. Accordingly, higher optical densities may be correlated with producing product at higher nitrogen concentrations while lower optical densities may be correlated with producing product at lower nitrogen concentrations. Further, bacteria may be used to produce product by simply placing them in solutions containing no nitrogen. In view of the above, an optical density of bacteria within a solution may be between or equal to 0.1 and 12, 0.7 and 12, or any other appropriate concentration including concentrations both larger and smaller than those noted above. Additionally, a concentration of nitrogen within the solution may be between or equal to 0 and 0.2 molar, 0.0001 and 0.1 molar, 0.0001 and 0.05 molar, 0.0001 and 0.03 molar, or any other appropriate composition including compositions greater and less than the ranges noted above.

While particular gasses and compositions have been detailed above, it should be understood that the gasses located with a headspace of a reactor as well as a solution within the reactor may include compositions and/or concentrations as the disclosure is not limited in this fashion.

Bacteria used in the systems and methods disclosed herein may be selected so that the bacteria both oxidize hydrogen as well as consume carbon dioxide. Accordingly, in some embodiments, the bacteria may include an enzyme capable of metabolizing hydrogen as an energy source such as with hydrogenase enzymes. Additionally, the bacteria may include one or more enzymes capable of performing carbon fixation such as Ribulose-1,5-bisphosphate carboxylase/oxygenase (RuBisCO). One possible class of bacteria that may be used in the systems and methods described herein to produce a product include, but are not limited to, chemolithoautotrophs. Additionally, appropriate chemolithoautotrophs may include any one or more of Ralstonia eutropha (R. eutropha) as well as Alcaligenes paradoxs I 360 bacteria, Alcaligenes paradoxs 12/X bacteria, Nocardia opaca bacteria, Nocardia autotrophica bacteria, Paracoccus denitrificans bacteria, Pseudomonas facilis bacteria, Arthrobacter species 11X bacteria, Xanthobacter autotrophicus bacteria, Azospirillum lipferum bacteria, Derxia Gummosa bacteria, Rhizobium japonicum bacteria, Microcyclus aquaticus bacteria, Microcyclus ebruneus bacteria, Renobacter vacuolatum bacteria, and any other appropriate bacteria.

Depending on the particular product that it is desired to make, a bacteria may either naturally include a production pathway, or may be appropriately engineered, to include a production pathway to produce any number of different products when placed under the appropriate growth conditions. Appropriate products include, but are not limited to: sugar (e.g., sucrose) feedstock solutions, fertilizer solutions (e.g., lipochitooligosaccharides) short, medium, and long chain alcohols including for example one or more of isopropanol (C3 alcohol), isobutanol (C4 alcohol), 3-methyl-1-butanol (C5 alcohol), or any other appropriate alcohol; short, medium, and long chain fatty acids; short, medium, and long chain alkanes; polymers such as polyhydroxyalkanoates (PHA) including medium-chain length PHA and poly(3-hydroxybutyrate) (PHB); amino acids, and/or any other appropriate product as the disclosure is not so limited.

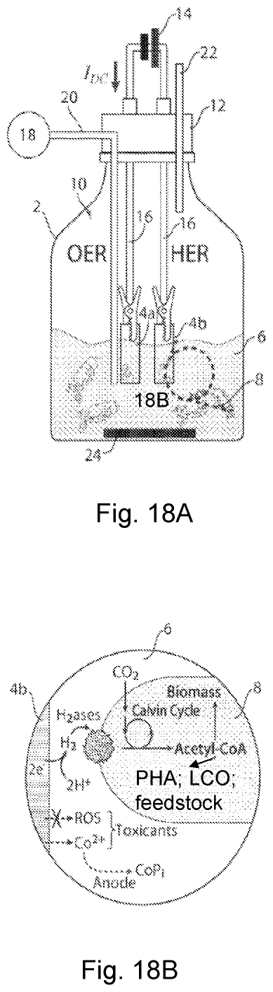

FIG. 18A shows a schematic of one embodiment of a system including one or more reactor chambers. In the depicted embodiment, a single-chamber reactor 2 houses one or more pairs of electrodes including an anode 4a and a cathode 4b immersed in a water based solution 6. Bacteria 8 are also included in the solution. A headspace 10 corresponding to a volume of gas that is isolated from an exterior environment is located above the solution within the reactor chamber. The gas volume may correspond to any appropriate composition including, but not limited to, carbon dioxide, nitrogen, hydrogen, oxygen, and any other appropriate gases as the disclosure is not so limited. Additionally, as detailed further below, the various gases may be present in any appropriate concentration as detailed previously. However, it should be understood that embodiments in which a reactor chamber is exposed to an external atmosphere that may either be a controlled composition and/or a normal atmosphere are also contemplated. The system may also include one or more temperature regulation devices such as a water bath, temperature controlled ovens, or other appropriate configurations and/or devices to maintain a reactor chamber at any desirable temperature range for bacterial growth.

In embodiments where a reactor chamber interior is isolated from an exterior environment, the system may include one or more seals 12. In the depicted embodiment, the seal corresponds to a cork, stopper, a threaded cap, a latched lid, or any other appropriate structure that seals an outlet from an interior of the reactor chamber. In this particular embodiment, a power source 14 is electrically connected to the anode and cathode via two or more electrical leads 16 that pass through one or more pass throughs in the seal to apply a potential to and pass a current IDC to split water within the solution into hydrogen and oxygen through an oxygen evolution reaction (OER) at the anode and a hydrogen evolution reaction (HER) at the cathode. While the leads have been depicted as passing through the seal, it should be understood that embodiments in which the leads pass through a different portion of the system, such as a wall of the reactor chamber, are also contemplated as the disclosure is so limited.

Depending on the particular embodiment, the above-described power source may correspond to any appropriate source of electrical current that is applied to the electrodes. However, in at least one embodiment, the power source may correspond to a renewable source of energy such as a solar cell, wind turbine, or any other appropriate source of current though embodiments in which a non-renewable energy source, such as a generator, battery, grid power, or other power source is used are also contemplated. In either case, a current from the power source is passed through the electrodes and solution to evolve hydrogen and oxygen. The current may be controlled to produce hydrogen and/or oxygen at a desired rate of production as noted above. In some embodiments of any of the aspects, a system comprising a renewable source of energy (e.g., a solar cell) can also be referred to as a “bionic leaf”.

Accordingly, in one aspect, described herein is a system comprising: (a) a reactor chamber with a solution contained therein, wherein the solution comprises hydrogen (H₂) and carbon dioxide (CO₂); (b) at least one of the following engineered bacteria in the solution: (i) an engineered bioplastics bacterium as described herein; (ii) an engineered sugar feedstock bacterium as described herein; (iii) an engineered heterotroph as described herein; or (iv) an engineered fertilizer solution bacterium as described herein; (c) a pair of electrodes in contact with the solution that split water to form the hydrogen; and (d) comprising a power source comprising a renewable source of energy.

In some embodiments, the electrodes may be coated with, or formed from, a water splitting catalyst to further facilitate water splitting and/or reduce the voltage applied to the solution. In some embodiments, the catalysts may be coated onto an electrode substrate including, for example, carbon fabrics, porous carbon foams, porous metal foams, metal fabrics, solid electrodes, and/or any other appropriate geometry or material as the disclosure is not so limited. In another embodiment, the electrodes may simply be made from a desired catalyst material. Several appropriate materials for use as catalysts include, but are not limited to, one or more of a cobalt-phosphorus (Co—P) alloy, cobalt phosphate (CoPi), cobalt oxide, cobalt hydroxide, cobalt oxyhydroxide, a NiMoZn alloy, or any other appropriate material. As noted further below, certain catalysts offer additional benefits as well. For example, in one specific embodiment, the electrodes may correspond to a cathode including a cobalt-phosphorus alloy and an anode including cobalt phosphate, which may help to reduce the presence of reactive oxygen species and/or metal ions within a solution. A composition of the CoPi coating and/or electrode may include phosphorous compositions between or equal to 0 weight percent (wt %) and 50 wt %. Additionally, the Co—P alloy may include between 80 wt % and 99 wt % Co as well as 1 wt % and 20 wt % P. However, embodiments in which different element concentrations are used and/or other types of catalysts and/or electrodes are used are also contemplated as the disclosure is not so limited. For example, stainless steel, platinum, and/or other types of electrodes may be used.

As also shown in FIG. 18A, in some embodiments, it may be desirable to either continuously, or periodically, bubble, i.e. sparge or flush, one or more gases through a solution 6 and/or to refresh a composition of gases located within a head space 10 of the reactor chamber 2 above a surface of the solution. In such an embodiment, a gas source 18 may be in fluid communication with one or more gas inlets 20 that pass through either a seal 12 and/or another portion of the reactor chamber 2 such as a side wall to place the gas source in fluid communication with an interior of the reactor chamber. Additionally, in some embodiments, one or more inlets discharge a flow of gas into the solution so that the gas will bubble through the solution. However, embodiments in which the one or more gas inlets discharge a flow of gas into the headspace of the reactor chamber instead are also contemplated as the disclosure is not so limited. Additionally, one or more corresponding gas outlets 22 may be formed in a seal and/or another portion of the reactor chamber to permit a flow of gas to flow from an interior to an exterior of the reactor chamber. It should be noted that gas inlets and outlets may correspond to any appropriate structure including, but not limited to, tubes, pipes, flow passages, ports in direct fluid communication with the reactor chamber interior, or any other appropriate structure.

Gas sources may correspond to any appropriate gas source capable of providing a pressurized flow of gas to the chamber through the inlet including, for example, one or more pressurized gas cylinders. While a gas source may include any appropriate composition of one or more gasses, in one embodiment, a gas source may provide one or more of hydrogen, nitrogen, carbon dioxide, and oxygen. The flow of gas provided by the gas source may have a composition equivalent to the range of gas compositions described above for the gas composition with a headspace of the reactor chamber. Further, in some embodiments, the gas source may simply be a source of carbon dioxide. Of course embodiments in which a different mix of gases, other including different gases and/or different concentrations than those noted above, is bubbled through a solution or otherwise input into a reactor chamber are also contemplated as the disclosure is not so limited. Additionally, the gas source may be used to help maintain operation of a reactor at, below, and/or above atmospheric pressure as the disclosure is not limited to any particular pressure range.

The above noted one or more gas inlets and outlets may also include one or more valves located along a flow path between the gas source and an exterior end of the one or more outlets. These valves may include for example, manually operated valves, pneumatically or hydraulically actuated valves, unidirectional valves (i.e. check valves) may also be incorporated in the one or more inlets and/or outlets to selectively prevent the flow of gases into or out of the reactor either entirely or in the upstream direction into the chamber and/or towards the gas source.

While the use of inlet and/or outlet gas passages have been described above, embodiments in which there are no inlet and/or outlets for gasses are present are also contemplated. For example, in one embodiment, a system including a sealable reactor may simply be flushed with appropriate gasses prior to being sealed. The system may then be flushed with an appropriate composition of gasses at periodic intervals to refresh the desired gas composition in the solution and/or headspace prior to resealing the reactor chamber. Alternatively, the head space may be sized to contain a gas volume sufficient for use during an entire production run.

In instances where electrodes are run at high enough rates and/or for sufficient durations, concentration may be formed within a solution in a reactor chamber. Accordingly, it may be desirable to either prevent and/or mitigate the presence of concentration gradients in the solution. Therefore, in some embodiments, a system may include a mixer such as a stir bar 24 illustrated in FIG. 18A. Alternatively, a shaker table, and/or any other way of inducing motion in the solution to reduce the presence of concentration gradients may also be used as the disclosure is not so limited.

While the above embodiment has been directed to an isolated reactor chamber, embodiments in which a flow-through reaction chamber with two or more corresponding electrodes immersed in a solution that is flowed through the reaction chamber and past the electrodes are also contemplated. For example, one possible embodiment, one or more corresponding electrodes may be suspended within a solution flowing through a chamber, tube, passage, or other structure. Similar to the above embodiment, the electrodes are electrically coupled with a corresponding power source to perform water splitting as the solution flows past the electrodes. Such a system may either be a single pass flow through system and/or the solution may be continuously flowed passed the electrodes in a continuous loop though other configurations are also contemplated as well.

Without wishing to be bound by theory, FIG. 18B illustrates one possible pathway for a system to produce one or more desired products. In the depicted embodiment, the hydrogen evolution reaction occurs at the cathode 4b. During the reaction at the cathode, two hydrogen ions (H^{f}) are combined with two electrons to form hydrogen gas H₂ that dissolves within the solution 6 along with carbon dioxide (CO₂), which dissolved in the solution as well. At the same time various toxicants such as reactive oxygen species (ROS) including, for example, hydrogen peroxide (H₂O₂), superoxides (O₂⁻), and/or hydroxyl radical (HO.) species as well as metallic ions may be generated at the cathode. For example, Co²⁺ ions may be dissolved into solution when a cobalt based cathode is used. As described further below, in some embodiments, the use of certain catalysts may help to reduce the production of ROS and the metallic ions leached into the solution may be deposited onto the anode using one or more elements located within the solution to form compounds such as a cobalt phosphate.

As also illustrated in FIG. 18B, once hydrogen and carbon dioxide are provided within a solution, bacteria 8 present within the solution may be used to transform these compounds into useful products. For example, in one embodiment, the bacteria uses hydrogenase to metabolize the dissolved hydrogen gas and one or more appropriate enzymes, such as RuBisCO or other appropriate enzyme, to provide a carbon fixation pathway. This may include absorbing the carbon dioxide and forming Acetyl-CoA through the Calvin cycle as shown in the figure. Further, depending on the concentration of nitrogen within the solution, the bacteria may either form biomass or one or more desired products. For instance, if a concentration of nitrogen within the solution is below a predetermined nitrogen concentration threshold, the bacteria may form one or more products such as bioplastics (e.g., PHA), fertilizer (e.g., LCO) solution, feedstock (e.g., sucrose) solution, the C3, C4, and/or C5 alcohols, PHB, and/or combinations of the above depicted in the figure.

Depending on the embodiment, a solution placed in the chamber of a reactor may include water with one or more additional solvents, compounds, and/or additives. For example, the solution may include: inorganic salts such as phosphates including sodium phosphates and potassium phosphates; trace metal supplements such as iron, nickel, manganese, zinc, copper, and molybdenum; or any other appropriate component in addition to the dissolved gasses noted above. In one such embodiment, a phosphate may have a concentration between 9 and 90 mM, 9 and 72 mM, 9 and 50 mM, or any other appropriate concentration. In a particular embodiment, a water based solution may include one or more of the following in the listed concentrations: 12 mM to 123 mM of Na₂HPO₄, 11 mM to 33 mM of KH₂PO₄, 1.25 mM to 15 mM of (NH₄)₂SO₄, 0.16 mM to 0.64 mM of MgSO₄, 2.4 μM to 5.8 μM of CaSO₄, 1 μM to 4 μM of NiSO₄, 0.81 μM to 3.25 μM molar concentration of Ferric Citrate, 60 mM to 240 mM molar concentration of NaHCO₃.

As noted above in regards to the discussion of FIG. 18B, reactive oxygen species (ROS) as well as metallic ions may be formed and/or dissolved into a solution during the hydrogen evolution reaction at the cathode. However, ROS and larger concentrations of the metallic ions within the solution may be detrimental to cell growth above certain concentrations. It is noted that the use of continuous hydrogen production within a reactor to form hydrogen for conversion into one or more desired products has been hampered by the production of these ROS and metallic ion concentrations because the bacteria used to form the desired products tend to be sensitive to these compounds and ions limiting the growth of, and above certain concentrations, killing the bacteria. Therefore, in some embodiments, it may be desirable to apply voltages, use electrodes that produce less ROS, remove and/or prevent the dissolution of metallic ions from the electrodes, and/or use bacteria that are resistant to the presence of these toxicants as detailed further below.

As noted above, it may be desirable to select one or more catalysts for use as the electrodes that produce fewer reactive oxygen species (ROS) during use. Specifically, a biocompatible catalyst system that is not toxic to the bacterium and lowers the overpotential for water splitting may be used in some embodiments. One such example of a catalyst includes a ROS-resistant cobalt-phosphorus (Co—P) alloy cathode. This cathode may be combined with a cobalt phosphate (CoPi) anode. This catalyst pair has the added benefit of the anode being self-healing. In other words, the catalyst pair helps to remove metallic Co²⁺ ions present with a solution in a reactor. Without wishing to be bound by theory, the electrode pair works in concert to remove extracted metal ions from the cathode by depositing them onto the anode which may help to maintain extraneous cobalt ions at relatively low concentrations within solution and to deliver a low applied electrical potential to split water to generate H₂. Without wishing to be bound by theory, it is believed that during electrolysis of the water, phosphorus and/or cobalt is extracted from the electrodes. The reduction potential of leached cobalt is such that formation of cobalt phosphate using phosphate available in the solution is energetically favored. Cobalt phosphate formed in solution then deposits onto the anode at a rate linearly proportional to free Co′, providing a self-healing process for the electrodes. In view of the above, the cobalt-phosphorus (Co—P) alloy and cobalt phosphate (CoPi) catalysts may be used to help mitigate the presence of both ROS and metal ions within the solution to help promote growth of bacteria within the reactor chamber.

It should be understood that any appropriate voltage may be applied to a pair of electrodes immersed in a solution to split water into hydrogen and oxygen. However, in some embodiments, the applied voltage may be limited to fall between upper and lower voltage thresholds. For example, the self-healing properties of a cobalt phosphate and cobalt phosphorous based alloy electrode pair may function at voltage potentials greater than about 1.42 V. Additionally, the thermodynamic minimum potential for splitting water is about 1.23 V. Therefore, depending on the particular embodiment, the voltage applied to the electrodes may be greater than or equal to about 1.23 V, 1.42 V, 1.5 V, 2 V, 2.2 V, 2.4 V, or any other appropriate voltage. Additionally, the applied voltage may be less than or equal to about 10 V, 5 V, 4 V, 3 V, 2.9 V, 2.8 V, 2.7 V, 2.6 V, 2.5 V, or any other appropriate voltage. Combinations of the above noted voltage ranges are contemplated including, for example, a voltage applied to a pair of electrodes may be between 1.23 V and 10 V, 1.42 V and 5 V, 2 V and 3 V, 2.3 V and 2.7 V as well as other appropriate ranges. Additionally, it should be understood that voltages both greater than and less than those noted above, as well as different combinations of the above ranges, are also contemplated as the disclosure is not so limited. In addition to the applied voltages, any appropriate current may be passed through the electrodes to perform water splitting which will depend on the desired rate of hydrogen generation for a given volume of a reactor being used. For example, in some embodiments, a current used to split water may be controlled to generate hydrogen at a rate substantially equal to a rate of hydrogen consumption by bacteria in the solution. However, embodiments in which hydrogen is produced at rates both greater than or less than consumption by the bacteria are also contemplated.

In addition to using catalysts, controlling the solution pH, and applying appropriate driving potentials, and/or controlling any other appropriate parameter to reduce the presence of reactive oxygen species (ROS) within the solution in a reaction chamber, it may also be desirable to use bacteria that are resistant to the presence of ROS and/or metallic ions present within the solution as noted previously. Specifically, a chemolithoautotrophic bacterium that is resistant to reactive oxygen species may be used. Further, in some embodiments a R. eutropha bacteria that is resistant to ROS as compared to a wild-type H16 R. eutropha may be used. US 2018/0265898 and Table 3 below detail several genetic polymorphisms found between the wild-type H16 R. eutropha and a ROS-tolerant BC4 strain that was purposefully evolved. Mutations of the BC4 strain relative to the wild type bacteria are detailed further below.

TABLE 3
Mutations in ROS-tolerant BC4 strain

| Mutatior | Position | Annotation | Gene | Description |
|---|---|---|---|---|
| G → T | 611,894 | R133R | acrC1 | cation/multidrug efflux |
| | | | | system outer |
| | | | | membrane protein |
| Δ45 bp | 611,905 | 344-388 | acrC1 | cation/multidrug efflux |
| | | of 1494 nt | | system outer |
| | | | | membrane protein |
| G → A | 2,563,281 | intergenic, | Hfq and | uncharacterized host |
| | | (−1/+210) | H16_A2360 | factor I protein/ |
| | | | | GTP-binding protein |
| Δ15 bp | 241,880 | 363-377 | H16_B0214 | transcriptional regulator, |
| | | of 957 nt | | LysR-Family |

Two single nucleotide polymorphisms and two deletion events have been observed. Without wishing to be bound by theory, the large deletion from acrC1 may indicate a decrease in overall membrane permeability, possibly affecting superoxide entry to the cell resulting in the observed ROS resistance. The genome sequences are accessible at the NCBI SRA database under the accession number SRP073266 and specific mutations of the BC4 strain are listed below in Table 1. The standard genome sequence for the wild-type H16 R. eutropha is also accessible at the RCSB Protein Data Bank under accession number AM260479 which the following mutations may also be referenced to.

In reference to the above table, an R. eutropha bacteria may include at least one to four mutations selected from the mutations noted above in Table 3 and may be selected in any combination. These specific mutations are listed below in more detail with mutations noted relative to the wild type R. eutropha bolded and underlined within the sequences given below.

The first noted mutation may correspond to the sequence listed below ranging from position 611790-611998 for Ralstonia eutropha H16 chromosome 1. The bolded, doubleunderlined text indicates a mutation (e.g., nt 105 of SEQ ID NO: 69).

(209 nt)
SEQ ID NO: 69
GCCTCGCTGCTTTCCACCTGGCGCCGCACGCGGCCCCAGACGTCGA
TTTCCCAGGTTGCGCCCAGGGTCGCGCTCTGCCCGTTGAGCGTGCT
GCCG CTGGCGCC [symbol] CGCGCGCGCGAGGCGCCGGCCTGTGCGTCGA
CGGTCGGGAAGAAGCCGGCGCGCGCGGCCTGCAGCGACGCCACCGC
CTGGCGGTACTGCGCCTCGGCGGCCTT

The second noted mutation may correspond to the sequence listed below ranging from position 611905-613399 for Ralstonia eutropha H16 chromosome 1. The bolded, doubleunderlined text indicates a mutation (e.g., nt 345-390 of SEQ ID NO: 70).

(1495 nt)
SEQ ID NO: 70
AGGCGCCGGCCTGTGCGTCGACGGTCGGGAAGAAGCCGGCGCGCG
CGGCCTGCAGCGACGCCACCGCCTGGCGGTACTGCGCCTCGGCGGCCTTG
ATGTTCTGGTTCGAGATCTGCACCTCGGACATCAGCGCGTCGAGCTGCGC
ATCGCCGAACACGGTCCACCAGTCGGCGCGTGCCAGCGCATCCTGCGGCT
CGGCGGGCTTCCAGTCGCCGGTCCAGGCGGGGGTGGCGGCATCGGCTTCC
TTGAAGGATGCGGAAACCGGCGCGTCGGGGCGCTGGTAGTCGGGGCCGAC
GGCGCAGCCGGCCAGCAGCAGCGCGCAGGCCAGCGACACCGGCAGGGCA [symbol] [symbol] [symbol]
CTAGA
CGGCGGCCGGCTGGTCAGGCGTGCCGGCACCACGGCGGCGCTGGCGCCAG
GCCTTGACCTTCAGGCGCCAGCGGTCCAGCGTCAGGTAGACCACCGGCGT
GGTGTACAGCGTCAGCAGCTGGCTTACCACCAGTCCGCCGACAATGGAGA
TGCCCAGCGGCGCGCGCAGTTCGGCGCCGTCGCCGCGGCCGATTGCCAGC
GGCACCGCGCCCAGCAGCGCGGCCATGGTGGTCATCAGGATCGGGCGGAA
GCGCAGCAGGCAGGCGCGGTAGATCGCGTCGCGCGGCGACAGGCCATCGC
GCCGTTCGGCATCGATGGCGAAGTCGATCATCATGATCGCGTTCTTTTTC
ACGATGCCGATCAGCAGGATCACGCCGATCAGCGCGATGATGCTGAAGTC
GGTCTTCGATGCCAGCAGCGCCAGCAGCGCGCCCACGCCGGCGGAGGGCA
GCGTCGACAGGATCGTCAGCGGATGCACATAGCTTTCATACAGCACGCCC
AGCACGATGTAGATCGTGATCAGCGCCGCCAGGATCAGGATCGGCTGACT
CTTGAGCGAATCCTGGAACGCCTTGGCGCCGCCCTGGAAGTTGGCGCGCA
GCGTCTCCGGCACGCCGATGCGCGCCATCTCGCGCGTGATCGCGTCGGTC
GCCTGCGACAGCGAAGTGCCCTCGGCCAGGTTGAACGAGATCGTCGAGGC
CGCGAACTGGCCCTGGTGGTTCACGCCCAGCGGCGTGCTGGACGGGGTCA
CGCGCGCGAACGCCGCCAGCGGCACGCGGTTGCCGTTGCCGGTGACCACG
TAGATGTCCTTGAGCGCATCGGGCCCTTGCAGGTATTCCTGGCTCAGCTC
CATCACCACGCGGTACTGGTTCAGCGGATGGTAGATGGTGGACACCAGCC
GCTGGCCGAAGGCATCGTTGAGCACCGCATCCACCTGCTGCGCGGTCACG
CCCAGGCGCGAGGCCGCGTCGCGGTCGATGATCACCGAGGTCTGCAGGCC
CTTGTCGTTGGTATCGGTGTCGATATCCTCCAGCCCCTTCAGGTTCGACA
ACGCGGCGCGCACCTTGGGCTCCCACGCGCGCAGCACTTCCAGGTCGTCC

The third noted mutation may correspond to the sequence listed below ranging from position 2563181-2563281 for Ralstonia eutropha H16 chromosome 1. The bolded, doubleunderlined text indicates a mutation (e.g., nt 101 of SEQ ID NO: 71).

(201 nt)
SEQ ID NO: 71
GCAGCTTGATGCCATTGACGAGGTAGATGGAAACCGGCACGTGCTC
TTTGCGCAGCGCGTTCAGGAACGGGCCTTGTAGCAGTTGCCCTTTGTTGC
TCAT [symbol] GCACACTCCAAATTTATAGGTTTAGTGGTGAATGATGGGGATGGA
AATCCCCGGTTCAAGTCAGGCGGCGCAAAAACGCGCCAGAAAAAAGATCA
AAAAC

The fourth noted mutation may correspond to the sequence listed below ranging from position 241880-242243 for Ralstonia eutropha H16 chromosome 1. The bolded, doubleunderlined text indicates a mutation (e.g., nt 364-379 of SEQ ID NO: 72).

(479 nt)
SEQ ID NO: 72
GAGGATGCCATGTCCGAAGCGCCTGTCCTTGCCCCCTCGACCTCAA
CCCAGCCGCCCGCCGCCGGCCAGCTCAACCTGATCCGCCCGCAGCCATAT
GCCGACTGGGCGCCGCAGGTCACGGCCGAAGAACGCGCCACGCTGCGCCG
CGAGCTGGAGCAGGGCGCCGTGCTGTACTTCCCGAACCTGAATTTCCGCT
TCCAGCCGGGCGAAGAGCGCTTCCTTGACAGCCGCTATTCCGACGGCAAG
TCCAAGAACATCAACCTGCGCGCCGACGACACCGCGGTGCGCGGCGCCCA
GGGCAGTCCGCAGGACCTGGCGGACCTGTACACGCTGATCCGCCGCTACG
CCGACAACAGCGAATTG [symbol] TCCCTGAATACATCCCG
CACATGACGCGCGCCGGCACCTCGCTGCGGCCCAGCGAGATCGCCGGGCG
CCCGGTCAGCTGGCGCAAGGACGACACCCGCCT

In the above sequences, it should be understood that a bacteria may include changes in one or more base pairs relative to the mutation sequences noted above that still produce the same functionality and/or amino acid within the bacteria. For example, a bacteria may include 95%, 96%, 97%, 98%, 99%, or any other appropriate percentage of the same mutation sequences listed above while still providing the noted enhanced ROS resistance.

As elaborated on in the examples, the systems described herein are capable of undergoing intermittent production. For example, when a driving potential is applied to the electrodes to generate hydrogen, the bacteria produce the desired product. Correspondingly, when the potential is removed and hydrogen is no longer generated, production of the product is ceased once the available hydrogen is consumed and a reduction in overall biomass is observed until the potential is once again applied to the electrodes to generate hydrogen. The system will then resume biomass and/or product formation. Thus, while a system may be run continuously to produce a desired product, in some modes of operation a driving potential may be intermittently applied to the electrodes to intermittently split water to form hydrogen and correspondingly intermittently produce a desired product. A frequency of the intermittently applied potential may be any frequency and may either be uniform or non-uniform as the disclosure is not so limited. This ability to intermittently produce a product may be desirable in applications such as when intermittent renewable energy sources are used to provide the power applied to the electrodes including, but not limited to, intermittent power sources such as solar and wind energy.

In some embodiments of any of the aspects, the systems or compositions described herein can be scaled up to meet bioproduction needs. As used herein, the term “scale up” refers to an increase in production capacity (e.g., of a system as described herein). In some embodiments of the aspects, a system (e.g., a bioreactor system) as described herein can be scaled up by at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, or at least 100-fold. In some embodiments of the aspects, a bioreactor system as described herein can be scaled up to at least a 100 ml reactor, at least a 500 ml reactor, at least a 1000 mL reactor, at least a 2 L reactor, at least a 5 L reactor, at least a 10 L reactor, at least a 25 L reactor, at least a 50 L reactor, at least a 100 L reactor, at least a 500 L reactor, or at least a 1,000 L reactor.

Described herein are bacteria engineered for the production of bioplastics (e.g., polyhydroxyalkanoates (PHA)). In one aspect, described herein is an engineered (e.g., Cupriavidus necator) bacterium, comprising: at least one exogenous copy of at least one functional PHA synthase gene; and at least one exogenous copy of at least one functional thioesterase gene.

In some embodiments of any of the aspects, the engineered bacterium comprises one or more of the following: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product; (b) at least one exogenous copy of at least one functional PHA synthase gene; (c) at least one exogenous copy of at least one functional thioesterase gene; and/or (d)(i) at least one endogenous beta-oxidation gene comprising at least one engineered inactivating modification or (d)(ii) at least one exogenous inhibitor of an endogenous beta-oxidation gene or gene product (e.g., mRNA, protein). In some embodiments, the engineered bacterium as described above is also referred to herein as an engineered bioplastics bacterium or an engineered PHA bacterium.

In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein). In some embodiments of any of the aspects, the engineered bacterium comprises: (b) at least one exogenous copy of at least one functional PHA synthase gene. In some embodiments of any of the aspects, the engineered bacterium comprises: (c) at least one exogenous copy of at least one functional thioesterase gene. In some embodiments of any of the aspects, the engineered bacterium comprises: (d)(i) at least one endogenous beta-oxidation gene comprising at least one engineered inactivating modification or (d)(ii) at least one exogenous inhibitor of an endogenous beta-oxidation gene or gene product (e.g., mRNA, protein).

In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); and (b) at least one exogenous copy of at least one functional PHA synthase gene. In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); and (c) at least one exogenous copy of at least one functional thioesterase gene. In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); and (d)(i) at least one endogenous beta-oxidation gene comprising at least one engineered inactivating modification or (d)(ii) at least one exogenous inhibitor of an endogenous beta-oxidation gene or gene product (e.g., mRNA, protein).

In some embodiments of any of the aspects, the engineered bacterium comprises: (b) at least one exogenous copy of at least one functional PHA synthase gene; and (c) at least one exogenous copy of at least one functional thioesterase gene. In some embodiments of any of the aspects, the engineered bacterium comprises: (b) at least one exogenous copy of at least one functional PHA synthase gene; and (d)(i) at least one endogenous beta-oxidation gene comprising at least one engineered inactivating modification or (d)(ii) at least one exogenous inhibitor of an endogenous beta-oxidation gene or gene product (e.g., mRNA, protein). In some embodiments of any of the aspects, the engineered bacterium comprises: (c) at least one exogenous copy of at least one functional thioesterase gene; and (d)(i) at least one endogenous beta-oxidation gene comprising at least one engineered inactivating modification or (d)(ii) at least one exogenous inhibitor of an endogenous beta-oxidation gene or gene product (e.g., mRNA, protein).

In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); (b) at least one exogenous copy of at least one functional PHA synthase gene; and (c) at least one exogenous copy of at least one functional thioesterase gene. In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); (b) at least one exogenous copy of at least one functional PHA synthase gene; and (d)(i) at least one endogenous beta-oxidation gene comprising at least one engineered inactivating modification or (d)(ii) at least one exogenous inhibitor of an endogenous beta-oxidation gene or gene product (e.g., mRNA, protein). In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); (c) at least one exogenous copy of at least one functional thioesterase gene; and (d)(i) at least one endogenous beta-oxidation gene comprising at least one engineered inactivating modification or (d)(ii) at least one exogenous inhibitor of an endogenous beta-oxidation gene or gene product (e.g., mRNA, protein). In some embodiments of any of the aspects, the engineered bacterium comprises: (b) at least one exogenous copy of at least one functional PHA synthase gene; (c) at least one exogenous copy of at least one functional thioesterase gene; and (d)(i) at least one endogenous beta-oxidation gene comprising at least one engineered inactivating modification or (d)(ii) at least one exogenous inhibitor of an endogenous beta-oxidation gene or gene product (e.g., mRNA, protein).

In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); (b) at least one exogenous copy of at least one functional PHA synthase gene; (c) at least one exogenous copy of at least one functional thioesterase gene; and (d)(i) at least one endogenous beta-oxidation gene comprising at least one engineered inactivating modification or (d)(ii) at least one exogenous inhibitor of an endogenous beta-oxidation gene or gene product (e.g., mRNA, protein). In some embodiments of any of the aspects, the engineered bacterium comprises: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); (b) at least one exogenous copy of at least one functional PHA synthase gene; (c) at least one exogenous copy of at least one functional thioesterase gene; or (d)(i) at least one endogenous beta-oxidation gene comprising at least one engineered inactivating modification or (d)(ii) at least one exogenous inhibitor of an endogenous beta-oxidation gene or gene product (e.g., mRNA, protein).

In some embodiments of any of the aspects, the engineered bacterium is a chemoautotroph. In some embodiments of any of the aspects, the engineered bacterium uses CO₂ as its sole carbon source, and/or said engineered bacteria uses H₂ as its sole energy source. In some embodiments of any of the aspects, the engineered bacterium is Cupriavidus necator.

In some embodiments of any of the aspects, the engineered bacterium produces medium chain length PHA (MCL-PHA). In some embodiments of any of the aspects, the MCL-PHA is produced and/or isolated using methods as described further herein.

In some embodiments of any of the aspects, the engineered bacterium comprises one or more of the following: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein); and/or (b) at least one exogenous copy of at least one functional PHA synthase gene. In some embodiments of any of the aspects, the engineered bacterium comprises (a)(i) at least one endogenous polyhydroxyalkanoate (PHA) synthase gene comprising at least one engineered inactivating modification. In some embodiments of any of the aspects, the engineered bacterium comprises (a)(ii) at least one exogenous inhibitor of an endogenous polyhydroxyalkanoate (PHA) synthase gene or gene product (e.g., mRNA, protein)

In some embodiments of any of the aspects, the engineered bacterium comprises at least one endogenous polyhydroxyalkanoate (PHA) synthase gene comprising: (i) at least one engineered inactivating modification or (ii) at least one inhibitor of an endogenous polyhydroxyalkanoate (PHA) synthase gene. In some embodiments of any of the aspects, the engineered bacterium comprises at least one exogenous copy of at least one functional PHA synthase gene. In some embodiments of any of the aspects, the engineered bacterium comprises (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene and (b) at least one exogenous copy of at least one functional PHA synthase gene.

In some embodiments of any of the aspects, the engineered bacterium comprises an engineered inactivating modification of an endogenous polyhydroxyalkanoate (PHA) synthase gene. In some embodiments of any of the aspects, the endogenous PHA synthase comprises phaC. PhaC is a class I poly(R)-hydroxyalkanoic acid synthase, and is the key enzyme in the polymerization of polyhydroxyalkanoates (PHAs). PhaC catalyzes the polymerization of 3-R-hydroxyalkyl CoA thioester to form PHAs with concomitant release of CoA. In some embodiments of any of the aspects, the endogenous PHA synthase comprises Cupriavidus necator phaC.

In some embodiments of any of the aspects, the engineered bacterium comprises an engineered inactivating modification of the endogenous Cupriavidus necator phaC gene. In some embodiments of any of the aspects, the nucleic acid sequence of the endogenous Cupriavidus necator phaC gene comprises SEQ ID NO: 1 or a sequence that is at least 95% (e.g., at least 95%, at least 96%, at least 97%, at least 98%, or at least 99%) identical to the sequence of SEQ ID NO: 1 that maintains the same functions as SEQ ID NO: 1 (e.g., PHA synthase).

| Cupriavidus necator N-1 chromosome 1, REGION: 1478083-1479852 | |
|---|---|
| GenBank: CP002877.1, 1770 bp DNA | |
| SEQ ID NO: 1 | |
| 1 | atggcgaccg gcaaaggcgc ggcagcttcc actcaggaag gcaagtccca accattcaag |
| 61 | ttcacgccgg ggccattcga tccagccaca tggctggaat ggtcccgcca gtggcagggc |
| 121 | actgaaggca acggccacgc ggccgcgtcc ggcattccgg gcctggatgc gctggcaggc |
| 181 | gtcaagatcg agccggcgca gctgggtgat atccagcagc gttacatgaa ggacttctca |
| 241 | gccctgtggc aggccatggc cgagggcaag gccgaggcca ccgggccgct gcacgaccgg |
| 301 | cgcttcgccg gcgacgcgtg gcgcaccaac ctgccatacc gcttcgctgc cgcgttctac |
| 361 | ctgctcaatg cgcgcgcctt gaccgagctg gccgatgctg ttgaggccga tgccaagacg |
| 421 | cgccagcgca tccgctttgc gatctcgcaa tgggtcgatg cgatgtcgcc cgccaacttc |
| 481 | ctcgccacga atcccgaggc gcagcgcctg ctgatcgagt cgggcggcga atcgctgcgt |
| 541 | gccggcgtgc gcaacatgat ggaagacctg acgcgcggca agatctcgca gaccgacgag |
| 601 | agcgcgtttg aggtcggccg caatgtcgcg gtgagcgaag gcgccgtagt cttcgagaac |
| 661 | gaatacttcc agctgttgca gtacaagccg ctgaccgaca aggtgcatgc gcgcccgctg |
| 721 | ctgatggtgc cgccgtgcat caacaagtac tacatcctgg acctgcagcc ggagagctcg |
| 781 | ctggtgcgtc atgtggtgga gcaggggcat acggtgttcc tggtgtcgtg gcgcaatccg |
| 841 | gacgccagca tggctggcag cacctgggac gactacatcg agcacgcggc catccgcgcc |
| 901 | atcgaagtcg cgcgcgacat cagcggccag gacaagatca acgtgctcgg cttctgcgtg |
| 961 | ggcggcacca ttgtgtcgac tgcgctggcg gtgatggccg cgcgcggcca gcacccggct |
| 1021 | gccagcgtca cgctgctgac cacgctgctg gactttgccg acaccggcat cctcgacgtc |
| 1081 | tttgtcgacg agggccatgt gcagctgcgc gaggccacgc tgggcggcgc cgccggcgcg |
| 1141 | ccgtgcgcgc tgctgcgcgg ccttgagctg gccaatacct tctcgttcct gcgcccgaac |
| 1201 | gacctggtgt ggaactacgt ggtcgacaac tacctgaagg gcaacacgcc ggtgccgttc |
| 1261 | gacctgctgt tctggaacgg cgacgccacc aacctgccgg ggccttggta ctgctggtac |
| 1321 | ctgcgccaca cctacctgca ggacgagctc aaggtgccgg gcaagctgac tgtgtgcggc |
| 1381 | gtgcccgtgg acctggccag catcgacgtg ccgacctaca tctacggctc gcgcgaagac |
| 1441 | catatcgtgc catggaccgc ggcctatgcc tcgaccgcgc tgctggcgaa caagctgcgc |
| 1501 | ttcgtgctgg gtgcgtcggg ccatatcgcc ggtgtgatca acccgccggc caagaacaag |
| 1561 | cgcagccact ggaccaacga tgcgctgccg gagtcgccgc agcaatggct ggctggcgcc |
| 1621 | accgagcatc acggcagctg gtggccggac tggaccgcat ggctggcagg ccaggccggc |
| 1681 | gcgaaacgtg ccgcgcccgc caactacggc aatgcgcgct atcccgcgat cgaacccgcg |
| 1741 | cctgggcgat acgtcaaagc caaggcatga |

In some embodiments of any of the aspects, the amino acid sequence encoded by the endogenous Cupriavidus necator phaC gene comprises SEQ ID NO: 2 or an amino acid sequence that is at least 95% (e.g., at least 95%, at least 96%, at least 97%, at least 98%, or at least 99%) identical to the sequence of SEQ ID NO: 2 that maintains the same functions as SEQ ID NO: 2 (e.g., PHA synthase).

| class I poly(R)-hydroxyalkanoic acid synthase [Cupriavidus necator], | |
|---|---|
| NCBI Reference Sequence: WP_013956451.1, 589 aa | |
| SEQ ID NO: 2 | |
| 1 | matgkgaaas tqegksqpfk ftpgpfdpat wlewsrqwqg tegnghaaas gipgldalag |
| 61 | vkiepaqlgd iqqrymkdfs alwqamaegk aeatgplhdr rfagdawrtn lpyrfaaafy |
| 121 | llnaraltel adaveadakt rqrirfaisq wvdamspanf latnpeaqrl liesggeslr |
| 181 | agvrnmmedl trgkisqtde safevgrnva vsegavvfen eyfqllqykp ltdkvharpl |
| 241 | lmvppcinky yildlqpess lvrhvveqgh tvflvswrnp dasmagstwd dyiehaaira |
| 301 | ievardisgq dkinvlgfcv ggtivstala vmaargqhpa asvtllttll dfadtgildv |
| 361 | fvdeghvqlr eatlggaaga pcallrglel antfsflrpn dlvwnyvvdn ylkgntpvpf |
| 421 | dllfwngdat nlpgpwycwy lrhtylqdel kvpgkltvcg vpvdlasidv ptyiygsred |
| 481 | hivpwtaaya stallanklr fvlgasghia gvinppaknk rshwtndalp espqqwlaga |
| 541 | tehhgswwpd wtawlagqag akraapanyg narypaiepa pgryvkaka |

In some embodiments of any of the aspects, the engineered inactivating modification of an endogenous polyhydroxyalkanoate (PHA) synthase gene comprises a point mutation. Non-limiting examples of inactivating point mutations of C. necator phaC (see e.g., SEQ ID NO: 2) include non-conservative substitutions of residues T323, C438, Y445, L446, or E267 (e.g., T323I, T323S, C438G, Y445F, L446K, or E267K). Additional non-limiting examples of point mutations of C. necator phaC (see e.g., SEQ ID NO: 2) include C319S, C459S, S260A, S260T, S546I, E267K, T323S, T323I, C438G, Y445F, L446K, W425A, D480N, H508Q, S35P, S80P, A154V, L231P, D306A, L358P, A391T, T393A, V470M, N519S, S546G, and A565E. In some embodiments of any of the aspects, the engineered inactivating modification of an endogenous polyhydroxyalkanoate (PHA) synthase gene comprises a deletion. Non-limiting examples include deletions of regions D281-D290, A372-C382, E578-A589 and/or V585-A589 of C. necator phaC (see e.g., SEQ ID NO: 2). See e.g., Rehm et al., Molecular characterization of the poly(3 hydroxybutyrate) (PHB) synthase from Ralstonia eutropha: in vitro evolution, site-specific mutagenesis and development of a PHB synthase protein model, Biochimica et Biophysica Acta 1594 (2002) 178-190, the content of which is incorporated herein by reference in its entirety. In some embodiments of any of the aspects, the engineered inactivating modification of an endogenous polyhydroxyalkanoate (PHA) synthase gene comprises a deletion of the entire coding sequence (e.g., a knockout of the endogenous phaC gene, denoted herein as ΔphaC).

In some embodiments of any of the aspects, the engineered bacterium comprises an engineered inactivating modification of an endogenous gene involved in the PHA synthesis pathway. In some embodiments of any of the aspects, the endogenous gene involved in the PHA synthesis pathway comprises phaA, phaB, and/or phaC (e.g., a Class I PHA synthase operon). In some embodiments of any of the aspects, the PHA synthesis pathway comprises Cupriavidus necator phaA, Cupriavidus necator phaB, and/or Cupriavidus necator phaC.

PhaA is an acetyl-CoA acetyltransferase that catalyzes the condensation of two acetyl-coA units to form acetoacetyl-CoA. PhaA is involved in the biosynthesis of PHAs (e.g., polyhydroxybutyrate (PHB)). PhaA also catalyzes the reverse reaction, i.e. the cleavage of acetoacetyl-CoA, and is therefore also involved in the reutilization of PHB.

In some embodiments of any of the aspects, the engineered bacterium comprises an engineered inactivating modification of the endogenous Cupriavidus necator phaA gene. In some embodiments of any of the aspects, the nucleic acid sequence of the endogenous Cupriavidus necator phaA gene comprises SEQ ID NO: 24 or a sequence that is at least 95% (e.g., at least 95%, at least 96%, at least 97%, at least 98%, or at least 99%) identical to the sequence of SEQ ID NO: 24 that maintains the same functions as SEQ ID NO: 24 (e.g., acetyl-CoA acetyltransferase).

| Cupriavidus necator phaA acetyl-CoA acetyltransferase, Cupriavidus | |
|---|---|
| necator H16 chromosome 1, complete sequence, GenBank: CP039287.1, | |
| REGION: 1557857-1559035, 1179 bp | |
| SEQ ID NO: 24 | |
| 1 | atgactgacg ttgtcatcgt atccgccgcc cgcaccgcgg tcggcaagtt tggcggctcg |
| 61 | ctggccaaga tcccggcacc ggaactgggt gccgtggtca tcaaggccgc gctggagcgc |
| 121 | gccggcgtca agccggagca ggtgagcgaa gtcatcatgg gccaggtgct gaccgccggt |
| 181 | tcgggccaga accccgcacg ccaggccgcg atcaaggccg gcctgccggc gatggtgccg |
| 241 | gccatgacca tcaacaaggt gtgcggctcg ggcctgaagg ccgtgatgct ggccgccaac |
| 301 | gcgatcatgg cgggcgacgc cgagatcgtg gtggccggcg gccaggaaaa catgagcgcc |
| 361 | gccccgcacg tgctgccggg ctcgcgcgat ggtttccgca tgggcgatgc caagctggtc |
| 421 | gacaccatga tcgtcgacgg cctgtgggac gtgtacaacc agtaccacat gggcatcacc |
| 481 | gccgagaacg tggccaagga atacggcatc acacgcgagg cgcaggatga gttcgccgtc |
| 541 | ggctcgcaga acaaggccga agccgcgcag aaggccggca agtttgacga agagatcgtc |
| 601 | ccggtgctga tcccgcagcg caagggcgac ccggtggcct tcaagaccga cgagttcgtg |
| 661 | cgccagggcg ccacgctgga cagcatgtcc ggcctcaagc ccgccttcga caaggccggc |
| 721 | acggtgaccg cggccaacgc ctcgggcctg aacgacggcg ccgccgcggt ggtggtgatg |
| 781 | tcggcggcca aggccaagga actgggcctg accccgctgg ccacgatcaa gagctatgcc |
| 841 | aacgccggtg tcgatcccaa ggtgatgggc atgggcccgg tgccggcctc caagcgcgcc |
| 901 | ctgtcgcgcg ccgagtggac cccgcaagac ctggacctga tggagatcaa cgaggccttt |
| 961 | gccgcgcagg cgctggcggt gcaccagcag atgggctggg acacctccaa ggtcaatgtg |
| 1021 | aacggcggcg ccatcgccat cggccacccg atcggcgcgt cgggctgccg tatcctggtg |
| 1081 | acgctgctgc acgagatgaa gcgccgtgac gcgaagaagg gcctggcctc gctgtgcatc |
| 1141 | ggcggcggca tgggcgtggc gctggcagtc gagcgcaaa |

In some embodiments of any of the aspects, the amino acid sequence encoded by the endogenous Cupriavidus necator phaA gene comprises SEQ ID NO: 25 or an amino acid sequence that is at least 95% (e.g., at least 95%, at least 96%, at least 97%, at least 98%, or at least 99%) identical to the sequence of SEQ ID NO: 25 that maintains the same functions as SEQ ID NO: 25 (e.g., PHA synthase).

SEQ ID NO: 25, phaA, acetyl-CoA
C-acetyltransferase [Cupriavidus], NCBI Reference
Sequence: WP_010810132.1, 393 aa
MTDVVIVSAARTAVGKFGGSLAKIPAPELGAVVIKAALERAGVKPEQVS
EVIMGQVLTAGSGQNPARQAAIKAGLPAMVPAMTINKVCGSGLKAVMLA
ANAIMAGDAEIVVAGGQENMSAAPHVLPGSRDGFRMGDAKLVDTMIVDG
LWDVYNQYHMGITAENVAKEYGITREAQDEFAVGSQNKAEAAQKAGKFD
EEIVPVLIPQRKGDPVAFKTDEFVRQGATLDSMSGLKPAFDKAGTVTAA
NASGLNDGAAAVVVMSAAKAKELGLTPLATIKSYANAGVDPKVMGMGPV
PASKRALSRAEWTPQDLDLMEINEAFAAQALAVHQQMGWDTSKVNVNGG
AIAIGHPIGASGCRILVTLLHEMKRRDAKKGLASLCIGGGMGVALAVER
K

In some embodiments of any of the aspects, the engineered inactivating modification of an endogenous gene involved in the PHA synthesis pathway comprises a deletion of the entire coding sequence (e.g., a knockout of the endogenous phaA gene, denoted herein as ΔphaA).

In some embodiments of any of the aspects, the engineered bacterium comprises an engineered inactivating modification of the endogenous Cupriavidus necator phaB gene. PhaB is an acetoacetyl-CoA reductase that catalyzes the chiral reduction of acetoacetyl-CoA to (R)-3-hydroxybutyryl-CoA. PhaB is involved in the biosynthesis of PHAs (e.g., polyhydroxybutyrate (PHB)). PhaB can also be referred to as phbB. In some embodiments of any of the aspects, the nucleic acid sequence of the endogenous Cupriavidus necator phaB gene comprises SEQ ID NO: 26 or a sequence that is at least 95% (e.g., at least 95%, at least 96%, at least 97%, at least 98%, or at least 99%) identical to the sequence of SEQ ID NO: 26 that maintains the same functions as SEQ ID NO: 26 (e.g., acetoacetyl-CoA reductase).

| SEQ ID NO: 26, Cupriavidus necator strain A-04 acetoacetyl-CoA | |
|---|---|
| reductase (phbB) gene, complete cds, GenBank: FJ897462.1, 741 bp | |
| 1 | atgactcagc gcattgcgta tgtgaccggc ggcatgggtg gtatcggaac cgccatttgc |
| 61 | cagcggctgg ccaaggatgg ctttcgtgtg gtggccggtt gcggccccaa ctcgccgcgc |
| 121 | cgcgaaaagt ggctggagca gcagaaggcc ctgggcttcg atttcattgc ctcggaaggc |
| 181 | aatgtggctg actgggactc gaccaagacc gcattcgaca aggtcaagtc cgaggtcggc |
| 241 | gaggttgatg tgctgatcaa caacgccggt atcacccgcg acgtggtgtt ccgcaagatg |
| 301 | acccgcgccg actgggatgc ggtgatcgac accaacctga cctcgctgtt caacgtcacc |
| 361 | aagcaggtga tcgacggcat ggccgaccgt ggctggggcc gcatcgtcaa catctcgtcg |
| 421 | gtgaacgggc agaagggcca gttcggccag accaactact ccaccgccaa ggccggcctg |
| 481 | catggcttca ccatggcact ggcgcaggaa gtggcgacca agggcgtgac cgtcaacacg |
| 541 | gtctctccgg gctatatcgc caccgacatg gtcaaggcga tccgccagga cgtgctcgac |
| 601 | aagatcgtcg cgacgatccc ggtcaagcgc ctgggcctgc cggaagagat cgcctcgatc |
| 661 | tgcgcctggt tgtcgtcgga ggagtccggt ttctcgaccg gcgccgactt ctcgctcaac |
| 721 | ggcggcctgc atatgggctg a |

In some embodiments of any of the aspects, the amino acid sequence encoded by the endogenous Cupriavidus necator phaC gene comprises SEQ ID NO: 27 or an amino acid sequence that is at least 95% (e.g., at least 95%, at least 96%, at least 97%, at least 98%, or at least 99%) identical to the sequence of SEQ ID NO: 27 that maintains the same functions as SEQ ID NO: 27 (e.g., e.g., acetoacetyl-CoA reductase).

phaB 3-ketoacyl-ACP reductase [Cupriavidus], NCBI Reference
Sequence: WP_010810131.1, 246 aa
SEQ ID NO: 27
MTQRIAYVTGGMGGIGTAICQRLAKDGFRVVAGCGPNSPRREKWLEQQKALGFDFIASEG
NVADWDSTKTAFDKVKSEVGEVDVLINNAGITRDVVFRKMTRADWDAVIDTNLTSLFNVT
KQVIDGMADRGWGRIVNISSVNGQKGQFGQTNYSTAKAGLHGFTMALAQEVATKGVTVNT
VSPGYIATDMVKAIRQDVLDKIVATIPVKRLGLPEEIASICAWLSSEESGFSTGADFSLN
GGLHMG

In some embodiments of any of the aspects, the engineered inactivating modification of an endogenous gene involved in the PHA synthesis pathway comprises a deletion of the entire coding sequence (e.g., a knockout of the endogenous phaB gene, denoted herein as ΔphaB).

In some embodiments of any of the aspects, the engineered bacterium comprises an engineered inactivating modification of an endogenous phaA (e.g., SEQ ID NOs: 1, 2), an engineered inactivating modification of an endogenous phaB (e.g., SEQ ID NOs: 24, 25), or an engineered inactivating modification of an endogenous phaC (e.g., SEQ ID NOs: 26, 27). In some embodiments of any of the aspects, the engineered bacterium comprises an engineered inactivating modification of an endogenous phaA (e.g., SEQ ID NOs: 1, 2). In some embodiments of any of the aspects, the engineered bacterium comprises an engineered inactivating modification of an endogenous phaB (e.g., SEQ ID NOs: 24, 25). In some embodiments of any of the aspects, the engineered bacterium comprises an engineered inactivating modification of an endogenous phaC (e.g., SEQ ID NOs: 26, 27).

In some embodiments of any of the aspects, the engineered bacterium comprises an engineered inactivating modification of an endogenous phaA (e.g., SEQ ID NOs: 1, 2), and an engineered inactivating modification of an endogenous phaB (e.g., SEQ ID NOs: 24, 25). In some embodiments of any of the aspects, the engineered bacterium comprises an engineered inactivating modification of an endogenous phaA (e.g., SEQ ID NOs: 1, 2) and an engineered inactivating modification of an endogenous phaC (e.g., SEQ ID NOs: 26, 27). In some embodiments of any of the aspects, the engineered bacterium comprises an engineered inactivating modification of an endogenous phaB (e.g., SEQ ID NOs: 24, 25) and an engineered inactivating modification of an endogenous phaC (e.g., SEQ ID NOs: 26, 27). In some embodiments of any of the aspects, the engineered bacterium comprises an engineered inactivating modification of an endogenous phaA (e.g., SEQ ID NOs: 1, 2), an engineered inactivating modification of an endogenous phaB (e.g., SEQ ID NOs: 24, 25), and an engineered inactivating modification of an endogenous phaC (e.g., SEQ ID NOs: 26, 27).

In some embodiments of any of the aspects, an organism can comprise alternative groups of genes involved in the PHA synthesis pathway. As an example, the Class II PHA synthase operon (e.g., in Pseudomonas oleovorans) comprises phaC1, phaZ, phaC2, and phaD. As another example, the Class III PHA synthase operon (e.g., in Allochromatium vinosum) comprises phaC, phaE, phaA, ORF4, phaP, and phaB. As such, an engineered bacterium can comprise an engineered inactivating modification and/or an inhibitor of at least one endogenous gene involved in the PHA synthesis pathway (e.g., phaC1, phaZ, phaC2, phaD, phaC, phaE, phaA, ORF4, phaP, and/or phaB).

In some embodiments of any of the aspects, the engineered bacterium comprises an inhibitor of an endogenous PHA synthase gene. Non-limiting examples of PHA synthase (e.g., PhaC) inhibitors include carbadethia CoA analogs, sT-CH₂-CoA, sTet-CH₂-CoA, and sT-aldehyde. See e.g., Zhang et al., Chembiochem. 2015 Jan. 2; 16(1): 156-166, the contents of which are incorporated herein in be reference in their entireties. In some embodiments of any of the aspects, the engineered bacterium comprises an inhibitor of at least one endogenous gene involved in the PHA synthesis pathway. Non-limiting examples of such inhibitors include an inhibitory RNA (e.g., siRNA, miRNA) against a gene involved in PHA synthesis (e.g., a PHA synthase, PhaC, PhaB, PhaA), a small molecule inhibitor of a gene involved in PHA synthesis (e.g., a PHA synthase, PhaC, PhaB, PhaA), and the like.

In some embodiments of any of the aspects, the inhibitor can also inhibit heterologous PHA synthase genes (e.g., P. aeruginosa phaC1/phaC2, Pseudomonas spp. 61-3 phaC1). In some embodiments of any of the aspects, the inhibitor does not inhibit heterologous PHA synthase genes (e.g., P. aeruginosa phaC1/phaC2, Pseudomonas spp. 61-3 phaC1), e.g., it is a specific inhibitor of one or more endogenous PHA synthase genes. In some embodiments of any of the aspects, the inhibitor preferentially inhibits one or more endogenous PHA synthase genes as compared to heterologous PHA synthase genes (e.g., P. aeruginosa phaC1/phaC2, Pseudomonas spp. 61-3 phaC1), e.g., the inhibitory effect on one or more endogenous PHA synthase genes is at least 200%, 300%, 400%, 500%, 1,000% or more of the inhibitory effect on heterologous PHA synthase genes.

In some embodiments of any of the aspects, the engineered bacterium comprises at least one exogenous copy of at least one functional PHA synthase gene. In some embodiments of any of the aspects, the functional PHA synthase gene preferentially produces medium-chain-length polyhydroxyalkanoate (MCL-PHA), as described herein. As such, the functional PHA synthase gene can be selected from any PHA synthase gene from any species that preferentially produces MCL-PHA. In some embodiments of any of the aspects, the functional PHA synthase gene is heterologous.

In some embodiments of any of the aspects, the functional heterologous PHA synthase gene comprises a Pseudomonas aeruginosa phaC gene. In some embodiments of any of the aspects, the Pseudomonas aeruginosa phaC gene comprises Pseudomonas aeruginosa phaC1 and/or Pseudomonas aeruginosa phaC2. In some embodiments of any of the aspects, the functional heterologous PHA synthase gene comprises Pseudomonas spp. 61-3 phaC1.

In some embodiments of any of the aspects, the engineered bacterium comprises a Pseudomonas aeruginosa phaC1 gene. In some embodiments of any of the aspects, the engineered bacterium comprises a Pseudomonas aeruginosa phaC2 gene. In some embodiments of any of the aspects, the engineered bacterium comprises a Pseudomonas spp. 61-3 phaC1 gene. In some embodiments of any of the aspects, the engineered bacterium comprises a Pseudomonas aeruginosa phaC1 gene, a Pseudomonas aeruginosa phaC2 gene, and/or a Pseudomonas spp. 61-3 phaC1 gene.

In some embodiments of any of the aspects, the engineered bacterium comprises at least one exogenous copy of at least one functional PHA synthase gene comprising SEQ ID NO: 3, SEQ ID NO: 4, SEQ ID NO: 5, SEQ ID NO: 6, SEQ ID NO: 81 or a nucleic acid sequence that is at least 95% (e.g., at least 95%, at least 96%, at least 97%, at least 98%, or at least 99%) identical to the sequence of at least one of SEQ ID NOs: 3-6 that maintains the same functions as at least one of SEQ ID NOs: 3-6 or 81 (e.g., PHA synthase).

| phaC1 poly(3-hydroxyalkanoic acid) synthase, Pseudomonas aeruginosa PAO1, | |
|---|---|
| complete genome, NCBI Reference Sequence: NC_002516.2, REGION: 5695366-5697045, | |
| 1680 bp | |
| SEQ ID NO: 3 | |
| 1 | atgagtcaga agaacaataa cgagcttccc aagcaagccg cggaaaacac gctgaacctg |
| 61 | aatccggtga tcggcatccg gggcaaggac ctgctcacct ccgcgcgcat ggtcctgctc |
| 121 | caggcggtgc gccagccgct gcacagcgcc aggcacgtgg cgcatttcag cctggagctg |
| 181 | aagaacgtcc tgctcggcca gtcggagcta cgcccaggcg atgacgaccg acgcttttcc |
| 241 | gatccggcct ggagccagaa tccactgtac aagcgctaca tgcagaccta cctggcctgg |
| 301 | cgcaaggagc tgcacagctg gatcagccac agcgacctgt cgccgcagga catcagtcgt |
| 361 | ggccagttcg tcatcaacct gctgaccgag gcgatgtcgc cgaccaacag cctgagcaac |
| 421 | ccggcggcgg tcaagcgctt cttcgagacc ggcggcaaga gcctgctgga cggcctcggc |
| 481 | cacctggcca aggacctggt gaacaacggc gggatgccga gccaggtgga catggacgcc |
| 541 | ttcgaggtgg gcaagaacct ggccaccacc gagggcgccg tggtgttccg caacgacgtg |

| 601 | ctggaactga tccagtaccg gccgatcacc gagtcggtgc acgaacgccc gctgctggtg |
|---|---|
| 661 | gtgccgccgc agatcaacaa gttctacgtc ttcgacctgt cgccggacaa gagcctggcg |
| 721 | cgcttctgcc tgcgcaacgg cgtgcagacc ttcatcgtca gttggcgcaa cccgaccaag |
| 781 | tcgcagcgcg aatggggcct gaccacctat atcgaggcgc tcaaggaggc catcgaggta |
| 841 | gtcctgtcga tcaccggcag caaggacctc aacctcctcg gcgcctgctc cggcgggatc |
| 901 | accaccgcga ccctggtcgg ccactacgtg gccagcggcg agaagaaggt caacgccttc |
| 961 | acccaactgg tcagcgtgct cgacttcgaa ctgaataccc aggtcgcgct gttcgccgac |
| 1021 | gagaagactc tggaggccgc caagcgtcgt tcctaccagt ccggcgtgct ggagggcaag |
| 1081 | gacatggcca aggtgttcgc ctggatgcgc cccaacgacc tgatctggaa ctactgggtc |
| 1141 | aacaactacc tgctcggcaa ccagccgccg gcgttcgaca tcctctactg gaacaacgac |
| 1201 | accacgcgcc tgcccgccgc gctgcacggc gagttcgtcg aactgttcaa gagcaacccg |
| 1261 | ctgaaccgcc ccggcgccct ggaggtctcc ggcacgccca tcgacctgaa gcaggtgact |
| 1321 | tgcgacttct actgtgtcgc cggtctgaac gaccacatca ccccctggga gtcgtgctac |
| 1381 | aagtcggcca ggctgctggg tggcaagtgc gagttcatcc tctccaacag cggtcacatc |
| 1441 | cagagcatcc tcaacccacc gggcaacccc aaggcacgct tcatgaccaa tccggaactg |
| 1501 | cccgccgagc ccaaggcctg gctggaacag gccggcaagc acgccgactc gtggtggttg |
| 1561 | cactggcagc aatggctggc cgaacgctcc ggcaagaccc gcaaggcgcc cgccagcctg |
| 1621 | ggcaacaaga cctatccggc cggcgaagcc gcgcccggaa cctacgtgca tgaacgatga |
| phaC2 poly(3-hydroxyalkanoic acid) synthase, Pseudomonas aeruginosa PAO1, | |
| complete genome, NCBI Reference Sequence: NC_002516.2, REGION: 5698359-5700041, | |
| 1683 bp | |
| SEQ ID NO: 4 | |
| 1 | atgcgagaaa agcaggaatc gggtagcgtg ccggtgcccg ccgagttcat gagtgcacag |
| 61 | agcgccatcg tcggcctgcg cggcaaggac ctgctgacga cggtccgcag cctggctgtc |
| 121 | cacggcctgc gccagccgct gcacagtgcg cggcacctgg tcgccttcgg aggccagttg |
| 181 | ggcaaggtgc tgctgggcga caccctgcac cagccgaacc cacaggacgc ccgcttccag |
| 241 | gatccatcct ggcgcctcaa tcccttctac cggcgcaccc tgcaggccta cctggcgtgg |
| 301 | cagaaacaac tgctcgcctg gatcgacgaa agcaacctgg actgcgacga tcgcgcccgc |
| 361 | gcccgcttcc tcgtcgcctt gctctccgac gccgtggcac ccagcaacag cctgatcaat |
| 421 | ccactggcgt taaaggaact gttcaatacc ggcgggatca gcctgctcaa tggcgtccgc |
| 481 | cacctgctcg aagacctggt gcacaacggc ggcatgccca gccaggtgaa caagaccgcc |
| 541 | ttcgagatcg gtcgcaacct cgccaccacg caaggcgcgg tggtgttccg caacgaggtg |
| 601 | ctggagctga tccagtacaa gccgctgggc gagcgccagt acgccaagcc cctgctgatc |
| 661 | gtgccgccgc agatcaacaa gtactacatc ttcgacctgt cgccggaaaa gagcttcgtc |
| 721 | cagtacgccc tgaagaacaa cctgcaggtc ttcgtcatca gttggcgcaa ccccgacgcc |
| 781 | cagcaccgcg aatggggcct gagcacctat gtcgaggccc tcgaccaggc catcgaggtc |
| 841 | agccgcgaga tcaccggcag ccgcagcgtg aacctggccg gcgcctgcgc cggcgggctc |
| 901 | accgtagccg ccttgctcgg ccacctgcag gtgcgccggc aactgcgcaa ggtcagtagc |
| 961 | gtcacctacc tggtcagcct gctcgacagc cagatggaaa gcccggcgat gctcttcgcc |
| 1021 | gacgagcaga ccctggagag cagcaagcgc cgctcctacc agcatggcgt gctggacggg |
| 1081 | cgcgacatgg ccaaggtgtt cgcctggatg cgccccaacg acctgatctg gaactactgg |
| 1141 | gtcaacaact acctgctcgg caggcagccg ccggcgttcg acatcctcta ctggaacaac |
| 1201 | gacaacacgc ggctgcccgc ggcgttccac ggcgaactgc tcgacctgtt caagcacaac |
| 1261 | ccgctgaccc gcccgggcgc gctggaggtc agcgggaccg cggtggacct gggcaaggtg |
| 1321 | gcgatcgaca gcttccacgt cgccggcatc accgaccaca tcacgccctg ggacgcggtg |
| 1381 | tatcgctcgg ccctcctgct gggcggccag cgccgcttca tcctgtccaa cagcgggcac |
| 1441 | atccagagca tcctcaaccc tcccggaaac cccaaggcct gctacttcga gaacgacaag |
| 1501 | ctgagcagcg atccacgcgc ctggtactac gacgccaagc gcgaagaggg cagctggtgg |
| 1561 | ccggtctggc tgggctggct gcaggagcgc tcgggcgagc tgggcaaccc tgacttcaac |
| 1621 | cttggcagcg ccgcgcatcc gcccctcgaa gcggccccgg gcacctacgt gcatatacgc |
| 1681 | tga |

Pseudomonas aeruginosa PAO1 phaC1 (1680 bp)
SEQ ID NO: 5
ATGTCGCAGAAGAACAACAACGAGCTGCCGAAGCAGGCCGCCGAGAACACCCTGAACCT
GAACCCGGTGATCGGCATTCGTGGTAAAGATCTGCTGACGTCGGCCCGCATGGTGCTGCT
GCAAGCAGTGCGTCAACCGCTGCATAGTGCACGTCATGTTGCGCATTTCTCGCTGGAGCT
GAAGAACGTGCTGCTGGGCCAGTCGGAACTGCGTCCGGGCGATGATGATCGTCGTTTCA
GTGATCCGGCATGGTCGCAAAATCCGCTGTACAAGCGCTACATGCAGACGTACCTGGCCT
GGCGCAAGGAACTGCACTCGTGGATCTCGCACTCGGATTTGTCGCCGCAGGATATTTCGC
GTGGCCAGTTCGTGATCAACCTGCTGACGGAGGCCATGTCGCCGACCAATTCGCTGTCGA
ATCCAGCAGCCGTGAAGCGCTTCTTCGAAACCGGCGGTAAGTCGCTGCTGGACGGTCTG
GGTCATCTGGCAAAGGACCTGGTGAACAATGGCGGTATGCCGTCGCAGGTGGACATGGA
CGCGTTCGAAGTGGGCAAGAATCTGGCCACCACCGAAGGTGCAGTGGTGTTCCGCAACG
ACGTGCTGGAGCTGATCCAGTATCGCCCGATTACCGAATCGGTGCACGAACGTCCGCTGC
TGGTTGTGCCGCCGCAGATCAACAAGTTCTACGTGTTCGACTTGTCGCCGGACAAGTCGC
TGGCCCGCTTTTGCCTGCGCAACGGCGTGCAGACCTTTATTGTGAGTTGGCGTAACCCGA
CCAAGTCGCAGCGCGAATGGGGTCTGACCACCTACATCGAGGCCCTGAAGGAAGCGATC
GAAGTGGTGCTGTCGATTACCGGCTCGAAGGACCTGAACCTGCTGGGTGCCTGCAGTGGT
GGCATTACAACCGCAACCCTGGTTGGCCATTACGTGGCATCGGGCGAAAAGAAGGTCAA
CGCGTTCACTCAGCTGGTGTCGGTGCTGGACTTCGAGCTGAACACCCAGGTGGCCCTGTT
TGCCGATGAAAAGACGCTGGAAGCCGCAAAGCGCCGCTCGTATCAATCGGGTGTGCTGG
AGGGCAAGGACATGGCAAAGGTGTTCGCATGGATGCGCCCGAACGACCTGATCTGGAAC
TACTGGGTGAACAACTACCTGCTGGGCAACCAACCGCCGGCCTTCGACATCCTGTACTGG
AACAACGACACCACCCGTTTGCCTGCAGCACTGCATGGCGAATTCGTGGAGCTGTTCAAG
TCGAACCCGCTGAATCGTCCGGGCGCACTGGAAGTGTCGGGTACGCCGATTGACCTGAA
GCAAGTGACCTGCGACTTCTATTGCGTGGCCGGCCTGAACGACCACATCACCCCGTGGGA
ATCGTGCTACAAGTCGGCACGTCTGTTAGGTGGTAAGTGCGAGTTCATCCTGTCGAACTC
GGGCCACATCCAGTCGATCCTGAACCCGCCGGGTAACCCGAAAGCACGCTTCATGACCA
ACCCAGAACTGCCGGCAGAACCGAAAGCATGGCTGGAACAGGCCGGCAAGCATGCCGA
TAGTTGGTGGCTGCATTGGCAGCAGTGGCTGGCAGAACGTTCGGGTAAAACGCGCAAGG
CACCGGCAAGTCTGGGCAACAAGACCTATCCGGCAGGCGAAGCAGCACCAGGCACATAT
GTGCATGAGCGCTAG
Pseudomonas aeruginosa PAO1 phaC2 (1707 bp)
SEQ ID NO: 6
ATGCGCGAGAAGCAGGAGTCGGGCTCAGTGCCAGTGCCGGCCGAGTTCATGTCGGCCCA
GTCGGCCATTGTGGGCCTGAGAGGCAAGGACCTGCTGACCACCGTGCGCTCGCTGGCAG
TGCATGGCCTGCGCCAACCACTGCATTCGGCCCGTCATCTGGTGGCCTTTGGCGGCCAAC
TGGGCAAGGTGCTGCTGGGCGACACCCTGCATCAGCCGAACCCGCAGGATGCCCGCTTC
CAGGATCCGTCGTGGCGCCTGAATCCGTTCTACCGCCGTACCCTGCAGGCCTACCTGGCC
TGGCAGAAGCAGCTGCTGGCCTGGATCGACGAGTCGAACCTGGACTGCGACGATCGCGC
ACGTGCCCGCTTCCTGGTGGCACTGCTGTCGGATGCCGTGGCACCGTCGAATTCGCTGAT
CAACCCGCTGGCCCTGAAGGAGCTGTTCAACACCGGCGGCATCTCGCTGCTGAACGGCG
TGCGCCACCTGCTGGAGGACCTGGTGCACAATGGCGGTATGCCGTCGCAGGTGAACAAG
ACCGCCTTCGAGATCGGCCGCAACCTGGCCACCACCCAAGGCGCCGTGGTGTTCCGCAA
CGAGGTGCTGGAGCTGATCCAGTACAAGCCGCTGGGCGAGCGCCAGTACGCCAAGCCGC
TGCTGATCGTGCCGCCGCAGATCAACAAGTACTACATCTTCGACCTGTCGCCGGAGAAGT
CGTTCGTGCAGTACGCCCTGAAGAACAACCTGCAGGTGTTCGTGATCTCGTGGCGCAACC
CGGACGCCCAGCACCGCGAGTGGGGCCTGTCGACCTACGTGGAAGCACTGGATCAGGCC
ATCGAAGTGTCGCGCGAGATCACCGGCTCGCGCTCGGTGAACCTGGCCGGCGCATGTGC
AGGTGGTCTGACTGTTGCAGCCCTGCTGGGTCACCTGCAGGTGCGCCGTCAACTGCGCAA
GGTGTCGTCGGTGACCTACCTGGTGTCGCTGCTGGACTCGCAGATGGAGTCGCCGGCCAT
GCTGTTCGCCGACGAGCAGACCCTGGAGTCGTCGAAACGCCGCTCGTACCAGCATGGCG
TGCTGGATGGCCGCGACATGGCCAAGGTGTTCGCCTGGATGCGCCCGAACGACCTGATCT
GGAACTACTGGGTGAACAACTACCTGCTGGGCCGCCAGCCGCCGGCCTTCGACATCCTGT
ACTGGAACAACGACAACACCCGCCTGCCAGCCGCCTTCCACGGCGAGCTGCTGGACCTG
TTCAAGCACAACCCGCTGACCAGACCAGGCGCCCTGGAGGTGTCAGGCACCGCCGTGGA
TCTGGGCAAGGTGGCCATTGACTCGTTCCATGTGGCCGGCATCACCGACCACATCACCCC
GTGGGACGCCGTGTACCGCTCGGCACTGCTGTTGGGTGGCCAACGCCGCTTCATCCTGTC
GAATTCGGGCCACATCCAGTCGATCCTGAACCCGCCGGGCAACCCGAAGGCCTGCTACTT
CGAGAACGACAAGCTGTCGTCGGACCCGCGCGCCTGGTACTACGACGCCAAGCGCGAGG
AGGGCTCATGGTGGCCAGTTTGGTTGGGTTGGCTGCAGGAGCGCTCGGGCGAACTGGGC
AACCCGGACTTCAACCTGGGCTCGGCCGCACATCCACCACTGGAAGCAGCACCGGGCAC
CTACGTGCACATCCGTGGCGGCCACCACCACCATCACCATTGA
Pseudomonas spp. 61-3 phaC1 (1680 bp),
SEQ ID NO: 81
ATGAGTAACAAGAATAGCGATGACTTGAATCGTCAAGCCTCGGAAAACACCTTGGGGCT
TAACCCTGTCATCGGCCTGCGTGGAAAAGATCTGCTGACTTCTGCCCGAATGGTTTTAAC
CCAAGCCATCAAACAACCCATTCACAGCGTCAAGCACGTCGCGCATTTTGGCATCGAGCT
GAAGAACGTGATGTTTGGCAAATCGAAGCTGCAACCGGAAAGCGATGACCGTCGTTTCA
ACGACCCCGCCTGGAGTCAGAACCCACTCTACAAACGTTATCTACAAACCTACCTGGCGT
GGCGCAAGGAACTCCACGACTGGATCGGCAACAGCAAACTGTCCGAACAGGACATCAAT
CGCGCTCACTTCGTGATCACCCTGATGACCGAAGCCATGGCCCCGACCAACAGTGCGGCC
AATCCGGCGGCGGTCAAACGCTTCTTCGAAACCGGCGGTAAAAGCCTGCTCGACGGCCT
CACACATCTGGCCAAGGACCTGGTAAACAACGGCGGCATGCCGAGCCAGGTGGACATGG
GCGCTTTCGAAGTCGGCAAGAGTCTGGGGACGACTGAAGGTGCAGTGGTTTTCCGCAAC
GACGTCCTCGAATTGATCCAGTACCGGCCGACCACCGAACAGGTGCATGAGCGACCGCT
GCTGGTGGTCCCACCGCAGATCAACAAGTTTTATGTGTTTGACCTGAGCCCGGATAAAAG
CCTGGCGCGCTTCTGCCTGAGCAACAACCAGCAAACCTTTATCGTCAGCTGGCGCAACCC
GACCAAGGCCCAGCGTGAGTGGGGTCTGTCGACTTACATCGATGCGCTCAAAGAAGCCG
TCGACGTAGTTTCCGCCATCACCGGCAGCAAAGACATCAACATGCTCGGCGCCTGCTCCG
GTGGCATTACCTGCACCGCGCTGCTGGGTCACTACGCCGCTCTCGGCGAGAAGAAGGTC
AATGCCCTGACCCTTTTGGTCAGCGTGCTCGACACCACCCTCGACTCCCAGGTTGCACTG
TTCGTCGATGAGAAAACCCTGGAAGCTGCCAAGCGTCACTCGTATCAGGCCGGCGTGCT
GGAAGGCCGCGACATGGCCAAAGTCTTCGCCTGGATGCGCCCTAACGACCTGATCTGGA
ACTACTGGGTCAACAACTACCTGCTGGGTAACGAGCCACCGGTCTTCGACATTCTTTTCT
GGAACAACGACACCACCCGGTTGCCTGCTGCGTTCCACGGCGATCTGATCGAAATGTTCA
AAAATAACCCACTGGTGCGCGCCAATGCACTCGAAGTGAGCGGCACGCCGATCGACCTC
AAACAGGTCACTGCCGACATCTACTCCCTGGCCGGCACCAACGATCACATCACGCCCTGG
AAGTCTTGCTACAAGTCGGCGCAACTGTTCGGTGGCAAGGTCGAATTCGTGCTGTCCAGC
AGTGGGCATATCCAGAGCATTCTGAACCCGCCGGGCAATCCGAAATCACGTTACATGAC
CAGCACCGACATGCCAGCCACCGCCAACGAGTGGCAAGAAAACTCAACCAAGCACACCG
ACTCCTGGTGGCTGCACTGGCAGGCCTGGCAGGCCGAGCGCTCGGGCAAACTGAAAAAG
TCCCCGACCAGCCTGGGCAACAAGGCCTATCCGTCAGGAGAAGCCGCGCCGGGCACGTA
TGTGCATGAACGTTAA

In some embodiments of any of the aspects, the amino acid sequence encoded by the functional PHA synthase gene comprises SEQ ID NOs: 7, 8, 82, 83, or an amino acid sequence that is at least 95% (e.g., at least 95%, at least 96%, at least 97%, at least 98%, or at least 99%) identical to the sequence of at least one of SEQ ID NOs: 7, 8, 82, 83 that maintains the same functions as at least one of SEQ ID NOs: 7, 8, 82, 83 (e.g., PHA synthase).

| phaC1 poly(3-hydroxyalkanoic acid) synthase, [Pseudomonas aeruginosa | |
|---|---|
| PAO1], NCBI Reference Sequence: NP_253743.1, 559 aa | |
| SEQ ID NO: 7 | |
| 1 | msqknnnelp kqaaentlnl npvigirgkd lltsarmvll qavrqplhsa rhvahfslel |
| 61 | knvllgqsel rpgdddrrfs dpawsqnply krymqtylaw rkelhswish sdlspqdisr |
| 121 | gqfvinllte amsptnslsn paavkrffet ggkslldglg hlakdlvnng gmpsqvdmda |
| 181 | fevgknlatt egavvfrndv leliqyrpit esvherpllv vppqinkfyv fdlspdksla |
| 241 | rfclrngvqt fivswrnptk sqrewgltty iealkeaiev vlsitgskdl nllgacsggi |
| 301 | ttatlvghyv asgekkvnaf tqlvsvldfe lntqvalfad ektleaakrr syqsgvlegk |
| 361 | dmakvfawmr pndliwnywv nnyllgnqpp afdilywnnd ttrlpaalhg efvelfksnp |
| 421 | lnrpgalevs gtpidlkqvt cdfycvagln dhitpwescy ksarllggkc efilsnsghi |
| 481 | qsilnppgnp karfmtnpel paepkawleq agkhadswwl hwqqwlaers gktrkapasl |
| 541 | gnktypagea apgtyvher |
| phaC2 poly(3-hydroxyalkanoic acid) synthase, Pseudomonas aeruginosa | |
| PAO1, NCBI Reference Sequence: NP_253745.1, 560 aa | |
| SEQ ID NO: 8 | |
| 1 | mrekqesgsv pvpaefmsaq saivglrgkd llttvrslav hglrqplhsa rhlvafggql |
| 61 | gkvllgdtlh qpnpqdarfq dpswrlnpfy rrtlqaylaw qkqllawide snldcddrar |
| 121 | arflvallsd avapsnslin plalkelfnt ggisllngvr hlledivhng gmpsqvnkta |
| 181 | feigrnlatt qgavvfrnev leliqykplg erqyakplli vppqinkyyi fdlspeksfv |
| 241 | qyalknnlqv fviswrnpda qhrewglsty vealdqaiev sreitgsrsv nlagacaggl |
| 301 | tvaallghlq vrrqlrkvss vtylvsllds qmespamlfa deqtlesskr rsyqhgvldg |
| 361 | rdmakvfawm rpndliwnyw vnnyllgrqp pafdilywnn dntrlpaafh gelldlfkhn |
| 421 | pltrpgalev sgtavdlgkv aidsfhvagi tdhitpwdav yrsalllggq rrfilsnsgh |
| 481 | iqsilnppgn pkacyfendk lssdprawyy dakreegsww pvwlgwlqer sgelgnpdfn |
| 541 | igsaahpple aapgtyvhir |

Pseudomonas aeruginosa PAO1 phaC2 (568 aa),
SEQ ID NO: 82
MREKQESGSVPVPAEFMSAQSAIVGLRGKDLLTTVRSLAVHGLRQPLHSARHLVAFGGQLG
KVLLGDTLHQPNPQDARFQDPSWRLNPFYRRTLQAYLAWQKQLLAWIDESNLDCDDRARA
RFLVALLSDAVAPSNSLINPLALKELFNTGGISLLNGVRHLLEDLVHNGGMPSQVNKTAFEIG
RNLATTQGAVVFRNEVLELIQYKPLGERQYAKPLLIVPPQINKYYIFDLSPEKSFVQYALKNN
LQVFVISWRNPDAQHREWGLSTYVEALDQAIEVSREITGSRSVNLAGACAGGLTVAALLGHL
QVRRQLRKVSSVTYLVSLLDSQMESPAMLFADEQTLESSKRRSYQHGVLDGRDMAKVFAW
MRPNDLIWNYWVNNYLLGRQPPAFDILYWNNDNTRLPAAFHGELLDLFKHNPLTRPGALEV
SGTAVDLGKVAIDSFHVAGITDHITPWDAVYRSALLLGGQRRFILSNSGHIQSILNPPGNPKAC
YFENDKLSSDPRAWYYDAKREEGSWWPVWLGWLQERSGELGNPDFNLGSAAHPPLEAAPG
TYVHIRGGHHHHHH
Pseudomonas spp. 61-3 phaC1 (see e.g., Genbank Ref No. GenBank:
BAA36200.1) (559 aa),
SEQ ID NO: 83
MSNKNSDDLNRQASENTLGLNPVIGLRGKDLLTSARMVLTQAIKQPIHSVKHVAHFGIELKN
VMFGKSKLQPESDDRRFNDPAWSQNPLYKRYLQTYLAWRKELHDWIGNSKLSEQDINRAHF
VITLMTEAMAPTNSAANPAAVKRFFETGGKSLLDGLTHLAKDLVNNGGMPSQVDMGAFEV
GKSLGTTEGAVVFRNDVLELIQYRPTTEQVHERPLLVVPPQINKFYVFDLSPDKSLARFCLSN
NQQTFIVSWRNPTKAQREWGLSTYIDALKEAVDVVSAITGSKDINMLGACSGGITCTALLGH
YAALGEKKVNALTLLVSVLDTTLDSQVALFVDEKTLEAAKRHSYQAGVLEGRDMAKVFAW
MRPNDLIWNYWVNNYLLGNEPPVFDILFWNNDTTRLPAAFHGDLIEMFKNNPLVRANALEV
SGTPIDLKQVTADIYSLAGTNDHITPWKSCYKSAQLFGGKVEFVLSSSGHIQSILNPPGNPKSR
YMTSTDMPATANEWQENSTKHTDSWWLHWQAWQAERSGKLKKSPTSLGNKAYPSGEAAP
GTYVHER

In some embodiments of any of the aspects, the engineered bacterium comprises Pseudomonas aeruginosa phaC1 (e.g., SEQ ID NOs: 3, 5, 7) or Pseudomonas aeruginosa phaC2 (e.g., SEQ ID NOs: 4, 6, 8, 82) or Pseudomonas spp. 61-3 phaC1 (e.g., SEQ ID NOs: 81, 83). In some embodiments of any of the aspects, the engineered bacterium comprises Pseudomonas aeruginosa phaC1 (e.g., SEQ ID NOs: 3, 5, 7). In some embodiments of any of the aspects, the engineered bacterium comprises Pseudomonas aeruginosa phaC2 (e.g., SEQ ID NOs: 4, 6, 8, 82). In some embodiments of any of the aspects, the engineered bacterium comprises Pseudomonas spp. 61-3 phaC1 (e.g., SEQ ID NOs: 81, 83). In some embodiments of any of the aspects, the engineered bacterium comprises Pseudomonas aeruginosa phaC1 (e.g., SEQ ID NOs: 3, 5, 7) and Pseudomonas aeruginosa phaC2 (e.g., SEQ ID NOs: 4, 6, 8, 82). In some embodiments of any of the aspects, the engineered bacterium comprises Pseudomonas aeruginosa phaC1 (e.g., SEQ ID NOs: 3, 5, 7) and Pseudomonas spp. 61-3 phaC1 (e.g., SEQ ID NOs: 81, 83). In some embodiments of any of the aspects, the engineered bacterium comprises Pseudomonas aeruginosa phaC2 (e.g., SEQ ID NOs: 4, 6, 8, 82) and Pseudomonas spp. 61-3 phaC1 (e.g., SEQ ID NOs: 81, 83). In some embodiments of any of the aspects, the engineered bacterium comprises Pseudomonas aeruginosa phaC1 (e.g., SEQ ID NOs: 3, 5, 7); Pseudomonas aeruginosa phaC2 (e.g., SEQ ID NOs: 4, 6, 8, 82); and Pseudomonas spp. 61-3 phaC1 (e.g., SEQ ID NOs: 81, 83).

In some embodiments of any of the aspects, the engineered bacterium comprises at least one exogenous copy of at least one functional PHA synthase gene comprising SEQ ID NO: 3, SEQ ID NO: 4, SEQ ID NO: 5, SEQ ID NO: 6, SEQ ID NO: 81, or a nucleic acid sequence that is at least 95% (e.g., at least 95%, at least 96%, at least 97%, at least 98%, or at least 99%) identical to the sequence of at least one of SEQ ID NOs: 3-6 or 81 that maintains the same functions as at least one of SEQ ID NOs: 3-6 or 81 (e.g., PHA synthase).

In some embodiments of any of the aspects, an organism can comprise alternative groups of genes involved in the PHA synthesis pathway. As an example, the Class II PHA synthase operon (e.g., in Pseudomonas oleovorans) comprises phaC1, phaZ, phaC2, and phaD. As another example, the Class III PHA synthase operon (e.g., in Allochromatium vinosum) comprises phaC, phaE, phaA, ORF4, phaP, and phaB. As such, an engineered bacterium can comprise at least one functional heterologous gene involved in the PHA synthesis pathway (e.g., phaC1, phaZ, phaC2, phaD, phaC, phaE, phaA, ORF4, phaP, and/or phaB).

In some embodiments of any of the aspects, an engineered bacterium comprises an engineered inactivating modification and/or an inhibitor of at least one endogenous gene involved in the PHA synthesis pathway (e.g., phaC1, phaZ, phaC2, phaD, phaC, phaE, phaA, ORF4, phaP, and/or phaB), and at least one functional heterologous gene involved in the PHA synthesis pathway (e.g., phaC1, phaZ, phaC2, phaD, phaC, phaE, phaA, ORF4, phaP, and/or phaB). In some embodiments of any of the aspects, the at least one functional heterologous gene involved in the PHA synthesis pathway corresponds to the same enzyme type or enzyme with the same function as the at least one endogenous gene involved in the PHA synthesis pathway.

In some embodiments of any of the aspects, the engineered bacterium comprises at least one exogenous copy of at least one functional thioesterase gene. In some embodiments of any of the aspects, the engineered bacterium does not comprise a functional endogenous thioesterase gene. Thioesterases are enzymes which belong to the esterase family. Esterases, in turn, are one type of the several hydrolases known. Thioesterases exhibit Esterase activity (e.g., splitting of an ester into acid and alcohol, in the presence of water) specifically at a thiol group. Thioesterases or thiolester hydrolases are identified as members of E.C.3.1.2.

Thioesterases (TEs) can determine the chain length of substrate fatty acids, for example in the synthesis of PHAs. As such, TEs can modulate polymer length and ratio or components of the PHA. In some embodiments of any of the aspects, the functional thioesterase gene preferentially produces or leads to the production of medium-chain-length polyhydroxyalkanoate (MCL-PHA), as described herein. As such, the functional thioesterase gene can be selected from any thioesterase gene from any species that preferentially produces or leads to the production of MCL-PHA. In some embodiments of any of the aspects, the functional thioesterase is an Acyl-Acyl Carrier Protein Thioesterase. In some embodiments of any of the aspects, the functional thioesterase gene is heterologous.

In some embodiments of any of the aspects, the functional heterologous thioesterase is from a plant species (e.g., Umbellularia californica, Cuphea palustris). In some embodiments of any of the aspects, the functional heterologous thioesterase gene comprises a Umbellularia californica FatB2 gene (i.e., UcFatB2), a Cuphea palustris FatB1 gene (i.e., CpFatB1), a Cuphea palustris FatB2 gene (i.e., CpFatB2), or a Cuphea palustris FatB2-FatB1 hybrid gene (i.e., CpFatB2-CpFatB1).

In some embodiments of any of the aspects, the engineered bacterium comprises at least one exogenous copy of at least one functional thioesterase gene comprising SEQ ID NO: 9, SEQ ID NO: 10, SEQ ID NO: 11, SEQ ID NO: 12, SEQ ID NO: 13, or a nucleic acid sequence that is at least 95% (e.g., at least 95%, at least 96%, at least 97%, at least 98%, or at least 99%) identical to the sequence of at least one of SEQ ID NOs: 9-13, that maintains the same functions as at least one of SEQ ID NOs: 9-13 (e.g., thioesterase).

| Umbellularia californica FatB2, complete cds, GenBank: U17097.1, 1426 bp | |
|---|---|
| SEQ ID NO: 9 | |
| 1 | aaaaaagtac aaactgtatg gtagccattt acatataact actctataat tttcaacatg |
| 61 | gtcaccacct ctttagcttc cgctttcttc tcgatgaaag ctgtaatgtt ggctcctgat |
| 121 | ggcagtggca taaaacccag gagcagtggt ttgcaggtga gggcgggaaa ggaacaaaac |
| 181 | tcttgcaaga tgatcaatgg gaccaaggtc aaagacacgg agggcttgaa agggcgcagc |
| 241 | acattgcatg gctggagcat gccccttgaa ttgatcacaa ccatcttttc ggctgctgag |
| 301 | aagcagtgga ccaatctagt tagtaagcca ccgcagttgc ttgatgacca tttaggtctg |
| 361 | catgggctag ttttcaggcg cacctttgca atcagatgca gtgaggttgg acctgaccgc |
| 421 | tccacatcca tagtggctgt tatgaattac ttgcaggaag ctgcatgtaa tcatgcggag |
| 481 | agtctgggac ttctaggaga tggattcggt gagacactag agatgagtag gagagatctg |
| 541 | atatgggttg tgagacgcac gcatgttgtt gtggaacggt accctgcttg gggcgatact |
| 601 | gttgaagtcg aggcctggat cggtgcagct ggaaacattg gcatgcgccg ccattttctt |
| 661 | gtccgcgact gcaaaactgg ccacattctt gcaagatgta ccagtgtttc agtgatgatg |
| 721 | aatatgagga caaggagatt gtccaaaatt ccccaagaag ttagagggga gattgaccct |
| 781 | cttttcatcg aaaagtttgc tgtcaaggaa ggggaaatta agaaattaca gaagttcaat |
| 841 | gatagcactg cagattacat tcaagggggt tggactccgc gatggaatga tttggatgtc |
| 901 | aatcagcacg tgaacaatat caaatacgtt ggctggattt ttaagagcgt cccagactct |
| 961 | atctatgaga atcatcatct ttctagcatc actctcgaat acaggagaga gtgcacaagg |
| 1021 | ggcagagcac tgcagtccct gaccactgtt tgtggtggct cgtccgaagc tgggatcata |
| 1081 | tgtgagcacc tactccagct tgaggatggg tctgaggttt tgaggggaag aacagattgg |
| 1141 | aggcccaagc gcaccgatag tttcgaaggc attagtgaga gattcccgca gcaagaaccg |
| 1201 | cataattaat gacagaagca tcagatatag tttctcctgt gctgttcctg agaatgcatc |
| 1261 | ttacaagtcg tggtttggat tgcttgtgca gaatcatggt ttgtgctttc agaagtatat |
| 1321 | ctaaattagt ccaagttata tgactccata ttggaaaata actcaatgag tcgtgctctt |
| 1381 | gaaatggtct tttaagcttt gaaataaagt tccacttaat ccatgt |
| Umbellularia californica FatB2 acyl-ACP thioesterase (942 bp) | |
| SEQ ID NO: 10 | |
| ATGACCAACCTGGAGTGGAAGCCGAAGCCGAAGCTGCCGCAGCTGCTGGACGACCACTT | |
| CGGCCTGCACGGCCTGGTGTTTCGTCGCACCTTCGCCATTCGCTCGTACGAGGTGGGCCC | |
| GGATCGTTCGACCTCGATCCTGGCCGTGATGAACCACATGCAGGAGGCCACCCTGAACC | |
| ACGCCAAGTCGGTGGGCATCCTGGGCGACGGCTTCGGCACTACCCTGGAGATGTCGAAG | |
| CGCGACCTGATGTGGGTGGTGCGCCGCACCCATGTGGCCGTGGAGCGCTATCCGACCTG | |
| GGGTGATACCGTGGAGGTGGAATGCTGGATCGGCGCCTCGGGCAACAACGGCATGCGCC | |
| GCGACTTCCTGGTGCGCGACTGCAAAACCGGCGAGATTCTGACCCGCTGCACCTCGCTGT | |
| CGGTGCTGATGAACACCCGCACCCGCCGCCTGTCGACCATCCCGGATGAAGTGCGCGGC | |
| GAAATTGGCCCGGCCTTCATCGACAACGTGGCCGTGAAGGACGACGAGATCAAGAAGCT | |
| GCAGAAGCTGAACGACTCGACCGCCGACTACATCCAGGGCGGCCTGACCCCGCGCTGGA | |
| ACGACCTGGACGTGAACCAGCACGTGAACAACCTGAAGTACGTGGCCTGGGTGTTCGAG | |
| ACCGTGCCGGACTCGATCTTCGAGTCGCACCACATCTCGTCGTTCACCCTGGAGTACCGC | |
| CGCGAGTGCACCCGCGACTCGGTGCTGCGTTCGCTGACCACCGTGTCAGGTGGCTCATCG | |
| GAAGCAGGCCTGGTGTGCGACCACCTGCTGCAGCTGGAAGGCGGCTCGGAGGTGCTGAG | |
| AGCACGCACTGAATGGCGCCCGAAGCTGACCGACTCGTTTCGCGGCATCTCGGTGATCCC | |
| GGCCGAACCGCGTGTGGGCTCATCAGGCGGCCACCACCATCACCACCACTGA | |
| Cuphea palustris FatB1, GenBank: U38188.1, 1236 bp, complete CDS | |
| SEQ ID NO: 11 | |
| 1 | atggtggctg ctgcagcaag ttctgcatgc ttccctgttc catccccagg agcctcccct |
| 61 | aaacctggga agttaggcaa ctggtcatcg agtttgagcc cttccttgaa gcccaagtca |
| 121 | atccccaatg gcggatttca ggttaaggca aatgccagtg cgcatcctaa ggctaacggt |
| 181 | tctgcagtaa ctctaaagtc tggcagcctc aacactcagg aggacacttt gtcgtcgtcc |
| 241 | cctcctcccc gggctttttt taaccagttg cctgattgga gtatgcttct gactgcaatc |
| 301 | acaaccgtct tcgtggcacc agagaagcgg tggactatgt ttgataggaa atctaagagg |
| 361 | cctaacatgc tcatggactc gtttgggttg gagagagttg ttcaggatgg gctcgtgttc |
| 421 | agacagagtt tttcgattag gtcttatgaa atatgcgctg atcgaacagc ctctatagag |
| 481 | acggtgatga accacgtcca ggaaacatca ctcaatcaat gtaagagtat aggtcttctc |
| 541 | gatgacggct ttggtcgtag tcctgagatg tgtaaaaggg acctcatttg ggtggttaca |
| 601 | agaatgaaga taatggtgaa tcgctatcca acttggggcg atactatcga ggtcagtacc |
| 661 | tggctctctc aatcggggaa aatcggtatg ggtcgcgatt ggctaataag tgattgcaac |
| 721 | acaggagaaa ttcttgtaag agcaacgagt gtgtatgcca tgatgaatca aaagacgaga |
| 781 | agattctcaa aactcccaca cgaggttcgc caggaatttg cgcctcattt tctggactct |
| 841 | cctcctgcca ttgaagacaa cgacggtaaa ttgcagaagt ttgatgtgaa gactggtgat |
| 901 | tccattcgca agggtctaac tccggggtgg tatgacttgg atgtcaatca gcacgtaagc |
| 961 | aacgtgaagt acattgggtg gattctcgag agtatgccaa cagaagtttt ggagactcag |
| 1021 | gagctatgtt ctctcaccct tgaatatagg cgggaatgcg gaagggacag tgtgctggag |
| 1081 | tccgtgacct ctatggatcc ctcaaaagtt ggagaccggt ttcagtaccg gcaccttctg |
| 1141 | cggcttgagg atggggctga tatcatgaag ggaagaactg agtggcggcc gaagaatgca |
| 1201 | ggaactaacg gggcgatatc aacaggaaag acttga |
| Cuphea palustris FatB2, complete CDS, GenBank: U38189.1, 1408 bp | |
| SEQ ID NO: 12 | |
| 1 | ccacgcgtcc gctgagtttg ctggttacca ttttccctgc gaacaaacat ggtggctgcc |
| 61 | gcagcaagtg ctgcattctt ctccgtcgca accccgcgaa caaacatttc gccatcgagc |
| 121 | ttgagcgtcc ccttcaagcc caaatcaaac cacaatggtg gctttcaggt taaggcaaac |
| 181 | gccagtgccc atcctaaggc taacggttct gcagtaagtc taaagtctgg cagcctcgag |
| 241 | actcaggagg acaaaacttc atcgtcgtcc cctcctcctc ggactttcat taaccagttg |
| 301 | cccgtctgga gtatgcttct gtctgcagtc acgactgtct tcggggtggc tgagaagcag |
| 361 | tggccaatgc ttgaccggaa atctaagagg cccgacatgc ttgtggaacc gcttggggtt |
| 421 | gacaggattg tttatgatgg ggttagtttc agacagagtt tttcgattag atcttacgaa |
| 481 | ataggcgctg atcgaacagc ctcgatagag accctgatga acatgttcca ggaaacatct |
| 541 | cttaatcatt gtaagattat cggtcttctc aatgacggct ttggtcgaac tcctgagatg |
| 601 | tgtaagaggg acctcatttg ggtggtcacg aaaatgcaga tcgaggtgaa tcgctatcct |
| 661 | acttggggtg atactataga ggtcaatact tgggtctcag cgtcggggaa acacggtatg |
| 721 | ggtcgagatt ggctgataag tgattgccat acaggagaaa ttcttataag agcaacgagc |
| 781 | gtgtgggcta tgatgaatca aaagacgaga agattgtcga aaattccata tgaggttcga |
| 841 | caggagatag agcctcagtt tgtggactct gctcctgtca ttgtagacga tcgaaaattt |
| 901 | cacaagcttg atttgaagac cggtgattcc atttgcaatg gtctaactcc aaggtggact |
| 961 | gacttggatg tcaatcagca cgttaacaat gtgaaataca tcgggtggat tctccagagt |
| 1021 | gttcccacag aagttttcga gacgcaggag ctatgtggcc tcacccttga gtataggcga |
| 1081 | gaatgcggaa gggacagtgt gctggagtcc gtgaccgcta tggatccatc aaaagaggga |
| 1141 | gaccggtctc tttaccagca ccttctccga ctcgaggacg gggctgatat cgtcaagggg |
| 1201 | agaaccgagt ggcggccgaa gaatgcagga gccaagggag caatattaac cggaaagacc |
| 1261 | tcaaatggaa actctatatc ttagaaggag gaagggacct ttccgagttg tgtgtttatt |
| 1321 | tgctttgctt tgattcactc cattgtataa taatactacg gtcagccgtc tttgtatttg |
| 1381 | ctaagacaaa tagcacagtc attaagtt |

Engineered chimera of C. palustris FatB1 (aa 1-218) and FatB2 (aa 219-316)
thioesterase Chimera 4 (981 bp)
SEQ ID NO: 13
ATGCTGCTGACCGCCATCACGACCGTGTTCGTGGCCCCGGAGAAGCGCTGGACCATGTTC
GACCGCAAGTCGAAGCGCCCGAACATGCTGATGGACTCGTTCGGCCTGGAGCGCGTGGT
GCAGGACGGCCTGGTGTTCCGCCAGTCGTTCTCGATCCGCTCGTACGAGATCTGCGCCGA
CCGCACCGCCTCGATCGAGACCGTGATGAACCACGTGCAGGAGACCTCGCTGAACCAGT
GCAAGTCGATCGGCCTGCTGGACGACGGCTTCGGCCGTTCGCCGGAGATGTGCAAGCGC
GACCTGATCTGGGTGGTGACCCGCATGAAGATCATGGTGAACCGCTACCCGACCTGGGG
CGACACCATCGAGGTGAGCACCTGGCTGTCGCAGTCGGGCAAGATCGGCATGGGCCGCG
ATTGGCTGATCTCGGACTGCAACACCGGCGAGATCCTGGTGCGCGCCACCTCGGTGTACG
CCATGATGAACCAGAAGACCCGCCGCTTCTCGAAGCTGCCGCACGAGGTGCGCCAGGAG
TTCGCCCCGCACTTCCTGGATTCACCACCGGCCATCGAGGACAATGACGGCAAGCTGCAG
AAGTTCGACGTGAAGACCGGCGACTCGATCCGCAAGGGCCTGACCCCGGGCTGGTACGA
CCTGGACGTGAACCAGCACGTGAACAACGTGAAGTACATCGGCTGGATCCTGCAGTCGG
TGCCGACCGAGGTGTTCGAGACCCAGGAGCTGTGCGGCCTGACCCTGGAGTATCGCCGC
GAATGCGGCCGCGATTCGGTGCTGGAATCGGTGACCGCCATGGACCCGTCGAAGGAGGG
CGATCGCTCGCTGTACCAGCACCTGCTGCGCCTGGAAGATGGCGCCGACATCGTGAAGG
GCCGCACCGAATGGCGCCCGAAGAATGCAGGCGCAAAGGGCGCCATTCTGACCGGCAAG
ACCTCAGGCGGCCACCACCACCACCATCATTGA

In some embodiments of any of the aspects, the amino acid sequence encoded by the functional thioesterase gene comprises one of SEQ ID NOs: 14-21, or an amino acid sequence that is at least 95% (e.g., at least 95%, at least 96%, at least 97%, at least 98%, or at least 99%) identical to the sequence of at least one of SEQ ID NOs: 14-21, that maintains the same functions as at least one of SEQ ID NOs: 14-21 (e.g., thioesterase).

In some embodiments of any of the aspects, the amino acid sequence encoded by the functional thioesterase gene comprises SEQ ID NO: 16, SEQ ID NO: 17, SEQ ID NO: 18, SEQ ID NO: 19, SEQ ID NO: 20, SEQ ID NO: 21, or an amino acid sequence that is at least 95% (e.g., at least 95%, at least 96%, at least 97%, at least 98%, or at least 99%) identical to the sequence of at least one of SEQ ID NOs: 16-21, that maintains the same functions as at least one of SEQ ID NOs: 16-21 (e.g., thioesterase).

Umbellularia californica FatB2 GenBank: AAC49001.1, 383 aa
SEQ ID NO: 14
MVTTSLASAFFSMKAVMLAPDGSGIKPRSSGLQVRAGKEQNSCKMINGTKVKDTEGLKGR
STLHGWSMPLELITTIFSAAEKQWTNLVSKPPQLLDDHLGLHGLVFRRTFAIRCSEVGPD
RSTSIVAVMNYLQEAACNHAESLGLLGDGFGETLEMSRRDLIWVVRRTHVVVERYPAWGD
TVEVEAWIGAAGNIGMRRHFLVRDCKTGHILARCTSVSVMMNMRTRRLSKIPQEVRGEID
PLFIEKFAVKEGEIKKLQKFNDSTADYIQGGWTPRWNDLDVNQHVNNIKYVGWIFKSVPD
SIYENHHLSSITLEYRRECTRGRALQSLTTVCGGSSEAGIICEHLLQLEDGSEVLRGRTD
WRPKRTDSFEGISERFPQQEPHN
Umbellularia californica FatB2 acyl-ACP thioesterase (313 aa),
SEQ ID NO: 15
MTNLEWKPKPKLPQLLDDHFGLHGLVFRRTFAIRSYEVGPDRSTSILAVMNHMQEATLNHA
KSVGILGDGFGTTLEMSKRDLMWVVRRTHVAVERYPTWGDTVEVECWIGASGNNGMRRDF
LVRDCKTGEILTRCTSLSVLMNTRTRRLSTIPDEVRGEIGPAFIDNVAVKDDEIKKLQKLNDST
ADYIQGGLTPRWNDLDVNQHVNNLKYVAWVFETVPDSIFESHHISSFTLEYRRECTRDSVLR
SLTTVSGGSSEAGLVCDHLLQLEGGSEVLRARTEWRPKLTDSFRGISVIPAEPRVGSSGGHHH
HHH
Engineered chimera of C. palustris FatB1 (aa 1-218) and FatB2 (aa 219-316)
thioesterase Chimera 4 (326 aa),
SEQ ID NO: 16
MLLTAITTVFVAPEKRWTMFDRKSKRPNMLMDSFGLERVVQDGLVFRQSFSIRSYEICADRT
ASIETVMNHVQETSLNQCKSIGLLDDGFGRSPEMCKRDLIWVVTRMKIMVNRYPTWGDTIEV
STWLSQSGKIGMGRDWLISDCNTGEILVRATSVYAMMNQKTRRFSKLPHEVRQEFAPHFLDS
PPAIEDNDGKLQKFDVKTGDSIRKGLTPGWYDLDVNQHVNNVKYIGWILQSVPTEVFETQEL
CGLTLEYRRECGRDSVLESVTAMDPSKEGDRSLYQHLLRLEDGADIVKGRTEWRPKNAGAK
GAILTGKTSGGHHHHHH
Cuphea palustris FatB1, GenBank: AAC49179.1, 411 aa; bolded text corresponds
to SEQ ID NO: 18 (e.g., residues 96-411 of SEQ ID NO: 17)
SEQ ID NO: 17
MVAAAASSACFPVPSPGASPKPGKLGNWSSSLSPSLKPKSIPNGGFQVKANASAHPKANG
SAVTLKSGSLNTQEDTLSSSPPPRAFFNQLPDWSM LLTAITTVFVAPEKRWTMFDRKSKR
PNMLMDSFGLERVVQDGLVFRQSFSIRSYEICADRTASIETVMNHVQETSLNQCKSIGLL
DDGFGRSPEMCKRDLIWVVTRMKIMVNRYPTWGDTIEVSTWLSQSGKIGMGRDWLIS
DCNTGEILVRATSVYAMMNQKTRRFSKLPHEVRQEFAPHFLDSPPAIEDNDGKLQKFDV
KTGD
SIRKGLTPGWYDLDVNQHVSNVKYIGWILESMPTEVLETQELCSLTLEYRRECGRDSVL
E
SVTSMDPSKVGDRFQYRHLLRLEDGADIMKGRTEWRPKNAGTNGAISTGKT
Cuphea palustris FatB1, fragment, 316 aa, corresponds to bolded text of SEQ ID
NO: 17 (e.g., residues 96-411 of SEQ ID NO: 17); italicized text corresponds to
portion in SEQ ID NO: 21 (e.g., residues 1-218 of SEQ ID NO: 18)
SEQ ID NO: 18
LLTAITTVFVAPEKRWTMFDRKSKRPNMLMDSFGLERWQDGLVFRQSFSIRSYEICADRTASIETV
MNHVQETSLNQCKSIGLLDDGFGRSPEMCKRDLIWWTRMKIMVNRYPTWGDTIEVSTWLSQSGK
IGMGRDWLISDCNTGEILVRATSVYAMMNQKTRRFSKLPHEVRQEFAPHFLDSPPAIEDNDGKLQ
KFDVKTGDSIRKGLTPGWYDLDVNQHVSNVKYIGWILESMPTEVLETQELCSLTLEYRRECGR
DSVLESVTSMDPSKVGDRFQYRHLLRLEDGADIMKGRTEWRPKNAGTNGAISTGKT
Cuphea palustris FatB2, GenBank: AAC49180.1,411 aa; bolded text corresponds
to SEQ ID NO: 20 (e.g., residues 90-404 of SEQ ID NO: 19)
SEQ ID NO: 19
MVAAAASAAFFSVATPRTNISPSSLSVPFKPKSNHNGGFQVKANASAHPKANGSAVSLKS
GSLETQEDKTSSSSPPPRTFINQLPVWSM LLSAVTTVFGVAEKQWPMLDRKSKRPDMLVE
PLGVDRIVYDGVSFRQSFSIRSYEIGADRTASIETLMNMFQETSLNHCKIIGLLNDGFGR
TPEMCKRDLIWVVTKMQIEVNRYPTWGDTIEVNTWVSASGKHGMGRDWLISDCHTGE
ILI
RATSVWAMMNQKTRRLSKIPYEVRQEIEPQFVDSAPVIVDDRKFHKLDLKTGDSICNGL
T
PRWTDLDVNQHVNNVKYIGWILQSVPTEVFETQELCGLTLEYRRECGRDSVLESVTAM
DP
SKEGDRSLYQHLLRLEDGADIVKGRTEWRPKNAGAKGAILTGKT SNGNSIS
Cuphea palustris FatB2, fragment, 315 aa, corresponds to bolded text of SEQ ID
NO: 19 (e.g., residues 90-404 of SEQ ID NO: 19); italicized text corresponds to
portion in SEQ ID NO: 21 (e.g., residues 218-315 of SEQ ID NO: 20)
SEQ ID NO: 20
LLSAVTTVFGVAEKQWPMLDRKSKRPDMLVEPLGVDRIVYDGVSFRQSFSIRSYEIGADRTA
SIETLMNMFQETSLNHCKIIGLLNDGFGRTPEMCKRDLIWVVTKMQIEVNRYPTWGDTIEVN
TWVSASGKHGMGRDWLISDCHTGEILIRATSVWAMMNQKTRRLSKIPYEVRQEIEPQFVDSA
PVIVDDRKFHKLDLKTGDSICNGLTPRWTDLDVNQHVNNVKYIGWILQSVPTEVFETQELCGLT
LEYRRECGRDSVLESVTAMDPSKEGDRSLYQHLLRLEDGADIVKGRTEWRPKNAGAKGAILTGKT
Cuphea palustris FatB2-FatB1 hybrid, 316 aa; bolded text corresponds to italicized
text of SEQ ID NO: 18 (e.g., residues 1-218 of SEQ ID NO: 18); and plain text
corresponds to italicized text of SEQ ID NO: 20 (e.g., residues 218-315 of SEQ
ID NO: 20)
SEQ ID NO: 21
LLTAITTVFVAPEKRWTMFDRKSKRPNMLMDSFGLERVVQDGLVFRQSFSIRSYEICAD
RTASIETVMNHVQETSLNQCKSIGLLDDGFGRSPEMCKRDLIWVVTRMKIMVNRYPTW
GDTIEVSTWLSQSGKIGMGRDWLISDCNTGEILVRATSVYAMMNQKTRRFSKLPHEVR
QEFAPHFLDSPPAIEDNDGKLQKFDVKTGDSIRKGLTPGWYDLDVNQHVNNVKYIGWIL
QSVPTEVFETQELCGLTLEYRRECGRDSVLESVTAMDPSKEGDRSLYQHLLRLEDGADIVKG
RTEWRPKNAGAKGAILTGKT

In some embodiments of any of the aspects, the engineered bacterium comprises a Umbellularia californica FatB2 gene (e.g., SEQ ID NOs: 9, 10, 14, 15), a Cuphea palustris FatB1 gene (e.g., SEQ ID NOs: 11, 17, 18), a Cuphea palustris FatB2 gene (e.g., SEQ ID NOs: 12, 19, 20), or a Cuphea palustris FatB2-FatB1 hybrid gene (e.g., SEQ ID NOs: 13, 16, 21). In some embodiments of any of the aspects, the engineered bacterium comprises a Umbellularia californica UcFatB2 gene (e.g., SEQ ID NO: 9, SEQ ID NO: 10). In some embodiments of any of the aspects, the engineered bacterium comprises a Cuphea palustris FatB1 gene (e.g., SEQ ID NO: 11). In some embodiments of any of the aspects, the engineered bacterium comprises a Cuphea palustris FatB2 gene (e.g., SEQ ID NO: 12). In some embodiments of any of the aspects, the engineered bacterium comprises a Cuphea palustris FatB2-FatB1 hybrid gene (e.g., SEQ ID NO: 13).

In some embodiments of any of the aspects, the engineered bacterium comprises (i) at least one endogenous beta-oxidation gene comprising at least one engineered inactivating modification; and/or (ii) at least one exogenous inhibitor of an endogenous beta-oxidation gene or gene product. In some embodiments of any of the aspects, the engineered bacterium comprises at least one endogenous beta-oxidation gene comprising at least one engineered inactivating modification. In some embodiments of any of the aspects, the engineered bacterium comprises at least one exogenous inhibitor of at least one endogenous beta-oxidation enzyme. In some embodiments of any of the aspects, the engineered bacterium comprises at least one endogenous beta-oxidation gene comprising at least one engineered inactivating modification and an inhibitor of an endogenous beta-oxidation enzyme.

Beta-oxidation is the catabolic process by which fatty acid molecules are broken down to generate acetyl-CoA. Beta-oxidation thus counteracts the formation of PHAs, and as such can be inhibited in order to increase PHA synthesis. Non-limiting examples of enzymes involved in beta oxidation include acyl-CoA ligase (or synthetase), acyl CoA dehydrogenase, enoyl CoA hydratase, 3-hydroxyacyl-CoA dehydrogenase, and β-ketothiolase. In some embodiments of any of the aspects, an engineered bacterium comprises an engineered inactivating modification and/or an inhibitor of an acyl-CoA ligase (or synthetase), acyl CoA dehydrogenase, an enoyl CoA hydratase, a 3-hydroxyacyl-CoA dehydrogenase, and/or a β-ketothiolase.

In some embodiments of any of the aspects, the endogenous beta-oxidation gene is a 3-hydroxyacyl-CoA dehydrogenase (e.g., fadB or a gene with a FadB-like function, e.g., a FadB homolog). 3-hydroxyacyl-CoA dehydrogenase is involved in the aerobic and anaerobic degradation of long-chain fatty acids via beta-oxidation cycle. 3-hydroxyacyl-CoA dehydrogenase catalyzes the formation of 3-oxoacyl-CoA from enoyl-CoA via L−3-hydroxyacyl-CoA. FadB can also use D−3-hydroxyacyl-CoA and cis-3-enoyl-CoA as substrate.

In some embodiments of any of the aspects, the engineered bacterium comprises an engineered inactivating modification of the endogenous Cupriavidus necator 3-hydroxyacyl-CoA dehydrogenase gene. In some embodiments of any of the aspects, the nucleic acid sequence of the endogenous Cupriavidus necator 3-hydroxyacyl-CoA dehydrogenase gene comprises SEQ ID NO: 22 or a sequence that is at least 95% (e.g., at least 95%, at least 96%, at least 97%, at least 98%, or at least 99%) identical to the sequence of SEQ ID NO: 22 that maintains the same functions as SEQ ID NO: 22 (e.g., beta-oxidation, 3-hydroxyacyl-CoA dehydrogenase).

| Cupriavidus necator N-1, 3-hydroxyacyl-CoA dehydrogenase, NCBI Reference | |
|---|---|
| Sequence: NC_015727.1, REGION: complement (968973-971117), 2145 bp | |
| SEQ ID NO: 22 | |
| 1 | atgcaagccc cgattcagta ccacaagacc gacgacggca tcgtcacgct gacgttcgat |
| 61 | gcgcctgagc aaagcgtcaa taccatgacc gatgagatgc ggcaatgtct ggcggacatg |
| 121 | gtgagccggc tggaagcgga gaaggaagcg gttagcggcg tcattcttac ctcggccaag |
| 181 | gagacgttct ttgcgggagg caatctcaat cgcctgtaca agctgcagcc ggcggatgcg |
| 241 | gctacgcagt tcgatgcctc ggagcgtgcc aagtctgcgc tgaggcggct cgaaacgctg |
| 301 | ggcaagccgg tggtggcggc gctcaatggc acggcgctgg gtggcggctt cgaaattgcg |
| 361 | ctggcctgcc accatcgcat tgcgctggac aagcccaaag tgcaattcgg cctgcccgaa |
| 421 | gcgacgctgg gcctgatgcc gggggcgggc ggcgtcgtgc gtctgaatcg gctgctgggg |
| 481 | cttgctgcga gccagcctta tttgcaggac agcaagctca tgtcgccggc agaggcgacc |
| 541 | aaggttgggc tggtgcatga gcttgcggac acacccgcgg cactgctgga gaaggcacga |
| 601 | gcatggatcg cggcccaccc ggaaagcaag cagccgtggg acaaggccgg ctacacgccg |
| 661 | ccgggaggct gggccgatgc gagtgaggcg cggcgctgga tctccacggc cgccgcgcag |
| 721 | gtgcgcgcca agaccaaagg ttgctaccct gcgccggaag ccatcttgtg cgcttcggtc |
| 781 | gaaggcatgc aggtggactt cgacaccgct agccgcattg agacgcgcta cttcgtgaag |
| 841 | cttgtgactg gccaggttgc gaagaacatc atcagcacct tctggttcca cgccaacggc |
| 901 | atcaagtcag gcgcgcagcg tcctgcaggg gtggccaagg gcaagatcaa gacggtgggc |
| 961 | gtgctgggcg cagggatgat gggcaagggg attgcgtatg tggcggcctc gcgtggtatc |
| 1021 | gaggtgtggg tcaaggatgc cacccttgcg caggccgaag gggcacgtgc caatgcggac |
| 1081 | caactgctgg ccaagcgtga ggagaagggg gaaattgatg ccgcgacccg ccgacagatt |
| 1141 | gtcgagcgca ttcacgcgac tgaccgctat gaggactttg cccatgtcga cctggtggtg |
| 1201 | gaagccatcc cagagaaccc tgcgcttaag gcggagatca cccggcaggc cgagcccgtg |
| 1261 | ctcggagatg gggcgatctg ggcctccaac acctcgacgc tgcccatcac cggcctggcc |
| 1321 | aaggcatcga gccggcccga gcgcttcgtc gggctgcact tcttctcgcc ggtgcaccgc |
| 1381 | atgcagttgg tggaagtgat taagggccag cagacctcgc cggagaccct ggcccatgcg |
| 1441 | ctggacttcg tgatgcagct tggcaagacg ccgatcgtcg tcaacgacaa ccgcggcttc |
| 1501 | tttaccagcc gggtattcag tactttcaca cgcgaagcag tggcgatgct gggtgagggg |
| 1561 | caggacccgg ccgccatcga ggcggcggcc atcctgtcag ggttccctgc cgggccgctg |
| 1621 | gcggtgctgg acgaggtcag cttgagcttg aactacaaca accggctcga gacgctcagg |
| 1681 | gcgcatgcgg aggagggtcg tccgctgccg ccacatccgg ccgacgcagt gatggagcgc |
| 1741 | atgctcaatg aattcggccg caaggggcgt gccgcgggtg gcggcttcta cgattatccg |
| 1801 | gccgacggca agaaggtgtt ctggagcggt ctggctaagc acttcctgcg cccggccgaa |
| 1861 | cagattccac agcgtgacaa acaggatcgg ttgttgttct gcatggccct ggagtcggtg |
| 1921 | cgtgtactgc aggatggcgt gctggacagc gcgggggacg gcaacattgg ctcggtactg |
| 1981 | gggattggct tcccgcgctg gagcggcggc gtgttccagt tcctgaacca gtatgggctg |
| 2041 | gaaaaggccg tggcacgtgc ggagtacctg gccgagcatt atggcgaacg gttcacgcca |
| 2101 | ccgcaattgc tacgggaaaa ggccaaacga gccgagccat tctga |

In some embodiments of any of the aspects, the amino acid sequence encoded by the endogenous Cupriavidus necator 3-hydroxyacyl-CoA dehydrogenase gene comprises SEQ ID NO: 23 or an amino acid sequence that is at least 95% (e.g., at least 95%, at least 96%, at least 97%, at least 98%, or at least 99%) identical to the sequence of SEQ ID NO: 23 that maintains the same functions as SEQ ID NO: 23 (e.g., beta-oxidation, 3-hydroxyacyl-CoA dehydrogenase).

| 3-hydroxyacyl-CoA dehydrogenase [Cupriavidus necator], NCBI | |
|---|---|
| Reference Sequence: WP_013959369.1, 714 aa | |
| SEQ ID NO: 23 | |
| 1 | mqapiqyhkt ddgivtltfd apeqsvntmt demrqcladm vsrleaekea vsgviltsak |
| 61 | etffaggnln rlyklqpada atqfdasera ksalrrletl gkpvvaalng talgggfeia |
| 121 | lachhriald kpkvqfglpe atlglmpgag gvvrlnrllg laasqpylqd sklmspaeat |
| 181 | kvglvhelad tpaallckar awiaahpesk qpwdkagytp pggwadasea rrwistaaaq |
| 241 | vraktkgcyp apeailcasv egmqvdfdta srietryfvk lvtgqvakni istfwfhang |
| 301 | iksgaqrpag vakgkiktvg vlgagmmgkg iayvaasrgi evwvkdatla qaegaranad |
| 361 | qllakreekg eidaatrrqi verihatdry edfahvdlvv eaipenpalk aeitrqaepv |
| 421 | lgdgaiwasn tstlpitgla kassrperfv glhffspvhr mqlvevikgq qtspetlaha |
| 481 | ldfvmqlgkt pivvndnrgf ftsrvfstft reavamlgeg qdpaaieaaa ilsgfpagpl |
| 541 | avldevslsl nynnrletlr ahaeegrplp phpadavmer mlnefgrkgr aagggfydyp |
| 601 | adgkkvfwsg lakhfirpae qipqrdkqdr llfcmalesv rvlqdgvlds agdgnigsvl |
| 661 | gigfprwsgg vfqflnqygl ekavaraeyl aehygerftp pqllrekakr aepf |

In some embodiments of any of the aspects, the engineered inactivating modification of an endogenous beta-oxidation gene comprises a deletion of the entire coding sequence (e.g., a knockout of an endogenous fadB gene, denoted herein as ΔfadB).

In some embodiments of any of the aspects, the engineered bacterium comprises an inhibitor of an endogenous beta-oxidation enzyme. In some embodiments of any of the aspects, the inhibitor of an endogenous beta-oxidation enzyme is acrylic acid. In some embodiments of any of the aspects, the inhibitor of an endogenous beta-oxidation enzyme comprises enzymes that catalyze the production of acrylic acid (e.g., malonyl-CoA reductase (MCR), malonate semialdehyde reductase (MSR), 3-hydroxypropionyl-CoA synthetase (3HPCS), and 3-hydroxypropionyl-CoA dehydratase (3HPCD) from Metallosphaera sedula; overexpressed succinyl-CoA synthetase (SCS) from E. coli). In some embodiments of any of the aspects, the engineered bacterium comprises at least one functional exogenous gene that catalyzes the production of acrylic acid (e.g., M. sedula MCR, M. sedula MSR, M. sedula 3HPCS, M. sedula 3HPCD, and/or E. coli SCS). See e.g., Liu and Liu, Production of acrylic acid and propionic acid by constructing a portion of the 3-hydroxypropionate/4-hydroxybutyrate cycle from Metallosphaera sedula in Escherichia coli; J Ind Microbiol Biotechnol. 2016 December, 43(12):1659-1670. Epub 2016 Oct. 8; the content of which is incorporated herein by reference in its entirety.

In some embodiments of any of the aspects, the inhibitor of an endogenous beta-oxidation enzyme is 2-bromooctanoic acid or 4-pentenoic acid; see e.g., Lee et al., Appl Environ Microbiol. 2001 November; 67(11):4963-74. Additional non-limiting examples of beta oxidation inhibitors include an inhibitory RNA (e.g., siRNA, miRNA) against a beta oxidation gene (e.g., FadB, a 3-hydroxyacyl-CoA dehydrogenase gene), a small molecule inhibitor of a beta oxidation gene (e.g., FadB, a 3-hydroxyacyl-CoA dehydrogenase gene), and the like.

Described herein are engineered bacteria and methods associated with the production of bioplastics, for example polyhydroxyalkanoate (PHA). PHAs have the general formula shown below, wherein x can range from 1-8 and n can range from 100-10,000. In a preferred embodiment, an engineered bacterium as described herein (e.g., C. necator) produces medium-chain-length PHAs (MCL-PHA), wherein the R group fatty acid is the longest linear string of carbons 6 to 14 (C6-C14). As used herein, the term “R group fatty acid” refers to the longest linear string of carbons in the PHA molecule (e.g., from the carbon of the carboxylic acid through the end of the R group indicated in Formula I below). In some embodiments of any of the aspects, an engineered bacterium as described herein (e.g., C. necator) produces short-chain-length PHAs, wherein the R group fatty acid is less than 6 carbons long (e.g., PHB comprising a 4 carbon long fatty acid). In some embodiments of any of the aspects, an engineered bacterium as described herein (e.g., C. necator) produces long-chain-length PHAs, wherein the R group fatty acid is greater than 14 carbons long. The use of different thioesterases with preferences for different length fatty acids (e.g., short, medium, or long fatty acids) can result in an engineered bacterium producing tailored PHAs (e.g., short-, medium-, or long-chain length PHAs). A thioesterase with the preferred activity can readily be selected by one of skill in the art, see, e.g., Cantu et al. Protein Science 2020 19:1281-1295; and Zeidman et al. Mol Membr Biol 2009 26:32-41, each of which is incorporated by reference herein in its entirety.

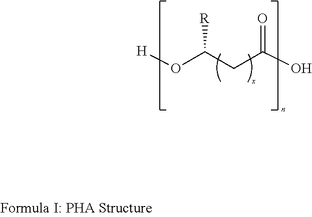

As such, in one aspect described herein is a method of producing medium-chain-length polyhydroxyalkanoate (MCL-PHA), comprising: (a) culturing an engineered bacterium as described herein (e.g., an engineered PHA synthesis bacterium) in a culture medium comprising CO₂ and/or H₂; and (b) isolating, collecting, or concentrating MCL-PHA from said engineered bacterium or from the culture medium of said engineered bacterium.

In some embodiments of any of the aspects, a method of producing medium-chain-length polyhydroxyalkanoate (MCL-PHA), comprises culturing an engineered bacterium as described herein in a culture medium as described herein. As used herein the term “culture medium” refers to a solid, liquid or semi-solid designed to support the growth of microorganisms or cells. In some embodiments of any of the aspects, the culture medium is a liquid. In some embodiments of any of the aspects, the culture medium comprises both the liquid medium and the bacterial cells within it.

In some embodiments of any of the aspects, the culture medium is a minimal medium. As used herein, the term “minimal medium” refers to a cell culture medium in which only few and necessary nutrients are supplied, such as a carbon source, a nitrogen source, salts and trace metals dissolved in water with a buffer. Non-limiting examples of components in a minimal medium include Na₂HPO₄ (e.g., 3.5 g/L), KH₂PO₄ (e.g., 1.5 g/L), (NH₄)₂SO₄ (e.g., 1.0 g/L), MgSO₄.7H₂O (e.g., 80 mg/L), CaSO₄.2H₂O (e.g., 1 mg/L), NiSO₄.7H₂O (e.g., 0.56 mg/L), ferric citrate (e.g., 0.4 mg/L), and NaHCO₃ (200 mg/L). In some embodiments of any of the aspects, a minimal medium can be used to promote lithotrophic growth, e.g., of a chemolithotroph.

In some embodiments of any of the aspects, the culture medium promotes PHA production. As a non-limiting example, nitrogen-limited culture medium can promote PHA production. In some embodiments of any of the aspects, the culture medium comprises a (NH₄)₂SO₄ concentration of at most 0.3 g/L (e.g., at most 0.1 g/L, at most 0.2 g/L, at most 0.3 g/L, at most 0.4 g/L, at most 0.5 g/L). In some embodiments of any of the aspects, the culture medium further comprises an antibiotic, e.g., for selection of engineered bacteria according to at least one selectable marker. Non-limiting examples of selection antibiotics include ampicillin, kanamycin, triclosan, and/or chloramphenicol.

In some embodiments of any of the aspects, the culture medium is a rich medium. As used herein, the term “rich medium” refers to a cell culture medium in which more than just a few and necessary nutrients are supplied, i.e., a non-minimal medium. In some embodiments of any of the aspects, rich culture medium can comprise nutrient broth (e.g., 17.5 g/L), yeast extract (7.5 g/L), and/or (NH₄)₂SO₄ (e.g., 5 g/L). In some embodiments of any of the aspects, a rich medium does necessarily promote lithotrophic growth.

In some embodiments of any of the aspects, the culture medium (e.g., for an engineered PHA synthesis bacterium) comprises CO₂ as the sole carbon source. In some embodiments of any of the aspects, CO₂ is at least 90%, at least 95%, at least 98%, at least 99% or more of the carbon sources present in the culture medium. In some embodiments of any of the aspects, the culture medium comprises CO₂ in the form of bicarbonate (e.g., HCO3⁻, NaHCO₃) and/or dissolved CO₂ (e.g., atmospheric CO₂; e.g., CO₂ provided by a cell culture incubator). In some embodiments of any of the aspects, the culture medium does not comprise organic carbon as a carbon source. Non-limiting example of organic carbon sources include fatty acids, gluconate, acetate, fructose, decanoate; see e.g., Jiang et al. Int J Mol Sci. 2016 July; 17(7): 1157).

In some embodiments of any of the aspects, the culture medium (e.g., for an engineered PHA synthesis bacterium) comprises H₂ as the sole energy source. In some embodiments of any of the aspects, H₂ is at least 90%, at least 95%, at least 98%, at least 99% or more of the energy sources present in the culture medium. In some embodiments of any of the aspects, H₂ is supplied by water-splitting electrodes in the culture medium, as described further herein (see e.g., US Patent Publication 2018/0265898, the contents of which are incorporated herein by reference in their entirety).

In some embodiments of any of the aspects, the culture medium further comprises an inhibitor of an endogenous PHA synthase gene. Non-limiting examples of PHA synthase (e.g., PhaC) inhibitors include carbadethia CoA analogs, sT-CH₂-CoA, sTet-CH₂-CoA, and sT-aldehyde. See e.g., Zhang et al., Chembiochem. 2015 Jan. 2; 16(1): 156-166, the contents of which are incorporated herein in be reference in their entireties. In some embodiments of any of the aspects, the culture medium further comprises an inhibitor of at least one endogenous gene involved in the PHA synthesis pathway. Non-limiting examples of such inhibitors include an inhibitory RNA (e.g., siRNA, miRNA) against a gene involved in PHA synthesis (e.g., a PHA synthase, PhaC, PhaB, PhaA, etc.), a small molecule inhibitor of a gene involved in PHA synthesis (e.g., a PHA synthase, PhaC, PhaB, PhaA, etc.), and the like.

In some embodiments of any of the aspects, the culture medium further comprises a beta-oxidation inhibitor, for example acrylic acid. Additional non-limiting examples of beta oxidation inhibitors include an inhibitory RNA (e.g., siRNA, miRNA) against a beta oxidation gene (e.g., FadB, a 3-hydroxyacyl-CoA dehydrogenase gene), a small molecule inhibitor of a beta oxidation gene (e.g., FadB, a 3-hydroxyacyl-CoA dehydrogenase gene), and the like.

In some embodiments of any of the aspects, a method of producing medium-chain-length polyhydroxyalkanoate (MCL-PHA), comprises isolating, collecting, or concentrating MCL-PHA from an engineered bacterium or from the culture medium of said engineered bacterium, as described herein.

In some embodiments of any of the aspects, the culture medium (e.g., for an engineered PHA bacterium) further comprises arabinose. In some embodiments of any of the aspects, arabinose acts as an inducer for genes in a pBAD vector. In some embodiments of any of the aspects, the culture medium further comprises at least 0.3% arabinose. As a non-limiting example, the culture medium further comprises at least 0.1% arabinose, at least 0.2% arabinose, at least 0.3% arabinose, at least 0.4% arabinose, at least 0.5% arabinose, 0.6% arabinose, at least 0.7% arabinose, at least 0.8% arabinose, at least 0.9% arabinose, or at least 1.0% arabinose.

In some embodiments of any of the aspects, methods described herein comprise isolating, collecting, or concentrating a product (e.g., PHA, MCL-PHA) from an engineered bacterium or from the culture medium of an engineered bacterium. Methods of isolating PHA are well-known in the art. Non-limiting examples of PHA isolation methods include solvent extraction, digestion methods, chemical digestion, enzymatic digestion, mechanical disruption, bead mill disruption, high pressure homogenization, disruption by using ultra-sonication, centrifugation and chemical treatment, supercritical fluid, methods using cell fragility, air classification, dissolved-air flotation, and spontaneous liberation, any of which or any combination of which can be used to isolate PHA. In some embodiments, the sample comprising PHA (e.g., cell cultures) can be pretreated prior to the PHA isolation method, e.g., to improve PHA yield. Non-limiting examples of pretreatments include heat pretreatment, alkaline pretreatment, salt pretreatment, and freezing. See e.g., Jacque et al., Isolation and purification of bacterial poly(3-hydroxyalkanoates), Biochemical Engineering Journal Volume 39, Issue 1, 1 Apr. 2008, Pages 15-27; Arikawa et al., Simple and rapid method for isolation and quantitation of polyhydroxyalkanoate by SDS-sonication treatment, J Biosci Bioeng. 2017 August; 124(2):250-25; the contents of each of which are incorporated herein by reference in their entireties.

As a non-limiting example, in order to isolate PHA, a sample (e.g., cell cultures) can be harvested, pelleted, and/or lyophilized. PHA can be purified from cell pellets with sodium hypochlorite (NaClO) (e.g., 13% NaClO-0.2 ml/mg dry cell weight (DCW) for 4 hr at 30° C.), washed (e.g., twice with deionized water (dH₂O)), washed (e.g., once with acetone), and/or dried (e.g., at 25° C. overnight). The sample can then be dissolved in a solution of methanol and/or HCl (e.g., 1:1 methanol and HCl in dioxane to a final volume of 3 ml with 1% pentadecanoate as internal standard) and incubated (e.g., in oil bath at 90° C. for 20 hr). The sample can then be cooled (e.g., with ice), dissolved in chloroform, and vigorously vortexed. dH₂O can then be added to the sample followed by extensive vortexing. The organic phase can then be separated by centrifugation (10 min, 4,000×g). The organic phase (comprising the purified PHA) can be removed and stored at −20° C. until further analysis. In some embodiments of any of the aspects, the isolated PHA can be analyzed using gas chromatography—mass spectrometry (GC-MS).

In some embodiments of any of the aspects, the isolated PHA comprises MCL-PHA. In some embodiments of any of the aspects, the isolated MCL-PHA comprises an R group fatty acid which is 6 to 14 carbons long (C6-C14). In some embodiments of any of the aspects, the isolated MCL-PHA comprises an R group fatty acid which is 8 to 14 carbons long (C8-C14). In some embodiments of any of the aspects, the isolated MCL-PHA comprises an R group fatty acid which is 10 to 14 carbons long (C10-C14). In some embodiments of any of the aspects, the isolated MCL-PHA comprises an R group fatty acid which is 12 to 14 carbons long (C12-C14).

In some embodiments of any of the aspects, the MCL-PHA produced by the engineered bacterium comprises an R group fatty acid which is 6 to 14 carbons long (C6-C14). In some embodiments of any of the aspects, the MCL-PHA produced by the engineered bacterium comprises an R group fatty acid which is 8 to 14 carbons long (C8-C14). In some embodiments of any of the aspects, the MCL-PHA produced by the engineered bacterium comprises an R group fatty acid which is 10 to 14 carbons long (C10-C14). In some embodiments of any of the aspects, the MCL-PHA produced by the engineered bacterium comprises an R group fatty acid which is 12 to 14 carbons long (C12-C14).

In some embodiments of any of the aspects, the major product of the engineered bacterium is MCL-PHA. In some embodiments of any of the aspects, the isolated PHA comprises a majority of MCL-PHA. In some embodiments of any of the aspects, the total PHA isolated comprises at least 50% MCL-PHA, at least 55% MCL-PHA, at least 60% MCL-PHA, at least 65% MCL-PHA, at least 70% MCL-PHA, at least 75% MCL-PHA, at least 80% MCL-PHA, at least 85% MCL-PHA, at least 90% MCL-PHA, at least 95% MCL-PHA, at least 96% MCL-PHA, at least 97% MCL-PHA, at least 98% MCL-PHA, or least 99% MCL-PHA.

In some embodiments of any of the aspects, the total PHA isolated comprises at least 95% MCL-PHA with an R group fatty acid of C10-C14. In some embodiments of any of the aspects, the total PHA isolated comprises at least 80% MCL-PHA with an R group fatty acid of C12-C14.

In one aspect, described herein is an engineered bacterium comprises one or more of the following: (a) at least one exogenous copy of at least one functional sugar synthesis gene; and/or (b) at least one exogenous copy of at least one functional sugar porin gene. In some embodiments, the engineered bacterium as described above is also referred to herein as an engineered feedstock solution bacterium or an engineered sucrose feedstock solution bacterium.

As used herein, the term “feedstock” refers to one or more raw materials, whether solid, liquid, gas, or any combination thereof. For example, the feedstock can include one or more carbonaceous materials. In some embodiments of any of the aspects, the feedstock comprises a feedstock solution. As used here, the term “feedstock solution” refers to a liquid feedstock comprising an organic carbon source. In some embodiments of any of the aspects, the organic carbon source of the feedstock solution is a sugar, as described further herein. In some embodiments of any of the aspects, the organic carbon source of the feedstock solution is sucrose. In some embodiments of any of the aspects, the liquid feedstock comprises a culture medium as described herein. In some embodiments of any of the aspects, the feedstock solution is produced by an engineered bacterium (e.g., an engineered feedstock solution bacterium) as described herein. In some embodiments of any of the aspects, the feedstock solution is utilized by an engineered heterotroph as described herein.

In some embodiments of any of the aspects, the engineered bacterium comprises (a) at least one exogenous copy of at least one functional sugar synthesis gene or (b) at least one exogenous copy of at least one functional sugar porin gene. In some embodiments of any of the aspects, the engineered bacterium comprises at least one exogenous copy of at least one functional sugar synthesis gene. In some embodiments of any of the aspects, the engineered bacterium comprises at least one exogenous copy of at least one functional sugar porin gene. In some embodiments of any of the aspects, the engineered bacterium comprises (a) at least one exogenous copy of at least one functional sugar synthesis gene and (b) at least one exogenous copy of at least one functional sugar porin gene.

In some embodiments of any of the aspects, the engineered bacterium is a chemoautotroph. In some embodiments of any of the aspects, the engineered bacterium uses CO₂ as its sole carbon source, and/or said engineered bacteria uses H₂ as its sole energy source. In some embodiments of any of the aspects, the engineered bacterium is Cupriavidus necator.

In some embodiments of any of the aspects, the engineered bacterium produces a feedstock solution, using methods as described further herein. In some embodiments of any of the aspects, the feedstock solution comprises a sucrose feedstock solution. In some embodiments of any of the aspects, said the engineered feedstock bacterium is co-cultured with a second microbe (e.g., an engineered heterotroph) that consumes the feedstock solution.

In some embodiments of any of the aspects, the engineered bacterium comprises at least one exogenous copy of at least one functional sugar synthesis gene. In some embodiments of any of the aspects, the at least one functional sugar synthesis gene comprises a glucose synthesis gene, fructose synthesis gene, galactose synthesis gene, lactose synthesis gene, maltose synthesis gene, or sucrose synthesis gene.

In some embodiments of any of the aspects, the at least one functional sugar synthesis gene comprises a sucrose synthesis gene. In some embodiments of any of the aspects, the at least one functional sugar synthesis gene is heterologous. In some embodiments of any of the aspects, the engineered bacterium comprises a sucrose phosphate synthase (SPS) gene or a sucrose phosphate phosphatase (SPP) gene. In some embodiments of any of the aspects, the engineered bacterium comprises a sucrose phosphate synthase (SPS) gene; in some embodiments of any of the aspects, an SPS gene is also referred to as a HAD-IIB family hydrolase. In some embodiments of any of the aspects, the engineered bacterium comprises a sucrose phosphate phosphatase (SPP) gene. In some embodiments of any of the aspects, the engineered bacterium comprises a sucrose phosphate synthase (SPS) gene and a sucrose phosphate phosphatase (SPP) gene.

In some embodiments of any of the aspects, the at least one functional heterologous sucrose synthesis gene comprises Anabaena cylindrica PCC 7122 sucrose phosphate synthase (SPS) or Anabaena cylindrica PCC 7122 sucrose phosphate phosphatase (SPP). In some embodiments of any of the aspects, the at least one functional heterologous sucrose synthesis gene comprises Synechococcus elongatus PCC7942 sucrose phosphate synthase (SPS) or Synechococcus elongatus PCC7942 sucrose phosphate phosphatase (SPP).

In some embodiments of any of the aspects, the at least one functional heterologous sucrose synthesis gene comprises Synechocystis sp. PCC 6803 sucrose phosphate synthase (SPS) or Synechocystis sp. PCC 6803 sucrose phosphate phosphatase (SPP). In some embodiments of any of the aspects, the at least one functional heterologous sucrose synthesis gene comprises Synechocystis sp. PCC 6803 sucrose phosphate synthase (SPS). In some embodiments of any of the aspects, the at least one functional heterologous sucrose synthesis gene comprises Synechocystis sp. PCC 6803 sucrose phosphate phosphatase (SPP). In some embodiments of any of the aspects, the at least one functional heterologous sucrose synthesis gene comprises Synechocystis sp. PCC 6803 sucrose phosphate synthase (SPS) and Synechocystis sp. PCC 6803 sucrose phosphate phosphatase (SPP).

In some embodiments of any of the aspects, the engineered bacterium comprises a functional sucrose phosphate synthase (SPS; e.g., from Synechocystis sp. PCC 6803) gene comprising SEQ ID NO: 28, SEQ ID NO: 29, or a nucleic acid sequence that is at least 95% (e.g., at least 95%, at least 96%, at least 97%, at least 98%, or at least 99%) identical to the sequence of at least one of SEQ ID NOs: 28-29 that maintains the same functions as at least one of SEQ ID NOs: 28-29 (e.g., sucrose phosphate synthase).

Synechocystis sp. IPPAS B-1465 chromosome, complete genome, GenBank:
CP028094.1, reverse complement of REGION: 3141554-3143716, 2163 bp
SEQ ID NO: 28
ATGAGCTATTCATCAAAATACATTTTACTAATTAGTGTCCATGGTTTAATTCGGGGAGAA
AACCTTGAGTTGGGCAGAGATGCCGACACCGGCGGGCAAACCAAATATGTGCTGGAACT
GGCCCGGGCCTTGGTAAAAAATCCCCAGGTGGCCAGGGTGGATTTGCTGACCCGTTTAAT
TAAAGATCCCAAAGTAGATGCAGATTATGCCCAGCCTAGAGAACTCATTGGCGATCGGG
CCCAGATTGTTCGCATTGAGTGCGGCCCGGAGGAATATATTGCCAAGGAAATGCTCTGG
GACTATTTGGATAATTTTGCTGACCATGCCCTGGACTATCTCAAAGAACAGCCCGAACTG
CCCGATGTCATCCATAGCCATTACGCCGATGCGGGTTACGTGGGCACCAGACTTTCTCAC
CAATTGGGTATTCCTTTGGTGCACACCGGACATTCCCTGGGTCGTAGTAAGCGCACCCGT
CTCCTGCTCAGTGGGATTAAAGCCGACGAAATTGAAAGCCGTTACAATATGGCCCGCCG
GATTAACGCGGAGGAAGAAACCCTAGGATCAGCGGCGAGGGTGATTACCAGTACCCATC
AGGAAATCGCAGAACAGTACGCCCAATACGACTATTACCAGCCAGACCAGATGTTGGTT
ATTCCCCCCGGCACTGATTTAGAAAAGTTTTATCCCCCCAAAGGGAACGAGTGGGAAAC
GCCCATTGTTCAAGAGTTGCAACGATTTCTACGGCATCCCCGTAAGCCTATTATCCTCGCT
TTGTCCCGACCGGATCCCCGCAAAAATATCCATAAATTAATTGCAGCCTATGGCCAGTCC
CCGCAGTTACAGGCCCAGGCCAATTTGGTCATTGTGGCGGGCAATCGGGATGACATCAC
GGATCTAGACCAGGGGCCGAGGGAAGTACTGACGGATTTACTGTTGACCATTGACCGTT
ACGATCTCTACGGCAAAGTGGCTTACCCCAAACAGAATCAGGCGGAGGATGTGTATGCT
TTGTTTCGCCTCACTGCTTTATCCCAGGGAGTATTTATCAATCCGGCTTTGACGGAACCCT
TTGGTTTAACTTTGATTGAAGCGGCGGCCTGTGGTGTGCCCATTGTGGCCACGGAGGATG
GGGGCCCGGTGGATATTATCAAAAATTGTCAGAATGGCTATCTAATTAATCCCCTCGATG
AAGTGGATATTGCGGATAAATTGCTCAAAGTACTAAACGACAAACAACAATGGCAATTC
CITTCTGAAAGTGGTCTAGAGGGAGTTAAGCGCCATTATTCTTGGCCTTCCCACGTTGAA
AGTTATTTAGAAGCCATCAACGCTCTGACCCAACAGACTTCAGTGCTGAAACGTAGTGAT
TTAAAGCGGCGGCGGACTTTGTACTATAACGGTGCCCTGGTTACTAGTTTGGACCAAAAT
TTACTGGGGGCATTACAGGGGGGATTACCGGGCGATCGCCAGACGTTGGACGAATTACT
GGAAGTGCTGTATCAACATCGAAAAAATGTCGGCTTTTGCATTGCCACTGGGAGAAGATT
GGATTCGGTGCTGAAAATTTTGCGGGAGTATCGCATTCCCCAACCGGATATGTTGATCAC
CAGCATGGGCACGGAAATTTATTCTTCCCCGGATTTGATCCCCGACCAGAGTTGGCGCAA
TCACATTGATTATTTGTGGAACCGTAACGCCATTGTGCGTATTTTGGGGGAATTACCCGG
TTTAGCCCTCCAACCCAAGGAAGAACTGAGCGCCTATAAAATTAGCTATTTCTACGATGC
GGCGATCGCCCCTAACCTAGAAGAAATTCGGCAACTGTTGCATAAAGGGGAACAAACCG
TAAATACCATCATTTCCTTTGGTCAATTTTTGGATATTCTGCCCATCCGAGCTTCCAAAGG
CTATGCTGTGCGTTGGTTGAGCCAACAGTGGAATATTCCCCTGGAGCACGTTTTCACCGC
CGGAGGATCGGGAGCCGACGAAGATATGATGCGGGGTAACACCCTTTCCGTCGTCGTGG
CTAACCGTCACCATGAGGAACTTTCTAATCTAGGGGAGATCGAACCGATTTATTTTTCCG
AAAAACGTTACGCCGCCGGTATTCTGGACGGTCTGGCCCATTACCGCTTCTTTGAGTTGT
TAGACCCCGTTTAA
Synechocystis sp. PCC 6803 sucrose phosphate synthase (SPS) codon-
optimized (2193 bp)
SEQ ID NO: 29
ATGCACCACCACCACCATCACGGAGGTGGCTCGTCATATTCGTCGAAGTACATCCTGCTG
ATCAGCGTGCACGGCCTGATTCGCGGCGAGAACCTGGAGCTGGGTAGAGATGCAGATAC
TGGTGGTCAAACCAAGTACGTGCTGGAACTGGCCCGCGCACTGGTGAAGAACCCGCAAG
TGGCAAGAGTGGATCTGCTGACACGTCTGATCAAAGACCCGAAGGTGGACGCCGACTAT
GCCCAACCGCGCGAACTGATTGGCGATCGTGCACAGATTGTGCGCATCGAATGTGGCCC
GGAGGAATACATCGCCAAGGAGATGCTGTGGGACTACCTGGACAACTTCGCCGACCATG
CACTGGACTACCTGAAGGAACAGCCGGAACTGCCGGACGTGATCCACTCGCATTATGCC
GATGCCGGCTATGTGGGTACCAGACTGTCGCATCAACTGGGCATCCCGCTGGTGCATACC
GGTCATTCACTGGGCAGATCGAAACGTACCCGTCTGCTGTTGTCGGGCATCAAGGCCGAC
GAGATCGAATCGCGCTACAACATGGCACGCCGCATCAATGCCGAGGAAGAAACCCTGGG
TTCGGCAGCACGCGTTATTACCTCGACCCATCAGGAGATCGCCGAACAGTACGCCCAGTA
CGACTACTACCAGCCGGACCAGATGCTGGTGATTCCACCAGGCACCGACCTGGAGAAGT
TCTATCCGCCGAAGGGCAATGAGTGGGAAACCCCGATCGTGCAAGAACTGCAGCGCTTC
CTGCGTCATCCGAGAAAGCCGATCATCCTGGCACTGTCACGCCCAGATCCGCGTAAGAA
CATCCACAAGCTGATCGCCGCCTATGGCCAATCACCGCAACTGCAAGCACAGGCCAACC
TGGTGATCGTTGCCGGCAATCGTGACGACATCACCGACCTGGATCAAGGCCCAAGAGAA
GTGCTGACCGACCTGCTGCTGACCATTGACCGCTACGACCTGTACGGCAAAGTGGCCTAC
CCGAAGCAGAATCAGGCCGAAGACGTGTACGCCCTGTTTCGTCTGACCGCACTGTCACA
AGGCGTGTTCATCAACCCAGCACTGACCGAGCCGTTTGGCCTGACCCTGATCGAAGCAGC
AGCATGTGGTGTGCCGATTGTTGCAACCGAAGATGGTGGCCCAGTGGACATCATCAAGA
ACTGCCAGAACGGCTACCTGATCAACCCGCTGGACGAGGTGGATATCGCCGACAAGCTG
CTGAAGGTGCTGAACGACAAGCAGCAGTGGCAGTTCCTGTCGGAATCGGGCTTGGAAGG
CGTGAAGCGCCATTACTCATGGCCGTCACACGTGGAGTCGTACCTGGAAGCCATCAACG
CACTGACCCAGCAAACCAGCGTGCTGAAGCGCTCAGACCTGAAAAGACGTCGCACCCTG
TACTACAATGGCGCCCTGGTGACCTCGCTGGACCAGAACCTGTTGGGTGCATTACAAGGC
GGTCTGCCAGGTGATAGACAGACCCTGGACGAGCTGCTGGAGGTGCTGTACCAACACCG
CAAAAATGTGGGCTTCTGCATCGCAACCGGCCGTCGTCTGGATTCGGTGCTGAAGATCCT
GCGCGAGTATAGAATCCCGCAACCGGATATGCTGATCACCTCGATGGGCACCGAGATCT
ACTCGTCGCCGGACCTGATTCCGGATCAATCGTGGCGCAACCATATCGACTACCTGTGGA
ACCGCAACGCCATTGTGCGCATCCTGGGCGAATTGCCTGGTCTGGCACTGCAACCGAAG
GAAGAACTGAGCGCCTACAAGATCTCGTACTTCTACGACGCCGCCATCGCCCCGAACCTG
GAGGAAATCCGTCAACTGCTGCACAAGGGCGAACAAACCGTTAACACCATCATCTCGTT
CGGCCAGTTCCTGGACATCCTGCCGATCCGCGCCTCGAAGGGCTATGCAGTTAGATGGCT
GTCGCAACAGTGGAATATCCCGCTGGAACACGTGTTCACCGCCGGCGGTAGTGGCGCAG
ATGAAGACATGATGCGCGGCAATACTCTGTCAGTTGTGGTGGCAAACCGCCACCACGAG
GAGCTGTCGAATCTGGGCGAAATCGAGCCGATCTACTTCTCGGAAAAGCGCTATGCAGC
CGGCATCCTGGACGGCTTGGCCCATTACCGCTTCTTCGAGCTGTTGGATCCGGTTTGA

In some embodiments of any of the aspects, the amino acid sequence encoded by the functional sucrose phosphate synthase (SPS; e.g., from Synechocystis sp. PCC 6803) gene comprises SEQ ID NO: 30, SEQ ID NO: 88, or an amino acid sequence that is at least 95% (e.g., at least 95%, at least 96%, at least 97%, at least 98%, or at least 99%) identical to the sequence of SEQ ID NO: 30 or SEQ ID NO: 88 that maintains the same functions as SEQ ID NO: 30 or SEQ ID NO: 88 (e.g., sucrose phosphate synthase).

| MULTISPECIES: HAD-IIB family hydrolase [unclassified Synechocystis], | |
|---|---|
| NCBI Reference Sequence: WP_010874006.1, 720 aa, | |
| SEQ ID NO: 30 | |
| 1 | msysskyill isvhglirge nlelgrdadt ggqtkyvlel aralvknpqv arvdlltrli |
| 61 | kdpkvdadya qpreligdra qivriecgpe eyiakemlwd yldnfadhal dylkeqpelp |
| 121 | dvihshyada gyvgtrlshq lgiplvhtgh slgrskrtrl llsgikadei esrynmarri |
| 181 | naeeetlgsa arvitsthqe iaeqyaqydy yqpdqmlvip pgtdlekfyp pkgnewetpi |
| 241 | vqelqrflrh prkpiilals rpdprknihk liaaygqspq lqaqanlviv agnrdditdl |
| 301 | dqgprevltd llltidrydl ygkvaypkqn qaedvyalfr ltalsqgvfi npaltepfgl |
| 361 | tlieaaacgv pivatedggp vdiikncqng ylinpldevd iadkllkvln dkqqwqflse |
| 421 | sglegvkrhy swpshvesyl eainaltqqt svlkrsdlkr rrtlyyngal vtsldqnllg |
| 481 | alqgglpgdr qtldellevl yqhrknvgfc iatgrrldsv lkilreyrip qpdmlitsmg |
| 541 | teiysspdli pdqswrnhid ylwnrnaivr ilgelpglal qpkeelsayk isyfydaaia |
| 601 | pnleeirqll hkgeqtvnti isfgqfldil piraskgyav rwlsqqwnip lehvftaggs |
| 661 | gadedmmrgn tlsvvvanrh heelsnlgei epiyfsekry aagildglah yrffelldpv |

Synechocystis sp. PCC 6803 sucrose phosphate synthase (SPS), 730 aa
SEQ ID NO: 88
MHHHHHHGGGSSYSSKYILLISVHGLIRGENLELGRDADTGGQTKYVLELARALVKNPQVA
RVDLLTRLIKDPKVDADYAQPRELIGDRAQIVRIECGPEEYIAKEMLWDYLDNFADHALDYL
KEQPELPDVIHSHYADAGYVGTRLSHQLGIPLVHTGHSLGRSKRTRLLLSGIKADEIESRYNM
ARRINAEEETLGSAARVITSTHQEIAEQYAQYDYYQPDQMLVIPPGTDLEKFYPPKGNEWETP
IVQELQRFLRHPRKPIILALSRPDPRKNIHKLIAAYGQSPQLQAQANLVIVAGNRDDITDLDQG
PREVLTDLLLTIDRYDLYGKVAYPKQNQAEDVYALFRLTALSQGVFINPALTEPFGLTLIEAA
ACGVPIVATEDGGPVDIIKNCQNGYLINPLDEVDIADKLLKVLNDKQQWQFLSESGLEGVKR
HYSWPSHVESYLEAINALTQQTSVLKRSDLKRRRTLYYNGALVTSLDQNLLGALQGGLPGD
RQTLDELLEVLYQHRKNVGFCIATGRRLDSVLKILREYRIPQPDMLITSMGTEIYSSPDLIPDQS
WRNHIDYLWNRNAIVRILGELPGLALQPKEELSAYKISYFYDAAIAPNLEEIRQLLHKGEQTV
NTIISFGQFLDILPIRASKGYAVRWLSQQWNIPLEHVFTAGGSGADEDMMRGNTLSVVVANR
HHEELSNLGEIEPIYFSEKRYAAGILDGLAHYRFFELLDPV

In some embodiments of any of the aspects, the engineered bacterium comprises a functional sucrose phosphate phosphatase (SPP; e.g., from Synechocystis sp. PCC 6803) gene comprising SEQ ID NO: 31, SEQ ID NO: 32, or a nucleic acid sequence that is at least 95% (e.g., at least 95%, at least 96%, at least 97%, at least 98%, or at least 99%) identical to the sequence of at least one of SEQ ID NOs: 31-32 that maintains the same functions as at least one of SEQ ID NOs: 31-32 (e.g., sucrose phosphatase).

| Synechocystis PCC6803 sucrose-phosphatase (spp) gene, complete cds, | |
|---|---|
| GenBank: AF300455.1, 735 bp, | |
| SEQ ID NO: 31 | |
| 1 | atgcgacagt tattgctaat ttctgacctg gacaatacct gggtcggaga tcaacaagcc |
| 61 | ctggaacatt tgcaagaata tctaggcgat cgccggggaa atttttattt ggcctatgcc |
| 121 | acggggcgtt cctaccattc cgcgagggag ttgcaaaaac aggtgggact catggaaccg |
| 181 | gactattggc tcaccgcggt ggggagtgaa atttaccatc cagaaggcct ggaccaacat |
| 241 | tgggctgatt acctctctga gcattggcaa cgggatatcc tccaggcgat cgccgatggt |
| 301 | tttgaggcct taaaacccca atctcccttg gaacaaaacc catggaaaat tagctatcat |
| 361 | ctcgatcccc aggcttgccc caccgtcatc gaccaattaa cggagatgtt gaaggaaacc |
| 421 | ggcatcccgg tgcaggtgat tttcagcagt ggcaaagatg tggatttatt gccccaacgg |
| 481 | agtaacaaag gtaacgccac ccaatatctg caacaacatt tagccatgga gccgtctcaa |
| 541 | accctggtgt gtggggactc cggcaatgat attggcttat ttgaaacttc cgctcggggt |
| 601 | gtcattgtcc gtaatgccca gccggaatta ttgcactggt atgaccaatg gggggattct |
| 661 | cgtcattatc gggcccaatc gagccatgct ggcgctatcc tagaggcgat cgcccatttc |
| 721 | gattttttga gctga |

Synechocystis sp. PCC 6803 sucrose phosphate phosphatase (SPP)
codon-optimized (765 bp)
SEQ ID NO: 32
ATGCACCACCACCACCATCACGGAGGTGGCTCGAGACAACTGCTGCTGATCAGCGACCT
GGATAACACTTGGGTGGGCGATCAACAGGCCCTGGAGCACCTGCAGGAATATCTGGGCG
ATAGACGCGGCAACTTCTATCTGGCCTATGCAACCGGCCGCTCGTACCATTCGGCAAGAG
AACTGCAGAAGCAGGTGGGCCTGATGGAACCGGATTATTGGCTGACCGCCGTGGGCTCG
GAGATCTATCATCCGGAAGGTCTGGACCAGCATTGGGCCGACTATCTGTCGGAACACTG
GCAGCGCGACATCTTGCAAGCAATCGCAGACGGCTTCGAAGCCCTGAAGCCGCAATCAC
CACTGGAGCAGAACCCGTGGAAGATCTCGTACCATCTGGACCCGCAAGCATGCCCAACC
GTGATCGATCAGCTGACCGAGATGCTGAAGGAAACCGGCATTCCGGTGCAGGTGATCTT
CTCGTCGGGCAAGGATGTGGACCTGCTGCCGCAACGTTCGAATAAGGGCAATGCCACCC
AGTACCTGCAGCAGCATCTGGCCATGGAACCGTCGCAGACCCTGGTGTGTGGTGATTCAG
GCAACGATATTGGCCTGTTCGAGACGTCGGCAAGAGGCGTGATCGTGCGCAACGCACAG
CCAGAACTGCTGCATTGGTATGACCAATGGGGCGATAGCCGCCATTATCGCGCACAGTC
GTCGCATGCAGGCGCAATCCTGGAAGCAATCGCACATTTCGACTTCCTGTCGTAG

In some embodiments of any of the aspects, the amino acid sequence encoded by the functional sucrose phosphate phosphatase (SPP; e.g., from Synechocystis sp. PCC 6803) gene comprises SEQ ID NO: 33, SEQ ID NO: 89, or an amino acid sequence that is at least 95% (e.g., at least 95%, at least 96%, at least 97%, at least 98%, or at least 99%) identical to the sequence of SEQ ID NO: 33 or SEQ ID NO: 89 that maintains the same functions as SEQ ID NO: 33 or SEQ ID NO: 89 (e.g., sucrose phosphatase).

MULTISPECIES: sucrose-phosphate phosphatase
[unclassified Synechocystis], NCBI Reference
Sequence: WP_010873040.1, 244 aa
SEQ ID NO: 33

| | |
|---|---|
| 1 | mrqlllisdl dntwvgdqqa lehlqeylgd rrgnfylaya |
| | tgrsyhsare lqkqvglmep |
| 61 | dywitavgse iyhpegldqh wadylsehwq rdilqaiadg |
| | fealkpqspl eqnpwkisyh |
| 121 | ldpqacptvi dqltemlket gipvqvifss gkdvdllpqr |
| | snkgnatqyl qqhlamepsq |
| 181 | tlvcgdsgnd iglfetsarg vivrnaqpel lhwydqwgds |
| | rhyraqssha gaileaiahf |
| 241 | dfls |

Synechocystis sp. PCC 6803 sucrose phosphate
phosphatase (SPP) (254 aa)
SEQ ID NO: 89
MHHHHHHGGGSRQLLLISDLDNTWVGDQQALEHLQEYLGDRRGNFYLAYA
TGRSYHSARELQKQVGLMEPDYWLTAVGSEIYHPEGLDQHWADYLSEHWQ
RDILQAIADGFEALKPQSPLEQNPWKISYHLDPQACPTVIDQLTEMLKET
GIPVQVIFSSGKDVDLLPQRSNKGNATQYLQQHLAMEPSQTLVCGDSGND
IGLFETSARGVIVRNAQPELLHWYDQWGDSRHYRAQSSHAGAILEAIAHF
DFLS

In some embodiments of any of the aspects, the engineered bacterium comprises a functional sucrose phosphate synthase (SPS; e.g., from Anabaena cylindrica PCC 7122) gene comprising SEQ ID NO: 73, or a nucleic acid sequence that is at least 95% (e.g., at least 95%, at least 96%, at least 97%, at least 98%, or at least 99%) identical to the sequence of SEQ ID NOs: 73 that maintains the same functions as SEQ ID NO: 73 (e.g., sucrose phosphate synthase).

Anabaena cylindrica PCC 7122 DNA, nearly
complete genome, GenBank: AP018166.1,
region: 2004268-2006469 (complement),
2202 bp
SEQ ID NO: 73
ATGTCAAACAGCCAAGGGTTGTACATTTTGCTGGTTAGTGTTCACGGTTT
AATTAGAGGTCATAATCTAGAATTAGGAAGAGATGCCGACACTGGTGGAC
AAACAAAATATGCAGTCGAACTTGCCACCACATTAGCCAAAAATCCTCAA
GTAGAAAGAGTGGATTTAGTTACTCGGTTGGTGAATGATCCAAAAGTTAG
TCCTGACTATGCTCAACCAATAGAAATTCTCTCAGATAAAGCTCAGATCA
TTCGTCTTGCTTGCGGGCCACGTCGCTATCTCCGCAAAGAAGTTCTCTGG
CAGCATTTAGATACCTTTGCAGATGAATTGCTCAGACACATTCGTAAAGT
TGGCAGAATACCAAATGTAATTCATACACACTACGCTGATGCAGGATATG
TTGGCAGTCGGGTTGCAGGTTGGTTAGGAACACCTCTTGTACATACTGGT
CACTCCCTGGGACGGGTTAAACAGCAAAAATTATTGGAACAGGGAACTAA
ACAGGAAGTGATTGAAGATCATTTTCATATTAGTACAAGAATTGAAGCAG
AAGAAATTACACTTGGTGGTGCAGCTTTAGTTATAGCCAGCACTAATCAA
GAAGTTGAGCAGCAGTATAGTGTGTACGATCGCTATCAACCAGAAAGAAT
GGTGGTGATTCCTCCTGGTGTAGACTTGGATCGATTTTACCTACCTGGAG
ATGATTGGCACAATCCACCGATTCAAAAAGAATTGGATCGATTTCTCAAA
GATCCGCAAAAGCCAATCATCATGGCAATTTCCCGTCCAGCTATTCGTAA
AAACGTCAGTAGTCTGATTAAGGCTTATGGTGAAGATCCTGAGTTGCGGA
AACTGGCAAACCTAGTCATAGTCCTGGGCAAGCGGGACGACATCATGACG
ATGGAATCGGGGCCACGTCAGGTATTTATAGAGATATTGCAATTAATAGA
TCGCTACGACCTCTACGGTCACATTGCTTATCCTAAACACCATAATGCTG
ACGATGTGCCAGATTTATATCGGCTGACAGCTAGAACACAAGGGGTATTC
ATTAATCCTGCCTTGACAGAACCATTTGGACTTACCTTAATTGAAGCGAG
TGCTTGCGGTGTACCTATTATTGCCACTGCTGACGGTGGCCCACGGGATA
TTTTGGCAGCTTGTGAGAATGGATTGCTAATTGATCCCTTAAATATTCAA
GAAATACAAAATGCTCTCCGCAAAGCCTTAACAGATAAGGAACAATGGCA
AAACTGGTCTAGCAATGGTTTAGTTAATGTTCGCAAATATTTCTCCTGGA
ATAGTCATGTAGAGAAATATTTAGAGAAAATACACCTATTTCCCCAACGA
CGAATTCAATCTCTACTTAGTCCTTTGCCGGCATCTCCTGCGACTGATCA
TCCAGAGTGGAATGTGCCAGATACCAACCGTTTACCTACCGCTGATCGTT
TCCTAGTTTGTGAAATTGATAATACGCTGCTGGGTGACAAAGAAGCCCTA
GAAAAATTGATTCAGCGCATTCGTAACGAAGGACATACAACTGGGGTTGG
TATTGCCACAGGTCGCACTCTAGAAAGTACTTTGAGTATGTTGGAAGAAT
GGCGATTCCCGATGCCAGATTTGTTAATTACATCAGCAGGTAGTGAAATT
TACTACGGGCCGCAGATAGTTACAGATACAAGTTGGCAAAAACACATTGG
TTACCAATGGCAAGCTGAAGCAATTCGGGCAGCAATGAAGAATATTCCCG
GTGTAGAATTGCAACCAGAAGAAGCTCAACGCAAGTTTAAGGTTAGCTAT
TTTGTTGATGAAGCTAAAGCGCCTAACTTCCGAGAAATTATCCGCCATTT
GCGTCGTCATCAACTACCTGTAAAAGGGATTTACAGCCACAATATGTATT
TGGATTTAGTTCCTATCCGGGCTTCTAAGGGCGATGCAATTCGCTATGTG
GCTTTGAAATGGGGTTTACCTGTTCAACGTTTCCTGGTAGCAGGGGCATC
AGGTAATGATGAAACCATGCTTGGTGGTAACACTTTAGGGGTTGTGGTGG
GGAATTACAGCCAAGAAATTGAAAAGCTGCGGGGTTATCCACAGATTTAT
TTTGCTCAAGGTAATTATGCCTGGGGTATTTTGGAAGCCTTGGATTATTA
CGACTTTTTTGGTAATCTGTCTCAAAAACAGCCAGAAATGGTGACGGTTT
AG,

In some embodiments of any of the aspects, the amino acid sequence encoded by the functional sucrose phosphate synthase (SPS; e.g., from Anabaena cylindrica PCC 7122) gene comprises SEQ ID NO: 74, or an amino acid sequence that is at least 95% (e.g., at least 95%, at least 96%, at least 97%, at least 98%, or at least 99%) identical to the sequence of SEQ ID NO: 74 that maintains the same functions as SEQ ID NO: 74 (e.g., sucrose phosphate synthase).

HAD-IIB family hydrolase [Anabaena
cylindrica], NCBI Reference Sequence:
WP_015217096.1, 733 aa
SEQ ID NO: 74

| | |
|---|---|
| 1 | msnsqglyil lvsvhglirg hnlelgrdad tggqtkyave |
| | lattlaknpq vervdlvtrl |
| 61 | vndpkvspdy aqpieilsdk aqiirlacgp rrylrkevlw |
| | qhldtfadel lrhirkvgri |
| 121 | pnvihthyad agyvgsrvag wlgtplvhtg hslgrvkqqk |
| | lleqgtkqev iedhfhistr |
| 181 | ieaeeitlgg aalviastnq eveqqysvyd ryqpermvvi |
| | ppgvdldrfy lpgddwhnpp |
| 241 | iqkeldrflk dpqkpiimai srpairknvs slikaygedp |
| | elrklanlvi vlgkrddimt |
| 301 | mesgprqvfi eilqlidryd lyghiaypkh hnaddvpdly |
| | ritartqgvf inpaltepfg |
| 361 | ltlieasacg vpiiatadgg prdilaacen gllidplniq |
| | eiqnalrkal tdkeqwqnws |
| 421 | snglvnvrky fswnshveky lekihlfpqr riqsllsplp |
| | aspatdhpew nvpdtnrlpt |
| 481 | adrflvceid ntligdkeal ekliqrirne ghttgvgiat |
| | grtlestlsm leewrfpmpd |
| 541 | llitsagsei yygpqivtdt swqkhigyqw qaeairaamk |
| | nipgvelqpe eaqrkfkvsy |
| 601 | fvdeakapnf reiirhlrrh qipvkgiysh nmyldivpir |
| | askgdairyv alkwglpvqr |
| 661 | flvagasgnd etmlggntlg vvvgnysqei eklrgypqiy |
| | faqgnyawgi lealdyydff |
| 721 | gnlsqkqpem vtv, |

In some embodiments of any of the aspects, the engineered bacterium comprises a functional sucrose phosphate phosphatase (SPP; e.g., from Anabaena cylindrica PCC 7122) gene comprising SEQ ID NO: 75, or a nucleic acid sequence that is at least 95% (e.g., at least 95%, at least 96%, at least 97%, at least 98%, or at least 99%) identical to the sequence of SEQ ID NO: 75 that maintains the same functions as SEQ ID NO: 75 (e.g., sucrose phosphatase).

Anabaena cylindrica PCC 7122 DNA, nearly
complete genome, GenBank: AP018166.1,
REGION: 2248244-2249029, 786 bp
SEQ ID NO: 75

| | |
|---|---|
| 1 | atgaaagcat ttctcttcgt tactgattta gatgacacgc |
| | tagtaggtga taaaaaatct |
| 61 | ttaaactttt tagagcaatt gaacgaagaa ttaataaatc |
| | atcgcaagac atatggaact |
| 121 | aaaattgttt atgccacggg gcgatctcta actttatacc |
| | agcaactgat aaacacacaa |
| 181 | gaacttttag aacctgatgc tttaattact gctgtaggaa |
| | ctgaaatata cttcaactct |
| 241 | aattacaaaa ctcccgactt aaaatggtct agtaagttat |
| | caattggctg gaatcgtgat |
| 301 | ttggtagaga aaatagccgc aaattttgaa gacttagttc |
| | ttcaaaaagc atctgaacaa |
| 361 | cttcctttta aagttagtta tcttgttaag gaagaagctg |
| | ctaaaaaagt cataccccaa |
| 421 | ctcgataatt tattaaaaaa ggaaaacatt aaatttaatt |
| | taatctacag tggaaacttt |
| 481 | agtggcagcg aaaacctgga aagcaaaaac ctggatattt |
| | tgccttttga taccgataaa |
| 541 | ggtttagcaa tgaagtattt acaaaatgaa tggggacatt |
| | ctgctcacga aacagttgtt |
| 601 | tgtggtgatt caggtaatga tattgcctta ttcagcagag |
| | gagaagaaag aggtattatt |
| 661 | gtaggaaatg cgctttctga attacgggaa tggtatacag |
| | caaacaaaac cgactatcgc |
| 721 | tatttagcaa aatcatttta tgctgaaggt attttagaag |
| | gtttgcgtca ttttcagttt |
| 781 | atttaa, |

In some embodiments of any of the aspects, the amino acid sequence encoded by the functional sucrose phosphate phosphatase (SPP; e.g., from Anabaena cylindrica PCC 7122) gene comprises SEQ ID NO: 76, or an amino acid sequence that is at least 95% (e.g., at least 95%, at least 96%, at least 97%, at least 98%, or at least 99%) identical to the sequence of SEQ ID NO: 76 that maintains the same functions as SEQ ID NO: 76 (e.g., sucrose phosphatase).

sucrose-phosphate phosphatase [Anabaena
cylindrica], NCBI Reference Sequence:
WP_015216897.1, 261 aa
SEQ ID NO: 76

| | |
|---|---|
| 1 | mkaflfvtdl ddtivgdkks lnfleqlnee linhrktygt |
| | kivyatgrsl tlyqqlintq |
| 61 | ellepdalit avgteiyfhs nyktpdlkws sklsigwnrd |
| | lvekiaanfe dlvlqkaseq |
| 121 | lpfkvsylvk eeaakkvipq ldnllkkeni kfhliysgnf |
| | sgsenleskn ldilpfdtdk |
| 181 | glamkylqne wghsahetvv cgdsgndial fsrgeergii |
| | vgnalselre wytanktdyr |
| 241 | ylaksfyaeg ileglrhfqf i, |

Synechococcus elongatus PCC7942 is reported to express a fusion enzyme that catalyzes the SPS and SPP reactions by a single protein (see e.g., Qiao et al., Effects of Reduced and Enhanced Glycogen Pools on Salt-Induced Sucrose Production in a Sucrose-Secreting Strain of Synechococcus elongatus PCC 7942, Appl Environ Microbiol. 2018 Jan. 2, 84(2). pii: e02023-17; De la Rosa, First evidence of sucrose biosynthesis by single cyanobacterial bimodular proteins, FEBS Lett. 2013 Jun. 5, 587(11): 1669-74).

Accordingly, in some embodiments of any of the aspects, the engineered bacterium comprises a functional sucrose phosphate synthase/sucrose-phosphate phosphatase (SPS/SPP; e.g., from Synechococcus elongatus PCC7942) gene comprising SEQ ID NO: 77, or a nucleic acid sequence that is at least 95% (e.g., at least 95%, at least 96%, at least 97%, at least 98%, or at least 99%) identical to the sequence of SEQ ID NO: 77 that maintains the same functions as SEQ ID NO: 77 (e.g., sucrose phosphate synthase and/or sucrose phosphatase).

Synechococcus elongatus PCC 7942, complete
genome, GenBank: CP000100.1, REGION:
800851-802980 (complement), 2130 bp
SEQ ID NO: 77
GTGGCAGCTCAAAATCTCTACATTCTGCACATTCAGACCCATGGTCTGCT
GCGAGGGCAGAACTTGGAACTGGGGCGAGATGCCGACACCGGCGGGCAGA
CCAAGTACGTCTTAGAACTGGCTCAAGCCCAAGCTAAATCCCCACAAGTC
CAACAAGTCGACATCATCACCCGCCAAATCACCGACCCCCGCGTCAGTGT
TGGTTACAGTCAGGCGATCGAACCCTTTGCGCCCAAAGGTCGGATTGTCC
GTTTGCCTTTTGGCCCCAAACGCTACCTCCGTAAAGAGCTGCTTTGGCCC
CATCTCTACACCTTTGCGGATGCAATTCTCCAATATCTGGCTCAGCAAAA
GCGCACCCCGACTTGGATTCAGGCCCACTATGCTGATGCTGGCCAAGTGG
GATCACTGCTGAGTCGCTGGTTGAATGTACCGCTAATTTTCACAGGGCAT
TCTCTGGGGCGGATCAAGCTAAAAAAGCTGTTGGAGCAAGACTGGCCGCT
TGAGGAAATTGAAGCGCAATTCAATATTCAACAGCGAATTGATGCGGAGG
AGATGACGCTCACTCATGCTGACTGGATTGTCGCCAGCACTCAGCAGGAA
GTGGAGGAGCAATACCGCGTTTACGATCGCTACAACCCAGAGCGCAAGCT
TGTCATTCCACCGGGTGTCGATACCGATCGCTTCAGGTTTCAGCCCTTGG
GCGATCGCGGTGTTGTTCTCCAACAGGAACTGAGCCGCTTTCTGCGCGAC
CCAGAAAAACCTCAAATTCTCTGCCTCTGTCGCCCCGCACCTCGCAAAAA
TGTACCGGCGCTGGTGCGAGCCTTTGGCGAACATCCTTGGCTGCGCAAAA
AAGCCAACCTTGTCTTAGTACTGGGCAGCCGCCAAGACATCAACCAGATG
GATCGCGGCAGTCGGCAGGTGTTCCAAGAGATTTTCCATCTGGTCGATCG
CTACGACCTCTACGGCAGCGTCGCCTATCCCAAACAGCATCAGGCTGATG
ATGTGCCGGAGTTCTATCGCCTAGCGGCTCATTCCGGCGGGGTATTCGTC
AATCCGGCGCTGACCGAACCTTTTGGTTTGACAATTTTGGAGGCAGGAAG
CTGCGGCGTGCCGGTGGTGGCAACCCATGATGGCGGCCCCCAGGAAATTC
TCAAACACTGTGATTTCGGCACTTTAGTTGATGTCAGCCGACCCGCTAAT
ATCGCGACTGCACTCGCCACCCTGCTGAGCGATCGCGATCTTTGGCAGTG
CTATCACCGCAATGGCATTGAAAAAGTTCCCGCCCATTACAGCTGGGATC
AACATGTCAATACCCTGTTTGAGCGCATGGAAACGGTGGCTTTGCCTCGT
CGTCGTGCTGTCAGTTTCGTACGGAGTCGCAAACGCTTGATTGATGCCAA
ACGCCTTGTCGTTAGTGACATCGACAACACACTGTTGGGCGATCGTCAAG
GACTCGAGAATTTAATGACCTATCTCGATCAGTATCGCGATCATTTTGCC
TTTGGAATTGCCACGGGGCGTCGCCTAGACTCTGCCCAAGAAGTCTTGAA
AGAGTGGGGCGTTCCTTCGCCAAACTTCTGGGTGACTTCCGTCGGCAGCG
AGATTCACTATGGCACCGATGCTGAACCGGATATCAGCTGGGAAAAGCAT
ATCAATCGCAACTGGAATCCTCAGCGAATTCGGGCAGTAATGGCACAACT
ACCCTTTCTTGAACTGCAGCCGGAAGAGGATCAAACACCCTTCAAAGTCA
GCTTCTTTGTCCGCGATCGCCACGAGACTGTGCTGCGAGAAGTACGGCAA
CATCTTCGCCGCCATCGCCTGCGGCTGAAGTCAATCTATTCCCATCAGGA
GTTTCTTGACATTCTGCCGCTAGCTGCCTCGAAAGGGGATGCGATTCGCC
ACCTCTCACTCCGCTGGCGGATTCCTCTTGAGAACATTTTGGTGGCAGGC
GATTCTGGTAACGATGAGGAAATGCTCAAGGGCCATAATCTCGGCGTTGT
AGTTGGCAATTACTCACCGGAATTGGAGCCACTGCGCAGCTACGAGCGCG
TCTATTTTGCTGAGGGCCACTATGCTAATGGCATTCTGGAAGCCTTAAAA
CACTATCGCTTTTTTGAGGCGATCGCTTAA,

In some embodiments of any of the aspects, the amino acid sequence encoded by the functional sucrose phosphate synthase/sucrose-phosphate phosphatase (SPS/SPP; e.g., from Synechococcus elongatus PCC7942) gene comprises SEQ ID NO: 78, or an amino acid sequence that is at least 95% (e.g., at least 95%, at least 96%, at least 97%, at least 98%, or at least 99%) identical to the sequence of SEQ ID NO: 78 that maintains the same functions as SEQ ID NO: 78 (e.g., sucrose phosphate synthase and/or sucrose phosphatase).

MULTISPECIES: HAD-IIB family hydrolase
[Synechococcus], NCBI Reference Sequence:
WP_011377738.1, 709 aa
SEQ ID NO: 78

| | |
|---|---|
| 1 | maaqnlyilh iqthgllrgq nlelgrdadt ggqtkyvlel |
| | aqaqakspqv qqvdiitrqi |
| 61 | tdprvsvgys qaiepfapkg rivrlpfgpk rylrkellwp |
| | hlytfadail qylaqqkrtp |
| 121 | twiqahyada gqvgsllsrw lnvpliftgh slgriklkkl |
| | leqdwpleei eaqfniqqri |
| 181 | daeemtltha dwivastqqe veeqyrvydr ynperklvip |
| | pgvdtdrfrf qplgdrgvvl |
| 241 | qqelsrflrd pekpqilclc rpaprknvpa ivrafgehpw |
| | irkkanlvlv lgsrqdinqm |
| 301 | drgsrqvfqe ifhlvdrydl ygsvaypkqh qaddvpefyr |
| | laahsggvfv npaltepfgl |
| 361 | tileagscgv pvvathdggp qeilkhcdfg tlvdvsrpan |
| | iatalatlls drdlwqcyhr |
| 421 | ngiekvpahy swdqhvntlf ermetvalpr rravsfvrsr |
| | krlidakrlv vsdidntllg |
| 481 | drqglenlmt yldqyrdhfa fgiatgrrld saqevlkewg |
| | vpspnfwvts vgseihygtd |
| 541 | aepdiswekh inrnwnpqri ravmaqipfl elqpeedqtp |
| | fkvsffvrdr hetvlrevrq |
| 601 | hlrrhrlrlk siyshqefld ilplaaskgd airhlslrwr |
| | iplenilvag dsgndeemlk |
| 661 | ghnlgvvvgn yspelepirs yervyfaegh yangilealk |
| | hyrffeaia, |

In some embodiments of any of the aspects, the engineered bacterium comprises at least one exogenous copy of at least one functional sugar porin gene. In some embodiments of any of the aspects, the at least one functional sugar porin gene comprises a glucose porin gene, fructose porin gene, galactose porin gene, lactose porin gene, maltose porin gene, or sucrose porin gene. In some embodiments of any of the aspects, the at least one functional sugar porin gene is heterologous. In some embodiments of any of the aspects, the functional sugar porin gene is a functional sucrose porin gene. In some embodiments of any of the aspects, the functional heterologous sucrose porin gene comprises E. coli sucrose porin (scrY).

In some embodiments of any of the aspects, the engineered bacterium comprises a functional sucrose porin gene comprising SEQ ID NO: 34, SEQ ID NO: 35, or a nucleic acid sequence that is at least 95% (e.g., at least 95%, at least 96%, at least 97%, at least 98%, or at least 99%) identical to the sequence of at least one of SEQ ID NOs: 34-35 that maintains the same functions as at least one of SEQ ID NOs: 34-35 (e.g., sucrose porin).

Escherichia coli ygcF gene, sucrose operon
(scrKYABR genes) and ygcE gene (partial),
strain T19, GenBank: AJ639630.1, REGION:
4904-6421 (complement), 1518 bp
SEQ ID NO: 34
ATGTATAAAAAAACAACTTTGGCAGTGTTAATTGCTTTGCTGACCGGTGC
TACAACGGTACATGCGCAAACGGATATTAGCAGTATCGAATCTCGACTGG
CGGCATTGGAACAACGTTTAAAAAATGCGGAATCCCGCGCCCAGGCGGCA
GAAGCAAGGGCCAAAACAGCTGAATTACAGGTTCAGAAACTGGCTGAAAC
ACAACAACAAAATCAGCTAACAACTCAAGAAGTAGCACAGAGAACAGTTC
AGCTCGAACAGAAATCCGCAGAAAACAGTGGTTTTGAGTTTCATGGCTAT
GCCCGTTCCGGGTTACTGATGAATGATGCCGGTTCCAGTAGCAAAAGTGG
GCCGTATCTGACTCCCGCAGGTGAAACTGGTGGAGCTGTTGGCCGTCTGG
GAAAAGAAGCCGATACCTATGTCGAGTTAAATGTAGAACATAAACAAACA
CTGGATAACGGTGCGACCACACGCTTTAAAGCAATGTTGGCTGACGGACA
AAGAGATTACAACGACTGGACTGGCGGCTCCAGTAACCTGAATATCCGAC
AGGCTTTTGCCGAACTGGGCGCATTACCAAGTTTTACCGGAGCATTCCAA
GACAGTACTGTCTGGGCTGGAAAACGCTTTGATCGCGACAATTTTGATAT
TCACTGGTTAGACTCCGATGTCGTATTTTTAGCGGGAACGGGCGGCGGTA
TCTATGACGTAAAATGGAACGATACATTCCGCAGTAACTTTTCTCTCTAC
GGACGTAATTTCGGCGATCTTGATGATATCGACAATAACGTTCAGAACTA
CATCCTCACCATGAATCATTATGCAGGCCCCTTCCAGTTGATGGTTAGCG
GATTAGGGGCAAAAGATAATGATGATCGAAAAGATGGCAATGGTGATCTC
ATTCAAACTGATGCTGCAAATACTGGCGTACATGCGTTAGTTGGTCTGCA
CAATGACACTTTCTATGGCCTGCGTGAAGGGACGGCAAAAACAGCACTGC
TATATGGCCATGGCCTGGGTGCGGAAGTCAAAGGGATTGGCTCCGATGGC
GCTCTGCTGTCTGAGGCGAATACCTGGCGCTTCGCATCTTACGGCACAAC
ACCTCTGGGAAGCGGTTGGTATGTTGCGCCAGCAATTCTCGCACAAAGCA
GTAAAGATCGTTACGTCAAAGGCGATAGCTACGAATGGGTGACCTTCAAT
ACACGTCTGATCAAAGAGGTAACACAGAATTTTGCTCTGGCCTTTGAGGG
TAGCTATCAATATATGGATCTGAAGCCAAAGGGGTATCAAAACCACAACG
CCGTAAACGGCAGCTTCTATAAACTCACCTTTGCTCCAACTCTAAAAGCT
AACGATATCAATAATTTCTTTAGCCGTCCGGAGCTTCGCCTGTTTGCCAC
CTGGATGGACTGGAGCAGCAAACTTGATGATTTTGCCAGCAATGACGCTT
TCGGCAGCAGTGGTTTCAATACTGGCGGAGAGTGGAATTTTGGTGTCCAA
ATGGAAACCTGGTTTTAA,
Escherichia coli ygcF codon-optimized
(1518 bp)
SEQ ID NO: 35
ATGTACAGGAAATCTACGCTAGCAATGCTAATTGCACTACTTACGTCTGC
GGCAAGCGCGCACGCCCAAACAGATATTTCGACAATAGAGGCCAGGCTTA
ATGCACTGGAGAAGCGATTGCAAGAAGCAGAAAACCGTGCCCAGACAGCT
GAAAACAGGGCAGGCGCAGCTGAAAAGAAAGTTCAGCAACTTACGGCACA
ACAGCAAAAAAATCAAAACTCAACGCAAGAAGTCGCGCAACGGACTGCCA
GGCTTGAGAAGAAAGCCGATGACAAGTCCGGTTTTGAATTTCACGGCTAC
GCTAGAAGTGGCGTTATTATGAATGATTCCGGCGCCTCAACCAAATCCGG
GGCATATATCACGCCTGCCGGCGAGACAGGGGGAGCTATTGGACGATTAG
GAAATCAAGCAGACACGTACGTAGAAATGAATCTAGAACACAAGCAAACT
CTTGACAATGGCGCCACAACTCGGTTTAAAGTAATGGTCGCGGACGGTCA
GACCAGTTATAACGATTGGACGGCATCGACGTCCGATCTGAACGTCCGCC
AGGCCTTCGTGGAATTAGGGAACCTCCCGACCTTCGCTGGTCCGTTCAAG
GGATCGACCCTATGGGCCGGGAAGCGCTTTGACCGAGATAACTTCGATAT
TCATTGGATAGATTCAGACGTGGTATTCCTCGCGGGGACCGGAGGTGGCA
TATATGACGTGAAATGGAACGACGGGTTGAGAAGTAATTTCTCGTTGTAT
GGCCGCAATTTTGGGGACATAGATGACTCTTCCAACTCCGTTCAGAACTA
TATTCTTACAATGAATCACTTCGCTGGACCCTTACAGATGATGGTATCTG
GTTTAAGGGCAAAAGATAACGACGAACGTAAAGATAGCAATGGAAATCTA
GTTAAGGGGGACGCTGCCAATACCGGCGTTCATGCGTTGTTAGGCCTCCA
TAATGACTCATTCTATGGCCTTAGAGATGGCAGTAGCAAGACCGCCCTTC
TCTATGGACACGGCTTGGGTGCTGAAGTTAAGGGAATTGGTTCCGACGGA
GCCTTACGGCCCGGAGCGGATACCTGGAGAATAGCGTCGTACGGTACGAC
ACCTCTCTCAGAAAACTGGAGTGTCGCGCCGGCAATGCTCGCTCAGCGCA
GCAAGGACCGGTATGCTGACGGCGACTCCTACCAATGGGCAACCTTTAAT
TTGCGCCTGATTCAGGCTATCAATCAGAATTTTGCGCTAGCCTACGAAGG
GTCATACCAATATATGGATCTTAAACCTGAAGGGTACAATGACCGCCAGG
CAGTCAATGGGTCATTCTATAAACTCACTTTTGCACCGACGTTCAAGGTG
GGATCCATTGGGGATTTTTTCTCCCGGCCTGAAATCCGATTTTATACTTC
CTGGATGGATTGGAGCAAGAAACTAAATAACTATGCTTCTGATGACGCGT
TGGGCTCAGATGGGTTTAACTCAGGTGGCGAGTGGAGTTTTGGTGTTCAG
ATGGAGGCCTGGTTCTAG

In some embodiments of any of the aspects, the amino acid sequence encoded by the functional sucrose porin gene comprises SEQ ID NO: 36, SEQ ID NO: 90, or an amino acid sequence that is at least 95% (e.g., at least 95%, at least 96%, at least 97%, at least 98%, or at least 99%) identical to the sequence of SEQ ID NO: 36 or SEQ ID NO: 90 that maintains the same functions as SEQ ID NO: 36 or SEQ ID NO: 90 (e.g., sucrose porin).

sucrose porin precursor [Escherichia coli],
GenBank: CAG25845.1, 505 aa
SEQ ID NO: 36

| | |
|---|---|
| 1 | mykkttlavl ialltgattv haqtdissie srlaaleqrl |
| | knaesraqaa earaktaelq |
| 61 | vqklaetqqq nqlttqevaq rtvqleqksa ensgfefhgy |
| | arsgllmnda gsssksgpyl |
| 121 | tpagetggav grigkeadty velnvehkqt idngattrfk |
| | amladgqrdy ndwtggssnl |
| 181 | nirqafaelg alpsftgafq dstvwagkrf drdnfdihwl |
| | dsdvvflagt gggiydvkwn |
| 241 | dtfrsnfsly grnfgdlddi dnnvqnyilt mnhyagpfql |
| | mvsglgakdn ddrkdgngdl |
| 301 | iqtdaantgv halvglhndt fyglregtak tallyghglg |
| | aevkgigsdg allseantwr |
| 361 | fasygttplg sgwyvapail aqsskdryvk gdsyewvtfn |
| | trlikevtqn falafegsyq |
| 421 | ymdlkpkgyq nhnavngsfy kltfaptlka ndinnffsrp |
| | elrlfatwmd wssklddfas |
| 481 | ndafgssgfn tggewnfgvq metwf, |

Escherichia coli ygcF (see also e.g.,
carbohydrate porin [Enterobacterales],
NCBI Reference Sequence: WP_001393599.1)
(505 aa)
SEQ ID NO: 90
MYRKSTLAMLIALLTSAASAHAQTDISTIEARLNALEKRLQEAENRAQTA
ENRAGAAEKKVQQLTAQQQKNQNSTQEVAQRTARLEKKADDKSGFEFHGY
ARSGVIMNDSGASTKSGAYITPAGETGGAIGRLGNQADTYVEMNLEHKQT
LDNGATTRFKVMVADGQTSYNDWTASTSDLNVRQAFVELGNLPTFAGPFK
GSTLWAGKRFDRDNFDIHWIDSDVVFLAGTGGGIYDVKWNDGLRSNFSLY
GRNFGDIDDSSNSVQNYILTMNHFAGPLQMMVSGLRAKDNDERKDSNGNL
VKGDAANTGVHALLGLHNDSFYGLRDGSSKTALLYGHGLGAEVKGIGSDG
ALRPGADTWRIASYGTTPLSENWSVAPAMLAQRSKDRYADGDSYQWATFN
LRLIQAINQNFALAYEGSYQYMDLKPEGYNDRQAVNGSFYKLTFAPTFKV
GSIGDFFSRPEIRFYTSWMDWSKKLNNYASDDALGSDGFNSGGEWSFGVQ
MEAWF

In one aspect described herein is an engineered heterotroph. In some embodiments of any of the aspects, the engineered heterotroph can use a sugar feedstock (e.g., produced by an engineered feedstock bacterium) to produce a secondary product (e.g., violacein, β-carotene). In some embodiments, the engineered heterotroph is an engineered bacterium (e.g., E. coli, B. subtilis). In some embodiments, the engineered heterotroph is an engineered yeast (e.g., S. cerevisiae, Yarrowia lipolytica).

As used herein, the term “secondary product” refers to a product produced from a feedstock solution (e.g., a sugar feedstock solution) as described herein. In some embodiments of any of the aspects, an engineered heterotroph as described herein utilizes a feedstock solution to produce a secondary product. In some embodiments of any of the aspects, the secondary product is a complex organic molecule derived from an organic carbon source in a feedstock solution as described herein. In some embodiments of any of the aspects, the secondary product is violacein. In some embodiments of any of the aspects, the secondary product is β-carotene.

Accordingly, in one aspect described herein is an engineered heterotroph, wherein the engineered heterotroph comprises one or more of the following: (a) at least one overexpressed functional sucrose catabolism gene; (b)(i) at least one endogenous sucrose catabolism repressor gene comprising at least one engineered inactivating modification or (b)(ii) at least one exogenous inhibitor of an endogenous sucrose catabolism repressor gene or gene product (e.g., mRNA, protein); (c)(i) at least one endogenous arabinose utilization gene comprising at least one engineered inactivating modification or (c)(ii) at least one exogenous inhibitor of an endogenous arabinose utilization gene or gene product (e.g., mRNA, protein); or (d) at least one exogenous copy of at least one functional secondary product synthesis gene.

In some embodiments of any of the aspects, the engineered heterotroph comprises at least one overexpressed functional sucrose catabolism gene. In some embodiments of any of the aspects, the engineered heterotroph comprises an engineered inactivating modification of an endogenous sucrose catabolism repressor gene or an inhibitor of an endogenous sucrose catabolism repressor. In some embodiments of any of the aspects, the engineered heterotroph comprises an engineered inactivating modification of an endogenous arabinose utilization gene or an inhibitor of an endogenous arabinose utilization gene. In some embodiments of any of the aspects, the engineered heterotroph comprises at least one exogenous copy of at least one functional secondary product synthesis gene.

In some embodiments of any of the aspects, the engineered heterotroph comprises (a) at least one overexpressed functional sucrose catabolism gene and (b)(i) at least one endogenous sucrose catabolism repressor gene comprising at least one engineered inactivating modification or (b)(ii) at least one exogenous inhibitor of an endogenous sucrose catabolism repressor gene or gene product (e.g., mRNA, protein). In some embodiments of any of the aspects, the engineered heterotroph comprises (a) at least one overexpressed functional sucrose catabolism gene and (c)(i) at least one endogenous arabinose utilization gene comprising at least one engineered inactivating modification or (c)(ii) at least one exogenous inhibitor of an endogenous arabinose utilization gene or gene product (e.g., mRNA, protein). In some embodiments of any of the aspects, the engineered heterotroph comprises (a) at least one overexpressed functional sucrose catabolism gene and (d) at least one exogenous copy of at least one functional secondary product synthesis gene.

In some embodiments of any of the aspects, the engineered heterotroph comprises (a) at least one overexpressed functional sucrose catabolism gene; (b)(i) at least one endogenous sucrose catabolism repressor gene comprising at least one engineered inactivating modification or (b)(ii) at least one exogenous inhibitor of an endogenous sucrose catabolism repressor gene or gene product (e.g., mRNA, protein); and (c)(i) at least one endogenous arabinose utilization gene comprising at least one engineered inactivating modification or (c)(ii) at least one exogenous inhibitor of an endogenous arabinose utilization gene or gene product (e.g., mRNA, protein). In some embodiments of any of the aspects, the engineered heterotroph comprises (a) at least one overexpressed functional sucrose catabolism gene; (b)(i) at least one endogenous sucrose catabolism repressor gene comprising at least one engineered inactivating modification or (b)(ii) at least one exogenous inhibitor of an endogenous sucrose catabolism repressor gene or gene product (e.g., mRNA, protein); and (d) at least one exogenous copy of at least one functional secondary product synthesis gene. In some embodiments of any of the aspects, the engineered heterotroph comprises (a) at least one overexpressed functional sucrose catabolism gene; (c)(i) at least one endogenous arabinose utilization gene comprising at least one engineered inactivating modification or (c)(ii) at least one exogenous inhibitor of an endogenous arabinose utilization gene or gene product (e.g., mRNA, protein); and (d) at least one exogenous copy of at least one functional secondary product synthesis gene. In some embodiments of any of the aspects, the engineered heterotroph comprises (b)(i) at least one endogenous sucrose catabolism repressor gene comprising at least one engineered inactivating modification or (b)(ii) at least one exogenous inhibitor of an endogenous sucrose catabolism repressor gene or gene product (e.g., mRNA, protein); (c)(i) at least one endogenous arabinose utilization gene comprising at least one engineered inactivating modification or (c)(ii) at least one exogenous inhibitor of an endogenous arabinose utilization gene or gene product (e.g., mRNA, protein); and (d) at least one exogenous copy of at least one functional secondary product synthesis gene.

In some embodiments of any of the aspects, the engineered heterotroph comprises (a) at least one overexpressed functional sucrose catabolism gene; (b)(i) at least one endogenous sucrose catabolism repressor gene comprising at least one engineered inactivating modification or (b)(ii) at least one exogenous inhibitor of an endogenous sucrose catabolism repressor gene or gene product (e.g., mRNA, protein); (c)(i) at least one endogenous arabinose utilization gene comprising at least one engineered inactivating modification or (c)(ii) at least one exogenous inhibitor of an endogenous arabinose utilization gene or gene product (e.g., mRNA, protein); and (d) at least one exogenous copy of at least one functional secondary product synthesis gene.

In some embodiments of any of the aspects, the engineered heterotroph is E. coli. In some embodiments of any of the aspects, the engineered heterotroph is E. coli strain W. In some embodiments of any of the aspects, the engineered heterotroph comprises enhanced sucrose utilization. As a non-limiting example, the engineered heterotroph can comprise at least 1%, at least 5%, at least 10%, at least 20%, at least 30%, at least 40%, at least 50%, at least 60%, at least 70%, at least 80%, at least 90%, or at least 100% enhanced (i.e., increased) sucrose utilization compared to a non-engineered heterotroph of the same or original species.

In some embodiments, the engineered heterotroph can grow at a lower sucrose density compared to a non-engineered heterotroph of the same or original species. As a non-limiting example, the engineered heterotroph can grow at a sucrose concentration that is 1.5× lower, 2× lower, 3× lower, 4× lower, 5× lower, 6× lower, 7× lower, 8× lower, 9× lower, or 10× lower than a non-engineered heterotroph of the same or original species.

Members of the species and genera described herein can be identified genetically and/or phenotypically. By way of non-limiting example, the engineered heterotroph as described herein comprises a 16S rDNA sequence at least 97% identical to a 16S rDNA sequence present in a reference strain operational taxonomic unit for E. coli. In some embodiments of any of the aspects, the engineered bacterium as described herein comprises a 16S rDNA that is at least 95% identical (e.g., at least 95%, at least 96%, at least 97%, at least 98%, or at least 99%) identical to the sequence of SEQ ID NO: 80 or SEQ ID NO: 92. In some embodiments of any of the aspects, the heterotroph is engineered from E. coli (e.g., strain W).

Escherichia coli 16S ribosomal RNA, complete
sequence, GenBank J01859.1, 1541 bp
SEQ ID NO: 80

| | |
|---|---|
| 1 | aaattgaaga gtttgatcat ggctcagatt gaacgctggc |
| | ggcaggccta acacatgcaa |
| 61 | gtcgaacggt aacaggaaga agcttgctct ttgctgacga |
| | gtggcggacg ggtgagtaat |
| 121 | gtctgggaaa ctgcctgatg gagggggata actactggaa |
| | acggtagcta ataccgcata |
| 181 | acgtcgcaag accaaagagg gggaccttcg ggcctcttgc |
| | catcggatgt gcccagatgg |
| 241 | gattagctag taggtggggt aacggctcac ctaggcgacg |
| | atccctagct ggtctgagag |
| 301 | gatgaccagc cacactggaa ctgagacacg gtccagactc |
| | ctacgggagg cagcagtggg |
| 361 | gaatattgca caatgggcgc aagcctgatg cagccatgcc |
| | gcgtgtatga agaaggcctt |
| 421 | cgggttgtaa agtactttca gcggggagga agggagtaaa |
| | gttaatacct ttgctcattg |
| 481 | acgttacccg cagaagaagc accggctaac tccgtgccag |
| | cagccgcggt aatacggagg |
| 541 | gtgcaagcgt taatcggaat tactgggcgt aaagcgcacg |
| | caggcggttt gttaagtcag |
| 601 | atgtgaaatc cccgggctca acctgggaac tgcatctgat |
| | actggcaagc ttgagtctcg |
| 661 | tagagggggg tagaattcca ggtgtagcgg tgaaatgcgt |
| | agagatctgg aggaataccg |
| 721 | gtggcgaagg cggccccctg gacgaagact gacgctcagg |
| | tgcgaaagcg tggggagcaa |
| 781 | acaggattag ataccctggt agtccacgcc gtaaacgatg |
| | tcgacttgga ggttgtgccc |
| 841 | ttgaggcgtg gcttccggag ctaacgcgtt aagtcgaccg |
| | cctggggagt acggccgcaa |
| 901 | ggttaaaact caaatgaatt gacgggggcc cgcacaagcg |
| | gtggagcatg tggtttaatt |
| 961 | cgatgcaacg cgaagaacct tacctggtct tgacatccac |
| | ggaagttttc agagatgaga |
| 1021 | atgtgccttc gggaaccgtg agacaggtgc tgcatggctg |
| | tcgtcagctc gtgttgtgaa |
| 1081 | atgttgggtt aagtcccgca acgagcgcaa cccttatcct |
| | ttgttgccag cggtccggcc |
| 1141 | gggaactcaa aggagactgc cagtgataaa ctggaggaag |
| | gtggggatga cgtcaagtca |
| 1201 | tcatggccct tacgaccagg gctacacacg tgctacaatg |
| | gcgcatacaa agagaagcga |
| 1261 | cctcgcgaga gcaagcggac ctcataaagt gcgtcgtagt |
| | ccggattgga gtctgcaact |
| 1321 | cgactccatg aagtcggaat cgctagtaat cgtggatcag |
| | aatgccacgg tgaatacgtt |
| 1381 | cccgggcctt gtacacaccg cccgtcacac catgggagtg |
| | ggttgcaaaa gaagtaggta |
| 1441 | gcttaacctt cgggagggcg cttaccactt tgtgattcat |
| | gactggggtg aagtcgtaac |
| 1501 | aaggtaaccg taggggaacc tgcggttgga tcacctcctt |
| | a, |

Escherichia coli W 16S ribosomal RNA (1554 bp)
SEQ ID NO: 92
AAATTGAAGAGTTTGATCATGGCTCAGATTGAACGCTGGCGGCAGGCCTA
ACACATGCAAGTCGAACGGTAACAGGAAGAAGCTTGCTTCTTTGCTGACG
AGTGGCGGACGGGTGAGTAATGTCTGGGAAACTGCCTGATGGAGGGGGAT
AACTACTGGAAACGGTAGCTAATACCGCATAACGTCGCAAGACCAAAGAG
GGGGACCTTCGGGCCTCTTGCCATCGGATGTGCCCAGATGGGATTAGCTA
GTAGGTGGGGTAACGGCTCACCTAGGCGACGATCCCTAGCTGGTCTGAGA
GGATGACCAGCCACACTGGAACTGAGACACGGTCCAGACTCCTACGGGAG
GCAGCAGTGGGGAATATTGCACAATGGGCGCAAGCCTGATGCAGCCATGC
CGCGTGTATGAAGAAGGCCTTCGGGTTGTAAAGTACTTTCAGCGGGGAGG
AAGGGAGTAAAGTTAATACCTTTGCTCATTGACGTTACCCGCAGAAGAAG
CACCGGCTAACTCCGTGCCAGCAGCCGCGGTAATACGGAGGGTGCAAGCG
TTAATCGGAATTACTGGGCGTAAAGCGCACGCAGGCGGTTTGTTAAGTCA
GATGTGAAATCCCCGGGCTCAACCTGGGAACTGCATCTGATACTGGCAAG
CTTGAGTCTCGTAGAGGGGGGTAGAATTCCAGGTGTAGCGGTGAAATGCG
TAGAGATCTGGAGGAATACCGGTGGCGAAGGCGGCCCCCTGGACGAAGAC
TGACGCTCAGGTGCGAAAGCGTGGGGAGCAAACAGGATTAGATACCCTGG
TAGTCCACGCCGTAAACGATGTCGACTTGGAGGTTGTGCCCTTGAGGCGT
GGCTTCCGGAGCTAACGCGTTAAGTCGACCGCCTGGGGAGTACGGCCGCA
AGGTTAAAACTCAAATGAATTGACGGGGGCCCGCACAAGCGGTGGAGCAT
GTGGTTTAATTCGATGCAACGCGAAGAACCTTACCTGGTCTTGACATCCA
CGGAAGTTTTCAGAGATGAGAATGTGCCTTCGGGAACCGTGAGACAGGTG
CTGCATGGCTGTCGTCAGCTCGTGTTGTGAAATGTTGGGTTAAGTCCCGC
AACGAGCGCAACCCTTATCCTTTGTTGCCAGCGGTCCGGCCGGGAACTCA
AAGGAGACTGCCAGTGATAAACTGGAGGAAGGTGGGGATGACGTCAAGTC
ATCATGGCCCTTACGACCAGGGCTACACACGTGCTACAATGGCGCATACA
AAGAGAAGCGACCTCGCGAGAGCAAGCGGACCTCATAAAGTGCGTCGTAG
TCCGGATTGGAGTCTGCAACTCGACTCCATGAAGTCGGAATCGCTAGTAA
TCGTGGATCAGAATGCCACGGTGAATACGTTCCCGGGCCTTGTACACACC
GCCCGTCACACCATGGGAGTGGGTTGCAAAAGAAGTAGGTAGCTTAACCT
TCGGGAGGGCGCTTACCACTTTGTGATTCATGACTGGGGTGAAGTCGTAA
CAAGGTAACCGTAGGGGAACCTGCGGTTGGATCACCTCCTTACCTTAAAG
AAGC,

In some embodiments of any of the aspects, the at least one engineered inactivating modification of an endogenous gene (e.g., sucrose catabolism repressor genes, arabinose utilization genes) or insertion of a heterologous gene (e.g., heterologous secondary product synthesis gene) in an engineered heterotroph is performed using phage transduction (e.g., P1 phage; see e.g., Thomason et al. E. coli genome manipulation by P1 transduction, Curr Protoc Mol Biol. 2007 July; Chapter 1: Unit 1.17). In some embodiments of any of the aspects, the heterotroph is engineered from a bacterial strain (e.g., E. coli) from the Keio collection, which comprises in-frame, single-gene knockout mutants; see e.g., Baba et al., Construction of Escherichia coli K-12 in-frame, single-gene knockout mutants: the Keio collection, Mol Syst Biol. 2006; 2: 2006.0008. The foregoing references are incorporated by reference herein in their entireties.

In some embodiments of any of the aspects, the engineered heterotroph comprises at least one overexpressed functional sucrose catabolism gene. In some embodiments of any of the aspects, the at least one overexpressed functional sucrose catabolism gene is an endogenous gene. In some embodiments of any of the aspects, the at least one overexpressed functional sucrose catabolism gene is a heterologous gene. In some embodiments of any of the aspects, the at least one functional sucrose catabolism comprises an invertase (e.g., CscA), a sucrose permease (e.g., CscB), or a fructokinase (e.g., CscK).

In some embodiments of any of the aspects, the engineered heterotroph comprises an invertase (e.g., CscA). In some embodiments of any of the aspects, the engineered heterotroph comprises a sucrose permease (e.g., CscB). In some embodiments of any of the aspects, the engineered heterotroph comprises a fructokinase (e.g., CscK). In some embodiments of any of the aspects, the engineered heterotroph comprises an invertase (e.g., CscA) and a sucrose permease (e.g., CscB). In some embodiments of any of the aspects, the engineered heterotroph comprises an invertase (e.g., CscA) and a fructokinase (e.g., CscK). In some embodiments of any of the aspects, the engineered heterotroph comprises a sucrose permease (e.g., CscB), and a fructokinase (e.g., CscK). In some embodiments of any of the aspects, the engineered heterotroph comprises an invertase (e.g., CscA), a sucrose permease (e.g., CscB), and a fructokinase (e.g., CscK).

In some embodiments of any of the aspects, the nucleic acid sequence of the functional sucrose catabolism gene (e.g., invertase, CscA) comprises SEQ ID NO: 43 or a sequence that is at least 95% (e.g., at least 95%, at least 96%, at least 97%, at least 98%, or at least 99%) identical to the sequence of SEQ ID NO: 43 that maintains the same functions as SEQ ID NO: 43 (e.g., invertase, sucrose-6-phosphate hydrolase).

Escherichia coli UMN026, complete genome,
NCBI Reference Sequence: NC_011751.1,
REGION: 2768873-2770306, 1434 bp
SEQ ID NO: 43

| | |
|---|---|
| 1 | atgacgcaat ctcgattgca tgcggcgcaa aacgcactag |
| | caaaacttca cgagcgccga |
| 61 | ggtaacactt tctatcccca ttttcacctc gcgcctcctg |
| | ccgggtggat gaacgatcca |
| 121 | aacggcctga tctggtttaa cgatcgttat cacgcgtttt |
| | atcaacatca cccgatgagc |
| 181 | gaacactggg ggccaatgca ctggggacat gccaccagcg |
| | acgatatgat ccactggcag |
| 241 | catgagccta ttgcgctagc gccaggagac gagaatgaca |
| | aagacggatg tttttcaggt |
| 301 | agtgctgtcg atgacaatgg tgtcctctca cttatctaca |
| | ccggacacgt ctggctcgat |
| 361 | agtgaaggta atgacgatgc aattcgcgaa gtacaatgtc |
| | tggctaccag tcgggatggt |
| 421 | attcatttcg agaaacaggg tgtgatcctc actccaccag |
| | aaggaatcat gcacttccgc |
| 481 | gatcctaaag tgtggcgtga agccgacaca tggtggatgg |
| | tagtcggggc gaaagaccca |
| 541 | ggcaacacgg ggcagatcct gctttatcgc ggcagttcat |
| | tgcgtgaatg gactttcgat |
| 601 | cgcgtactgg cccacgctga tgcgggtgaa agctatatgt |
| | gggaatgtcc ggactttttc |
| 661 | agccttggcg atcagcatta tctgatgttt tccccgcagg |
| | gaatgaatgc cgagggatac |
| 721 | agttatcgaa atcgctttca aagtggcgta atacccggaa |
| | tgtggtcgcc aggacgactt |
| 781 | tttgcacaat ccgggcattt tactgaactt gataacgggc |
| | atgactttta tgcaccacaa |
| 841 | agctttgtag cgaaggatgg tcggcgtatt gttatcggct |
| | ggatggatat gtgggaatcg |
| 901 | ccaatgccct caaaacgtga aggctgggca ggctgcatga |
| | cgctggcgcg cgagctatca |
| 961 | gagagcaatg gcaaactcct acaacgcccg gtacacgaag |
| | ctgagtcgtt acgccagcag |
| 1021 | catcaatcta tctctccccg cacaatcagc aataaatatg |
| | ttttgcagga aaacgcgcaa |
| 1081 | gcagttgaga ttcagttgca gtgggagctg aagaacagtg |
| | atgccgaaca ttacggatta |
| 1141 | caactcggca caggaatgcg gctgtatatt gataaccaat |
| | ctgagcgact tgttttgtgg |
| 1201 | cgatattacc cacacgagaa tttagacggc taccgtagta |
| | ttcccctccc gcagggtgac |
| 1261 | acgctcgccc taaggatatt tatcgataca tcatccgtgg |
| | aagtatttat taacgacggg |
| 1321 | gaaacggtga tgagtagccg aatctatccg cagccagaag |
| | aacgggaact gtcgctctat |
| 1381 | gcctcccacg gagtggctgt gctgcaacat ggagcactct |
| | ggcaactggg ttaa, |

In some embodiments of any of the aspects, the amino acid sequence encoded by the functional sucrose catabolism gene (e.g., invertase, CscA) comprises SEQ ID NO: 44 or an amino acid sequence that is at least 95% (e.g., at least 95%, at least 96%, at least 97%, at least 98%, or at least 99%) identical to the sequence of SEQ ID NO: 44 that maintains the same functions as SEQ ID NO: 44 (e.g., invertase, sucrose-6-phosphate hydrolase).

sucrose-6-phosphate hydrolase [Escherichia
coli UMN026], NCBI Reference Sequence:
YP_002413400.2, 477 aa
SEQ ID NO: 44

| | |
|---|---|
| 1 | mtqsrlhaaq nalaklherr gntfyphfhl appagwmndp |
| | ngliwfndry hafyqhhpms |
| 61 | ehwgpmhwgh atsddmihwq hepialapgd endkdgcfsg |
| | savddngvls liytghvwld |
| 121 | segnddaire vqclatsrdg ihfekqgvil tppegimhfr |
| | dpkvwreadt wwmvvgakdp |
| 181 | gntgqillyr gsslrewtfd rvlahadage symwecpdff |
| | slgdqhylmf spqgmnaegy |
| 241 | syrnrfqsgv ipgmwspgrl faqsghftel dnghdfyapq |
| | sfvakdgrri vigwmdmwes |
| 301 | pmpskregwa gcmtlarels esngkllqrp vheaeslrqq |
| | hqsisprtis nkyvlqenaq |
| 361 | aveiqlqwel knsdaehygl qlgtgmrlyi dnqserlvlw |
| | ryyphenldg yrsiplpqgd |
| 421 | tlalrifidt ssvevfindg etvmssriyp qpeerelsly |
| | ashgvavlqh galwqlg, |

In some embodiments of any of the aspects, the nucleic acid sequence of the functional sucrose catabolism gene (e.g., a sucrose permease, CscB) comprises SEQ ID NO: 45 or a sequence that is at least 95% (e.g., at least 95%, at least 96%, at least 97%, at least 98%, or at least 99%) identical to the sequence of SEQ ID NO: 45 that maintains the same functions as SEQ ID NO: 45 (e.g., sucrose permease).

Escherichia coli UMN026, complete genome,
NCBI Reference Sequence: NC_011751.1,
REGION: complement (2766425-2767672),
1248 bp
SEQ ID NO: 45

| | |
|---|---|
| 1 | atggcactga atattccatt cagaaatgcg tactatcgtt |
| | ttgcatccag ttactcattt |
| 61 | ctctttttta tttcctggtc gctgtggtgg tcgttatacg |
| | ctatttggct gaaaggacat |
| 121 | ctaggattaa cagggacgga attaggtaca ctttattcgg |
| | tcaaccagtt taccagcatt |
| 181 | ctatttatga tgttctacgg catcgttcag gataaactcg |
| | gtctgaagaa accgctcatc |
| 241 | tggtgtatga gtttcattct ggtcttgacc ggaccgttta |
| | tgatttacgt ttatgaaccg |
| 301 | ttactgcaaa gcaatttttc tgtaggtcta attctggggg |
| | cgctcttttt tggcctgggg |
| 361 | tatctggcgg gatgtggttt gcttgacagc ttcactgaaa |
| | aaatggcgcg aaattttcat |
| 421 | ttcgaatatg gaacagcgcg cgcctgggga tcttttggct |
| | atgctattgg cgcgttcttt |
| 481 | gccggcatat tttttagtat cagtccccat atcaacttct |
| | ggctggtctc gctatttggc |
| 541 | gctgtattta tgatgatcaa catgcgtttt aaagataagg |
| | gtcaccagtg tgtagcggcg |
| 601 | gatgcgggag gggtaaaaaa agaggatttt atcgcagttt |
| | tcaaggatcg aaacttctgg |
| 661 | gtttttgtca tatttattgt ggggacgtgg tctttctata |
| | acatttttga tcaacaactc |
| 721 | tttcctgtct tttatgcagg tttattcgaa tcacacgatg |
| | taggaacgcg cctgtatggt |
| 781 | tatctcaact cattccaggt ggtactcgaa gcgctgtgca |
| | tggcgattat tcctttcttt |
| 841 | gtgaatcggg tagggccaaa aaatgcatta cttatcggtg |
| | ttgtgattat ggcgttgcgt |
| 901 | atcctttcct gcgcgttgtt cgttaacccc tggattattt |
| | cattagtgaa gctgttacat |
| 961 | gccattgagg ttccactttg tgtcatatcc gtcttcaaat |
| | acagcgtggc aaactttgat |
| 1021 | aagcgcctgt cgtcgacgat ctttctgatt ggttttcaaa |
| | ttgccagttc gcttgggatt |
| 1081 | gtgctgcttt caacgccgac tgggatactc tttgaccacg |
| | caggctacca gacagttttc |
| 1141 | ttcgcaattt cgggtattgt ctgcctgatg ttgctatttg |
| | gcattttctt cctgagtaaa |
| 1201 | aaacgcgagc aaatagttat ggaaacgcct gtaccttcag |
| | caatatag, |

In some embodiments of any of the aspects, the amino acid sequence encoded by the functional sucrose catabolism gene (e.g., a sucrose permease, CscB) comprises SEQ ID NO: 46 or an amino acid sequence that is at least 95% (e.g., at least 95%, at least 96%, at least 97%, at least 98%, or at least 99%) identical to the sequence of SEQ ID NO: 46 that maintains the same functions as SEQ ID NO: 46 (e.g., sucrose permease).

sucrose permease [Escherichia coli UMN026],
NCBI Reference Sequence: YP_002413398.1,
415 aa
SEQ ID NO: 46

| | |
|---|---|
| 1 | malnipfrna yyrfassysf iffiswslww slyaiwlkgh |
| | lgltgtelgt lysvnqftsi |
| 61 | ifmmfygivq dklglkkpli wcmsfilvlt gpfmiyvyep |
| | llqsnfsvgl ilgalffglg |
| 121 | ylagcgilds ftekmarnfh feygtarawg sfgyaigaff |
| | agiffsisph infwlvslfg |
| 181 | avfmminmrf kdkghqcvaa daggvkkedf iavfkdrnfw |
| | vfvifivgtw sfynifdqql |
| 241 | fpvfyaglfe shdvgtrlyg ylnsfqvvle alcmaiipff |
| | vnrvgpknal ligvvimalr |
| 301 | ilscalfvnp wiislvkllh aievplcvis vfkysvanfd |
| | krisstifli gfqiasslgi |
| 361 | vllstptgil fdhagyqtvf faisgivclm llfgifflsk |
| | kreqivmetp vpsai, |

In some embodiments of any of the aspects, the nucleic acid sequence of the functional sucrose catabolism gene (e.g., fructokinase, CscK) comprises SEQ ID NO: 47 or a sequence that is at least 95% (e.g., at least 95%, at least 96%, at least 97%, at least 98%, or at least 99%) identical to the sequence of SEQ ID NO: 47 that maintains the same functions as SEQ ID NO: 47 (e.g., fructokinase).

Escherichia coli UMN026, complete genome,
NCBI Reference Sequence: NC_011751.1, REGION:
complement (2767734-2768657), 924 bp
SEQ ID NO: 47

| | |
|---|---|
| 1 | atgtcagcca aagtatgggt tttaggggat gcggtcgtag |
| | atctcttgcc agaatcagac |
| 61 | gggcggctac tgccttgtcc tggcggcgcg ccagctaacg |
| | ttgcggtggg aatcgccaga |
| 121 | ttaggcggaa caagtgggtt tataggtcgg gtcggtgatg |
| | atccttttgg tgcgttaatg |
| 181 | caaagaacgc tgctaactga gggtgtcgat atcacgtatc |
| | tgaagcaaga tgaatggcac |
| 241 | cggacatcca cggtgcttgt cgatctgaac gatcaaggag |
| | aacgttcatt tacgtttatg |
| 301 | gtccgcccca gtgccgatct ttttttagag acgacagact |
| | tgccctgctg gcgacatggc |
| 361 | gaatggttac atctctgttc aattgcgttg tctgccgagc |
| | cttcgcgtac cagcgcattt |
| 421 | actgcgatga cggcgatccg gcatgccgga ggttttgtca |
| | gcttcgatcc caatattcgt |
| 481 | gaagatctat ggcaagacga gcatttgctc cgcttgtgtt |
| | tgcggcaggc gctacaactg |
| 541 | gcggatgtcg tcaagctctc ggaagaagaa tggcgactta |
| | tcagtggaaa aacacagaac |
| 601 | gatcgggata tatgcgccct ggcaaaagat tatgagatcg |
| | ccatgctgtt ggtgactaaa |
| 661 | ggtgcagaag gggtggtggt ctgttatcga ggacaagtcc |
| | accattttgc tggaatgtct |
| 721 | gtgaattgtg tcgatagcac tggggcggga gatgcgttcg |
| | ttgccgggtt actcacaggt |
| 781 | ctgtcctctt cgggattatc tacagatgag agagaaatgc |
| | gacgaattat cgatctcgct |
| 841 | caacgttgcg gagcgcttgc agtaacagcg aaaggggcaa |
| | tgacagcgct gccatgtcga |
| 901 | caagaactgg aaagtgagaa gtaa, |

In some embodiments of any of the aspects, the amino acid sequence encoded by the functional sucrose catabolism gene (e.g., fructokinase, CscK) comprises SEQ ID NO: 48 or an amino acid sequence that is at least 95% (e.g., at least 95%, at least 96%, at least 97%, at least 98%, or at least 99%) identical to the sequence of SEQ ID NO: 48 that maintains the same functions as SEQ ID NO: 48 (e.g., fructokinase).

```
SEQ ID NO: 48, fructokinase [Escherichia coli
UMN026], NCBI Reference Sequence
YP_002413399.1, 307 aa
    1 msakvwvlgd avvdllpesd grilpcpgga
        panvavgiar Iggtsgfigr vgddpfgalm
  61 qrtlltegvd itylkqdewh rtstvlvdln
        dqgersftfm vrpsadlfle ttdlpcwrhg
121 ewlhlcsial saepsrtsaf tamtairhag
        gfvsfdpnir edlwqdehll rlclrqalql
181 advvklseee wrlisgktqn drdicalakd
        yeiamllvtk gaegvvvcyr gqvhhfagms
241 vncvdstgag dafvaglltg Isssglstde
        remrriidla qrcgalavta kgamtalpcr
301 qelesek
```

In some embodiments of any of the aspects, the engineered heterotroph comprises (i) at least one endogenous sucrose catabolism repressor gene comprising at least one engineered inactivating modification; and/or (ii) at least one exogenous inhibitor of an endogenous sucrose catabolism repressor gene or gene product. In some embodiments of any of the aspects, the engineered heterotroph comprises (i) at least one endogenous sucrose catabolism repressor gene comprising at least one engineered inactivating modification. In some embodiments of any of the aspects, the engineered heterotroph comprises (ii) at least one exogenous inhibitor of an endogenous sucrose catabolism repressor gene or gene product. In some embodiments of any of the aspects, the endogenous sucrose catabolism repressor gene comprises the repressor E. coli CscR. See e.g., Arifin et al., J Biotechnol. 2011 Dec. 20; 156(4):275-8, the content of which is incorporated herein by reference in its entirety.

In some embodiments of any of the aspects, the engineered bacterium comprises an engineered inactivating modification of an endogenous sucrose catabolism repressor gene (e.g., CscR). In some embodiments of any of the aspects, the nucleic acid sequence of the endogenous sucrose catabolism repressor gene (e.g., CscR) comprises SEQ ID NO: 49 or a sequence that is at least 95% (e.g., at least 95%, at least 96%, at least 97%, at least 98%, or at least 99%) identical to the sequence of SEQ ID NO: 49 that maintains the same functions as SEQ ID NO: 49 (e.g., sucrose catabolism repressor).

```
SEQ ID NO: 49, Escherichia coli UMNO26,
complete genome, NCBI Reference
Sequence: NC_011751.1, REGION:
complement (2770314-2771309), 996 bp
    1 atggcttcat taaaggatgt cgcacgcctg
        gcgggagtgt cgatgatgac agtctcccgg
  61 gtgatgcata atgcagaatc tgtgcgtcct
        gcaacgcgta accgcgtatt gcaggcaatc
121 cagaccctga attatgttcc tgatctttcc
        gcccgtaaga tgcgcgctca aggacgtaag
181 ccgtcgactc tcgccgtgct ggcgcaggac
        acggctacca ctcctttctc tgttgatatt
241 ctgcttgcca ttgagcaaac cgccagcgag
        ttcggctgga atagtttttt aatcaatatt
301 ttttctgaag atgacgctgc ccgcgcggca
        cgtcagctgc ttgcccaccg tccggatggc
361 attatctata ctacaatggg gctgcgacat
        atcacgctgc ctgagtctct gtatggtgaa
421 aatattgtat tggcgaactg tgttgcggat
        gacccagcgt tacccagtta tatccctgat
481 gattacactg cacaatatga atcaacacag
        catttgctcg cggcgggcta tcgtcaaccg
541 ttatgcttct ggctaccgga aagtgcgttg
        gcaacagggt atcgtcggca gggatttgag
601 caggcctggc gtgatgctgg acgagatctg
        gctgaggtga aacaatttca catggcaaca
661 ggtgatgatc actacaccga tctcgcaagt
        ttactcaatg accacttcaa atctggcaaa
721 ccagattttg atgttctgat atgtggtaac
        gatcgcgcag cctttgtcgc ttatcaggtt
781 ctcctggcga agggggtacg tatcccgcag
        gatgtcgccg taatgggctt tgataatctg
841 gttggcgtcg ggcatctgtt tttaccgccg
        ctgaccacaa ttcagcttcc acatgacatt
901 atcgggcggg aagctgcatt gcatattatt
        gaaggtcgtg aagggggaag agtgacgcgg
961 atcccttgcc cgctgttgat ccgttgttcc
        acctga
```

In some embodiments of any of the aspects, the amino acid sequence encoded by the endogenous sucrose catabolism repressor gene (e.g., CscR) comprises SEQ ID NO: 50 or an amino acid sequence that is at least 95% (e.g., at least 95%, at least 96%, at least 97%, at least 98%, or at least 99%) identical to the sequence of SEQ ID NO: 50 that maintains the same functions as SEQ ID NO: 50 (e.g., sucrose catabolism repressor).

```
SEQ ID NO: 50, csc operon repressor
[Escherichia coli UMN026], NCBI Reference
Sequence:YP_002413401.2, 331 aa
    1 maslkdvarl agvsmmtvsr vmhnaesvrp
        atrnrvlqai qtlnyvpdls arkmraqgrk
  61 pstlavlaqd tattpfsvdi llaieqtase
        fgwnsflini fseddaaraa rqllahrpdg
121 iiyttmglrh itlpeslyge nivlancvad
        dpalpsyipd dytaqyestq hllaagyrqp
181 icfwlpesal atgyrrqgfe qawrdagrdl
        aevkqfhmat gddhytdlas llndhfksgk
241 pdfdvlicgn draafvayqv llakgvripq
        dvavmgfdnl vgvghlflpp Ittiqlphdi
301 igreaalhii egreggrvtr ipcpllircs
        t
```

In some embodiments of any of the aspects, the engineered heterotroph comprises (i) at least one endogenous arabinose utilization gene comprising at least one engineered inactivating modification; and/or (ii) at least one exogenous inhibitor of an endogenous arabinose utilization gene or gene product. In some embodiments of any of the aspects, the engineered heterotroph comprises (i) at least one endogenous arabinose utilization gene comprising at least one engineered inactivating modification. In some embodiments of any of the aspects, the entered heterotroph comprises (ii) at least one exogenous inhibitor of an endogenous arabinose utilization gene or gene product.

In some embodiments of any of the aspects, the inactivated and/or inhibited endogenous arabinose utilization gene comprises araB, araA, or araD. In some embodiments of any of the aspects, the endogenous arabinose utilization gene comprises araB. In some embodiments of any of the aspects, the endogenous arabinose utilization gene comprises araA. In some embodiments of any of the aspects, the endogenous arabinose utilization gene comprises araD. In some embodiments of any of the aspects, the endogenous arabinose utilization gene comprises araB and araA. In some embodiments of any of the aspects, the endogenous arabinose utilization gene comprises araB and araD. In some embodiments of any of the aspects, the endogenous arabinose utilization gene comprises araA and araD. In some embodiments of any of the aspects, the endogenous arabinose utilization gene comprises araB, araA, and araD. In some embodiments of any of the aspects, the endogenous arabinose utilization gene comprises the araBAD operon, including the promoter for araB, araA, and araD. In some embodiments of any of the aspects, the endogenous arabinose utilization gene comprises the araC regulatory gene. In some embodiments of any of the aspects, the endogenous arabinose utilization gene comprises the araBAD operon and araC.

In some embodiments of any of the aspects, the nucleic acid sequence of the endogenous arabinose utilization gene comprises SEQ ID NO: 93-96 or a sequence that is at least 95% (e.g., at least 95%, at least 96%, at least 97%, at least 98%, or at least 99%) identical to the sequence of SEQ ID NO: 93-96 that maintains the same functions as SEQ ID NO: 93-96 (e.g., arabinose utilization).

```
araB, Escherichia coli str.
K-12 substr. MG1655, complete genome,
NCBI Reference Sequence: NC_000913.3
REGION: complement (68348-70048), 1701 bp
SEQ ID NO: 93
      1 atggcgattg caattggcct cgattttggc
          agtgattctg tgcgagcttt ggcggtggac
    61 tgcgctaccg gtgaagagat cgccaccagc
          gtagagtggt atccccgttg gcagaaaggg
  121 caattttgtg atgccccgaa taaccagttc
          cgtcatcatc cgcgtgacta cattgagtca
  181 atggaagcgg cactgaaaac cgtgcttgca
          gagcttagcg tcgaacagcg cgcagctgtg
  241 gtcgggattg gcgttgacag taccggctcg
          acgcccgcac cgattgatgc cgacggaaac
  301 gtgctggcgc tgcgcccgga gtttgccgaa
          aacccgaacg cgatgttcgt attgtggaaa
  361 gaccacactg cggttgaaga agcggaagag
          attacccgtt tgtgccacgc gccgggcaac
  421 gttgactact cccgctacat tggtggtatt
          tattccagcg aatggttctg ggcaaaaatc
  481 ctgcatgtga ctcgccagga cagcgccgtg
          gcgcaatctg ccgcatcgtg gattgagctg
  541 tgcgactggg tgccagctct gctttccggt
          accacccgcc cgcaggatat tcgtcgcgga
  601 cgttgcagcg ccgggcataa atctctgtgg
          cacgaaagct ggggcggcct gccgccagcc
  661 agtttctttg atgagctgga cccgatcctc
          aatcgccatt tgccttcccc gctgttcact
  721 gacacttgga ctgccgatat tccggtgggc
          accttatgcc cggaatgggc gcagcgtctc
  781 ggcctgcctg aaagcgtggt gatttccggc
          ggcgcgtttg actgccatat gggcgcagtt
  841 ggcgcaggcg cacagcctaa cgcactggta
          aaagttatcg gtacttccac ctgcgacatt
  901 ctgattgccg acaaacagag cgttggcgag
          cgggcagtta aaggtatttg cggtcaggtt
  961 gatggcagcg tggtgcctgg atttatcggt
          ctggaagcag gccaatcggc gtttggtgat
1021 atctacgcct ggtttggtcg cgtactcggc
          tggccgctgg aacagcttgc cgcccagcat
1081 ccggaactga aaacgcaaat caacgccagc
          cagaaacaac tgcttccggc gctgaccgaa
1141 gcatgggcca aaaatccgtc tctggatcac
          ctgccggtgg tgctcgactg gtttaacggc
1201 cgccgcacac cgaacgctaa ccaacgcctg
          aaaggggtga ttaccgatct taacctcgct
1261 accgacgctc cgctgctgtt cggcggtttg
          attgctgcca ccgcctttgg cgcacgcgca
1321 atcatggagt gctttaccga tcaggggatc
          gccgttaata acgtgatggc actgggcggc
1381 atcgcgcgga aaaaccaggt cattatgcag
          gcctgctgcg acgtgctgaa tcgcccgctg
1441 caaattgttg cctctgacca gtgctgtgcg
          ctcggtgcgg cgatttttgc tgccgtcgcc
1501 gcgaaagtgc acgcagacat cccatcagct
          cagcaaaaaa tggccagtgc ggtagagaaa
1561 accctgcaac cgtgcagcga gcaggcacaa
          cgctttgaac agctttatcg ccgctatcag
1621 caatgggcga tgagcgccga acaacactat
          cttccaactt ccgccccggc acaggctgcc
1681 caggccgttg cgactctata a
araA, Escherichia coli str.
K-12 substr. MG1655, complete genome,
NCBI Reference Sequence: NC_000913.3,
REGION: complement (66835-68337), 1503 bp
SEQ ID NO: 94
      1 atgacgattt ttgataatta tgaagtgtgg
          tttgtcattg gcagccagca tctgtatggc
    61 ccggaaaccc tgcgtcaggt cacccaacat
          gccgagcacg tcgttaatgc gctgaatacg
  121 gaagcgaaac tgccctgcaa actggtgttg
          aaaccgctgg gcaccacgcc ggatgaaatc
  181 accgctattt gccgcgacgc gaattacgac
          gatcgttgcg ctggtctggt ggtgtggctg
  241 cacaccttct ccccggccaa aatgtggatc
          aacggcctga ccatgctcaa caaaccgttg
  301 ctgcaattcc acacccagtt caacgcggcg
          ctgccgtggg acagtatcga tatggacttt
  361 atgaacctga accagactgc acatggcggt
          cgcgagttcg gcttcattgg cgcgcgtatg
  421 cgtcagcaac atgccgtggt taccggtcac
          tggcaggata aacaagccca tgagcgtatc
  481 ggctcctgga tgcgtcaggc ggtctctaaa
          caggataccc gtcatctgaa agtctgccga
  541 tttggcgata acatgcgtga agtggcggtc
          accgatggcg ataaagttgc cgcacagatc
  601 aagttcggtt tctccgtcaa tacctgggcg
          gttggcgatc tggtgcaggt ggtgaactcc
  661 atcagcgacg gcgatgttaa cgcgctggtc
          gatgagtacg aaagctgcta caccatgacg
  721 cctgccacac aaatccacgg caaaaaacga
          cagaacgtgc tggaagcggc gcgtattgag
  781 ctggggatga agcgtttcct ggaacaaggt
          ggcttccacg cgttcaccac cacctttgaa
  841 gatttgcacg gtctgaaaca gcttcctggt
          ctggccgtac agcgtctgat gcagcagggt
  901 tacggctttg cgggcgaagg cgactggaaa
          actgccgccc tgcttcgcat catgaaggtg
  961 atgtcaaccg gtctgcaggg cggcacctcc
          tttatggagg actacaccta tcacttcgag
1021 aaaggtaatg acctggtgct cggctcccat
          atgctggaag tctgcccgtc gatcgccgca
1081 gaagagaaac cgatcctcga cgttcagcat
          ctcggtattg gtggtaagga cgatcctgcc
1141 cgcctgatct tcaataccca aaccggccca
          gcgattgtcg ccagcttgat tgatctcggc
1201 gatcgttacc gtctactggt taactgcatc
          gacacggtga aaacaccgca ctccctgccg
1261 aaactgccgg tggcgaatgc gctgtggaaa
          gcgcaaccgg atctgccaac tgcttccgaa
1321 gcgtggatcc tcgctggtgg cgcgcaccat
          accgtcttca gccatgcact gaacctcaac
1381 gatatgcgcc aattcgccga gatgcacgac
          attgaaatca eggtgattga taacgacaca
1441 cgcctgccag cgtttaaaga cgcgctgcgc
          tggaacgaag tgtattacgg gtttcgtcgc
1501 taa
araD, Escherichia coli str.
K-12 substr. MG1655, complete genome,
NCBI Reference Sequence: NC_000913.3,
REGION: complement (65855-66550), 696 bp
SEQ ID NO: 95
      1 atgttagaag atctcaaacg ccaggtatta
          gaagccaacc tggcgctgcc aaaacacaac
    61 ctggtcacgc tcacatgggg caacgtcagc
          gccgttgatc gcgagcgcgg cgtctttgtg
  121 atcaaacctt ccggcgtcga ttacagcgtc
          atgaccgctg acgatatggt cgtggttagc
  181 atcgaaaccg gtgaagtggt tgaaggtacg
          aaaaagccct cctccgacac gccaactcac
  241 cggctgctct atcaggcatt cccctccatt
          ggcggcattg tgcatacgca ctcgcgccac
  301 gccaccatct gggcgcaggc gggtcagtcg
          attccagcaa ccggcaccac ccacgccgac
  361 tatttctacg gcaccattcc ctgcacccgc
          aaaatgaccg acgcagaaat caacggcgaa
  421 tatgagtggg aaaccggtaa cgtcatcgta
          gaaacctttg aaaaacaggg tatcgatgca
  481 gcgcaaatgc ccggcgttct ggtccattcc
          cacggcccgt ttgcatgggg caaaaatgcc
  541 gaagatgcgg tgcataacgc catcgtgctg
          gaagaggtcg cttatatggg gatattctgc
  601 cgtcagttag cgccgcagtt accggatatg
          cagcaaacgc tgctggataa acactatctg
  661 cgtaagcatg gcgcgaaggc atattacggg
          cagtaa
araC, Escherichia coli str. K-12
substr. MG1655, complete genome,
NCBI Reference Sequence: NC_000913.3,
REGION: 70387-71265, 879 bp
SEQ ID NO: 96
      1 atggctgaag cgcaaaatga tcccctgctg
          ccgggatact cgtttaacgc ccatctggtg
    61 gcgggtttaa cgccgattga ggccaacggt
          tatctcgatt tttttatcga ccgaccgctg
  121 ggaatgaaag gttatattct caatctcacc
          attcgcggtc agggggtggt gaaaaatcag
  181 ggacgagaat ttgtctgccg accgggtgat
          attttgctgt tcccgccagg agagattcat
  241 cactacggtc gtcatccgga ggctcgcgaa
          tggtatcacc agtgggttta ctttcgtccg
  301 cgcgcctact ggcatgaatg gcttaactgg
          ccgtcaatat ttgccaatac gggtttcttt
  361 cgcccggatg aagcgcacca gccgcatttc
          agcgacctgt ttgggcaaat cattaacgcc
  421 gggcaagggg aagggcgcta ttcggagctg
          ctggcgataa atctgcttga gcaattgtta
  481 ctgcggcgca tggaagcgat taacgagtcg
          ctccatccac cgatggataa tcgggtacgc
  541 gaggcttgtc agtacatcag cgatcacctg
          gcagacagca attttgatat cgccagcgtc
  601 gcacagcatg tttgcttgtc gccgtcgcgt
          ctgtcacatc ttttccgcca gcagttaggg
  661 attagcgtct taagctggcg cgaggaccaa
          cgcattagtc aggcgaagct gcttttgagc
  721 actacccgga tgcctatcgc caccgtcggt
          cgcaatgttg gttttgacga tcaactctat
  781 ttctcgcgag tatttaaaaa atgcaccggg
          gccagcccga gcgagtttcg tgccggttgt
  841 gaagaaaaag tgaatgatgt agccgtcaag
          ttgtcataa
```

In some embodiments of any of the aspects, the amino acid sequence encoded by the endogenous arabinose utilization gene comprises SEQ ID NO: 97-100 or an amino acid sequence that is at least 95% (e.g., at least 95%, at least 96%, at least 97%, at least 98%, or at least 99%) identical to the sequence of SEQ ID NO: 97-100 that maintains the same functions as SEQ ID NO: 97-100 (e.g., arabinose utilization).

```
araB, ribulokinase [Escherichia coli
str. K-12 substr. MG1655], NCBI
Reference Sequence: NP_414605.1, 566 aa
SEQ ID NO: 97
    1 maiaigldfg sdsvralavd catgeeiats
        vewyprwqkg qfcdapnnqf rhhprdyies
  61 meaalktvla elsveqraav vgigvdstgs
        tpapidadgn vlalrpefae npnamfvlwk
121 dhtaveeaee itrlchapgn vdysryiggi
        yssewfwaki ihvtrqdsav aqsaaswiel
181 cdwvpallsg ttrpqdirrg rcsaghkslw
        heswggippa sffdeldpil nrhlpsplft
241 dtwtadipvg tlcpewaqrl glpeswisg
        gafdchmgav gagaqpnalv kvigtstcdi
301 liadkqsvge ravkgicgqv dgsvvpgfig
        leagqsafgd iyawfgrvlg wpleqlaaqh
361 pelktqinas qkqllpalte awaknpsldh
        lpvvldwfng rrtpnanqrl kgvitdlnla
421 tdapllfggl iaatafgara imecftdqgi
        avnnvmalgg iarknqvimq accdvlnrpl
481 qivasdqcca Igaaifaava akvhadipsa
        qqkmasavek tlqpcseqaq rfeqlyrryq
541 qwamsaeqhy iptsapaqaa qavatl
araA, L-arabinose isomerase
[Escherichia coli str. K-12 substr.
MG1655]NCBI Reference Sequence:
NP_414604.1, 500 aa
SEQ ID NO: 98
    1 mtifdnyevw fvigsqhlyg petlrqvtqh
        aehvvnalnt eaklpcklvl kplgttpdei
  61 taicrdanyd drcaglvvwl htfspakmwi
        ngltmlnkpl lqfhtqfnaa ipwdsidmdf
121 mnlnqtahgg refgfigarm rqqhavvtgh
        wqdkqaheri gswmrqavsk qdtrhlkvcr
181 fgdnmrevav tdgdkvaaqi kfgfsvntwa
        vgdlvqvvns isdgdvnalv deyescytmt
241 patqihgkkr qnvleaarie igmkrfleqg
        gfhaftttfe dlhglkqlpg lavqrlmqqg
301 ygfagegdwk taallrimkv mstglqggts
        fmedytyhfe kgndlvlgsh mlevcpsiaa
361 eekpildvqh igiggkddpa rlifntqtgp
        aivaslidlg dryrlivnci dtvktphslp
421 kipvanalwk aqpdlptase awilaggahh
        tvfshalnln dmrqfaemhd ieitvidndt
481 rlpafkdalr wnevyygfrr
araD, L-ribulose-5-
phosphate 4-epimerase AraD
[Escherichia coli str. K-
SEQ ID NO: 99
12 substr. MG1655], NCBI Reference
Sequence: NP 414603.1, 231 aa
    1 mledlkrqvl eanlalpkhn Ivtltwgnvs
        avdrergvfv ikpsgvdysv mtaddmvvvs
  61 ietgevvegt kkpssdtpth rllyqafpsi
        ggivhthsrh atiwaqagqs ipatgtthad
121 yfygtipctr kmtdaeinge yewetgnviv
        etfekqgida aqmpgvlvhs hgpfawgkna
181 edavhnaivl eevaymgifc rqlapqlpdm
        qqtlldkhyl rkhgakayyg q
araC, DNA-binding
transcriptional dual regulator AraC
[Escherichia coli str. K-12 substr.
MG1655], NCBI Reference Sequence:
NP 414606.1, 292 aa
SEQ ID NO: 100
    1 maeaqndpll pgysfnahlv agitpieang
        yldffidrpl gmkgyilnlt irgqgvvknq
  61 grefverpgd illfppgeih hygrhpeare
        wyhqwvyfrp raywhewinw psifantgff
121 rpdeahqphf sdlfgqiina gqgegrysel
        lainlleqll Irrmeaines Ihppmdnrvr
181 eacqyisdhl adsnfdiasv aqhvclspsr
        ishlfrqqlg isvlswredq risqakllls
241 ttrmpiatvg rnvgfddqly fsrvfkkctg
        aspsefragc eekvndvavk is
```

In some embodiments of any of the aspects, the engineered heterotroph comprises an inhibitor of arabinose utilization gene. Non-limiting examples of arabinose utilization gene (e.g., araB, araA, araD, araBAD operon) inhibitors include xylose and fucose; see e.g., Koirala et al., Journal of Bacteriology (2016) 198(3), 386-393; Wilcox et al., Journal of Biological Chemistry (1974) 249(9), 2946-2952.

In some embodiments of any of the aspects, the engineered heterotroph comprises at least one exogenous copy of at least one functional secondary product synthesis gene. In some embodiments of any of the aspects, the at least one functional secondary product synthesis gene is heterologous. In some embodiments of any of the aspects, the at least one functional secondary product synthesis gene comprises a violacein synthesis gene. In some embodiments of any of the aspects, the at least one functional secondary product synthesis gene comprises a β-carotene synthesis gene.

In some embodiments of any of the aspects, the engineered heterotroph comprises at least one synthesis gene for a secondary product that can be synthesized from sucrose (e.g., from the sucrose feedstock). Non-limiting examples of secondary products that can be synthesized from sucrose include: violacein, β-carotene, ethanol (e.g., bioethanol), or biofuels (e.g., biodiesel).

In some embodiments of any of the aspects, the engineered heterotroph comprises at least one exogenous copy of at least one functional secondary product synthesis gene. In some embodiments of any of the aspects, the at least one functional secondary product synthesis gene comprises a violacein synthesis gene. Violacein is a naturally-occurring bis-indole pigment with antibiotic (anti-bacterial, anti-viral, anti-fungal and anti-tumor) properties. Violacein occurs in several species of bacteria and accounts for their striking purple hues. See e.g., Balibar and Walsh, In vitro biosynthesis of violacein from L-tryptophan by the enzymes VioA-E from Chromobacterium violaceum, Biochemistry. 2006 Dec. 26; 45(51):15444-5; the contents of which are incorporated herein by reference in their entirety.

In some embodiments of any of the aspects, the engineered heterotroph comprises VioA, VioB, VioC, VioD, VioE, or any combination thereof. In some embodiments of any of the aspects, the engineered heterotroph comprises Chromobacterium violaceum VioA, Chromobacterium violaceum VioB, Chromobacterium violaceum VioC, Chromobacterium violaceum VioD, Chromobacterium violaceum VioE, or any combination thereof. In some embodiments of any of the aspects, the engineered heterotroph comprises Chromobacterium violaceum VioA. In some embodiments of any of the aspects, the engineered heterotroph comprises Chromobacterium violaceum VioB. In some embodiments of any of the aspects, the engineered heterotroph comprises Chromobacterium violaceum VioC. In some embodiments of any of the aspects, the engineered heterotroph comprises Chromobacterium violaceum VioD. In some embodiments of any of the aspects, the engineered heterotroph comprises Chromobacterium violaceum VioE. In some embodiments of any of the aspects, the engineered heterotroph comprises Chromobacterium violaceum VioA, Chromobacterium violaceum VioB, Chromobacterium violaceum V ioC, Chromobacterium violaceum VioD, and Chromobacterium violaceum VioE.

In some embodiments of any of the aspects, the engineered heterotroph comprises a functional violacein synthesis gene (e.g., Chromobacterium violaceum VioA) comprising SEQ ID NO: 51, or a nucleic acid sequence that is at least 95% (e.g., at least 95%, at least 96%, at least 97%, at least 98%, or at least 99%) identical to the sequence of SEQ ID NO: 51 that maintains the same functions as SEQ ID NO: 51 (e.g., L-tryptophan oxidase).

```
SEQ ID NO: 51, Chromobacterium violaceum
ATCC 12472, complete genome, NCBI
Reference Sequence: NC_005085.1,
REGION: complement (3565032-3566288),
1257 bp
      1 atgaagcatt cttccgatat ctgcattgtc
          ggcgccggca tcagcggcct gacctgcgcc
    61 agccatctgc tcgactcgcc cgcttgccgc
          ggcctgtcgc tgcgcatctt cgacatgcag
  121 caggaggcgg gcggccgcat ccgctcgaag
          atgctggatg gcaaggcgtc gatagagctg
  181 ggcgcggggc gatactcccc gcagctgcac
          ccgcatttcc agagcgcgat gcagcattac
  241 agccagaaga gcgaggtgta tccgttcacc
          cagctgaaat tcaagagcca tgtccagcag
  301 aagctgaagc gggcgatgaa cgagttgtcg
          cccaggctga aagagcatgg caaggaatcc
  361 tttctccagt tcgtcagccg ctaccagggc
          catgacagcg cggtgggcat gatccgctcc
  421 atgggctacg acgcgctgtt cctgcccgac
          atctcggccg agatggccta cgacatcgtc
  481 ggcaagcacc cggaaatcca gagcgtgacc
          gataacgacg ccaaccagtg gttcgcggcg
  541 gaaacgggct ttgcgggcct gatccagggc
          atcaaggcca aggtcaaggc tgccggcgcg
  601 cgcttcagcc tgggttaccg gctgctgtcg
          gtgaggacgg acggcgacgg ctacctgctg
  661 caactggccg gcgacgacgg ctggaagctg
          gaacaccgga cccgccatct gatcctggcc
  721 attcctccgt cggcgatggc cgggctcaat
          gtcgacttcc ccgaggcgtg gagcggcgcg
  781 cgctacggct cgctgccgct gttcaagggt
          ttcctcacct acggcgagcc atggtggctg
  841 gactacaagc tggacgacca ggtgctgatc
          gtcgacaacc cgctgcgcaa gatctacttc
  901 aagggcgaca agtacctgtt cttctacacc
          gacagcgaga tggccaatta ctggcgcggc
  961 tgcgtggccg aaggagagga cggctacctg
          gagcagatcc gcacccatct ggccagcgcg
1021 ctgggcatcg ttcgcgagcg cattccccag
          cccctcgccc atgtgcacaa gtattgggcg
1081 catggcgtgg agttctgccg cgacagcgat
          atcgaccatc cgtccgcgct cagccaccgc
1141 gacagcggca tcatcgcctg ttcggacgcc
          tacaccgagc actgcggctg gatggagggc
1201 ggcctgctca gcgcccgcga agccagccgt
          ctgctgctgc agcgcatcgc cgcgtga
```

In some embodiments of any of the aspects, the amino acid sequence encoded by the functional violacein synthesis gene (e.g., Chromobacterium violaceum VioA) comprises SEQ ID NO: 52, or an amino acid sequence that is at least 95% (e.g., at least 95%, at least 96%, at least 97%, at least 98%, or at least 99%) identical to the sequence of SEQ ID NO: 52 that maintains the same functions as SEQ ID NO: 52 (e.g., L-tryptophan oxidase).

```
SEQ ID NO: 52, L-tryptophan oxidase
VioA[Chromobacterium violaceum], NCBI
Reference Sequence: WP_011136821.1,
418 aa
    1 mkhssdiciv gagisgitca shlldspacr
        glslrifdmq qeaggrirsk midgkasiel
  61 gagryspqlh phfqsamqhy sqksevypft
        qlkfkshvqq klkramnels prlkehgkes
121 flqfvsryqg hdsavgmirs mgydalflpd
        isaemaydiv gkhpeiqsvt dndanqwfaa
181 etgfagliqg ikakvkaaga rfslgyrlls
        vrtdgdgyll qlagddgwkl ehrtrhlila
241 ippsamagin vdfpeawsga rygslplfkg
        fltygepwwl dyklddqvli vdnplrkiyf
301 kgdkylffyt dsemanywrg cvaegedgyl
        eqirthlasa lgivreripq plahvhkywa
361 hgvefcrdsd idhpsalshr dsgiiacsda
        ytehcgwmeg gllsareasr lllqriaa
```

In some embodiments of any of the aspects, the engineered heterotroph comprises a functional violacein synthesis gene (e.g., Chromobacterium violaceum VioB) comprising SEQ ID NO: 53, or a nucleic acid sequence that is at least 95% (e.g., at least 95%, at least 96%, at least 97%, at least 98%, or at least 99%) identical to the sequence of SEQ ID NO: 53 that maintains the same functions as SEQ ID NO: 53 (e.g., iminophenyl-pyruvate dimer synthase).

```
SEQ ID NO: 53, Chromobacterium violaceum
ATCC 12472, complete genome, NCBI
Reference Sequence: NC_005085.1,
REGION: complement (3561961-3564957),
2997 bp
      1 atgagcattc tggattttcc acgcatccat
          ttccgcggct gggcgcgggt caacgcgccc
    61 accgccaacc gcgatccgca cggccacatc
          gacatggcca gcaatacggt ggccatggca
  121 ggcgaaccgt tcgacctcgc gcgccatccg
          accgagttcc accgccacct gcggtcgctg
  181 gggccgcgtt tcggcctgga cggccgggct
          gacccggaag ggccgttcag cctggccgag
  241 ggctacaacg cggccggcaa caaccatttc
          tcctgggaga gcgccaccgt cagccacgtg
  301 cagtgggatg gcggcgaagc ggaccgcggc
          gacggcctgg tcggcgccag gctggcgctg
  361 tgggggcatt acaacgatta cctgcgcacc
          accttcaacc gcgcgcgctg ggtggacagc
  421 gaccccaccc gccgcgacgc ggcgcagatc
          tacgccgggc agttcacgat cagcccggcc
  481 ggcgccggac cgggcacgcc ctggctgttc
          accgccgaca tcgacgacag ccacggcgcg
  541 cgctggacgc gcggcggcca catcgccgag
          cgcggcggcc atttcctgga cgaggagttc
  601 ggcctggcgc ggctgttcca gttctcggtg
          cccaaagacc atccgcactt cctgttccac
  661 ccggggccat tcgattccga agcctggcgc
          aggctgcagc tggcgctgga ggacgacgac
  721 gtgctcggcc tgacggtgca gtacgcgctg
          ttcaatatgt cgacgccgcc gcaacccaac
  781 tcgccggtgt tccacgacat ggtcggcgtg
          gtcggcctgt ggcggcgcgg cgaactggcc
  841 agctacccgg ccggccggct gctgcgtccg
          cgccagcccg ggctgggcga tctgacgctg
  901 cgcgtaagcg gcggccgcgt ggcgctgaat
          ctggcctgcg ccattccgtt ctccacccgg
  961 gcggcgcagc cgtccgcgcc ggacaggctg
          acgcccgatc tcggggccaa gctgccgttg
1021 ggcgacctgc tgctgcgcga cgaggacggc
          gcgttgctgg cgcgggtgcc gcaggcgctt
1081 taccaggatt actggacgaa ccacggcatc
          gtcgacctgc cgctgctgcg cgagcccagg
1141 ggctcgctga cgctgtccag cgagctggcc
          gaatggcgcg agcaggactg ggtcacgcag
1201 tccgacgcct ccaatctttatttggaagcg
          ccggaccgcc gccacggccg tttctttccg
1261 gaaagcatcg cgctgcgcag ctatttccgc
          ggcgaggccc gcgcgcgccc ggacattccc
1321 caccggatcg aggggatggg tctggtcggc
          gtggagtcgc gccaggacgg cgatgccgcc
1381 gaatggcggc tgaccggcct gcggcccggc
          ccggcgcgca tcgtgctcga cgacggcgcg
1441 gaggcgatcc cgctgcgggt gctgccggac
          gactgggcgt tggacgacgc gacggtggag
1501 gaggtcgatt acgccttcct gtaccggcac
          gtgatggcct attacgagct ggtctacccg
1561 ttcatgtccg acaaggtgtt cagcctggcc
          gaccgctgca agtgcgagac ctacgccagg
1621 ctgatgtggc agatgtgcga tccgcagaac
          cggaacaaga gctactacat gcccagcacc
1681 cgcgagctgt cggcgcccaa ggccaggctg
          ttcctcaaat acctggccca tgtcgagggc
1741 caggccaggc tgcaggcgcc gccgccggcc
          gggccggcgc gcatcgagag caaggcccag
1801 ctggcggccg agctgcgcaa ggcggtggat
          ctggagttgt cggtgatgct gcagtacctg
1861 tacgccgcct attccattcc caattacgcc
          cagggccagc agcgggtgcg cgacggcgcg
1921 tggacggcgg agcagctgca gctggcctgc
          ggcagcggcg accggcgccg cgacggcggc
1981 atccgcgccg cgctgctgga gatcgcccac
          gaggagatga tccattacct ggtggtcaac
2041 aacctgctga tggcgctggg cgagccgttc
          tacgccggcg tgccgctgat gggcgaggcg
2101 gcgcggcagg cgttcggcct ggacaccgaa
          ttcgcgctgg agccgttctc cgagtcgacg
2161 ctggcgcgct tcgtccggct ggaatggccg
          cacttcatcc ctgcgccggg caaatccatc
2221 gccgactgct acgccgccat ccgccaggcc
          tttctcgatc tgcccgacct gttcggcggc
2281 gaggccggca agcgcggcgg cgagcaccac
          ttgttcctca acgagctgac caaccgcgcc
2341 catcccggct accagctgga ggtgttcgat
          cgcgacagcg cgctgttcgg catcgccttc
2401 gtcaccgacc agggcgaggg cggggcgctg
          gactcgccgc attacgagca ttegcatttc
2461 cagcggctgc gggagatgtc ggccaggatc
          atggcgcagt ccgcgccgtt cgagccggcg
2521 ttgccggcgc tgcgcaaccc ggtgctggac
          gagtcgccgg gctgccagcg cgtggcggac
2581 ggacgggcgc gcgcgctgat ggcgctgtac
          cagggcgtgt acgagctgat gttcgcgatg
2641 atggcgcagc acttcgcggt caagccgctg
          ggcagcctca ggcgctcgcg gctgatgaac
2701 gcggcgatcg acctgatgac cggcctgctc
          aggccgctgt cctgcgcgct gatgaacctg
2761 ccgtcgggca tcgccggacg caccgccggg
          ccgccgctgc cggggccggt ggatacccgc
2821 agctacgacg actacgcgct gggctgccgg
          atgctggcgc ggcgctgcga gcgcctgctg
2881 gagcaggcgt cgatgctgga gccgggctgg
          ctgcccgacg cgcaaatgga actgctggat
2941 ttctaccgcc ggcagatgct ggatttggct
          tgtggaaagc tttctagaga ggcctga
```

In some embodiments of any of the aspects, the amino acid sequence encoded by the functional violacein synthesis gene (e.g., Chromobacterium violaceum VioB) comprises SEQ ID NO: 54, or an amino acid sequence that is at least 95% (e.g., at least 95%, at least 96%, at least 97%, at least 98%, or at least 99%) identical to the sequence of SEQ ID NO: 54 that maintains the same functions as SEQ ID NO: 54 (e.g., iminophenyl-pyruvate dimer synthase).

```
SEQ ID NO: 54, iminophenyl-pyruvate dimer
synthase VioB [Chromobacterium
violaceum], NCBI Reference
Sequence: WP_011136820.1, 998 aa
    1 msildfprih frgwarvnap tanrdphghi
        dmasntvama gepfdlarhp tefhrhlrsl
  61 gprfgldgra dpegpfslae gynaagnnhf
        swesatvshv qwdggeadrg dgivgarlal
121 wghyndylrt tfnrarwvds dptrrdaaqi
        yagqftispa gagpgtpwlf tadiddshga
181 rwtrgghiae rgghfldeef glarlfqfsv
        pkdhphflfh pgpfdseawr rlqlaleddd
241 vlgltvqyal fnmstppqpn spvfhdmvgv
        vglwrrgela sypagrllrp rqpglgdltl
301 rvsggrvaln lacaipfstr aaqpsapdrl
        tpdlgaklpl gdlllrdedg allarvpqal
361 yqdywtnhgi vdlpllrepr gsltlssela
        ewreqdwvtq sdasnlylea pdrrhgrffp
421 esialrsyfr geararpdip hriegmglvg
        vesrqdgdaa ewrltglrpg parivlddga
481 eaiplrvlpd dwalddatve evdyaflyrh
        vmayyelvyp fmsdkvfsla drckcetyar
541 Imwqmcdpqn rnksyympst relsapkarl
        flkylahveg qarlqapppa gparieskaq
601 laaelrkavd lelsvmlqyl yaaysipnya
        qgqqrvrdga wtaeqlqlac gsgdrrrdgg
661 iraalleiah eemihylvvn nllmalgepf
        yagvplmgea arqafgldte falepfsest
721 larfvrlewp hfipapgksi adcyaairqa
        fldlpdlfgg eagkrggehh iflneltnra
781 hpgyqlevfd rdsalfgiaf vtdqgeggal
        dsphyehshf qrlremsari maqsapfepa
841 Ipalrnpvld espgcqrvad graralmaly
        qgvyelmfam maqhfavkpl gslrrsrlmn
901 aaidlmtgll rplscalmnl psgiagrtag
        pplpgpvdtr syddyalgcr mlarrcerll
961 eqasmlepgw Ipdaqmelld fyrrqmldla
        cgklsrea
```

In some embodiments of any of the aspects, the engineered heterotroph comprises a functional violacein synthesis gene (e.g., Chromobacterium violaceum VioC) comprising SEQ ID NO: 55, or a nucleic acid sequence that is at least 95% (e.g., at least 95%, at least 96%, at least 97%, at least 98%, or at least 99%) identical to the sequence of SEQ ID NO: 55 that maintains the same functions as SEQ ID NO: 55 (e.g., violacein synthase).

SEQ ID NO: 55, Chromobacterium violaceum
ATCC 12472, complete genome, NCBI
Reference Sequence: NC_005085.1,
(3560670-3561959, complement), 1290 aa
ATGAAAAGAGCAATCATAGTCGGAGGCGGGCTCGCC
GGCGGGCTGACCGCCATCTACCTGGCGAAGCGCGG
CTACGAGGTCCACGTGGTGGAAAAGCGCGGCGACC
CGCTGCGGGACCTGTCTTCCTACGTGGATGTGGTC
AGCTCGCGGGCGATAGGCGTCAGCATGACCGTGCG
CGGCATCAAGTCGGTGCTGGCGGCCGGCATTCCGC
GCGCGGAGCTGGACGCCTGCGGCGAACCCATCGTG
GCGATGGCGTTTTCCGTCGGCGGCCAGTACCGGAT
GCGGGAGCTCAAGCCGCTGGAGGATTTCCGCCCGC
TGTCGCTGAACCGCGCGGCGTTTCAGAAGCTGCTG
AACAAGTACGCCAACCTGGCCGGCGTCCGCTACTA
CTTCGAGCACAAGTGCCTGGACGTGGATCTGGACG
GCAAGTCGGTGCTGATCCAGGGCAAGGACGGCCAG
CCGCAGCGCTTGCAGGGCGATATGATCATCGGCGC
CGACGGCGCGCACTCGGCGGTGCGGCAGGCGATGC
AGAGCGGGTTGCGCCGCTTCGAATTCCAGCAGACT
TTCTTCCGCCACGGCTACAAGACGCTGGTGCTGCC
GGACGCGCAGGCGCTGGGCTACCGCAAGGACACGC
TGTATTTCTTCGGCATGGACTCCGGCGGCCTGTTC
GCCGGCCGCGCCGCCACCATCCCGGACGGCAGCGT
CAGCATCGCGGTCTGCCTGCCGTACAGCGGCAGCC
CCAGCCTGACCACCACCGACGAGCCGACGATGCGC
GCCTTTTTCGACCGTTACTTCGGCGGCCTGCCGCG
GGACGCGCGCGACGAGATGCTGCGCCAGTTCCTGG
CCAAGCCCAGCAACGACCTGATCAACGTCCGTTCC
AGCACCTTCCACTACAAGGGCAATGTGCTGCTGCT
GGGCGACGCCGCCCACGCCACCGCGCCTTTCCTCG
GCCAGGGCATGAACATGGCGCTGGAGGACGCGCGC
ACCTTCGTCGAGCTGCTGGACCGCCACCAGGGCGA
CCAGGACAAGGCCTTTCCCGAGTTCACCGAGCTGC
GCAAGGTGCAGGCCGACGCGATGCAGGACATGGCG
CGCGCCAACTACGACGTGCTCAGCTGCTCCAATCC
CATCTTCTTCATGCGGGCCCGCTACACCCGCTACA
TGCATAGCAAGTTTCCCGGCCTTTACCCGCCGGAC
ATGGCGGAGAAGCTGTACTTCACGTCCGAGCCGTA
CGACAGACTGCAGCAGATCCAGAGAAAACAGAACG
TTTGGTACAAGATAGGGAGGGTCAACTGA

In some embodiments of any of the aspects, the amino acid sequence encoded by the functional violacein synthesis gene (e.g., Chromobacterium violaceum VioC) comprises SEQ ID NO: 56, or an amino acid sequence that is at least 95% (e.g., at least 95%, at least 96%, at least 97%, at least 98%, or at least 99%) identical to the sequence of SEQ ID NO: 56 that maintains the same functions as SEQ ID NO: 56 (e.g., violacein synthase, monooxygenase).

```
SEQ ID NO: 56, FAD-dependent monooxygenase
[Chromobacterium violaceum], NCBI Reference
Sequence: WP_011136819.1, 429 aa
    1 mkraiivggg laggltaiyl akrgyevhvv
        ekrgdplrdl ssyvdvvssr aigvsmtvrg
  61 iksvlaagip raeldacgep ivamafsvgg
        qyrmrelkpl edfrplslnr aafqkllnky
121 anlagvryyf ehkcldvdld gksvliqgkd
        gqpqrlqgdm iigadgahsa vrqamqsglr
181 rfefqqtffr hgyktlvlpd aqalgyrkdt
        lyffgmdsgg ifagraatip dgsvsiavcl
241 pysgspsltt tdeptmraff dryfgglprd
        ardemlrqfl akpsndlinv rsstfhykgn
301 vlllgdaaha tapflgqgmn maledartfv
        elldrhqgdq dkafpeftel rkvqadamqd
361 maranydvls csnpiffmra rytrymhskf
        pglyppdmae klyftsepyd rlqqiqrkqn
421 vwykigrvn
```

In some embodiments of any of the aspects, the engineered heterotroph comprises a functional violacein synthesis gene (e.g., Chromobacterium violaceum VioD) comprising SEQ ID NO: 57, or a nucleic acid sequence that is at least 95% (e.g., at least 95%, at least 96%, at least 97%, at least 98%, or at least 99%) identical to the sequence of SEQ ID NO: 57 that maintains the same functions as SEQ ID NO: 57 (e.g., tryptophan hydroxylase, monooxygenase).

```
SEQ ID NO: 57, Chromobacterium violaceum
ATCC 12472, complete genome, NCBI
Reference Sequence: NC_005085.1,
REGION: complement (3559549-3560670),
1122 bp
      1 atgaagattc tggtcatcgg cgcggggccg
          gccggcctgg tgttcgccag ccaactgaaa
    61 caggcgcgtc cgctgtgggc gatagacatc
          gtcgaaaaga acgacgagca ggaagtgctg
  121 ggctggggcg tggtgctgcc cggccggccc
          ggccagcatc cggccaatcc gctgtcctac
  181 ctggacgcgc cggagaggct gaatccgcag
          ttcctggaag acttcaagct ggtccaccac
  241 aacgagccca gcctgatgag caccggcgtg
          ctgctgtgcg gcgtggagcg ccgcggcctg
  301 gtgcacgcct tgcgcgacaa gtgccgctcg
          cagggcatcg ccatccgctt cgaatcgccg
  361 ctgctggagc atggcgagct gccgctggcc
          gactacgacc tggtggtgct ggccaacggc
  421 gtcaatcaca agaccgccca cttcaccgag
          gcgctggtgc cgcaggtgga ctacggccgc
  481 aacaagtaca tctggtacgg caccagccag
          ctgttcgacc agatgaacct ggtgttccgc
  541 acccacggca aggacatttt catcgcccac
          gcctacaagt actcggacac gatgagcacc
  601 ttcatcgtcg agtgcagcga ggagacctat
          gcccgcgccc gcctgggcga gatgtcggaa
  661 gaggcgtcgg ccgaatacgt cgccaaggtg
          ttccaggccg agctgggcgg ccacggcctg
  721 gtgagccagc ccggcctcgg ctggcgcaac
          ttcatgaccc tgagccacga ccgctgccac
  781 gacggcaagc tggtgctgct gggcgacgcg
          ctgcagtccg gccacttctc catcggccac
  841 ggcaccacga tggcggtggt ggtggcgcag
          ctgctggtga aggcgctgtg caccgaggac
  901 ggcgtgccgg ccgcgctgaa gcgcttcgag
          gagcgcgcgc tgccgctggt ccagctgttc
  961 cgcggccatg ccgacaacag ccgggtctgg
          ttcgagacgg tggaggagcg catgcacctg
1021 tccagcgccg agttcgtgca gagcttcgac
          gcgcgccgca agtcgctgcc gccgatgccg
1081 gaagcgctgg cgcagaacct gcgctacgcg
          ctgcaacgct ga
```

In some embodiments of any of the aspects, the amino acid sequence encoded by the functional violacein synthesis gene (e.g., Chromobacterium violaceum VioD) comprises SEQ ID NO: 58, or an amino acid sequence that is at least 95% (e.g., at least 95%, at least 96%, at least 97%, at least 98%, or at least 99%) identical to the sequence of SEQ ID NO: 58 that maintains the same functions as SEQ ID NO: 58 (e.g., tryptophan hydroxylase).

```
SEQ ID NO: 58, tryptophan hydroxylase
VioD [Chromobacterium violaceum], NCBI
Reference Sequence: WP_011136818.1,
373 aa
    1 mkilvigagp aglvfasqlk qarplwaidi
        vekndeqevl gwgvvlpgrp gqhpanplsy
  61 idaperlnpq fledfklvhh nepslmstgv
        llcgverrgl vhalrdkcrs qgiairfesp
121 llehgelpla dydlvvlang vnhktahfte
        alvpqvdygr nkyiwygtsq ifdqmnlvfr
181 thgkdifiah aykysdtmst fivecseety
        ararlgemse easaeyvakv fqaelgghgl
241 vsqpglgwrn fintlshdrch dgklvllgda
        iqsghfsigh gttmavvvaq livkalcted
301 gvpaalkrfe eralplvqlf rghadnsrvw
        fetveermhl ssaefvqsfd arrkslppmp
361 ealaqnlrya iqr
```

In some embodiments of any of the aspects, the engineered heterotroph comprises a functional violacein synthesis gene (e.g., Chromobacterium violaceum VioE) comprising SEQ ID NO: 59, or a nucleic acid sequence that is at least 95% (e.g., at least 95%, at least 96%, at least 97%, at least 98%, or at least 99%) identical to the sequence of SEQ ID NO: 59 that maintains the same functions as SEQ ID NO: 59 (e.g., violacein biosynthesis).

```
SEQ ID NO: 59, Chromobacterium violaceum
ATCC 12472, complete genome, NCBI
Reference Sequence: NC_005085.1,
REGION: complement (3558964-3559539),
576 bp
    1 atggaaaacc gggaaccgcc gctgctgccg
        gcgcgctgga gcagcgccta tgtgtcgtac
  61 tggagtccga tgctgccgga tgaccagctg
        acgtccggct actgctggtt cgactacgag
121 cgcgacatct gtcggataga cggcctgttc
        aatccctggt cggagcgcga caccggctac
181 cggctgtgga tgtccgaggt cggcaacgcc
        gccagcggcc gcacctggaa gcagaaggtg
241 gcctatggcc gcgagcggac cgccctgggc
        gagcagctgt gcgagcggcc gctggacgac
301 gagaccggcc cgttcgccga gctgttcctg
        ccgcgcgacg tgctgcgccg gctgggcgcc
361 cgccatatcg gccgccgcgt ggtgctgggc
        agggaagccg acggctggcg ctaccagcgt
421 ccgggcaagg ggccgtccac gttgtacctg
        gacgccgcca gcggtacgcc gctgaggatg
481 gtgaccgggg acgaggcgtc gcgcgcgtcg
        ctgcgcgatt tccccaacgt cagcgaggcc
541 gagattcccg acgccgtctt cgccgccaag
        cgctag
```

In some embodiments of any of the aspects, the amino acid sequence encoded by the functional violacein synthesis gene (e.g., Chromobacterium violaceum VioE) comprises SEQ ID NO: 60, or an amino acid sequence that is at least 95% (e.g., at least 95%, at least 96%, at least 97%, at least 98%, or at least 99%) identical to the sequence of SEQ ID NO: 60 that maintains the same functions as SEQ ID NO: 60 (e.g., violacein biosynthesis).

```
SEQ ID NO: 60, violacein biosynthesis
enzyme VioE [Chromobacterium violaceum],
NCBI Reference Sequence: WP_011136817.1,
191 aa
    1 menreppllp arwssayvsy wspmlpddql
        tsgycwfdye rdicridglf npwserdtgy
  61 rlwmsevgna asgrtwkqkv aygrertalg
        eqlcerpldd etgpfaelfl prdvlrrlga
121 rhigrrvvlg readgwryqr pgkgpstlyl
        daasgtplrm vtgdeasras Irdfpnvsea
181 eipdavfaak r
```

In some embodiments of any of the aspects, the engineered heterotroph comprises at least one exogenous copy of at least one functional secondary product synthesis gene. In some embodiments of any of the aspects, the at least one functional secondary product synthesis gene comprises a β-carotene synthesis gene. β-Carotene is an organic, strongly colored red-orange pigment abundant in plants and fruits. It is a member of the carotenes, which are terpenoids, synthesized biochemically from eight isoprene units and thus having 40 carbons. See e.g., Lemuth et al., Engineering of a plasmid-free Escherichia coli strain for improved in vivo biosynthesis of astaxanthin, Microb Cell Fact. 2011 Apr. 26; 10:29.

In some embodiments of any of the aspects, the engineered heterotroph comprises a geranylgeranyl diphosphate synthase (e.g., CrtE), a phytoene synthase (e.g., CrtB), a phytoene desaturase (e.g., CrtI), a lycopene cyclase (e.g., CrtY), or any combination thereof. In some embodiments of any of the aspects, the engineered heterotroph comprises Pantoea ananatis CrtE, Pantoea ananatis CrtB, Pantoea ananatis CrtI, Pantoea ananatis CrtY, or any combination thereof In some embodiments of any of the aspects, the engineered heterotroph comprises Pantoea ananatis CrtE. In some embodiments of any of the aspects, the engineered heterotroph comprises Pantoea ananatis CrtB. In some embodiments of any of the aspects, the engineered heterotroph comprises Pantoea ananatis CrtI. In some embodiments of any of the aspects, the engineered heterotroph comprises Pantoea ananatis CrtY.

In some embodiments of any of the aspects, the engineered heterotroph comprises a functional violacein synthesis gene (e.g., Pantoea ananatis CrtE) comprising SEQ ID NO: 61, or a nucleic acid sequence that is at least 95% (e.g., at least 95%, at least 96%, at least 97%, at least 98%, or at least 99%) identical to the sequence of SEQ ID NO: 61 that maintains the same functions as SEQ ID NO: 61 (e.g., geranylgeranyl diphosphate synthase).

| Pantoea ananatis LMG 20103, complete genome, NCBI Reference | |
|---|---|
| Sequence: NC_013956.2, REGION: 4621138-4622046, 909 bp, | |
| SEQ ID NO: 61 | |
| 1 | atgacggtct gcgcaaaaaa acacgttcat ctcactcgcg atgctgcgga gcagttactg |
| 61 | gctgatattg atcgacgcct tgatcagtta ttgcccgtgg agggagaacg ggatgttgtg |
| 121 | ggtgccgcga tgcgtgaagg tgcgctggca ccgggaaaac gtattcgccc catgttgctg |
| 181 | ttgctgaccg cccgcgatct gggttgcgct gtcagccatg acggattact ggatttggcc |
| 241 | tgtgcggtgg aaatggtcca cgcggcttcg ctgatccttg acgatatgcc ctgcatggac |
| 301 | gatgcgaagc tgcggcgcgg acgccctacc attcattctc attacggaga gcatgtggca |
| 361 | atactggcgg cggttgcctt gctgagtaaa gcctttggcg taattgccga tgcagatggc |
| 421 | ctcacgccgc tggcaaaaaa tcgggcggtt tctgaactgt caaacgccat cggcatgcaa |
| 481 | ggattggttc agggtcagtt caaggatctg tctgaagggg ataagccgcg cagcgctgaa |
| 541 | gctattttga tgacgaatca ctttaaaacc agcacgctgt tttgtgcctc catgcagatg |
| 601 | gcctcgattg ttgcgaatgc ctccagcgaa gcgcgtgatt gcctgcatcg tttttcactt |
| 661 | gatcttggtc aggcatttca actgctggac gatttgaccg atggcatgac cgacaccggt |
| 721 | aaggatagca atcaggacgc cggtaaatcg acgctggtca atctgttagg ccctagggcg |
| 781 | gttgaagaac gtctgagaca acatcttcat cttgccagtg agcatctctc tgcggcctgc |
| 841 | caacacgggc acgccactca acattttatt caggcctggt ttgacaaaaa actcgctgcc |
| 901 | gtcagttaa |

In some embodiments of any of the aspects, the amino acid sequence encoded by the functional β-carotene synthesis gene (e.g., Pantoea ananatis CrtE) comprises SEQ ID NO: 62, or an amino acid sequence that is at least 95% (e.g., at least 95%, at least 96%, at least 97%, at least 98%, or at least 99%) identical to the sequence of SEQ ID NO: 62 that maintains the same functions as SEQ ID NO: 62 (e.g., geranylgeranyl diphosphate synthase).

| MULTISPECIES: polyprenyl synthetase family protein | |
|---|---|
| [Pantoea], NCBI Reference Sequence: WP_014333254.1, 302 aa, | |
| SEQ ID NO: 62 | |
| 1 | mtvcakkhvh ltrdaaeqll adidrrldql lpvegerdvv gaamregala pgkrirpmll |
| 61 | lltardlgca vshdglldla cavemvhaas lilddmpcmd daklrrgrpt ihshygehva |
| 121 | ilaavallsk afgviadadg ltplaknrav selsnaigmq glvqgqfkdl segdkprsae |
| 181 | ailmtnhfkt stlfcasmqm asivanasse ardclhrfsl dlgqafqlld dltdgmtdtg |
| 241 | kdsnqdagks tlvnllgpra veerlrqhlh lasehlsaac qhghatqhfi qawfdkklaa |
| 301 | vs |

In some embodiments of any of the aspects, the engineered heterotroph comprises a functional violacein synthesis gene (e.g., Pantoea ananatis CrtB) comprising SEQ ID NO: 63, or a nucleic acid sequence that is at least 95% (e.g., at least 95%, at least 96%, at least 97%, at least 98%, or at least 99%) identical to the sequence of SEQ ID NO: 63 that maintains the same functions as SEQ ID NO: 63 (e.g., phytoene synthase).

| Pantoea ananatis LMG 20103, complete genome, NCBI Reference | |
|---|---|
| Sequence: NC_013956.2, REGION: 4625970-4626899, 930 bp, | |
| SEQ ID NO: 63 | |
| 1 | ttgaataatc cgtcgttact caatcatgcg gtcgaaacga tggcagttgg ctcgaaaagt |
| 61 | tttgcgacag cctcaaagtt atttgatgca aaaacccggc gcagcgtact gatgctctac |
| 121 | gcctggtgcc gccattgtga cgatgttatt gacgaccaga cgctgggctt ccaggcccgg |
| 181 | cagcctgcct tacaaacgcc cgaacaacgt ctgatgcaac ttgagatgaa aacgcgccag |
| 241 | gcctatgcag gatcgcagat gcacgaaccg gcgtttgcgg cttttcagga agtggctatg |
| 301 | gctcatgata tcgccccggc ttacgegttt gatcatctgg aaggcttcgc catggatgta |
| 361 | cgcgaagcgc aatacagcca actggacgat acgctgcgct attgctatca cgttgcaggc |
| 421 | gttgtcggct tgatgatggc gcaaatcatg ggcgtacggg ataacgccac gctggaccgc |
| 481 | gcctgtgacc ttgggctggc atttcagttg accaatattg ctcgcgatat tgtggacgat |
| 541 | gcgcatgcgg gccgctgtta tctgccggca agctggctgg agcatgaagg tctgaacaaa |
| 601 | gagaattatg cggcacctga aaaccgtcag gcgctgagcc gtatcgcccg tcgtttggtg |
| 661 | caggaagcag aaccttacta tttgtctgcc acagcgggcc tggctgggtt gcccctgcgt |
| 721 | tcggcctggg caatcgctac ggcgaagcag gtttaccgga aaataggtgt caaagttgaa |
| 781 | caggccggtc agcaagcctg ggatcagcgg cagtcaacga ccacgcccga aaaattaacg |
| 841 | ctgctgctgg ccgcctctgg tcaggccctt acttcccgga tgcgggctca tcctccccgc |
| 901 | cctgcgcatc tctggcagcg cccgctctag |

In some embodiments of any of the aspects, the amino acid sequence encoded by the functional β-carotene synthesis gene (e.g., Pantoea ananatis CrtB) comprises SEQ ID NO: 64, or an amino acid sequence that is at least 95% (e.g., at least 95%, at least 96%, at least 97%, at least 98%, or at least 99%) identical to the sequence of SEQ ID NO: 64 that maintains the same functions as SEQ ID NO: 64 (e.g., phytoene synthase).

| MULTISPECIES: phytoene/squalene synthase family protein | |
|---|---|
| [Pantoea], NCBI Reference Sequence: WP_013027995.1, 309 aa, | |
| SEQ ID NO: 64 | |
| 1 | mnnpsllnha vetmavgsks fatasklfda ktrrsvlmly awcrhcddvi ddqtlgfqar |
| 61 | qpalqtpeqr lmqlemktrq ayagsqmhep afaafqevam ahdiapayaf dhlegfamdv |
| 121 | reaqysqldd tlrycyhvag vvglmmaqim gvrdnatldr acdlglafql tniardivdd |
| 181 | ahagrcylpa swleheglnk enyaapenrq alsriarrlv qeaepyylsa taglaglplr |
| 241 | sawaiatakq vyrkigvkve qagqqawdqr qstttpeklt lllaasgqal tsrmrahppr |
| 301 | pahlwqrpl |

In some embodiments of any of the aspects, the engineered heterotroph comprises a functional violacein synthesis gene (e.g., Pantoea ananatis CrtI) comprising SEQ ID NO: 65, or a nucleic acid sequence that is at least 95% (e.g., at least 95%, at least 96%, at least 97%, at least 98%, or at least 99%) identical to the sequence of SEQ ID NO: 65 that maintains the same functions as SEQ ID NO: 65 (e.g., phytoene desaturase).

| Pantoea ananatis LMG 20103, complete genome, NCBI Reference | |
|---|---|
| Sequence: NC_013956.2, REGION: 4624495-4625973, 1479 bp, | |
| SEQ ID NO: 65 | |
| 1 | atgaaaccaa ctacggtaat tggtgcaggc ttcggtggcc tggcactggc aattcgtcta |
| 61 | caggctgcgg ggatccccgt cttactgctt gaacaacgtg ataaacccgg cggtcgggct |
| 121 | tatgtctacg aggatcaggg gtttaccttt gatgcaggcc cgacggttat caccgatccc |
| 181 | agtgccattg aagaactgtt tgcactggca ggaaaacagt taaaagagta tgtcgaactg |
| 241 | ctgccggtta cgccgtttta ccgcctgtgt tgggagtcag ggaaggtctt taattacgat |
| 301 | aacgatcaaa cccggctcga agcgcagatt cagcagttta atccccgcga tgtcgaaggt |
| 361 | tatcgtcagt ttctggacta ttcacgcgcg gtgtttaaag aaggctatct gaagctcggt |
| 421 | actgtccctt ttttatcgtt cagagacatg cttcgcgccg cacctcaact ggcgaaactg |
| 481 | caggcatgga gaagcgttta cagtaaggtt gccagttaca tcgaagatga acatctgcgc |
| 541 | caggcgtttt ctttccactc gctgttggtg ggcggcaatc ccttcgccac ctcatccatt |
| 601 | tatacgttga tacacgcgct ggagcgtgag tggggcgtct ggtttccgcg tggcggcacc |
| 661 | ggcgcattag ttcaggggat gataaagctg tttcaggatc tgggtggtga agtcgtgtta |
| 721 | aacgccagag tcagccatat ggaaacgaca ggaaacaaga ttgaagccgt gcatttagag |
| 781 | gacggtcgca ggttcctgac gcaagccgtc gcgtcaaatg cagatgtggt tcatacctat |
| 841 | cgcgacctgt taagccagca ccctgccgcg gttaagcagt ccaacaaact gcagactaag |
| 901 | cgtatgagta actctctgtt tgtgctctat tttggtttga atcaccatca tgatcagctc |
| 961 | gcgcatcaca cggtttgttt cggcccgcgt taccgcgaac tgattgacga gatttttaat |
| 1021 | catgatggcc tcgcagaaga cttctcactt tatctgcacg cgccctgtgt cacggattcg |
| 1081 | tcactggcgc ctgaaggttg cggcagttac tatgtgttgg cgccggtgcc gcatttaggc |
| 1141 | accgcgaacc tcgactggac ggttgagggg ccaaaactac gcgaccgtat ttttgagtac |
| 1201 | cttgagcagc attacatgcc tggcttacgg agtcagctgg tcacgcacca gatgtttacg |
| 1261 | ccgtttgatt ttcgcgacca gcttaatgcc tatcagggct cagecttttc tgtggagccc |
| 1321 | gttcttaccc agagcgcctg gtttcggccg cataaccgcg ataaaaccat tactaatctc |
| 1381 | tacctggtcg gcgcaggcac gcatcccggc gcaggcattc ctggcgtcat cggctcggca |
| 1441 | aaagcgacag caggtttgat gctggaggat ctgatttga |

In some embodiments of any of the aspects, the amino acid sequence encoded by the functional β-carotene synthesis gene (e.g., Pantoea ananatis CrtI) comprises SEQ ID NO: 66, or an amino acid sequence that is at least 95% (e.g., at least 95%, at least 96%, at least 97%, at least 98%, or at least 99%) identical to the sequence of SEQ ID NO: 66 that maintains the same functions as SEQ ID NO: 66 (e.g., phytoene desaturase).

| phytoene desaturase [Pantoea ananatis], NCBI Reference Sequence | |
|---|---|
| WP_013027994.1, 492 aa, | |
| SEQ ID NO: 66 | |
| 1 | mkpttvigag fgglalairl qaagipvlll eqrdkpggra yvyedqgftf dagptvitdp |
| 1 | saieelfala gkqlkeyvel lpvtpfyrlc wesgkvfnyd ndqtrleaqi qqfnprdveg |
| 121 | yrqfldysra vfkegylklg tvpflsfrdm lraapqlakl qawrsvyskv asyiedehlr |
| 181 | qafsfhsllv ggnpfatssi ytlihalere wgvwfprggt galvqgmikl fqdlggevvl |
| 241 | narvshmett gnkieavhle dgrrfltqav asnadvvhty rdllsqhpaa vkqsnklqtk |
| 301 | rmsnslfvly fglnhhhdql ahhtvcfgpr yrelideifn hdglaedfsl ylhapcvtds |
| 361 | slapegcgsy yvlapvphlg tanldwtveg pklrdrifey leqhympglr sqlvthqmft |
| 421 | pfdfrdqlna yqgsafsvep vltqsawfrp hnrdktitnl ylvgagthpg agipgvigsa |
| 481 | kataglmled li |

In some embodiments of any of the aspects, the engineered heterotroph comprises a functional violacein synthesis gene (e.g., Pantoea ananatis CrtY) comprising SEQ ID NO: 67, or a nucleic acid sequence that is at least 95% (e.g., at least 95%, at least 96%, at least 97%, at least 98%, or at least 99%) identical to the sequence of SEQ ID NO: 67 that maintains the same functions as SEQ ID NO: 67 (e.g., lycopene cyclase).

| Pantoea ananatis LMG 20103, complete genome, NCBI Reference | |
|---|---|
| Sequence: NC_013956.2, REGION: 4623335-4624483, 1149 bp, | |
| SEQ ID NO: 67 | |
| 1 | atgcaaccgc attatgatct gattctcgtg ggggctggac tcgcgaatgg ccttatcgcc |
| 61 | ctgcgtcttc agcagcagca acctgatatg cgtattttgc ttatcgacgc cgcaccccag |
| 121 | gcgggcggga atcatacgtg gtcatttcac cacgatgatt tgactgagag ccaacatcgt |
| 181 | tggatagctt cgctggtggt tcatcactgg cccgactatc aggtacgctt tcccacacgc |
| 241 | cgtcgtaagc tgaacagcgg ctacttctgt attacttctc agcgtttcgc tgaggtttta |
| 301 | cagcgacagt ttggcccgca cttgtggatg gataccgcgg tcgcagaggt taatgcggaa |
| 361 | tctgttcggt tgaaaaaggg tcaggttatc ggtgcccgcg eggtgattga cgggcggggt |
| 421 | tatgcggcaa actcagcact gagcgtgggc ttccaggcgt ttattggcca ggaatggcga |
| 481 | ttgagccacc cgcatggttt atcgtctccc attatcatgg atgccacggt cgatcagcaa |
| 541 | aatggttatc gcttcgtgta cagcctgccg ctctcgccga ccagattgtt aattgaagac |
| 601 | acgcactata tcgataatgc gacattagat cctgaacgcg cgcggcaaaa tatttgcgac |
| 661 | tatgccgcgc aacagggttg gcagcttcag acattgctgc gtgaagaaca gggcgcctta |
| 721 | cccatcaccc tgtcgggcaa tgccgaggca ttctggcagc agcgccccct ggcctgtagt |
| 781 | ggattacgtg ccggtctgtt ccatcctacc accggctatt cactgccgct ggcggttgcc |
| 841 | gtggccgacc gcctgagcgc acttgatgtc tttacgtcgg cctcaattca ccaggctatt |
| 901 | aggcattttg cccgcgagcg ctggcagcag cagcgctttt tccgcatgct gaatcgcatg |
| 961 | ctgtttttag ccggacccgc cgattcacgc tggcgggtta tgcagcgttt ttatggttta |
| 1021 | cctgaagatt taattgcccg tttttatgcg ggaaaactca cgctgaccga tcggctacgt |
| 1081 | attctgagcg gcaagccgcc tgttccggta ttagcagcat tgcaagccat tatgacgact |
| 1141 | catcgttaa |

In some embodiments of any of the aspects, the amino acid sequence encoded by the functional β-carotene synthesis gene (e.g., Pantoea ananatis CrtY) comprises SEQ ID NO: 68, or an amino acid sequence that is at least 95% (e.g., at least 95%, at least 96%, at least 97%, at least 98%, or at least 99%) identical to the sequence of SEQ ID NO: 68 that maintains the same functions as SEQ ID NO: 68 (e.g., lycopene cyclase).

| MULTISPECIES: lycopene beta-cyclase CrtY [Pantoea], NCBI | |
|---|---|
| Reference Sequence: WP_029571297.1, 382 aa, | |
| SEQ ID NO: 68 | |
| 1 | mqphydlilv gaglanglia lrlqqqqpdm rillidaapq aggnhtwsfh hddltesqhr |
| 61 | wiaslvvhhw pdyqvrfptr rrklnsgyfc itsqrfaevl qrqfgphlwm dtavaevnae |
| 121 | svrlkkgqvi garavidgrg yaansalsvg fqafigqewr lshphglssp iimdatvdqq |
| 181 | ngyrfvyslp lsptrllied thyidnatld perarqnicd yaaqqgwqlq tllreeqgal |
| 241 | pitlsgnaea fwqqrplacs glraglfhpt tgyslplava vadrisaldv ftsasihqai |
| 301 | rhfarerwqq qrffrmlnrm lflagpadsr wrvmqrfygl pedliarfya gkltltdrlr |
| 361 | ilsgkppvpv laalqaimtt hr |

In one aspect, described herein is a method of producing a feedstock solution, comprising: (a) culturing an engineered bacterium as described herein (e.g., an engineered feedstock bacterium) in a culture medium comprising CO₂ and/or H₂; and (b) isolating, collecting, or concentrating a feedstock solution from said engineered bacterium or from the culture medium of said engineered bacterium.

In some embodiments of any of the aspects, the feedstock solution comprises a sugar solution, comprising glucose, fructose, galactose, lactose, maltose, and/or sucrose.

In some embodiments of any of the aspects, the feedstock solution comprises at least 100 mg/L sucrose. In some embodiments of any of the aspects, the feedstock solution comprises at least 150 mg/L sucrose. As a non-limiting example, the feedstock solution comprises at least 50 mg/L sucrose, at least 60 mg/L sucrose, at least 70 mg/L sucrose, at least 80 mg/L sucrose, at least 90 mg/L sucrose, at least 100 mg/L sucrose, at least 110 mg/L sucrose, at least 120 mg/L sucrose, at least 130 mg/L sucrose, at least 140 mg/L sucrose, at least 150 mg/L sucrose, 160 mg/L sucrose, at least 170 mg/L sucrose, at least 180 mg/L sucrose, at least 190 mg/L sucrose, at least 200 mg/L sucrose, at least 210 mg/L sucrose, at least 220 mg/L sucrose, at least 230 mg/L sucrose, at least 240 mg/L sucrose, at least 250 mg/L sucrose, 260 mg/L sucrose, at least 270 mg/L sucrose, at least 280 mg/L sucrose, at least 290 mg/L sucrose, or at least 300 mg/L sucrose.

In some embodiments of any of the aspects, the culture medium (e.g., for an engineered feedstock solution bacterium) comprises CO₂ as the sole carbon source. In some embodiments of any of the aspects, CO₂ is at least 90%, at least 95%, at least 98%, at least 99% or more of the carbon sources present in the culture medium. In some embodiments of any of the aspects, the culture medium comprises CO₂ in the form of bicarbonate (e.g., HCO₃⁻, NaHCO₃) and/or dissolved CO₂ (e.g., atmospheric CO₂; e.g., CO₂ provided by a cell culture incubator). In some embodiments of any of the aspects, the culture medium does not comprise organic carbon as a carbon source. Non-limiting example of organic carbon sources include fatty acids, gluconate, acetate, fructose, decanoate; see e.g., Jiang et al. Int J Mol Sci. 2016 July; 17(7): 1157).

In some embodiments of any of the aspects, the culture medium (e.g., for an engineered feedstock solution bacterium) comprises H₂ as the sole energy source. In some embodiments of any of the aspects, H₂ is at least 90%, at least 95%, at least 98%, at least 99% or more of the energy sources present in the culture medium. In some embodiments of any of the aspects, H₂ is supplied by water-splitting electrodes in the culture medium, as described further herein (see e.g., US Patent Publication 2018/0265898, the contents of which are incorporated herein by reference in their entirety).

In some embodiments of any of the aspects, the culture medium (e.g., for an engineered feedstock solution bacterium) further comprises arabinose. In some embodiments of any of the aspects, the culture medium further comprises at least 0.3% arabinose. As a non-limiting example, the culture medium further comprises at least 0.1% arabinose, at least 0.2% arabinose, at least 0.3% arabinose, at least 0.4% arabinose, at least 0.5% arabinose, 0.6% arabinose, at least 0.7% arabinose, at least 0.8% arabinose, at least 0.9% arabinose, or at least 1.0% arabinose.

Methods of isolating, collecting, or concentrating sucrose are well known in the art. As a non-limiting example, such isolation methods can comprise ethanol extraction, centrifugation, ultracentrifugation, density gradient centrifugation, evaporation, boiling, and the like. In some embodiments of any the aspects, the isolated sucrose from the sucrose solution comprises a sucrose extract. Such a sucrose extract can be in the form of a powder, pill, capsule, lyophilized substance, ethanol extract solution, and the like.

In some embodiments of any of the aspects, the feedstock solution comprises a sucrose feedstock for at least one heterotroph. In some embodiments of any of the aspects, the at least one heterotroph comprises an organism with enhanced sugar utilization. In some embodiments of any of the aspects, the at least one heterotroph comprises an organism with enhanced sucrose utilization. In some embodiments of any of the aspects, the at least one heterotroph comprises E. coli, S. cerevisiae, B. subtilis, or Y. lipolytica. In some embodiments of any of the aspects, the at least one heterotroph comprises E. coli. In some embodiments of any of the aspects, the at least one heterotroph comprises S. cerevisiae. In some embodiments of any of the aspects, the at least one heterotroph comprises B. subtilis. In some embodiments of any of the aspects, the at least one heterotroph comprises Y. lipolytica. In some embodiments of any of the aspects, the at least one heterotroph comprises E. coli and S. cerevisiae. In some embodiments of any of the aspects, the at least one heterotroph comprises a heterotroph that can utilize sucrose as a carbon source.

In some embodiments of any of the aspects, the at least one heterotroph comprises an engineered bacterium as described herein (e.g., an engineered heterotroph with enhanced sucrose utilization). In some embodiments of any of the aspects, the at least one heterotroph comprises a mutant bacterium or a mutant yeast with enhanced sucrose utilization (e.g., yeast strain (PAS844) S. cerevisiae W303Clump comprising mutations in at least one of CSE2, IRA1, MTH1, UBR1, and ACE2). In some embodiments of any of the aspects, the engineered heterotroph has enhanced sucrose utilization as compared to the same heterotroph lacking the engineered sucrose catabolism gene(s), sucrose catabolism repressor(s), arabinose utilization gene(s), and/or secondary product synthesis gene(s). In some embodiments of any of the aspects, enhanced sucrose metabolism can be quantified by measuring the sucrose concentration in a culture medium over time.

In some embodiments of any of the aspects, the feedstock solution comprises at least one heterotroph. In some embodiments of any of the aspects, the feedstock solution comprises at least one engineered heterotroph (e.g., with enhanced sucrose utilization). In some embodiments of any of the aspects, the feedstock solution does not comprise a heterotroph. In some embodiments of any of the aspects, the feedstock solution is isolated, collected, or concentrated prior to providing to at least one engineered heterotroph (e.g., with enhanced sucrose utilization) or mutant heterotroph (e.g., with enhanced sucrose utilization).

In another aspect, described herein is a sustainable method of producing a product from a heterotroph, wherein the culture medium comprises CO₂ and/or H₂. Accordingly, in one aspect described herein is a method of a method of producing a heterotroph product (e.g., violacein, carotene), comprising: (a) culturing an engineered bacterium as described herein (e.g., an engineered feedstock solution bacterium) in a culture medium comprising CO₂ and/or H₂; (b) adding to the culture medium a second engineered bacterium as described herein (e.g., an engineered heterotroph); and (c) isolating, collecting, or concentrating the product (e.g., violacein and/or β-carotene) from the second engineered bacterium or from the culture medium.

In some embodiments of any of the aspects, the culture medium (e.g., for an engineered feedstock solution bacterium and/or the engineered heterotroph) comprises CO₂ as the sole carbon source. In some embodiments of any of the aspects, CO₂ is at least 90%, at least 95%, at least 98%, at least 99% or more of the carbon sources present in the culture medium. In some embodiments of any of the aspects, the culture medium comprises CO₂ in the form of bicarbonate (e.g., HCO3⁻, NaHCO₃) and/or dissolved CO₂ (e.g., atmospheric CO₂; e.g., CO₂ provided by a cell culture incubator). In some embodiments of any of the aspects, the culture medium does not comprise organic carbon as a carbon source. Non-limiting example of organic carbon sources include fatty acids, gluconate, acetate, fructose, decanoate; see e.g., Jiang et al. Int J Mol Sci. 2016 Jul.; 17(7): 1157).

In some embodiments of any of the aspects, the culture medium (e.g., for an engineered feedstock solution bacterium and/or the engineered heterotroph) comprises H₂ as the sole energy source. In some embodiments of any of the aspects, H₂ is at least 90%, at least 95%, at least 98%, at least 99% or more of the energy sources present in the culture medium. In some embodiments of any of the aspects, H₂ is supplied by water-splitting electrodes in the culture medium, as described further herein (see e.g., US Patent Publication 2018/0265898, the contents of which are incorporated herein by reference in their entirety).

In some embodiments of any of the aspects, the culture medium further (e.g., for an engineered feedstock solution bacterium and/or the engineered heterotroph) comprises arabinose. In some embodiments of any of the aspects, the culture medium further comprises at least 0.3% arabinose. As a non-limiting example, the culture medium further comprises at least 0.1% arabinose, at least 0.2% arabinose, at least 0.3% arabinose, at least 0.4% arabinose, at least 0.5% arabinose, 0.6% arabinose, at least 0.7% arabinose, at least 0.8% arabinose, at least 0.9% arabinose, or at least 1.0% arabinose.

In some embodiments of any of the aspects, the engineered heterotroph produces violacein, which can be isolated from the culture medium. In some embodiments of any of the aspects, the culture medium comprises at least 50 μg/L violacein. As a non-limiting example, the culture medium comprises at least 10 μg/L violacein, at least 20 μg/L violacein, at least 30 μg/L violacein, at least 40 μg/L violacein, at least 50 μg/L violacein, at least 60 μg/L violacein, at least 70 μg/L violacein, at least 80 μg/L violacein, at least 90 μg/L violacein, at least 100 μg/L violacein, at least 110 μg/L violacein, at least 120 μg/L violacein, at least 130 μg/L violacein, at least 140 μg/L violacein, at least 150 μg/L violacein, at least 160 μg/L violacein, at least 170 μg/L violacein, at least 180 μg/L violacein, at least 190 μg/L violacein, or at least 200 μg/L violacein.

In some embodiments of any of the aspects, the engineered heterotroph produces β-carotene, which can be isolated from the culture medium. In some embodiments of any of the aspects, the culture medium comprises at least 50 μg/L β-carotene. As a non-limiting example, the culture medium comprises at least 10 μg/L β-carotene, at least 20 μg/L β-carotene, at least 30 μg/L β-carotene, at least 40 μg/L β-carotene, at least 50 μg/L β-carotene, at least 60 μg/L β-carotene, at least 70 μg/L β-carotene, at least 80 μg/L β-carotene, at least 90 μg/L β-carotene, at least 100 μg/L β-carotene, at least 110 μg/L β-carotene, at least 120 μg/L β-carotene, at least 130 μg/L β-carotene, at least 140 μg/L β-carotene, at least 150 μg/L β-carotene, at least 160 μg/L β-carotene, at least 170 μg/L β-carotene, at least 180 μg/L β-carotene, at least 190 μg/L β-carotene, or at least 200 μg/L β-carotene.

Methods of isolating, collecting, or concentrating a secondary product (e.g., violacein or β-carotene) from a culture medium are well known in the art. As a non-limiting example, such isolation methods can comprise ethanol extraction, centrifugation, ultracentrifugation, density gradient centrifugation, evaporation, boiling, and the like.

In one aspect, described herein is an engineered bacterium comprising at least one exogenous copy of at least one functional lipochitooligosaccharide synthesis gene. In some embodiments, the engineered bacterium as described above is also referred to herein as an engineered fertilizer solution bacterium or an engineered LCO bacterium.

As used herein, the term “fertilizer solution” refers to a solution that increases or enhances the growth a plant, and/or increases the yield of a plant; as such fertilizer solution can also be referred to herein as a “plant growth enhancer.” Methods of measuring the growth of a plant are described further herein. Non-limiting examples of measuring the growth of a plant include plant weight, plant component (e.g., stem, root, fruit, leaf, and the like) weight, plant height, plant component (e.g., stem, root, fruit, leaf, and the like) height, plant number, or plant component (e.g., stem, root, fruit, leaf, and the like) number. Additional methods of measuring the growth of a plant are known to those of skill in the art. As used herein, the term “yield” refers the full amount of an agricultural or industrial product. In some embodiments of any of the aspects, yield comprises the agricultural product produced by a plant (e.g., crops, fruits, vegetables, etc.). In some embodiments of any of the aspects, yield comprises the product (e.g., bioplastic, feedstock, fertilizer, secondary product, etc.) produced by an engineered bacterium as described herein.

In some embodiments of any of the aspects, the fertilizer solution comprises lipochitooligosaccharides. In some embodiments of any of the aspects, the fertilizer solution is produced by an engineered bacterium (e.g., an engineered fertilizer solution bacterium) as described herein.

In some embodiments of any of the aspects, the engineered bacterium is a chemoautotroph. In some embodiments of any of the aspects, the engineered bacterium uses CO₂ as its sole carbon source, and/or said engineered bacteria uses H₂ as its sole energy source. In some embodiments of any of the aspects, the engineered bacterium is Cupriavidus necator. In some embodiments of any of the aspects, the engineered bacterium produces a fertilizer solution. In some embodiments of any of the aspects, the engineered bacteria produces lipochitooligosaccharide.

In some embodiments of any of the aspects, the engineered bacterium comprises at least one exogenous copy of at least one functional lipochitooligosaccharide synthesis gene. In some embodiments of any of the aspects, the at least one functional lipochitooligosaccharide synthesis gene comprises an N-acetylglucosaminyltransferase gene, a deacetylase gene, or an acetyltransferase gene. In some embodiments of any of the aspects, the at least one functional lipochitooligosaccharide synthesis gene is heterologous.

In some embodiments of any of the aspects, the engineered bacterium comprises (a) a N-acetylglucosaminyltransferase gene, (b) a deacetylase gene, or (c) an acetyltransferase gene. In some embodiments of any of the aspects, the engineered bacterium comprises a N-acetylglucosaminyltransferase gene. In some embodiments of any of the aspects, the engineered bacterium comprises a deacetylase gene. In some embodiments of any of the aspects, the engineered bacterium comprises an acetyltransferase gene.

In some embodiments of any of the aspects, the engineered bacterium comprises (a) a N-acetylglucosaminyltransferase gene and (b) a deacetylase gene. In some embodiments of any of the aspects, the engineered bacterium comprises (a) a N-acetylglucosaminyltransferase gene and (c) an acetyltransferase gene. In some embodiments of any of the aspects, the engineered bacterium comprises (b) a deacetylase gene and (c) an acetyltransferase gene. In some embodiments of any of the aspects, the engineered bacterium comprises (a) a N-acetylglucosaminyltransferase gene, (b) a deacetylase gene, and (c) an acetyltransferase gene.

In some embodiments of any of the aspects, the engineered bacterium comprises a first functional heterologous lipochitooligosaccharide synthesis gene (e.g., B. japonicum NodC), a second functional heterologous lipochitooligosaccharide synthesis gene (e.g., B. japonicum NodB), and/or a third functional heterologous lipochitooligosaccharide synthesis gene (e.g., B. japonicum NodA).

In some embodiments of any of the aspects, the at least one functional heterologous lipochitooligosaccharide synthesis gene comprises Bradyrhizobium japonicum NodC, Bradyrhizobium japonicum NodB, or Bradyrhizobium japonicum NodA. In some embodiments of any of the aspects, the engineered bacterium comprises B. japonicum NodC, B. japonicum NodB, or B. japonicum NodA. In some embodiments of any of the aspects, the engineered bacterium comprises B. japonicum NodC. In some embodiments of any of the aspects, the engineered bacterium comprises B. japonicum NodB. In some embodiments of any of the aspects, the engineered bacterium comprises B. japonicum NodA. In some embodiments of any of the aspects, the engineered bacterium comprises B. japonicum NodC and B. japonicum NodB. In some embodiments of any of the aspects, the engineered bacterium comprises B. japonicum NodC and B. japonicum NodA. In some embodiments of any of the aspects, the engineered bacterium comprises B. japonicum NodB and B. japonicum NodA. In some embodiments of any of the aspects, the engineered bacterium comprises B. japonicum NodC, B. japonicum NodB, and B. japonicum NodA.

In some embodiments of any of the aspects, the engineered bacterium comprises at least one functional lipochitooligosaccharide synthesis locus (e.g., NodABC) comprising SEQ ID NO: 84, or a nucleic acid sequence that is at least 95% (e.g., at least 95%, at least 96%, at least 97%, at least 98%, or at least 99%) identical to the sequence of SEQ ID NO: 84 that maintains the same functions as SEQ ID NO: 84.

NodABC (2761 bp; B. japonicum)
SEQ ID NO: 84
ATGAACATCGCCGTCTCCCCTACAGCAGAAGGCTCATCGGGCAGAGCACAAGTCCAGTG
GTCGCTGAGATGGGAATCGGAACTGCAGCTGGCCGATCATGCAGAACTGGCCGAGTTCT
TCCGCAAGTCGTATGGCCCTACAGGCGCCTTCAATGCACAGCCGTTTGAAAGATCGCGCT
CGTGGGCAGGCGCAAGACCTGAACTGAGAGTCATCGGCTATGATGCCCGTGGTGTTGCA
GCACATATCGGCCTGCTGCGCCGCTTCATCAAGGTTGGCGAAGTCGATTTACCGGTCGCC
GAACTGGGCCTTTATGCCGTCAGACCGGACCTGGAAGGTCATGGTATCGGCCATGCAAT
GCGTGTCATGTATCCGGCCCTGCAGGAATTAGGCGTCCCGTTCGGTTTCGGTGCAGTCAG
ATCGGCACTGGAGAAGCATCTGACCCGCCTGGTCGAAAGACAAGGCCTTGCAACCCTTA
TGCGCGGCATCAGAGTCCGCTCGACACTTCCTGACGTCTACCCGAATCTTTCGCCGACCC
GCATCGAAGACGTCATCGTCGTCGTCTTCCCTGTCGGTCGCTCAATCTCGGAATGGCCTG
CAGGTACCGTCATCGATCGCAATGGCCCGGAGCTGTGACCGAACGTTCAACCCTCTCAAC
CGTCCGCTGCGATTATGCCGATGCAGGTGGCTCAAGAGCCGTCCATCTGACCTTCGATGA
TGGCCCGAATCCGTTCTGTACCCCGGAAGTCCTGGACGTCCTTGCCCAACATAGAGTCCC
TGCAACCTTCTTCGTCATCGGCACCTATGCCACCGAGCATCCGGAGCTTATCCGCCGCAT
GATCGCAGAAGGCCATGAAGTCGCCAACCACACAATGACCCATCCGGACCTTTCGAGAT
GTGGCCCGACCGAGCTGCATGACGAAGTCCTGACCGCATCAGAAGCCATCAGACTTGCA
TGTCCGTTAGCATCGCCGCGCCACATGAGAGCCCCGTATGGCATCTGGACCCGTGATGTC
TTAGCAGTTGCAGCATCAGCAGGCCTGACCGCCTTACACTGGTCGGTCGATCCTCGCGAT
TGGTCGAGACCTGGTGTTGATGCAATCGTCAACTCGGTCCTGGCGAATGTCAGACCTGGC
GCCATCGTCCTGCTGCATGACGGCTATCCTCCTGATGAAGAAGGCCTTCCTTCGGGTTCG
ACCCTTCGCGATCAAACCCGCACCGCACTGGCATACCTGATTCCTGCACTTCAAAGACGC
GGCTTCGTCATCCGCCCTTTACCGCAGCTGCACTGAAAAAACGACACCCCATGGATCTGC
TTGCTACCACCTCTGCAGCAGCAGTTTCGTCATATGCACTGTTATCGACCATCTACAAGTC
AGTCCAAGCCCTGTATGCACAACCGGCCATCAACTCGTCGCTGGATAATTTAGGCCAAGC
AGAAGTTGTTGTTCCTGCCGTTGATGTCATCGTCCCGTGCTTCAACGAGAACCCTAATAC
CCTTGCAGAATGCTTAGAATCGATCGCCTCGCAGGATTATGCCGGCAAGATGCAGGTCTA
TGTCGTCGATGATGGTTCGGCAAATAGAGATGTCGTTGCACCTGTTCATCGCATCTATGC
CTCGGATCCGAGATTCTCGTTCATCCTGCTTGCCAATAATGTCGGCAAAAGAAAGGCCCA
GATTGCAGCAATCCGCTCATCGTCAGGCGATTTAGTCCTGAACGTTGATTCGGATACCAT
CTTAGCCGCAGATGTTGTCACCAAGCTGGTCCTGAAGATGCATGATCCTGGTATTGGTGC
AGCAATGGGTCAGCTTATCGCATCAAACCGCAATCAGACCTGGCTTACCAGACTGATCG
ATATGGAGTACTGGCTTGCCTGCAACGAAGAAAGAGCAGCACAAGCGAGATTTGGCGCA
GTCATGTGTTGTTGTGGTCCTTGTGCCATGTATAGAAGATCGGCCTTAGCACTGCTGTTAG
ACCAGTACGAAGCCCAGTTCTTCAGAGGTAAGCCGTCAGATTTTGGCGAAGATCGCCATC
TGACCATCCTGATGTTAAAGGCCGGCTTTAGAACCGAGTATGTCCCTGATGCAATTGCAG
CAACCGTCGTCCCTCATTCGTTAAGACCGTATCTGAGACAGCAGTTAAGATGGGCAAGAT
CAACCTTCCGCGACACCTTCCTTGCCTGGAGACTTTTACCGGAATTAGACGGCTATTTAA
CCCTGGATGTCATCGGCCAGAATCTTGGTCCGCTGCTGCTTGCAATCTCATCATTAGCCG
CATTAGCACAACTGCTGATTGATGGCTCAATTCCTTGGTGGACCGGTCTGACCATTGCAG
CAATGACCACTGTCAGATGTTGTGTTGCAGCATTAAGAGCCAGAGAACTGCGCTTCATCG
GTTTCTCGCTGCATACCCCGATCAATATCTGTCTTCTTCTTCCGCTTAAGGCATATGCCCT
GTGTACCCTGTCGAACTCGGATTGGCTTTCACGCAAAGTTACCGATATGCCTACCGAAGA
AGGCAAGCAACCTGTCATCCTTCACCCGAATGCTGGTAGATCACCTGCAGGTGTTGGTGG
TAGACTTCTTCTTTTCGTCAGACGCAGATATCGCTCGCTGCATAGAGCATGGCGTAGAAG
ACGCGTTTTTCCTGTTGCAATCGTCAGACTTTCGACCAACAAGTGGTCGGCAGATGATTC
GGGCAGAAAGCCTTCAGTCATTAGAGCAAGAGTCGGTTGCAGAAGACCTGTTGCACCGC
GTCATTAA

In some embodiments of any of the aspects, the engineered bacterium comprises a functional lipochitooligosaccharide synthesis gene (e.g., an acetylglucosaminyltransferase gene, NodC) comprising SEQ ID NO: 37 or SEQ ID NO: 85, or a nucleic acid sequence that is at least 95% (e.g., at least 95%, at least 96%, at least 97%, at least 98%, or at least 99%) identical to the sequence of SEQ ID NO: 37 or SEQ ID NO: 85 that maintains the same functions as SEQ ID NO: 37 or SEQ ID NO: 85 (e.g., acetylglucosaminyltransferase, chitooligosaccharide synthase).

| Bradyrhizobium japonicum USDA 6 DNA, complete genome, NCBI Reference | |
|---|---|
| Sequence: NC_017249.1, REGION: complement (8091092-8092549), 1458 bp, | |
| SEQ ID NO: 37 | |
| 1 | atggacctgc tcgcgacgac cagtgctgcc gccgtttcat cttatgcgct cctatcgacg |
| 61 | atctataaga gcgtgcaagc gctttatgct cagccggcga tcaactcatc gctggacaac |
| 121 | cttggacaag ccgaggtggt cgttcctgct gtggacgtga tcgtgccgtg cttcaacgag |
| 181 | aatccgaaca cactcgccga atgtctggag tcgattgcca gtcaagacta cgccggaaag |
| 241 | atgcaggtat atgtggtcga tgacggatcg gcaaaccgcg acgttgtcgc gcctgtacac |
| 301 | cggatatatg cgagcgatcc gagattcagt tttatcttgt tggcgaacaa tgtgggaaag |
| 361 | cgcaaggcgc agatcgcagc gatacgcagc tcatccggtg atctggttct caacgtcgat |
| 421 | tccgatacga tacttgctgc cgacgtcgtc acgaagcttg tattgaagat gcatgacccg |
| 481 | ggaatcggtg cggccatggg tcagctgatc gcgagcaatc gcaaccagac ctggctgacc |
| 541 | aggctgatcg acatggaata ttggctcgcg tgcaacgaag agcgcgcggc acaggcgcgc |
| 601 | ttcggtgccg tcatgtgttg ctgcggccca tgtgccatgt atcggcgttc cgcgctcgcc |
| 661 | ttgcttcttg atcaatatga agcccaattc tttcgtggga agccgagcga tttcggcgag |
| 721 | gaccgccacc taacgatact catgctcaag gcggggtttc gaaccgaata cgttccggac |
| 781 | gccatagcag ccacagtcgt cccgcacagt cttcggccat atctacgaca gcaactccgc |
| 841 | tgggcgcgaa gtacctttcg agatacgttt cttgcttggc gcctgctgcc agagctcgat |
| 901 | ggttatttga cgctagacgt tatcgggcaa aatctcggcc cattgctcct cgccatttca |
| 961 | tcacttgctg cgctcgcaca gctcctgatc gatggctcta taccctggtg gacgggattg |
| 1021 | acgattgctg caatgactac ggtccggtgc tgtgtggcag cgcttcgtgc ccgcgagctg |
| 1081 | cggtttatcg gcttctcgct ccacacgccg atcaatatct gtctcttact gcctttgaag |
| 1141 | gcctatgcgc tttgtacatt gagcaatagc gattggctat ctcggaaagt caccgatatg |
| 1201 | ccgacggaag aggggaaaca gcctgtcatc ctgcacccga atgccggacg aagtcctgct |
| 1261 | ggtgtagggg ggcgcctgct cctattcgta aggcggcgtt atcgcagcct ccatcgagcc |
| 1321 | tggcggcgac ggagagtgtt tccggtcgcg atcgttcgac tgtctacaaa taagtggtcg |
| 1381 | gctgatgact caggacgaaa accatcagtt attagagcga gagttggctg tcgacgaccc |
| 1441 | gtggcgcctc gacactag |

NodC (1458 bp; B. japonicum)
SEQ ID NO: 85
ATGGATCTGCTTGCTACCACCTCTGCAGCAGCAGTTTCGTCATATGCACTGTTATCGACC
ATCTACAAGTCAGTCCAAGCCCTGTATGCACAACCGGCCATCAACTCGTCGCTGGATAAT
TTAGGCCAAGCAGAAGTTGTTGTTCCTGCCGTTGATGTCATCGTCCCGTGCTTCAACGAG
AACCCTAATACCCTTGCAGAATGCTTAGAATCGATCGCCTCGCAGGATTATGCCGGCAAG
ATGCAGGTCTATGTCGTCGATGATGGTTCGGCAAATAGAGATGTCGTTGCACCTGTTCAT
CGCATCTATGCCTCGGATCCGAGATTCTCGTTCATCCTGCTTGCCAATAATGTCGGCAAA
AGAAAGGCCCAGATTGCAGCAATCCGCTCATCGTCAGGCGATTTAGTCCTGAACGTTGAT
TCGGATACCATCTTAGCCGCAGATGTTGTCACCAAGCTGGTCCTGAAGATGCATGATCCT
GGTATTGGTGCAGCAATGGGTCAGCTTATCGCATCAAACCGCAATCAGACCTGGCTTACC
AGACTGATCGATATGGAGTACTGGCTTGCCTGCAACGAAGAAAGAGCAGCACAAGCGAG
ATTTGGCGCAGTCATGTGTTGTTGTGGTCCTTGTGCCATGTATAGAAGATCGGCCTTAGC
ACTGCTGTTAGACCAGTACGAAGCCCAGTTCTTCAGAGGTAAGCCGTCAGATTTTGGCGA
AGATCGCCATCTGACCATCCTGATGTTAAAGGCCGGCTTTAGAACCGAGTATGTCCCTGA
TGCAATTGCAGCAACCGTCGTCCCTCATTCGTTAAGACCGTATCTGAGACAGCAGTTAAG
ATGGGCAAGATCAACCTTCCGCGACACCTTCCTTGCCTGGAGACTTTTACCGGAATTAGA
CGGCTATTTAACCCTGGATGTCATCGGCCAGAATCTTGGTCCGCTGCTGCTTGCAATCTC
ATCATTAGCCGCATTAGCACAACTGCTGATTGATGGCTCAATTCCTTGGTGGACCGGTCT
GACCATTGCAGCAATGACCACTGTCAGATGTTGTGTTGCAGCATTAAGAGCCAGAGAAC
TGCGCTTCATCGGTTTCTCGCTGCATACCCCGATCAATATCTGTCTTCTTCTTCCGCTTAA
GGCATATGCCCTGTGTACCCTGTCGAACTCGGATTGGCTTTCACGCAAAGTTACCGATAT
GCCTACCGAAGAAGGCAAGCAACCTGTCATCCTTCACCCGAATGCTGGTAGATCACCTGC
AGGTGTTGGTGGTAGACTTCTTCTTTTCGTCAGACGCAGATATCGCTCGCTGCATAGAGC
ATGGCGTAGAAGACGCGTTTTTCCTGTTGCAATCGTCAGACTTTCGACCAACAAGTGGTC
GGCAGATGATTCGGGCAGAAAGCCTTCAGTCATTAGAGCAAGAGTCGGTTGCAGAAGAC
CTGTTGCACCGCGTCATTAA

In some embodiments of any of the aspects, the amino acid sequence encoded by the functional lipochitooligosaccharide synthesis gene (e.g., an acetylglucosaminyltransferase gene, NodC) comprises SEQ ID NO: 38, or an amino acid sequence that is at least 95% (e.g., at least 95%, at least 96%, at least 97%, at least 98%, or at least 99%) identical to the sequence of SEQ ID NO: 38 that maintains the same functions as SEQ ID NO: 38 (e.g., acetylglucosaminyltransferase, chitooligosaccharide synthase).

| MULTISPECIES: chitooligosaccharide synthase NodC | |
|---|---|
| [Bradyrhizobium], NCBI Reference Sequence: WP_011084824.1, 485 aa, | |
| SEQ ID NO: 38 | |
| 1 | mdllattsaa avssyallst iyksvqalya qpainssldn lgqaevvvpa vdvivpcfne |
| 61 | npntlaecle siasqdyagk mqvyvvddgs anrdvvapvh riyasdprfs fillannvgk |
| 121 | rkaqiaairs ssgdlvlnvd sdtilaadvv tklvlkmhdp gigaamgqli asnrnqtwlt |
| 181 | rlidmeywla cneeraaqar fgavmcccgp camyrrsala llldqyeaqf frgkpsdfge |
| 241 | drhltilmlk agfrteyvpd aiaatvvphs lrpylrqqlr warstfrdtf lawrllpeld |
| 301 | gyltldvigq nlgplllais slaalaqlli dgsipwwtgl tiaamttvrc cvaalrarel |
| 361 | rfigfslhtp iniclllplk ayalctlsns dwlsrkvtdm pteegkqpvi lhpnagrspa |
| 421 | gvggrlllfv rrryrslhra wrrrrvfpva ivrlstnkws addsgrkpsv irarvgcrrp |
| 481 | vaprh |

In some embodiments of any of the aspects, the engineered bacterium comprises a functional lipochitooligosaccharide synthesis gene (e.g., a deacetylase gene, NodB) comprising SEQ ID NO: 39 or SEQ ID NO: 86, or a nucleic acid sequence that is at least 95% (e.g., at least 95%, at least 96%, at least 97%, at least 98%, or at least 99%) identical to the sequence of SEQ ID NO: 39 or SEQ ID NO: 86 that maintains the same functions as SEQ ID NO: 39 or SEQ ID NO: 86 (e.g., deacetylase, chitooligosaccharide deacetylase).

| Bradyrhizobium japonicum USDA 6 DNA, complete genome, NCBI Reference | |
|---|---|
| Sequence: NC_017249.1, REGION: complement (8092564-8093223), 660 bp, | |
| SEQ ID NO: 39 | |
| 1 | gtgacagagc gttccaccct atctactgtc cgctgcgact acgctgacgc gggcggaagt |
| 61 | cgagctgtcc atttgacctt tgacgatggg ccaaatccat tttgtacgcc agaggtgctc |
| 121 | gatgtcctgg cgcaacatcg ggtccccgcg acattcttcg tcatcgggac gtacgcgacg |
| 181 | gagcatcctg aactcatccg acgaatgatt gcggaagggc atgaggttgc gaaccatacg |
| 241 | atgacccatc ctgatctatc cagatgcgga cctacggagc tacacgacga ggtgctgacg |
| 301 | gcgagcgaag ccatccgtct ggcgtgcccg ctggcctcgc ccaggcatat gcgagcgcct |
| 361 | tacggcatat ggacgcgaga tgtgctcgca gtggcggcga gcgctggtct cacggctctg |
| 421 | cactggtcgg tcgaccctag agattggtcc cgccccgggg ttgatgcaat tgtgaattcc |
| 481 | gtactggcga acgtacgccc gggtgcaatt gtgctcctgc acgacggata tcctcccgat |
| 541 | gaggagggat tgcccagtgg ctctacgctg cgcgatcaga ccaggacggc gctggcatat |
| 601 | ctcattccag cactacaacg gcgcgggttt gtaatccgtc cactccctca actccactga |

NodB (660 bp; B. Japonicum)
SEQ ID NO: 86
GTGACCGAACGTTCAACCCTCTCAACCGTCCGCTGCGATTATGCCGATGCAGGTGGCTCA
AGAGCCGTCCATCTGACCTTCGATGATGGCCCGAATCCGTTCTGTACCCCGGAAGTCCTG
GACGTCCTTGCCCAACATAGAGTCCCTGCAACCTTCTTCGTCATCGGCACCTATGCCACC
GAGCATCCGGAGCTTATCCGCCGCATGATCGCAGAAGGCCATGAAGTCGCCAACCACAC
AATGACCCATCCGGACCTTTCGAGATGTGGCCCGACCGAGCTGCATGACGAAGTCCTGA
CCGCATCAGAAGCCATCAGACTTGCATGTCCGTTAGCATCGCCGCGCCACATGAGAGCCC
CGTATGGCATCTGGACCCGTGATGTCTTAGCAGTTGCAGCATCAGCAGGCCTGACCGCCT
TACACTGGTCGGTCGATCCTCGCGATTGGTCGAGACCTGGTGTTGATGCAATCGTCAACT
CGGTCCTGGCGAATGTCAGACCTGGCGCCATCGTCCTGCTGCATGACGGCTATCCTCCTG
ATGAAGAAGGCCTTCCTTCGGGTTCGACCCTTCGCGATCAAACCCGCACCGCACTGGCAT
ACCTGATTCCTGCACTTCAAAGACGCGGCTTCGTCATCCGCCCTTTACCGCAGCTGCACT
GA

In some embodiments of any of the aspects, the amino acid sequence encoded by the functional lipochitooligosaccharide synthesis gene (e.g., a deacetylase gene, NodB) comprises SEQ ID NO: 40, or an amino acid sequence that is at least 95% (e.g., at least 95%, at least 96%, at least 97%, at least 98%, or at least 99%) identical to the sequence of SEQ ID NO: 40 that maintains the same functions as SEQ ID NO: 40 (e.g., deacetylase, chitooligosaccharide deacetylase).

| MULTISPECIES: chitooligosaccharide deacetylase NodB | |
|---|---|
| [Bradyrhizobium], NCBI Reference Sequence: WP_011084823.1, 219 aa, | |
| SEQ ID NO: 40 | |
| 1 | mterstlstv rcdyadaggs ravhltfddg pnpfctpevl dvlaqhrvpa tffvigtyat |
| 61 | ehpelirrmi aeghevanht mthpdlsrcg ptelhdevlt aseairlacp lasprhmrap |
| 121 | ygiwtrdvla vaasagltal hwsvdprdws rpgvdaivns vlanvrpgai vllhdgyppd |
| 181 | eeglpsgstl rdqtrtalay lipalqrrgf virplpqlh |

In some embodiments of any of the aspects, the engineered bacterium comprises a functional lipochitooligosaccharide synthesis gene (e.g., an acetyltransferase gene, NodA) comprising SEQ ID NO: 41 or SEQ ID NO: 87, or a nucleic acid sequence that is at least 95% (e.g., at least 95%, at least 96%, at least 97%, at least 98%, or at least 99%) identical to the sequence of SEQ ID NO: 41 or SEQ ID NO: 87 that maintains the same functions as SEQ ID NO: 41 or SEQ ID NO: 87 (e.g., acetyltransferase).

| Bradyrhizobium japonicum USDA 6 DNA, complete genome, NCBI Reference | |
|---|---|
| Sequence: NC_017249.1, REGION: complement (8093220-8093852), 633 bp, | |
| SEQ ID NO: 41 | |
| 1 | atgaacattg ccgtctcccc gactgcggaa ggatcttctg ggcgcgctca agtgcagtgg |
| 61 | agccttcgtt gggaaagtga actgcagctc gccgatcatg ccgagctcgc ggagttcttc |
| 121 | cgcaagagtt acggaccgac gggtgcgttc aatgcgcagc cgtttgagcg gagccgaagt |
| 181 | tgggctggag caagacccga gctccgcgta attggttacg acgcgcgcgg ggtagcggct |
| 241 | cacatcgggc tactgcgccg cttcatcaaa gttggtgaag tcgatctccc tgtggccgaa |
| 301 | ctggggttgt atgcggtgcg ccccgatctc gaggggcacg ggataggcca cgcaatgcgc |
| 361 | gtgatgtatc ccgcactcca ggagctcggc gttccattcg gatttggcgc ggttcgctcg |
| 421 | gccctcgaaa aacatttgac ccgactggtc gaaaggcagg ggctcgccac cctgatgcgt |
| 481 | ggcattcgcg tccgctccac cttgccggat gtctatccaa atttatcgcc gacgcgcatc |
| 541 | gaagacgtga tcgtcgtggt gtttccggtc ggacgctcga taagcgaatg gccggccggg |
| 601 | actgtcatcg atcgtaacgg gcctgagttg tga |

NodA (633 bp; B. Japonicum)
SEQ ID NO: 87
ATGAACATCGCCGTCTCCCCTACAGCAGAAGGCTCATCGGGCAGAGCACAAGTCCAGTG
GTCGCTGAGATGGGAATCGGAACTGCAGCTGGCCGATCATGCAGAACTGGCCGAGTTCT
TCCGCAAGTCGTATGGCCCTACAGGCGCCTTCAATGCACAGCCGTTTGAAAGATCGCGCT
CGTGGGCAGGCGCAAGACCTGAACTGAGAGTCATCGGCTATGATGCCCGTGGTGTTGCA
GCACATATCGGCCTGCTGCGCCGCTTCATCAAGGTTGGCGAAGTCGATTTACCGGTCGCC
GAACTGGGCCTTTATGCCGTCAGACCGGACCTGGAAGGTCATGGTATCGGCCATGCAAT
GCGTGTCATGTATCCGGCCCTGCAGGAATTAGGCGTCCCGTTCGGTTTCGGTGCAGTCAG
ATCGGCACTGGAGAAGCATCTGACCCGCCTGGTCGAAAGACAAGGCCTTGCAACCCTTA
TGCGCGGCATCAGAGTCCGCTCGACACTTCCTGACGTCTACCCGAATCTTTCGCCGACCC
GCATCGAAGACGTCATCGTCGTCGTCTTCCCTGTCGGTCGCTCAATCTCGGAATGGCCTG
CAGGTACCGTCATCGATCGCAATGGCCCGGAGCTGTGA

In some embodiments of any of the aspects, the amino acid sequence encoded by the functional lipochitooligosaccharide synthesis gene (e.g., an acetyltransferase gene, NodA) comprises SEQ ID NO: 42, or an amino acid sequence that is at least 95% (e.g., at least 95%, at least 96%, at least 97%, at least 98%, or at least 99%) identical to the sequence of SEQ ID NO: 42 that maintains the same functions as SEQ ID NO: 42 (e.g., acetyltransferase).

| MULTISPECIES: NodA family N-acyltransferase [Bradyrhizobium], | |
|---|---|
| NCBI Reference Sequence: WP_011084822.1, 210 aa, | |
| SEQ ID NO: 42 | |
| 1 | mniavsptae gssgraqvqw slrweselql adhaelaeff rksygptgaf naqpfersrs |
| 61 | wagarpelrv igydargvaa higllrrfik vgevdlpvae lglyavrpdl eghgighamr |
| 121 | vmypalqelg vpfgfgavrs alekhltrlv erqglatlmr girvrstlpd vypnlsptri |
| 181 | edvivvvfpv grsisewpag tvidrngpel |

In one aspect, described herein is a method of producing a fertilizer solution. In some embodiments of any of the aspects, the fertilizer solution comprises lipochitooligosaccharide (LCO). LCO is a glycolipid, derived from chitooligosaccharide, that is a bacterial nodulation factor. In some embodiments of any of the aspects, the LCO comprises Nod Cn-V (C18:1).

Accordingly, in one aspect described herein is a method of producing a fertilizer solution, comprising: (a) culturing an engineered bacterium as described herein (e.g., an engineered fertilizer solution bacterium) in a culture medium comprising CO₂ and/or H₂; and (b) isolating, collecting, or concentrating a fertilizer solution from said engineered bacterium or from the culture medium of said engineered bacterium.

In some embodiments of any of the aspects, the culture medium (e.g., for an engineered fertilizer solution bacterium) comprises CO₂ as the sole carbon source. In some embodiments of any of the aspects, CO₂ is at least 90%, at least 95%, at least 98%, at least 99% or more of the carbon sources present in the culture medium. In some embodiments of any of the aspects, the culture medium comprises CO₂ in the form of bicarbonate (e.g., HCO₃⁻, NaHCO₃) and/or dissolved CO₂ (e.g., atmospheric CO₂; e.g., CO₂ provided by a cell culture incubator). In some embodiments of any of the aspects, the culture medium does not comprise organic carbon as a carbon source. Non-limiting example of organic carbon sources include fatty acids, gluconate, acetate, fructose, decanoate; see e.g., Jiang et al. Int J Mol Sci. 2016 July; 17(7): 1157).

In some embodiments of any of the aspects, the culture medium (e.g., for an engineered fertilizer solution bacterium) comprises H₂ as the sole energy source. In some embodiments of any of the aspects, H₂ is at least 90%, at least 95%, at least 98%, at least 99% or more of the energy sources present in the culture medium. In some embodiments of any of the aspects, H₂ is supplied by water-splitting electrodes in the culture medium, as described further herein (see e.g., US Patent Publication 2018/0265898, the contents of which are incorporated herein by reference in their entirety).

In some embodiments of any of the aspects, the fertilizer solution comprises a lipochitooligosaccharide concentration of at least 1 mg/L. As a non-limiting example, the fertilizer solution can comprise a lipochitooligosaccharide concentration of at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, or at least 5 mg/L.

In some embodiments of any of the aspects, the culture medium (e.g., for an engineered fertilizer solution bacterium) further comprises arabinose. In some embodiments of any of the aspects, the culture medium further comprises at least 0.3% arabinose. As a non-limiting example, the culture medium further comprises at least 0.1% arabinose, at least 0.2% arabinose, at least 0.3% arabinose, at least 0.4% arabinose, at least 0.5% arabinose, 0.6% arabinose, at least 0.7% arabinose, at least 0.8% arabinose, at least 0.9% arabinose, or at least 1.0% arabinose.

In some embodiments of any of the aspects, methods described herein comprise isolating, collecting, or concentrating a product (e.g., LCO) from an engineered bacterium or from the culture medium of an engineered bacterium. Methods of isolating LCO are well-known in the art. Non-limiting examples of LCO isolation methods include butanol extraction, centrifugation, and/or evaporation. As a non-limiting example, cultures can be extracted with HPLC-grade 1-butanol, e.g., by shaking vigorously (e.g., for 10 min). The material can then be centrifuged (e.g., for 10 min at 4000 rpm). The upper butanol phase can be separated and dried in a rotary evaporator under vacuum at 55° C. The LCO can be re-dissolved (e.g., in 20% acetonitrile) and analyzed (e.g., by HPLC).

In some embodiments, the fertilizer solution (e.g., LCO) is administered to a plant in order to increase growth and/or yield. In some embodiments, the fertilizer solution (e.g., LCO) is administered to a spinach (S. oleracea), corn (Z. mays), and/or soybean (G. max) in order to increase growth and/or yield.

Non-limiting examples of plant species to which the fertilizer solution (e.g., LCO) can be administered include: corn (e.g., Zea mays), soybean (e.g., Glycine max), tomato (e.g., Solanum lycopersicum), squash (e.g., Cucurbita argyrosperma, Cucurbita maxima, Cucurbita moschata, Cucurbita pepo), cotton (e.g., Gossypium hirsutum, Gossypium barbadense, Gossypium arboreum, Gossypium herbaceum), wheat (e.g., Triticum aestivum, Triticum aethiopicum, Triticum araraticum, Triticum boeoticum, Triticum carthlicum, Triticum compactum, Triticum dicoccoides, Triticum dicoccon, Triticum durum, Triticum ispahanicum, Triticum karamyschevii, Triticum macha, Triticum militinae, Triticum monococcum, Triticum polonicum, Triticum spelta, Triticum sphaerococcum, Triticum timopheevii, Triticum turanicum, Triticum turgidum, Triticum Urartu, Triticum vavilovii, Triticum zhukovskyi), sunflower (e.g., Helianthus annuus, Helianthis agrestis, Helianthus angustifolius, Helianthus anomalus, Helianthus argophyllus, Helianthus arizonensis, Helianthus atrorubens, Helianthus bolanderi, Helianthus californicus, Helianthus carnosus, Helianthus ciliaris, Helianthus cinereus, Helianthus cusickii, Helianthus debilis, Helianthus decapetalus, Helianthus deserticola, Helianthus divaricatus, Helianthus eggertii, Helianthus floridanus, Helianthus giganteus, Helianthus glaucophyllus, Helianthus gracilentus, Helianthus grosseserratus, Helianthus heterophyllus, Helianthus hirsutus, Helianthus laciniatus, Helianthus laetiflorus, Helianthus laevigatus, Helianthus longifolius, Helianthus maximiliani, Helianthus microcephalus, Helianthus mollis, Helianthus multiflorus, Helianthus neglectus, Helianthus niveus, Helianthus nuttallii, Helianthus occidentalis, Helianthus paradoxus, Helianthus pauciflorus, Helianthus petiolaris, Helianthus porter, Helianthus praecox, Helianthus praetermissus, Helianthus pumilus, Helianthus radula, Helianthus resinosus, Helianthus salicifolius, Helianthus schweinitzii, Helianthus silphioides, Helianthus simulans, Helianthus smithii, Helianthus strumosus, Helianthus tuberosus), grape (e.g., Vitis vinifera, Vitis vinifera, Vitis labrusca, Vitis riparia, Vitis rotundifolia, Vitis rupestris, Vitis aestivalis, Vitis mustangensis, or any multi-species hybrids), cowpea (e.g., Vigna unguiculata), Chrysanthemum (e.g., Chrysanthemum indicum), Eucalyptus (e.g., Eucalyptus obliqua or any of the approximately 700 other species in the Eucalyptus genus), flax (e.g., Phormium tenax, Phormium cookianum), sesame (e.g., Sesamum radiatum), pepper (e.g., Capsicum annuum, Capsicum baccatum, Capsicum chinense, Capsicum frutescens, Capsicum pubescens), rice (e.g., Oryza sativa, including any one of the more than 40,000 varieties of this species), potato (e.g., Solanum tuberosum), cassava (e.g., Manihot esculenta), rye (e.g., Secale cereale), barley (e.g., Hordeum vulgare), alfalfa (e.g., Medicago sativa), or rapeseed (e.g., Brassica napus). A plant species can include any subspecies, cultivars, multi-species hybrids, strains, or any other variations or varieties that are known in the art.

In some embodiments, one or more of the genes described herein is expressed in a recombinant expression vector or plasmid. As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector is pBadT. In some embodiments of any of the aspects, pBadT is an expression vector for at least one functional, heterologous gene.

Without limitations, the genes described herein can be included in one vector or separate vectors. For example, the functional heterologous PHA synthase gene (e.g., Pseudomonas aeruginosa phaC1 and/or Pseudomonas aeruginosa phaC2 and/or Pseudomonas spp. 61-3 phaC1) and/or the functional heterologous thioesterase gene (e.g., Umbellularia californica FatB2 gene, a Cuphea palustris FatB1 gene, a Cuphea palustris FatB2 gene, or a Cuphea palustris FatB2-FatB1 hybrid gene) can be included in the same vector.

In some embodiments, the functional heterologous PHA synthase genes (e.g., Pseudomonas aeruginosa phaC1 and/or Pseudomonas aeruginosa phaC2 and/or Pseudomonas spp. 61-3 phaC1) can be included in a first vector, and the functional heterologous thioesterase gene (e.g., Umbellularia californica FatB2 gene, a Cuphea palustris FatB1 gene, a Cuphea palustris FatB2 gene, or a Cuphea palustris FatB2-FatB1 hybrid gene) can be included in a second vector.

In some embodiments, a first functional heterologous PHA synthase gene (e.g., Pseudomonas aeruginosa phaC1 and/or Pseudomonas spp. 61-3 phaC1) can be included in a first vector, a second functional heterologous PHA synthase gene (e.g., Pseudomonas aeruginosa phaC2 and/or Pseudomonas spp. 61-3 phaC1) can be included in a second vector, and the functional heterologous thioesterase gene (e.g., Umbellularia californica FatB2 gene, a Cuphea palustris FatB1 gene, a Cuphea palustris FatB2 gene, or a Cuphea palustris FatB2-FatB1 hybrid gene) can be included in a third vector.

Without limitations, the genes described herein can be included in one vector or separate vectors. For example, the first functional heterologous sucrose synthesis gene (e.g., Synechocystis sp. PCC 6803 sucrose phosphate synthase (SPS)) or the second functional heterologous sucrose synthesis gene (e.g., Synechocystis sp. PCC 6803 sucrose phosphate phosphatase (SPP)) or a functional heterologous sugar porin gene (e.g., E. coli scrY) can be included in the same vector; or the first functional heterologous sucrose synthesis gene (e.g., Synechocystis sp. PCC 6803 SPS) and the second functional heterologous sucrose synthesis gene (e.g., Synechocystis sp. PCC 6803 SPP) can be included in the same vector; or the first functional heterologous sucrose synthesis gene (e.g., Synechocystis sp. PCC 6803 SPS) and the first functional heterologous sugar porin gene (e.g., E. coli scrY) can be included in the same vector; or the second functional heterologous sucrose synthesis gene (e.g., Synechocystis sp. PCC 6803 SPP) and the functional heterologous sugar porin gene (e.g., E. coli scrY) can be included in the same vector.

In some embodiments, the first functional heterologous sucrose synthesis gene (e.g., Synechocystis sp. PCC 6803 SPS) can be included in a first vector, the second functional heterologous sucrose synthesis gene (e.g., Synechocystis sp. PCC 6803 SPP) can be included in a second vector, and the functional heterologous sugar porin gene (e.g., E. coli scrY) can be included in a third vector.

Without limitations, the genes described herein can be included in one vector or separate vectors. For example, a first functional heterologous lipochitooligosaccharide synthesis gene (e.g., B. japonicum NodC) or a second functional heterologous lipochitooligosaccharide synthesis gene (e.g., B. japonicum NodB) or a third functional heterologous lipochitooligosaccharide synthesis gene (e.g., B. japonicum NodA) can be included in the same vector; or the first functional heterologous lipochitooligosaccharide synthesis gene (e.g., B. japonicum NodC) and the second functional heterologous lipochitooligosaccharide synthesis gene (e.g., B. japonicum NodB) can be included in the same vector; or the first functional heterologous lipochitooligosaccharide synthesis gene (e.g., B. japonicum NodC) and the third functional heterologous lipochitooligosaccharide synthesis gene (e.g., B. japonicum NodA) can be included in the same vector; or the second functional heterologous lipochitooligosaccharide synthesis gene (e.g., B. japonicum NodB) and the third functional heterologous lipochitooligosaccharide synthesis gene (e.g., B. japonicum NodA) can be included in the same vector.

In some embodiments, the first functional heterologous lipochitooligosaccharide synthesis gene (e.g., B. japonicum NodC) can be included in a first vector, the second functional heterologous lipochitooligosaccharide synthesis gene (e.g., B. japonicum NodB) can be included in a second vector, and the third functional heterologous lipochitooligosaccharide synthesis gene (e.g., B. japonicum NodA) can be included in a third vector.

In some embodiments, the vector is pET21b.

Without limitations, the genes described herein can be included in one vector or separate vectors. For example, the violacein synthesis genes (e.g., Chromobacterium violaceum VioA, VioB, VioC, VioD, VioE) can all be included in the same vector, or can be grouped and included in any combination of up to five vectors.

In some embodiments, the Chromobacterium violaceum VioA gene can be included in a first vector, the Chromobacterium violaceum VioB gene can be included in a second vector, the Chromobacterium violaceum VioC gene can be included in a third vector, the Chromobacterium violaceum VioD gene can be included in a fourth vector, and the Chromobacterium violaceum VioE gene can be included in a fifth vector.

In some embodiments, the vector is pSB1C3.

Without limitations, the genes described herein can be included in one vector or separate vectors. For example, the beta-carotene synthesis genes (e.g., Pantoea ananatis CrtE, CrtB, CrtI, CrtY) can all be included in the same vector, or can be grouped and included in any combination of up to four vectors.

In some embodiments, the Pantoea ananatis CrtE gene can be included in a first vector, the Pantoea ananatis CrtB gene can be included in a second vector, the Pantoea ananatis CrtI gene can be included in a third vector, and the Pantoea ananatis CrtY gene can be included in a fourth vector.

In some embodiments, the vector is pCR2.1.

Without limitations, the genes described herein can be included in one vector or separate vectors. For example, the functional sucrose catabolism genes (e.g., CscA, CscB, CscK) can be included in the same vector; or the CscA gene and the CscB gene can be included in the same vector; or the CscA gene and the CscK gene can be included in the same vector; or the CscB gene and the CscK gene can be included in the same vector.

In some embodiments, the CscA gene can be included in a first vector, the CscB gene can be included in a second vector, and the CscK gene can be included in a third vector.

In some other embodiments, the vector is pT18mobsacB. In some embodiments of any of the aspects, pT18mobsacB is an integration vector that can be used to engineer at least one inactivating modification of at least one endogenous gene in a bacterium.

Without limitations, the genes described herein can be included in one vector or separate vectors. For example, an endogenous polyhydroxyalkanoate (PHA) synthase gene (e.g., phaC) comprising an engineered inactivating modification and/or an endogenous beta-oxidation gene (e.g., 3-hydroxyacyl-CoA dehydrogenase) comprising an engineered inactivating modification can be included in the same vector.

In some embodiments, the endogenous polyhydroxyalkanoate (PHA) synthase gene (e.g., phaC) comprising an engineered inactivating modification can be included in a first vector, and the endogenous beta-oxidation gene (e.g., 3-hydroxyacyl-CoA dehydrogenase) comprising an engineered inactivating modification can be included in a second vector.

In some embodiments, an endogenous sucrose catabolism repressor gene (e.g., E. coli CscR) comprising an engineered inactivating modification can be included in a vector as described herein or known in the art.

In some embodiments, an endogenous arabinose utilization gene (e.g., araB, araA, araD, araC) comprising an engineered inactivating modification can be included in a vector as described herein or known in the art.

In some embodiments, one or more of the recombinantly expressed gene can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as conjugation or transformation including chemical transformation and electroporation, transduction, particle bombardment, etc. Expressing the nucleic acid molecule encoding the enzymes of the claimed invention also may be accomplished by integrating the nucleic acid molecule into the genome.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

In microbiology, “16S sequencing” or “16S rRNA” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to a second isolate using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi.

The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75(10):4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to the reference sequence and identifying the hypervariable regions based on similarity to the reference hypervariable regions.

“Operational taxonomic unit (OTU, plural OTUs)” refers to a terminal leaf in a phylogenetic tree and is defined by a specific genetic sequence and all sequences that share a specified degree of sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S rRNA sequence or a portion of the 16S rRNA sequence, or it may be a functionally conserved housekeeping gene found broadly across the eubacterial kingdom. OTUs generally share at least 95%, 96%, 97%, 98%, or 99% sequence identity. OTUs are frequently defined by comparing sequences between organisms. Sequences with less than the specified sequence identity (e.g., less than 97%) are not considered to form part of the same OTU.

“Clade” refers to the set of OTUs or members of a phylogenetic tree downstream of a statistically valid node in a phylogenetic tree. The clade comprises a set of terminal leaves in the phylogenetic tree that is a distinct monophyletic evolutionary unit.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein. Preferably, the subject is a mammal. The mammal can be a human, non-human primate, mouse, rat, dog, cat, horse, or cow, but is not limited to these examples. A subject can be male or female. In some embodiments, the subject is a plant. In some embodiments, the subject is a bacterium.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding a polypeptide as described herein (e.g. a PHA synthase polypeptide, a thioesterase polypeptide, a beta-oxidation enzyme) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. An engineered Cupriavidus necator bacterium, comprising: at least one exogenous copy of at least one functional polyhydroxyalkanoate (PHA) synthase gene; and at least one exogenous copy of at least one functional thioesterase gene.
2. The engineered bacterium of paragraph 1, further comprising: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product.
3. The engineered bacterium of paragraph 1, further comprising: (i) at least one endogenous beta-oxidation gene comprising at least one engineered inactivating modification; or (ii) at least one exogenous inhibitor of an endogenous beta-oxidation gene or gene product.
4. The engineered bacterium of any one of paragraphs 1-3, wherein said engineered bacteria is a chemoautotroph.
5. The engineered bacterium of any one of paragraphs 1-4, wherein said engineered bacteria uses CO₂ as its sole carbon source, and/or said engineered bacteria uses H₂ as its sole energy source.
6. The engineered bacterium of paragraph 2, wherein the endogenous PHA synthase comprises phaC.
7. The engineered bacterium of any one of paragraphs 1-6, wherein the functional PHA synthase gene is heterologous.
8. The engineered bacterium of paragraph 7, wherein the functional heterologous PHA synthase gene comprises a Pseudomonas aeruginosa phaC1, a Pseudomonas aeruginosa phaC2 gene, and/or Pseudomonas spp. 61-3 phaC 1.
9. The engineered bacterium of any one of paragraphs 1-8, wherein the functional thioesterase gene is heterologous.
10. The engineered bacterium of paragraph 9, wherein the functional heterologous thioesterase gene comprises a Umbellularia californica FatB2 gene, a Cuphea palustris FatB1 gene, a Cuphea palustris FatB2 gene, or a Cuphea palustris FatB2-FatB1 hybrid gene.
11. The engineered bacterium of paragraph 3, wherein the endogenous beta-oxidation gene is 3-hydroxyacyl-CoA dehydrogenase (fadB) or acyl-CoA ligase.
12. The engineered bacterium of any one of paragraphs 1-11, wherein an engineered inactivating modification of a gene comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.
13. The engineered bacterium of paragraph 3, wherein the inhibitor of an endogenous beta-oxidation enzyme is acrylic acid.
14. The engineered bacterium of any one of paragraphs 1-13, wherein said engineered bacteria produces medium chain length PHA.
15. A method of producing medium-chain-length polyhydroxyalkanoate (MCL-PHA), comprising:
   a) culturing the engineered bacterium of any of paragraphs 1-14 in a culture medium comprising CO₂ and/or H₂; and
   b) isolating, collecting, or concentrating MCL-PHA from said engineered bacterium or from the culture medium of said engineered bacterium.
16. The method of paragraph 15, wherein the isolated MCL-PHA comprises an R group fatty acid which is 6 to 14 carbons long (C6-C14).
17. The method of any one of paragraphs 15-16, wherein the total PHA isolated comprises at least 50% MCL-PHA.
18. The method of any one of paragraphs 15-17, wherein the total PHA isolated comprises at least 80% MCL-PHA.
19. The method of any one of paragraphs 15-18, wherein the total PHA isolated comprises at least 95% MCL-PHA.
20. The method of any one of paragraphs 15-19, wherein the total PHA isolated comprises at least 98% MCL-PHA.
21. The method of any one of paragraphs 15-20, wherein the total PHA isolated comprises at least 95% MCL-PHA with an R group fatty acid of C10-C14.
22. The method of any one of paragraphs 15-21, wherein the total PHA isolated comprises at least 80% MCL-PHA with an R group fatty acid of C12-C14.
23. The method of any one of paragraphs 15-22, wherein the culture medium comprises CO₂ as the sole carbon source, and/or the culture medium comprises H₂ as the sole energy source.
24. An engineered C. necator bacterium, comprising one or more of the following:
   a) at least one exogenous copy of at least one functional sugar synthesis gene; and/or
   b) at least one exogenous copy of at least one functional sugar porin gene.
25. The engineered bacterium of paragraph 24, wherein said engineered bacteria is a chemoautotroph.
26. The engineered bacterium of any one of paragraphs 24-25, wherein said engineered bacteria uses CO₂ as its sole carbon source, and/or said engineered bacteria uses H₂ as its sole energy source.
27. The engineered bacterium of any one of paragraphs 24-26, wherein the at least one functional sugar synthesis gene is heterologous.
28. The engineered bacterium of any one of paragraphs 24-27, wherein the at least one functional sugar synthesis gene comprises at least one functional sucrose synthesis gene.
29. The engineered bacterium of any one of paragraphs 24-28, wherein the at least one functional heterologous sucrose synthesis gene comprises Synechocystis sp. PCC 6803 sucrose phosphate synthase (SPS) and/or Synechocystis sp. PCC 6803 sucrose phosphate phosphatase (SPP).
30. The engineered bacterium of any one of paragraphs 24-29, wherein the functional sugar porin gene is heterologous.
31. The engineered bacterium of any one of paragraphs 24-30, wherein the functional sugar porin gene is a functional sucrose porin gene.
32. The engineered bacterium of any one of paragraphs 24-31, wherein the functional heterologous sucrose porin gene comprises E. coli sucrose porin (scrY).
33. The engineered bacterium of any one of paragraphs 24-32, wherein said engineered bacteria produces a feedstock solution.
34. The engineered bacterium of any one of paragraphs 24-33, wherein said bacterium is co-cultured with a second microbe that consumes the feedstock solution.
35. An engineered heterotroph, comprising one or more of the following:
   a) at least one overexpressed functional sucrose catabolism gene;
   b) (i) at least one endogenous sucrose catabolism repressor gene comprising at least one engineered inactivating modification; or (ii) at least one exogenous inhibitor of an endogenous sucrose catabolism repressor gene or gene product;
   c) (i) at least one endogenous arabinose utilization gene comprising at least one engineered inactivating modification; or (ii) at least one exogenous inhibitor of an endogenous arabinose utilization gene or gene product; and/or
   d) at least one exogenous copy of at least one functional secondary product synthesis gene.
36. The engineered heterotroph of paragraph 35, wherein the engineered heterotroph is E. coli.
37. The engineered heterotroph of any one of paragraphs 35-36, wherein the at least overexpressed functional sucrose catabolism gene is endogenous.
38. The engineered heterotroph of any one of paragraphs 35-37, wherein the at least overexpressed functional sucrose catabolism gene comprises an invertase (CscA), a sucrose permease (CscB), and/or a fructokinase (CscK).
39. The engineered heterotroph of any one of paragraphs 35-38, wherein the endogenous sucrose catabolism repressor gene comprises the repressor (CscR).
40. The engineered heterotroph of any one of paragraphs 35-39, wherein the endogenous arabinose utilization gene comprises araB, araA, araD, and/or araC.
41. The engineered heterotroph of any one of paragraphs 35-40, wherein the at least one functional secondary product synthesis gene is heterologous.
42. The engineered heterotroph of any one of paragraphs 35-41, wherein the at least one functional secondary product synthesis gene comprises a violacein synthesis gene.
43. The engineered heterotroph of any one of paragraphs 35-42, wherein the at least one functional violacein synthesis gene comprises VioA, VioB, VioC, VioD, and/or VioE.
44. The engineered heterotroph of any one of paragraphs 35-43, wherein the at least one functional secondary product synthesis gene comprises a β-carotene synthesis gene.
45. The engineered heterotroph of any one of paragraphs 35-44, wherein the at least one functional β-carotene synthesis gene comprises CrtE, CrtB, CrtI, and/or CrtY.
46. The engineered heterotroph of any one of paragraphs 35-45, wherein the engineered heterotroph has enhanced sucrose utilization as compared to the same heterotroph lacking the engineered sucrose catabolism gene(s), sucrose catabolism repressor(s), arabinose utilization gene(s), and/or secondary product synthesis gene(s).
47. A method of producing a feedstock solution, comprising:
   a) culturing the engineered bacterium of any of paragraphs 24-34 in a culture medium comprising CO₂ and/or H₂; and
   b) isolating, collecting, or concentrating a feedstock solution from said engineered bacterium or from the culture medium of said engineered bacterium.
48. The method of any of paragraph 47, wherein the culture medium comprises CO₂ as the sole carbon source, and/or the culture medium comprises H₂ as the sole energy source.
49. The method of any one of paragraphs 47-48, wherein the culture medium further comprises arabinose.
50. The method of any one of paragraphs 47-49, wherein the feedstock solution comprises a sucrose concentration of at least 100 mg/mL.
51. The method of any one of paragraphs 47-50, wherein the feedstock solution comprises a sucrose concentration of at least 150 mg/mL.
52. The method of any one of paragraphs 47-51, wherein the feedstock solution comprises a sucrose feedstock for at least one heterotroph.
53. The method of any one of paragraphs 47-52, wherein the at least one heterotroph comprises an organism with enhanced sucrose utilization.
54. The method of any one of paragraphs 47-53, wherein the at least one heterotroph comprises E. coli and/or S. cerevisiae.
55. The method of any one of paragraphs 47-54, wherein the at least one heterotroph comprises an engineered bacterium of any one of paragraphs 35-46.
56. An engineered C. necator bacterium comprising at least one exogenous copy of at least one functional lipochitooligosaccharide synthesis gene.
57. The engineered bacterium of paragraph 56, wherein said engineered bacteria is a chemoautotroph.
58. The engineered bacterium of any one of paragraphs 56-57, wherein said engineered bacteria uses CO₂ as its sole carbon source, and/or said engineered bacteria uses H₂ as its sole energy source.
59. The engineered bacterium of any one of paragraphs 56-58, wherein the at least one functional lipochitooligosaccharide synthesis gene comprises an N-acetylglucosaminyltransferase gene, a deacetylase gene, and/or an acetyltransferase gene.
60. The engineered bacterium of any one of paragraphs 56-59, wherein the at least one functional lipochitooligosaccharide synthesis gene is heterologous.
61. The engineered bacterium of any one of paragraphs 56-60, wherein the at least one functional heterologous lipochitooligosaccharide synthesis gene comprises B. japonicum NodC, B. japonicum NodB, and/or B. japonicum NodA.
62. The engineered bacterium of any one of paragraphs 56-61, wherein said engineered bacteria produces lipochitooligosaccharide.
63. A method of producing a fertilizer solution, comprising:
   a) culturing the engineered bacterium of any of paragraphs 56-62 in a culture medium comprising CO₂ and/or H₂; and
   b) isolating, collecting, or concentrating a fertilizer solution from said engineered bacterium or from the culture medium of said engineered bacterium.
64. The method of any of paragraph 63, wherein the culture medium comprises CO₂ as the sole carbon source, and/or the culture medium comprises H₂ as the sole energy source.
65. The method of any one of paragraphs 63-64, wherein the fertilizer comprises lipochitooligosaccharides.
66. The method of any one of paragraphs 63-65, the fertilizer solution comprises a lipochitooligosaccharide concentration of at least 1 mg/L.
67. A system comprising:
   a) a reactor chamber with a solution contained therein, wherein the solution comprises hydrogen (H₂) and carbon dioxide (CO₂); and
   b) at least one of the following engineered bacteria in the solution:
      i) the engineered bioplastics bacterium of any of paragraphs 1-14;
      ii) the engineered sugar feedstock bacterium of any of paragraphs 24-34;
      iii) the engineered heterotroph of any of paragraphs 35-46; or
      iv) the engineered fertilizer solution bacterium of any of paragraphs 56-62.
68. The system of paragraph 67, further comprising a pair of electrodes in contact with the solution that split water to form the hydrogen.
69. The system of any one of paragraphs 67-68, further comprising an isolated gas volume above a surface of the solution within a head space of a reactor chamber.
70. The system of any one of paragraphs 67-69, wherein the isolated gas volume comprises primarily carbon dioxide.
71. The system of any one of paragraphs 67-70, further comprising a power source comprising a renewable source of energy.
72. The system of any one of paragraphs 67-71, wherein the renewable source of energy comprises a solar cell, wind turbine, generator, battery, or grid power.

### EXAMPLES

### Example 1

Valorization of CO₂ Through Lithoautotrophic Production of Sustainable Chemicals in C. necator

A sustainable future relies, in part, on minimizing the use of petrochemicals and reducing greenhouse gas (GHG) emissions. Modern society relies on fossil fuels for power, transportation, and chemical production but lacks clear paths towards viable substitutes. As industrial bioproduction industry has grown, economies of scale and use of cheaper feedstocks show promising trends towards commodities. Some of the cheapest and most sustainable feedstocks are gases (e.g., CO, CO₂, H₂, CH₄) from various point sources (e.g., steel mills, ethanol production plants, steam reforming plants, biogas). Compared to commonly-used carbohydrate-based feedstocks, these gas sources deliver carbon and energy sources to microbes in gas fermentation, and are more cost-effective, use land more efficiently, and have a smaller carbon footprint. Synthetic biology has developed a multitude of tools that permit the synthesis of complex molecules along with the means to domesticate non-model microbes. Herein is provided evidence that such an organism, Cupriavidus necator, is well-suited to produce a diverse set of compounds from gaseous sources. Use of the genetic tools described herein for autotrophic strains can lead to widespread adoption of gas fermentation and advance bioproduction of commodities.

C. necator H16 (formerly known as Ralstonia eutropha H16) is an attractive species for industrial gas fermentation. It is a facultative Knallgass bacterium that derives its energy from H₂ and carbon from CO₂, is genetically tractable, can be cultured with inexpensive minimal media components, is non-pathogenic, has a high-flux carbon storage pathway, and fixes the majority of fed CO₂ into biomass. See e.g., Steinbüchel, A. Polyhydroxyalkanoic acids. in Biomaterials (ed. Byrom, D.) 123-213 (Palgrave Macmillan UK, 1991); Brigham Appl. Microbiol. Biotechnol. 103 2113-2120 (2019); Brigham et al. Manipulation of Ralstonia eutropha Carbon Storage Pathways to Produce Useful Bio-Based Products. In Reprogramming Microbial Metabolic Pathways (eds. Wang, X., Chen, J. & Quinn, P.) vol. 64 343-366 (Springer Netherlands, 2012). C. necator has been successfully used industrially for polyhydroxyalkanoate (PHA) production, but this has seen limited commercial success. In addition to PHAs, C. necator has been engineered to produce a variety of compounds including: 2-methylcitric acid, ferulic acid, isopropanol and 3-methyl-1-butanol—under heterotrophic conditions. More recently, lithotrophic conversion of CO₂ (via aerobic oxidation of hydrogen) by engineered strains has produced: 600 mg/L isopropanol, and 220 mg/L C4+C5 fusel alcohols, alkanes and alkenes at 4.4 mg/L, methyl ketones at 180 mg/L, and stable-isotope-labeled arginine at 7.1% of dry cell weight (DCW). See e.g., Ewering et al. Metab. Eng. 8, 587-602 (2006); Overhage et al. Appl. Environ. Microbiol. 68, 4315-4321 (2002); Lu et al. Appl. Microbiol. Biotechnol. 96, 283-297 (2012); Liu et al. Science 352, 1210-1213 (2016); Crépin et al. Metab. Eng. 37, 92-101 (2016); Muller et al. Appl. Environ. Microbiol. 79, 4433-4439 (2013); Lütte et al. Appl. Environ. Microbiol. 78, 7884-7890 (2012). Lithotrophic production of C₃ and C₄+C₅ alcohols has been demonstrated in a hybrid biological-inorganic system that supplies CO₂ and generates H₂ from water-splitting electrodes directly in the culture medium. C. necator can be engineered to produce a larger diversity of products that seek to promote the sustainable development of industrial bioproduction.

C. necator bridges the gap between cheap gaseous feedstocks and versatile bioproduction. Three avenues for bioproduction were selected for their ability to reduce GHG emissions, e.g., when industrially scaled. First, for bioproduction to play a major role in replacing unsustainable industries existing infrastructure must be provided for by producing feedstocks for heterotrophs from CO₂ rather than from plant material. Second, to demonstrate the versatility of commodity products C. necator is well-positioned to address, the types of PHA co-polymers that can be made lithotrophically was diversified—beyond polyhydroxybutyrate (PHB or P(3HB)). Third, C. necator was used to produce a plant growth enhancer to promote crop yields and offset fertilizer use. Implementation of these three avenues can reduce the demands set on agriculture to generate bioproducts while increasing land-use efficiency for food.

### Sugar Feedstock Production

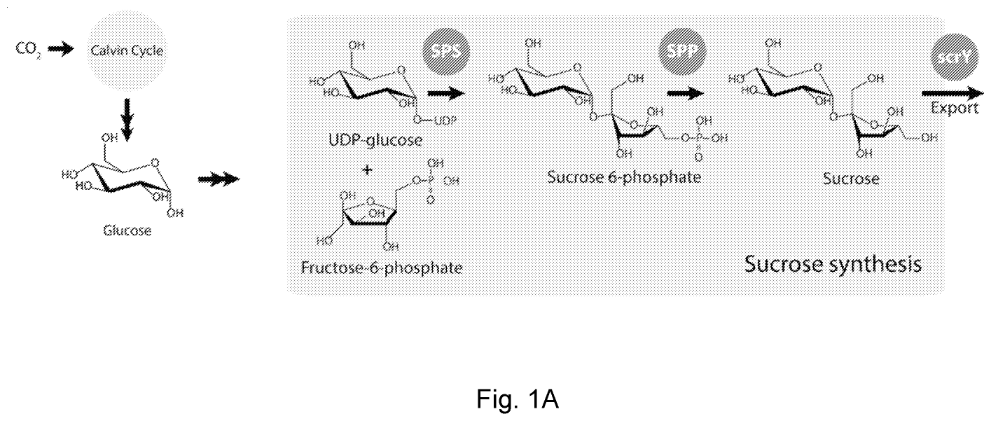

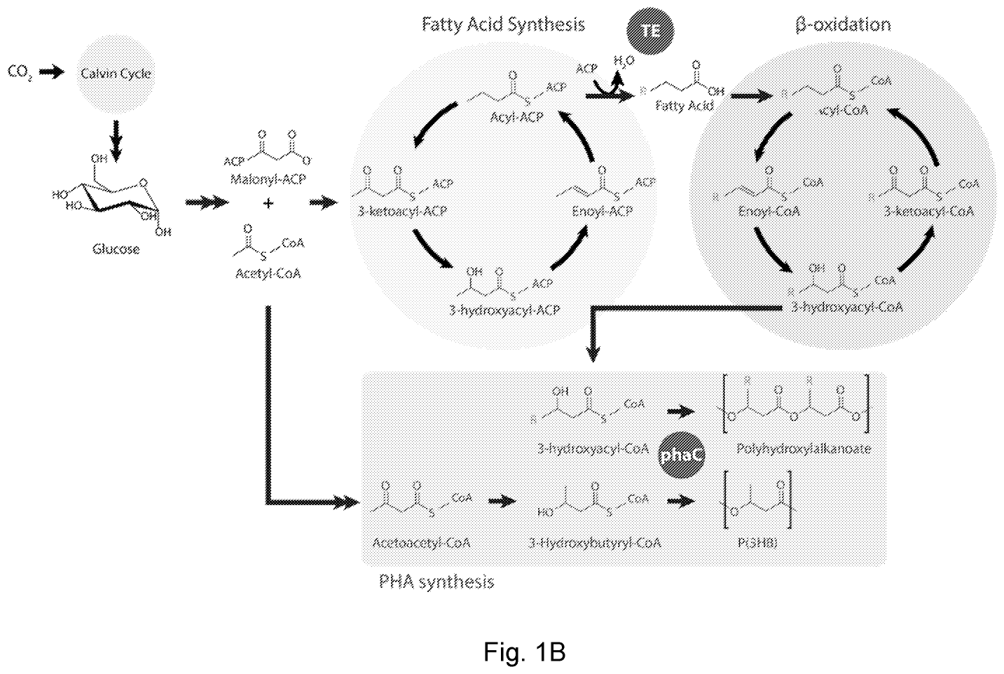

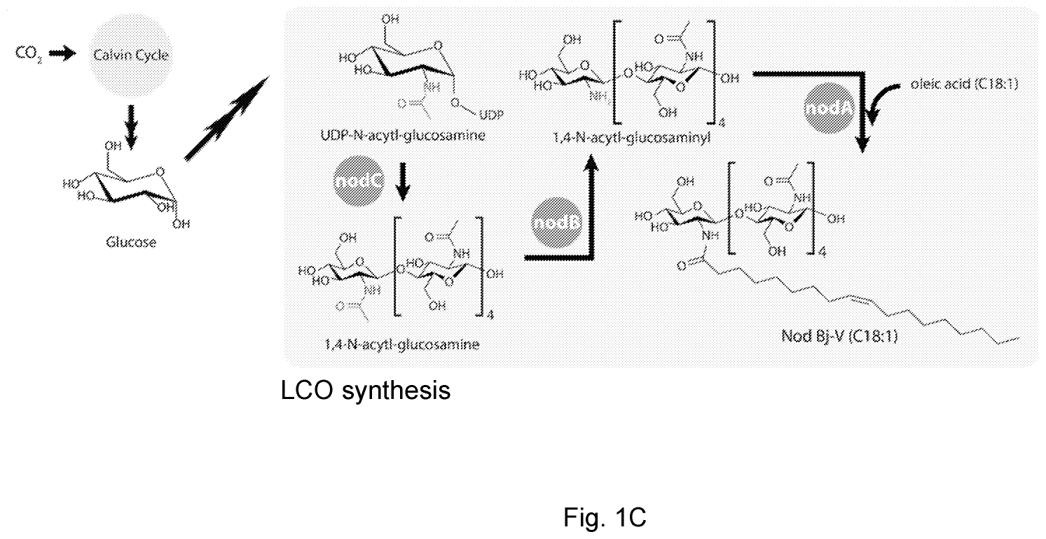

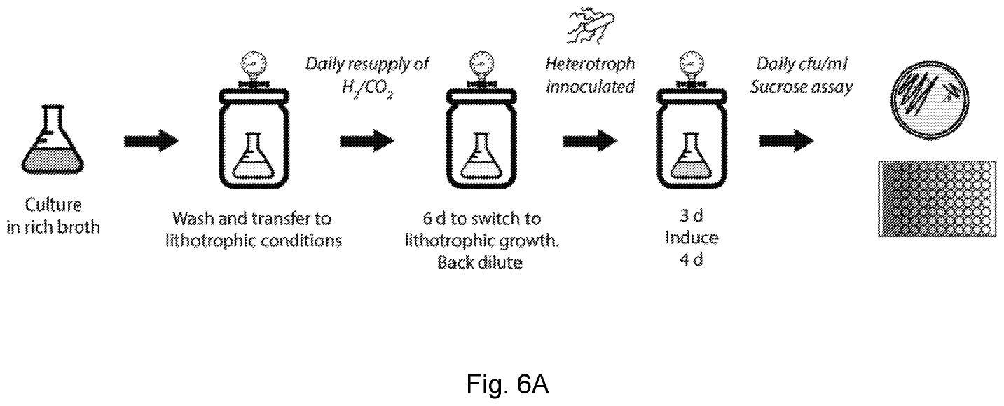

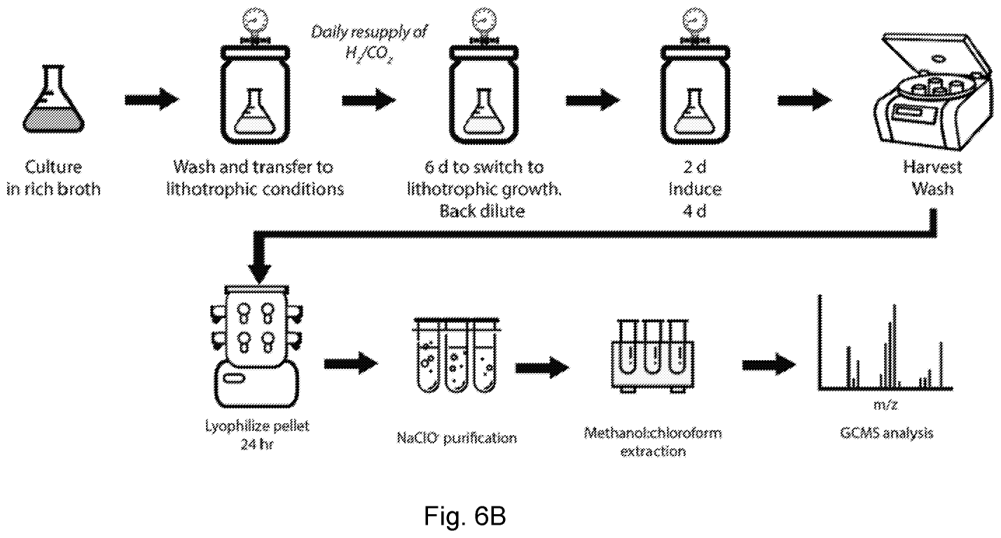

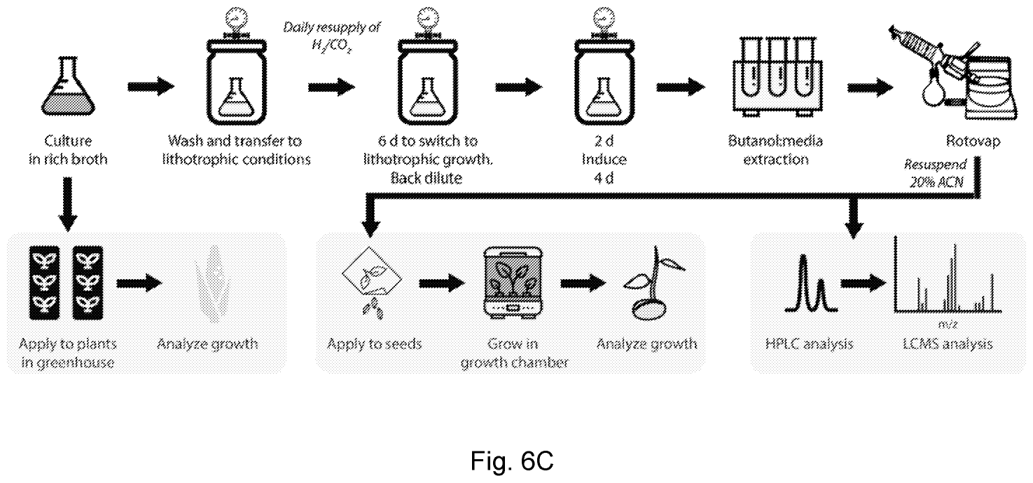

C. necator was engineered to convert CO₂ into sucrose as feedstock for the heterotrophs E. coli and S. cerevisiae (for experimental setup, see e.g., FIG. 6A). Sucrose has high energy-density and is commonly used by a variety of heterotrophs excluding C. necator. Here, the cyanobacterial sucrose synthesis enzymes in combination with a sucrose porin were express in C. necator to facilitate the export of sucrose into the media. Sucrose phosphate synthase (SPS) catalyzes the conversion of UDP-glucose and fructose 6-phosphate into sucrose 6-phosphate and sucrose phosphate phosphatase (SPP) hydrolyzes the phosphate to yield sucrose (see e.g., FIG. 1A).

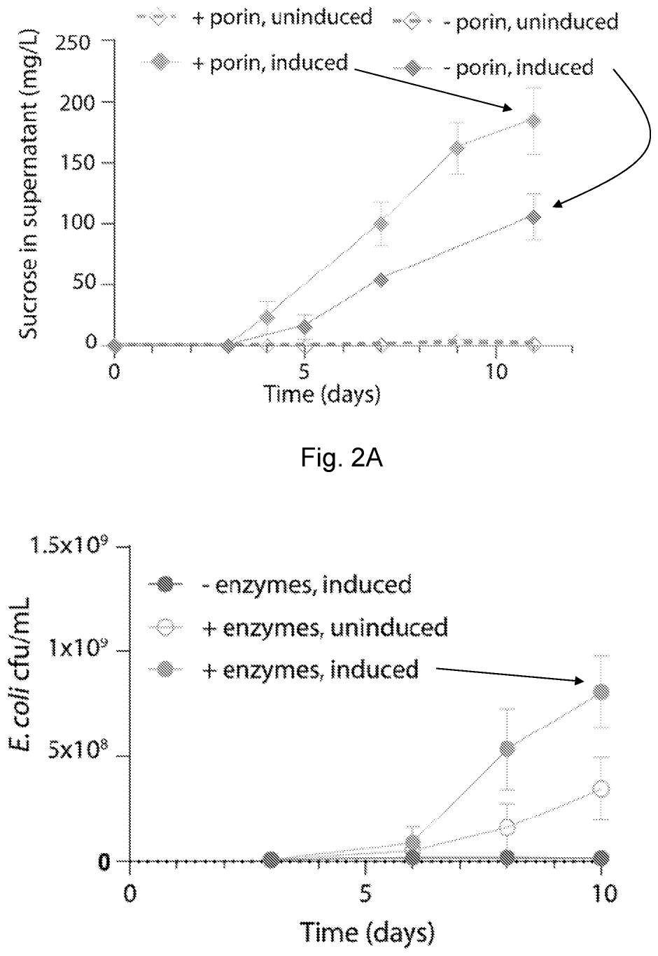

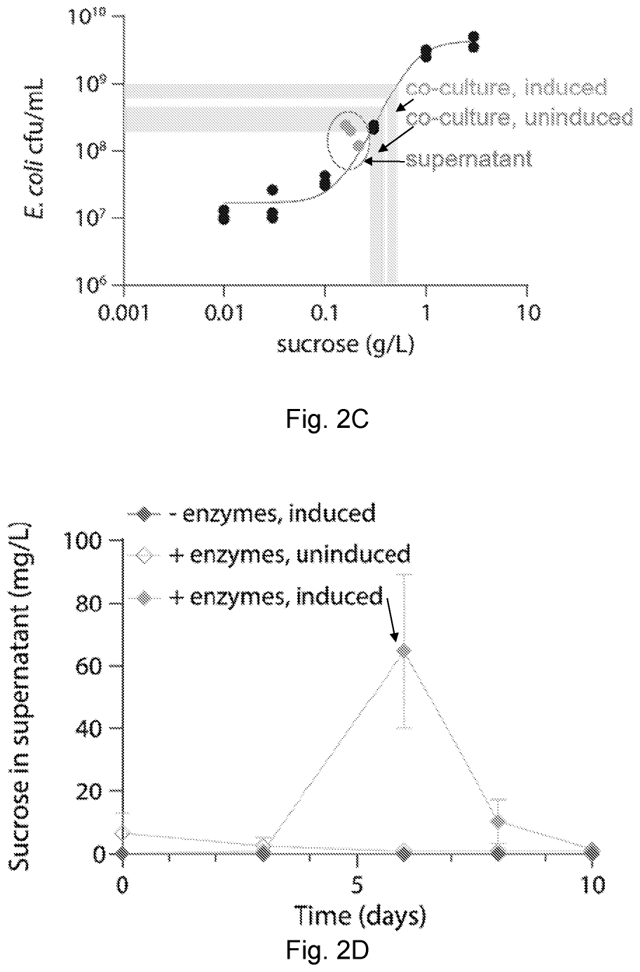

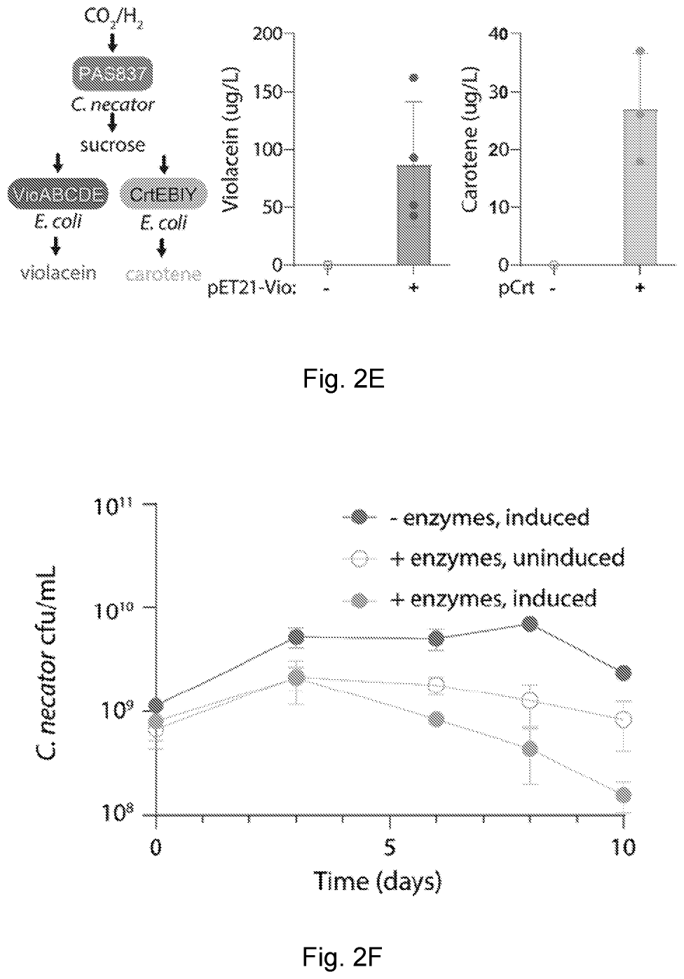

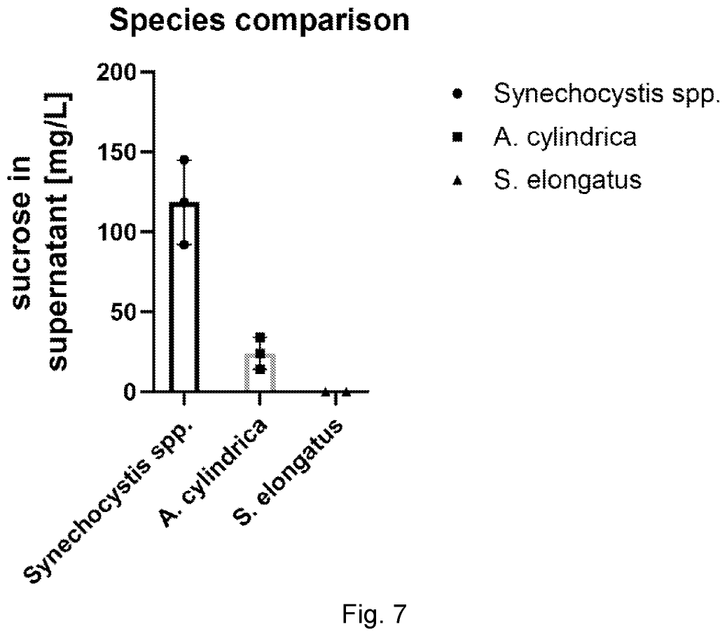

To choose the optimal enzymes for engineering, titers were initially compared of overexpressed SPS and SPP from Anabaena cylindrica PCC 7122, overexpressed SPS and SPP from Synechocystis sp. PCC 6803, and a fusion SPS/SPP from Synechococcus elongatus PCC7942 in C. necator. The Synechocystis enzymes led to 10-fold higher titers compared to those from A. cylindrica (see e.g., FIG. 7). No production was detected from the S. elongatus enzymes. All subsequent experiments were done in C. necator strains containing the Synechocystis sucrose synthesis enzymes. Expression of only SPS and SPP led to sucrose titers of about 100 mg/L (11 days) (see e.g., FIG. 2A). When the genes encoding the sucrose porin scrY from E. coli were added, an 80% increase was detected in titers in the supernatant, to ˜180 mg/L and cell viability post-induction improved (data not shown). Increased titers indicate that export of sucrose drives the enzymatic reactions by reducing intracellular concentrations; without wishing to be bound by theory, the increased viability could be a result of decreased osmotic pressure or lowering of the inhibitive effects of product accumulation.

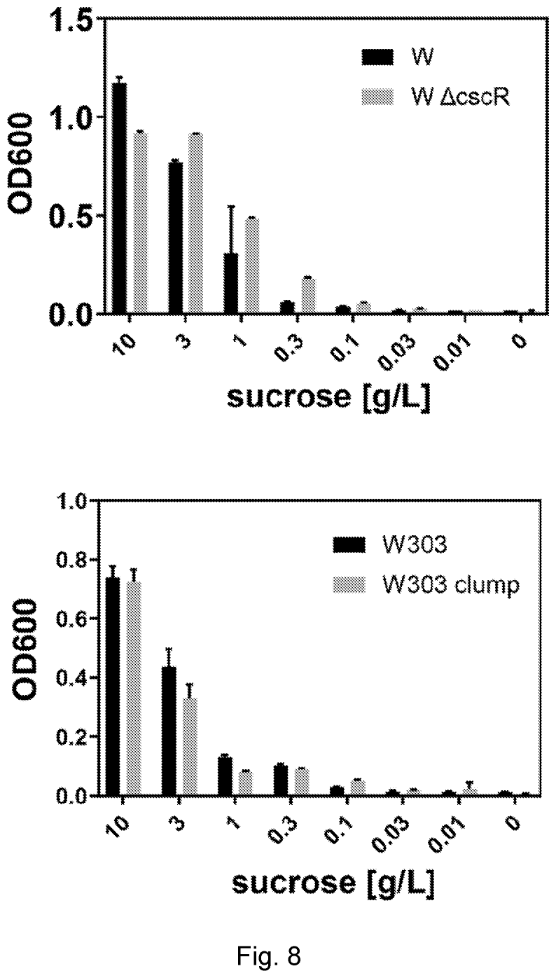

Heterotrophs can utilize sucrose produced by C. necator as a carbon source. To test the heterotrophs' growth response, E. coli and S. cerevisiae strains were engineered to exhibit enhanced sucrose utilization: E. coli ΔcscR W (PAS842) and S. cerevisiae W303 Clump (PAS844). The E. coli ΔcscR W strain (PAS842) was engineered with a genomically-integrated sucrose catabolism (csc) operon containing an invertase (CscA), a sucrose permease (CscB), and a fructokinase (CscK); the repressor (CscR) was knocked-out. This strain is able to grow to equivalent densities from ˜5× lower sucrose than the parent W strain (see e.g., FIG. 8). Similarly, the yeast strain (PAS844), S. cerevisiae w303^{Clump}, was derived from a low-sucrose directed evolution experiment that conferred increased sucrose utilization and fitness through mutations in CSE2 (a subunit of RNA Pol II mediator), IRA1 (the Inhibitory Regulator of the RAS-cAMP pathway), MTH1 (involved in glucose-regulated gene expression), UBR1 (an E3 ubiquitin ligase), and ACE2 (involved in full septation of budding) (see e.g., FIG. 8). These mutations collectively led to increased local cell densities, which enhances cell growth via extracellular sucrose cleavage. Additionally, because the induction system (pBAD) is under the control of arabinose—a carbon source for both E. coli and S. cerevisiae—arabinose utilization was knocked out from the E. coli strain (PAS842), and the S. cerevisiae strain (PAS844) was grown in anaerobic conditions such that arabinose was not consumed. See e.g., Sabri et al. Appl. Environ. Microbiol. 79, 478-487 (2013); Arifin et al. J. Biotechnol. 156, 275-278 (2011); Hays et al. J. Biol. Eng. 11, (2017); Koschwanez et al. PLOS Biol. 9, e1001122 (2011); Barnett, J. A. The Utilization of Sugars by Yeasts. in Advances in Carbohydrate Chemistry and Biochemistry (ed. Tipson, R. S.) vol. 32 125-234 (Academic Press, 1976).

To verify whether the sucrose titers were sufficient to grow heterotrophs, E. coli and S. cerevisiae were first grown in the spent lithotrophic minimal media from 9 days of PAS837 growth with and without induction of the sucrose synthesis genes. The E. coli was inoculated at OD600=0.01 and S. cerevisiae at OD600=0.05 and grown anaerobically for 48 hr at 30° C. while shaking (see e.g., FIG. 9). Both E. coli and S. cerevisiae cultures grow in spent media from induced PAS837 by two doublings but showed no growth in uninduced PAS837 (later supplemented with 0.3% arabinose during heterotroph growth; see e.g., FIG. 9). This result validated that engineered C. necator can support the growth of these heterotrophs.

E. coli grew in co-culture with sucrose producing C. necator. Subsequent experiments focused on E. coli because C. necator is aerobic and the S. cerevisiae strain would be able to utilize the arabinose inducer in aerobic conditions. Once C. necator PAS837 was stably growing in lithotrophic conditions (30:15: balance, H₂:CO₂:air), the culture was back-diluted to OD600=0.5, and cells were allowed to grow for 72 hr, at which time the C. necator cultures were both induced and co-inoculated with E. coli PAS842 and allowed to grow for an additional 7 days (see e.g., FIG. 2B, 2D, 2F). E. coli PAS842 in co-culture with induced C. necator PAS837 grew to 22× higher cfu/mL compared to WT C. necator and 2× higher compared to uninduced C. necator PAS837. Growth of E. coli in induced co-culture coincided with a sharp decrease of sucrose measured in the supernatant from 60 mg/L to less than 10 mg/L (see e.g., FIGS. 2B and 2D). Sucrose was not observed in the supernatant of either WT or uninduced C. necator co-cultures. Overall, C. necator WT cfu/mL was higher compared to engineered strains and all strains growth declined over time (see e.g., FIG. 2F).

Notably, the growth of E. coli in co-culture with C. necator exceeded expected growth based on monoculture sucrose production by C. necator. In order to determine E. coli sucrose requirements, E. coli was grown in increasing concentrations of sucrose and the resulting E. coli cfu/mL was correlated after 2 days (see e.g., FIG. 2C). E. coli growth was then compared in supernatant with growth in co-culture. E. coli growth in co-culture corresponded to 2.5-fold higher sucrose concentrations than those measured in C. necator monocultures (see e.g., FIG. 2C). The growth patterns are consistent with a model in which a symbiotic heterotroph acts as a thermodynamic sink for feedstock production (see e.g., Hu et al., PNAS 113, 3773-3778 (2016)), which without wishing to be bound by theory could explain the increased growth and higher sucrose production. Co-culturing a heterotroph directly with a feedstock producer thus increased the growth and increased the yield of the product made by the heterotroph.

E. coli produced violacein in co-culture with sucrose producing C. necator. Violacein pET21b-VioABCDE (PAS845) and β-carotene-expressing pSB1C3-CrtEBIY (PAS846) plasmids were introduced into the E. coli strain (see e.g., Balibar et al., Biochem. 45, 15444-15457 (2006); Lemuth et al., Microb. Cell Factories 10, 29 (2011)). For production, 100 mL of C. necator was grown lithotrophically for 3 days before induction and addition of violacein-producing E. coli at OD600 0.01 or β-carotene-producing E. coli at OD600=0.01. Gas was replenished every three days, and the cultures were harvested 10 days after induction. After cultivation, cells were harvested by centrifugation and products were extracted in 1 mL of 100% ethanol. Violacein and β-carotene were quantified by comparison to an analytical standard. The engineered E. coli produced 80-250 ug/L violacein and 50 ug/L carotene from CO₂ (see e.g., FIG. 2E). E. coli grew by about 2 orders of magnitude while C. necator growth was reduced by one half of an order of magnitude. Due to H₂ safety requirements, the C. necator strains rarely exceeded densities above OD600 of 3. In sum, violacein and beta-carotene were produced from CO₂ and H₂ as sole sources of carbon and energy in this system.

### Polyhydroxyalkanoate (PHA) Production

Prior work has been done to understand and optimize C. necator PHA bioplastic production and has achieved yields up to 1.5 g/L/hr in lithotrophic conditions (see e.g., Tanaka et al., Biotechnol. Bioeng. 45, 268-275 (1995)). The most commonly produced PHA, polyhydroxybutyrate (P(3HB), PHB), is a brittle thermoplastic with a narrow processing window and suboptimal material properties. To extend the utility of C. necator for bioplastic production, gas fermentation and genetic engineering were combined herein, allowing for the production of tailored and more versatile PHAs from CO₂ and H₂.

To produce these tailored biopolyesters, thioesterases (TEs) were expressed with PHA synthases (phaCs) (see e.g., FIG. 1B). As the last step in PHA synthesis, phaCs assemble the polymer based on their intrinsic substrate specificities and enzymatic rates. By introducing two heterologous enzymes (i.e., a TE and a phaC) that have variable specificities for different chain-length fatty acids, bioplastics can be generated with tailored compositions. Additionally, further alterations to composition and production can be made by either removing or complementing the native PHA synthase (phaC_{Cn}) or modulating β-oxidation.

PHA co-polymers are generally derived through the introduction of the co-monomer precursors as feedstocks, of which saturated fatty acids are the most common. Rather than feeding these precursors TEs were introduced to produce fatty acids to then be integrated into the co-polymer. Two acyl carrier protein (ACP) TEs were selected: plant TE U. californica FatB2 a 12:0 acyl-ACP TE and an engineered chimera of C. palustris FatB1(aa 1-218) and FatB2 (aa 219-316)—Chimera 4 (chim4). See e.g., Voelker & Davies, J. Bacteriol. 176, 7320-7327 (1994); Torella et al. PNAS 110, 11290-11295 (2013); Ziesack, M. et al. Appl. Environ. Microbiol. 84, (2018). Chim4 couples the specificity of CpFatB1 as a C8:0 acyl-ACP TE with the high activity of CpFatB2, which is natively a predominantly C14:0 TE. Previous work established that when overexpressed in E. coli CpFatB1 produced 84 ug/ml of C8:0 (78% total FFA produced) and CpFatB2 372 ug/ml C14:0 (75% total FFA). The engineered chim4 produced 394 ug/ml of C8:0 (90% FFA). By combining these TEs with phaC polymerizing enzymes PHA co-polymers were produced directly from CO₂ that were equivalent to those made from heterotrophic conditions. The three phaCs that we used were: P. aeruginosa phaC1 and phaC2 and Pseudomonas spp 61-3 phaC1_{Ps}, which were co-expressed with the ACP TEs to generate the medium-chain length (mcl) PHAs. As previously reported, when phaC1_{Pa} is overexpressed in PHA-producing E. coli and fed C₁₂ fatty acid, the PHA co-polymer was composed of approximately 45% C12, 50% C10, 10% C8 molar proportion. In phaC2_{Pa}-overexpressing E. coli when fed C12 fatty acid a polymer composed of 35% C₁₂, 55% C₁₀, 10% C₈ hydroxy acids was produced. When this E. coli strain was fed C₈ fatty acid, the polymer was instead composed of 15% C10, 40% C8, 35% C6, 10% C5 hydroxy acids. When fed C8 fatty acid, P. putida GPp104 expressing the PHA synthesis pathway with Pseudomonas spp 61-3 phaC1 Ps produced 2% C10, 77% C8, 16% C6, 3% C4 hydroxy acids. See e.g., Antonio et al. FEMS Microbiol. Lett. 182, 111-117 (2000); Abedi et al. Adv. Biomed. Res. 5, (2016); Qi et al. FEMS Microbiol. Lett. 157, 155-162 (1997); Matsusaki et al. J. Bacteriol. 180, 6459-6467 (1998).

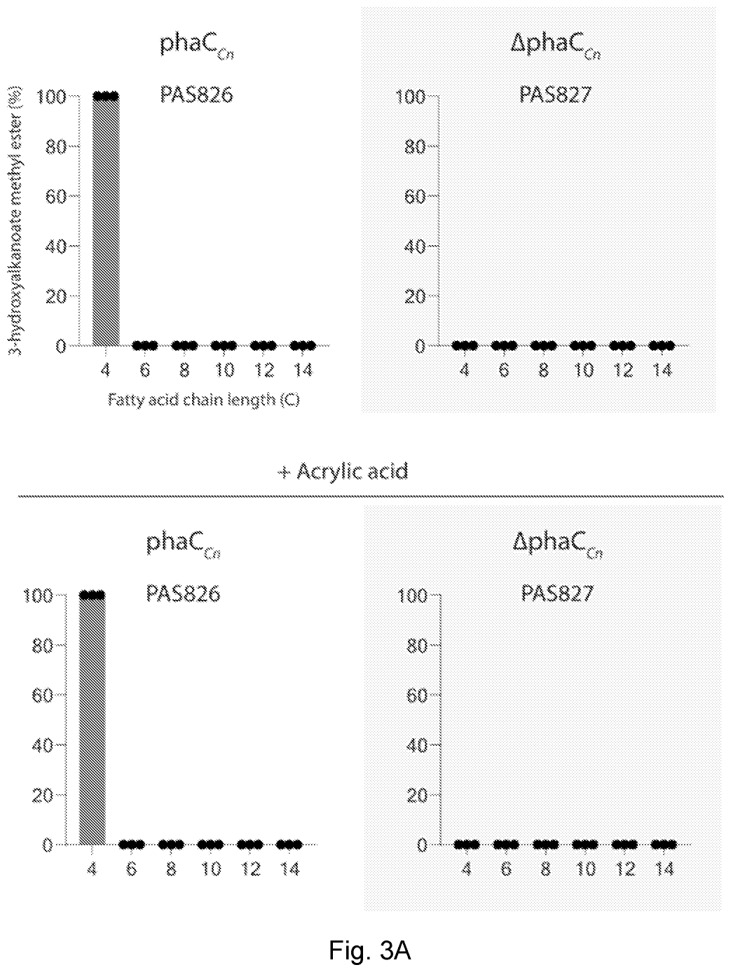

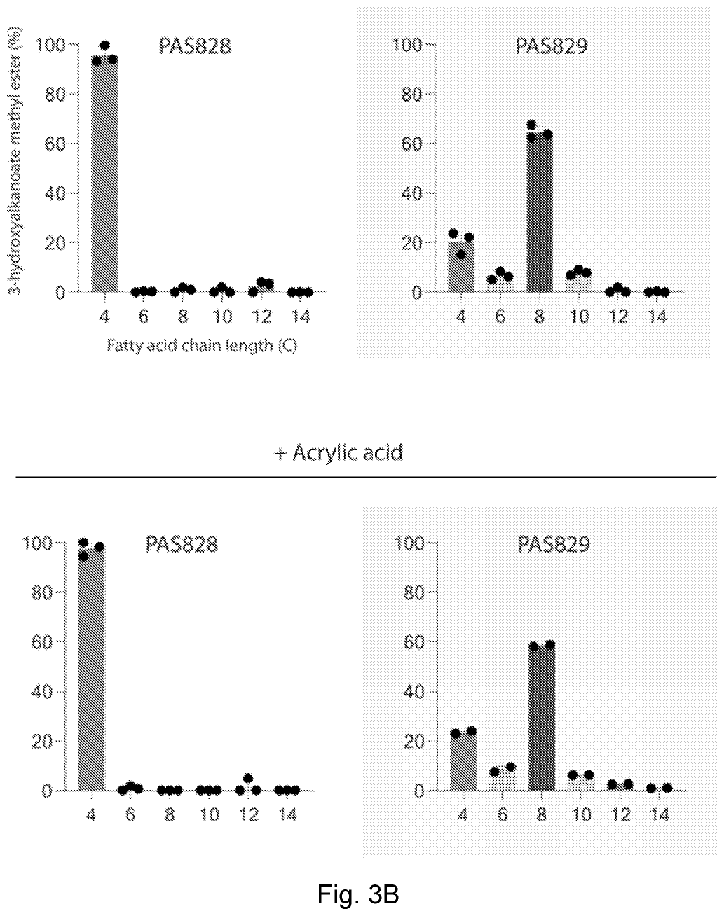

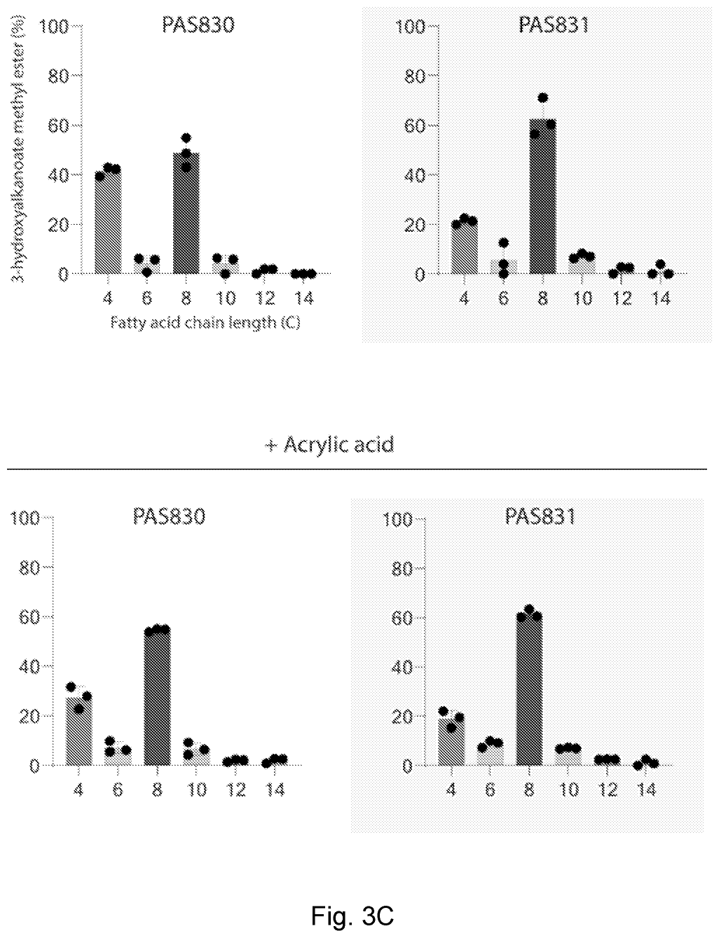

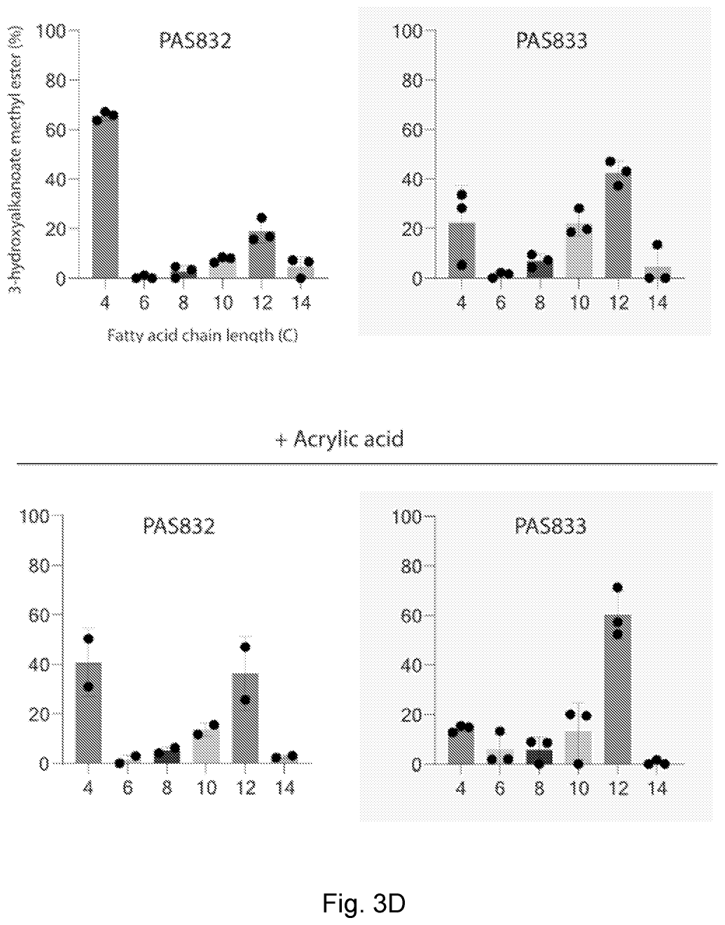

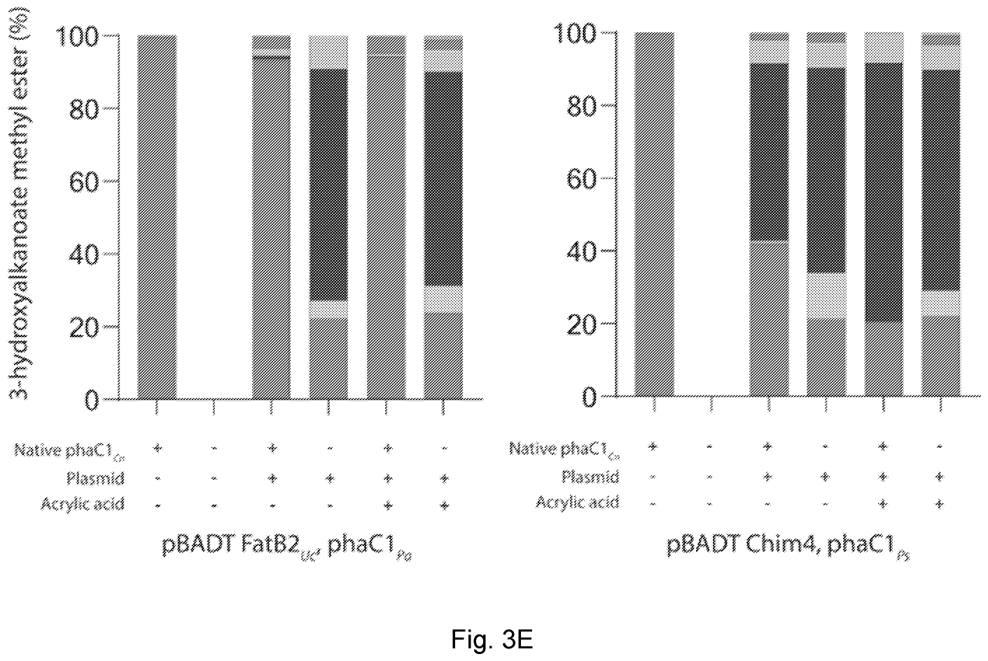

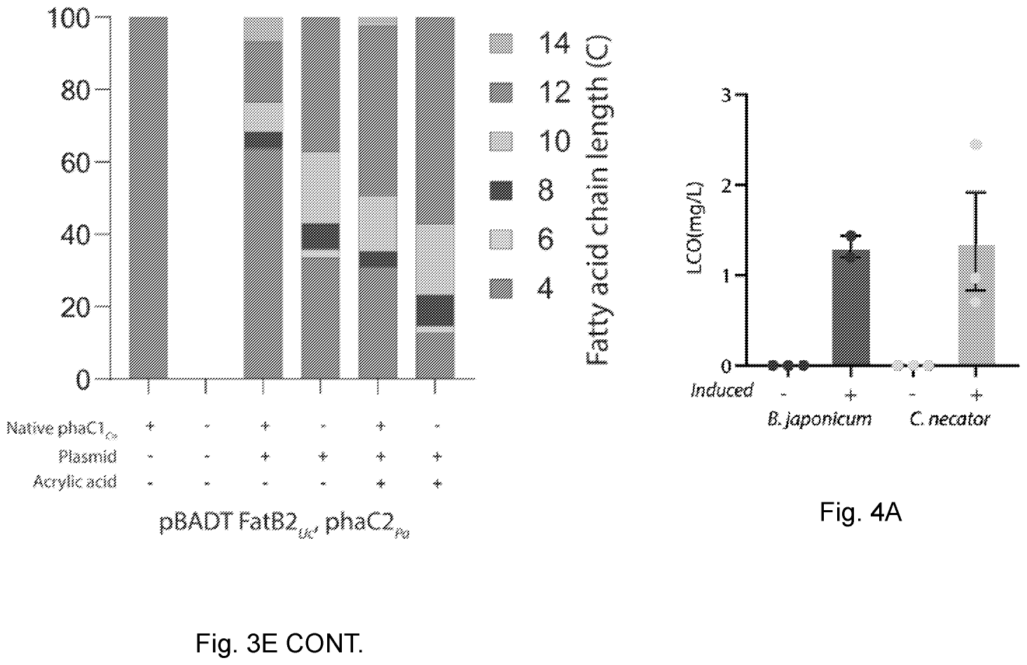

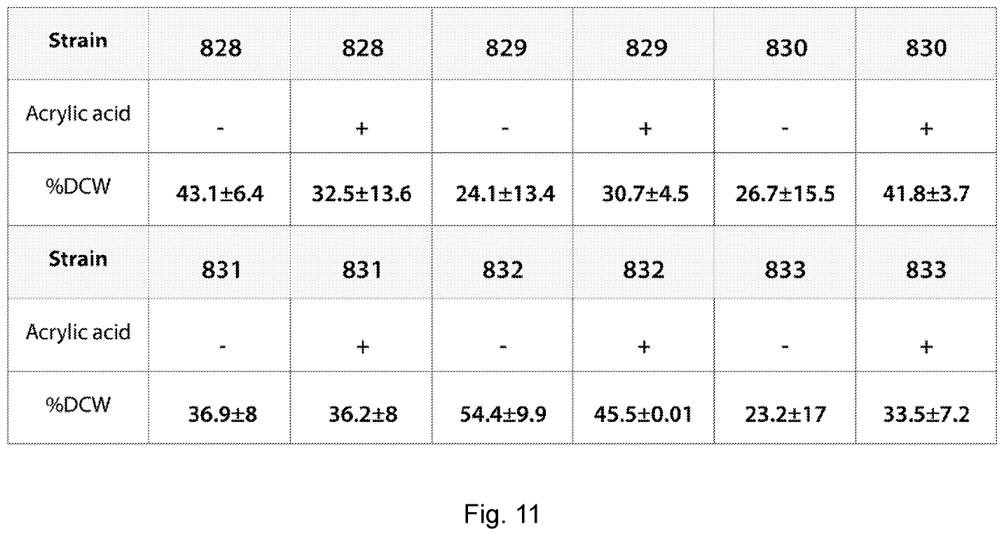

During lithotrophic growth C. necator strains produced a variety of expected co-polymers based on the combination of genetic background, genes of the expression plasmid, and pharmacological manipulation (e.g., β-oxidation inhibition; see e.g., Qi et a., FEMS Microbiol. Lett. 167, 89-94 (1998)). The PHA was purified through established NaClO⁻-based protocols and underwent subsequent methanolysis before GCMS analysis of medium chain-length hydroxy acids (mcl-3HA) at m/z=103 (see e.g., FIG. 6A). PHA titers based on percent dry cell weight (% DCW) (see e.g., FIG. 11) are representative of n=3 (all subsequent percentages are reported as the mean from n=3 unless otherwise noted). C. necator with the vector control ΔphaCc, (PAS827) failed to produce detectable PHAs whereas wildtype cells (PAS826) produced 100% 3HB; with no effect from acrylic acid in either condition (see e.g., FIG. 3A).

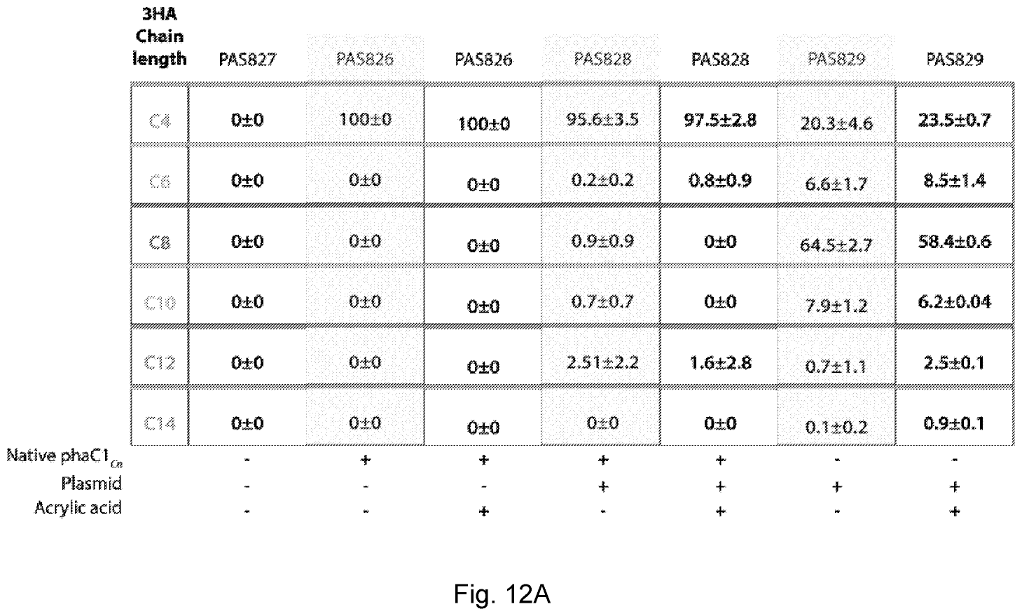

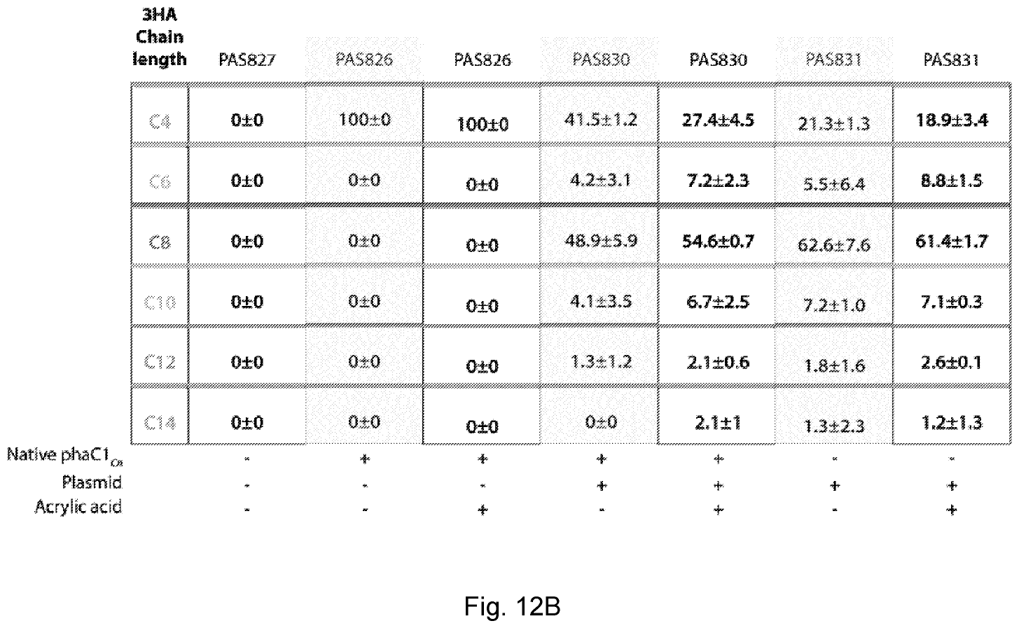

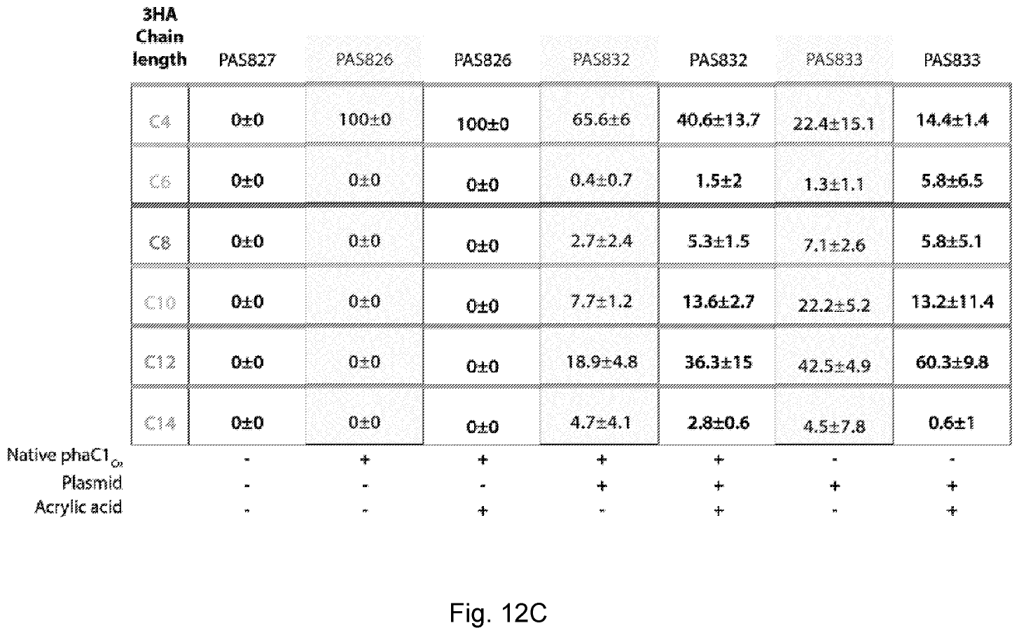

In the native phaC_{Cn} background, this enzyme pair (PAS828) produced very little mcl-3HA: μ=4.4% of total (see e.g., FIG. 3B top left panel, FIG. 12A). In the ΔphaC Cn background (PAS829), this plasmid produced μ=79.7% mcl-3HA, of which 64.5% was 3HO (see e.g., FIG. 3B, top right panel). Acrylic acid did not contribute significantly to the PHA composition; when added to PAS828, the proportion of 3HO and 3HD decreased to undetectable levels (see e.g., FIG. 3B, bottom left panel). PAS829 in the acrylic acid condition, produced a copolymer that was slightly enriched for longer-chain fatty acids compared to no-acrylic acid condition (see e.g., FIG. 3B, bottom right panel). Overall, while there were striking differences between the native and ΔphaC strains, acrylic acid did not seem to affect the proportion of fatty acids in this strain.

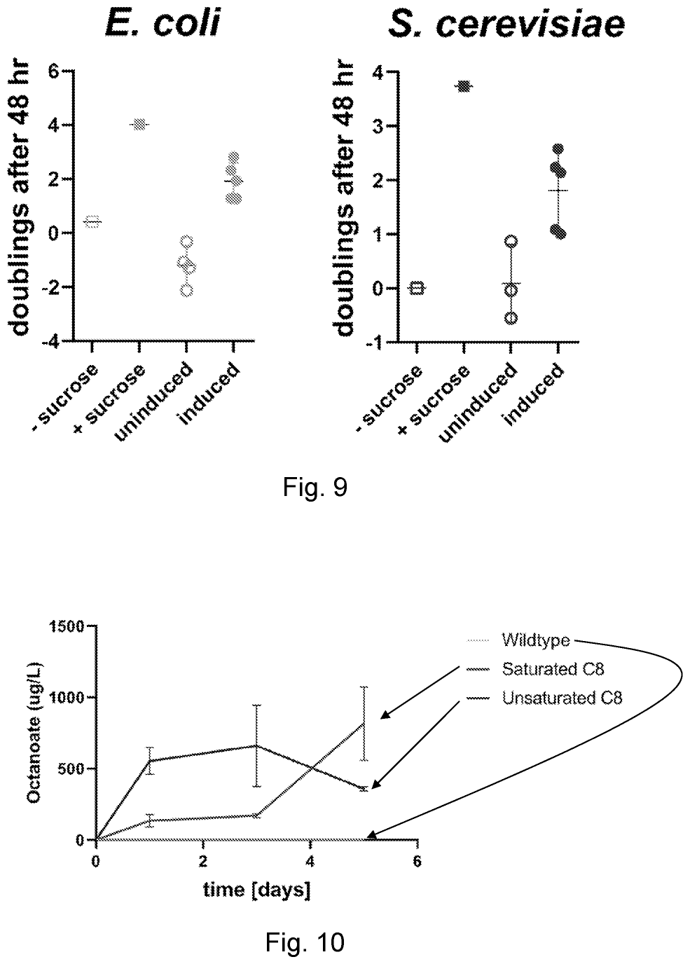

The second group of experiments used Pseudomonas spp 61-3 phaC1_{Ps} and the engineered chim4 TE enzyme with high activity and selectivity for octanoate production (see e.g., FIG. 10). This plasmid in the native phaC Cn background (PAS830) produced μ=58.5% mcl-3HA, of which μ=48.9% was 3HO (see e.g., FIG. 3C top left panel, FIG. 12B). Surprisingly, in the ΔphaC Cn background (PAS831) the composition of mcl-3HAs compared to the native background (PAS830) was not as striking as PAS828 to PAS829; with μ=78.7% being mcl-3HA with μ=62.6% as 3HO (see e.g., FIG. 3C, top right panel). The strongest effect of acrylic acid was observed on a strain with the native phaC, with the accumulation of longer chain fatty acids: μ=72.6% mcl-3HA, of which μ=54.6% was 3HO (see e.g., FIG. 3C, bottom left panel). After the addition of acrylic acid, there was a minor enrichment of mcl-3HAs in PAS831: μ=61.4% 3H0, =7.1% 3HD, μ=2.6% 3HDD, μ=1.2% 3HTD (n=2) (FIG. 3C, bottom right panel).

In a third set of experiments, the tunability of different combinations of TEs and phaCs were explored by exchanging the phaC1_{Pa} with its paralog phaC2_{Pa} while maintaining the Uc FatB2. The plasmid containing TE Uc FatB2 and phaC2_{Pa} in the native phaC Cn background (PAS832) mcl-3HAs comprised approximately μ=34.5% of the polymer, of which μ=18.9% of 3-hydroxydodecanoate (3HDD) (see e.g., FIG. 3D top left panel, FIG. 12C).

In strains lacking the native phaC Cn and expressing the above plasmid (PAS833), a greater accumulation of mcl-3HA was observed; μ=77.6% was mcl-3HA, of which μ=42.5% was 3HDD (see e.g., FIG. 3D, top right panel). When acrylic acid, an inhibitor of 3-ketoacyl CoA thiolase in β-oxidation, was added to PAS832, mcl-3HAs accumulated to a similar extent as seen in the ΔphaC strain (PAS833) without acrylic acid, μ=59.4 with μ=36.3% 3HDD (n=3) (see e.g., FIG. 3D, bottom left panel). Likewise, in PAS833, a marked increase in the proportion of 3HDD predominated; 60.3% 3HDD (see e.g., FIG. 3D, bottom right panel).

Together, these experiments indicate that there are many levers with which the composition of the PHA polymer can be modified (see e.g., FIG. 3E).

### Lipochitooligosaccharide Production

C. necator was engineered to convert CO₂ into lipochitooligosaccharides (LCOs), a plant growth enhancer. C. necator was engineered using a pBAD-based plasmid containing NodC protein, an N-acetylglucosaminyltransferase that builds the backbone, NodB, a deacetylase that acts on the non-reducing end, and NodA, an acetyltransferase that attaches a fatty acid (see e.g., FIG. 1C). This pathway produced a basic LCO molecule composed of a 5 (V) acylated chitin backbone and oleic acid at the non-reducing terminal N-acetylglucosamine monomer, which, following naming convention, is called Nod Cn-V (C_{18:1}).

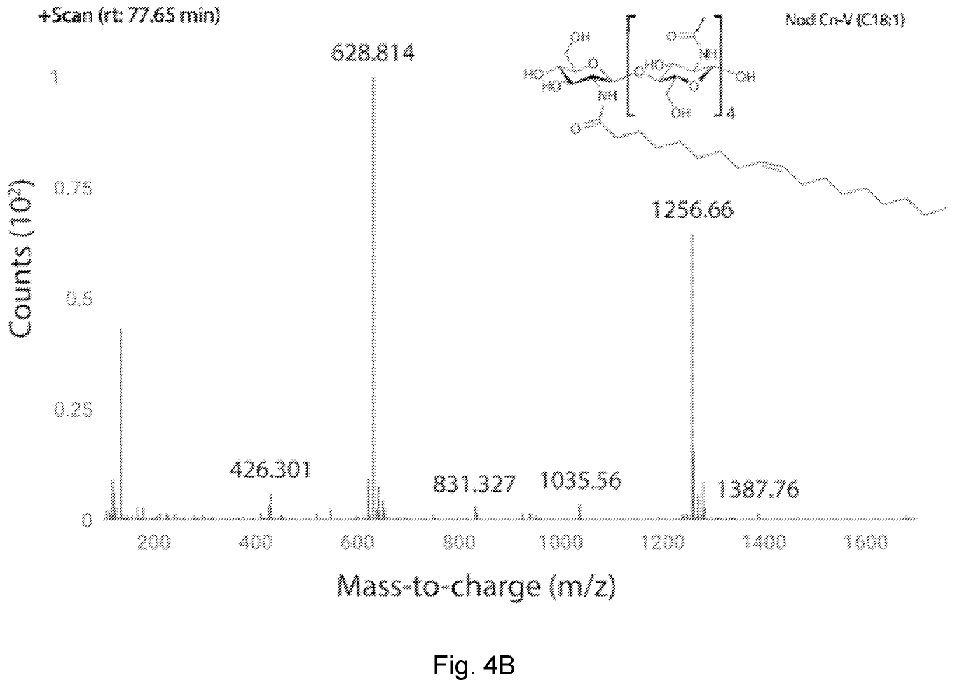

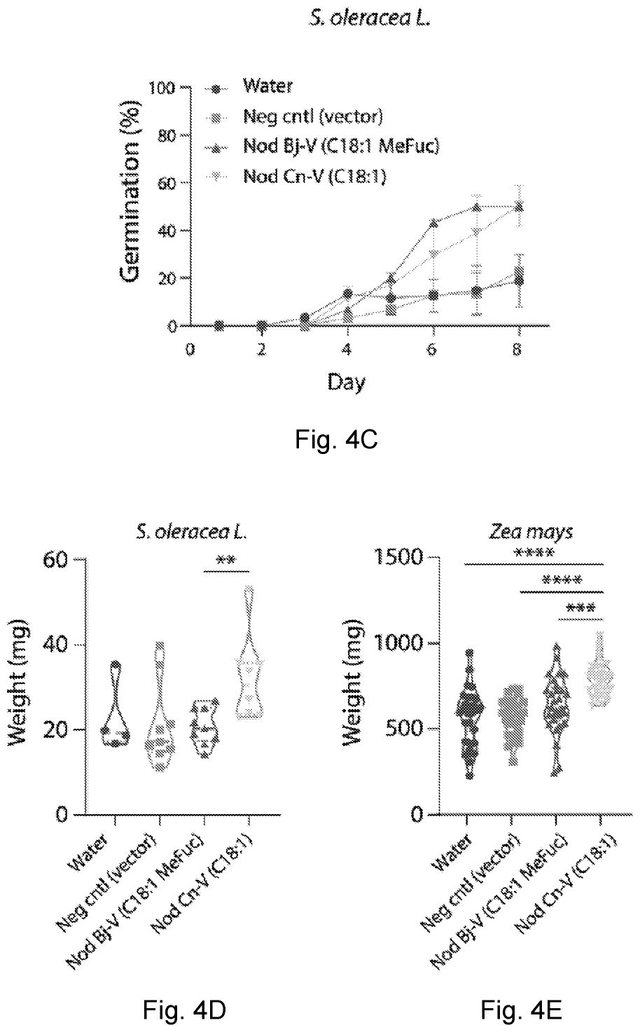

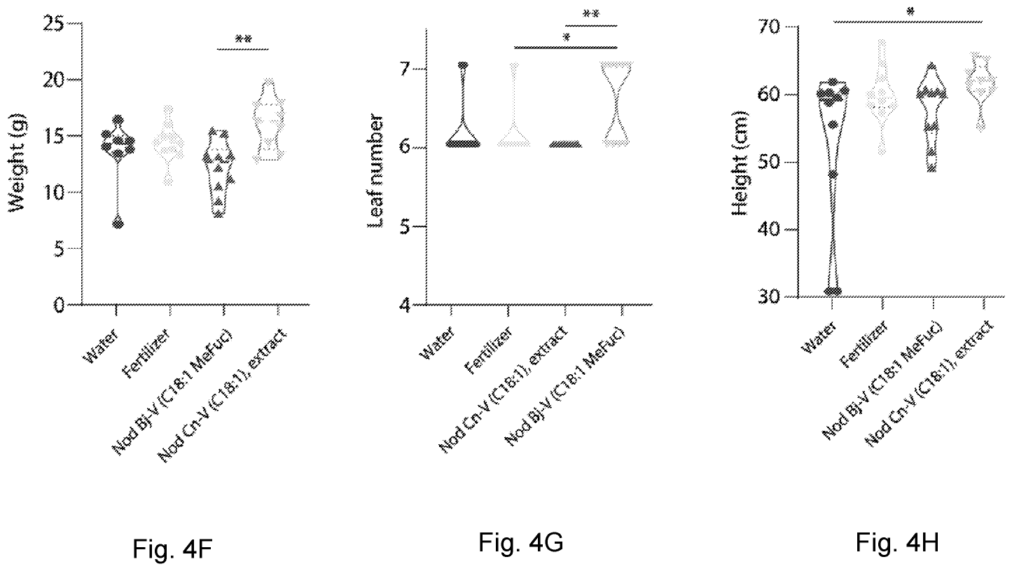

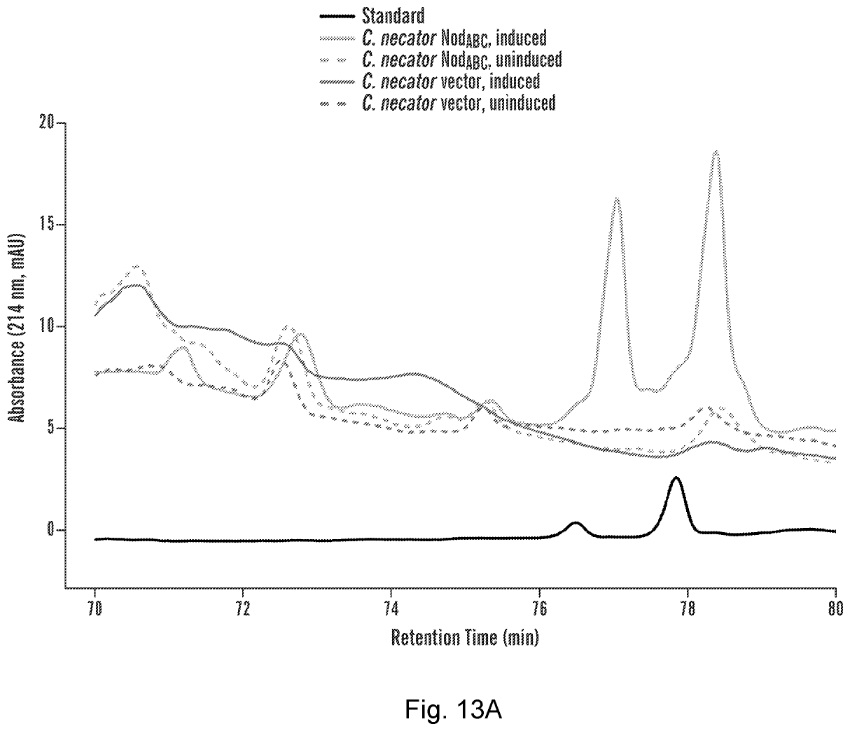

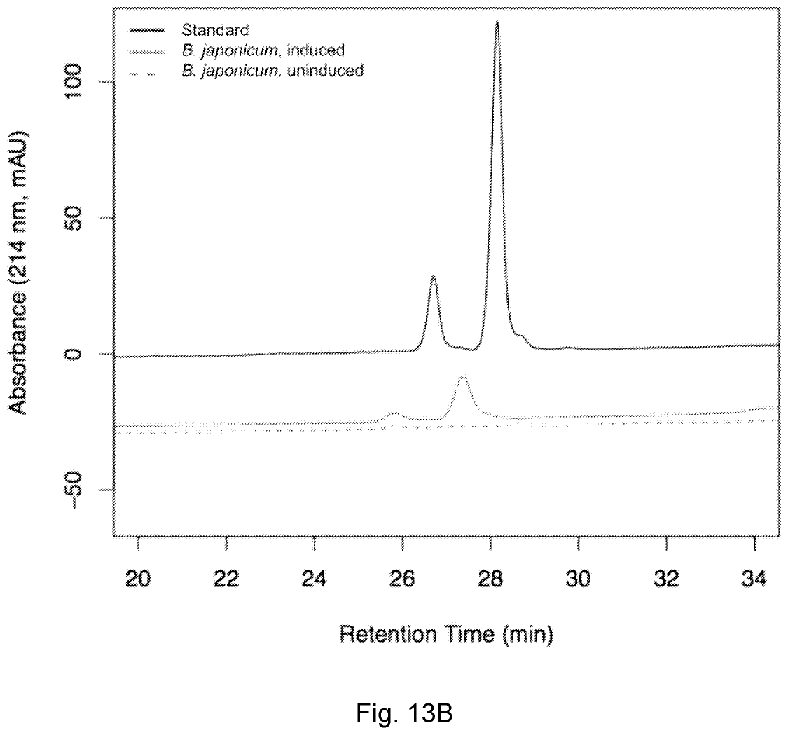

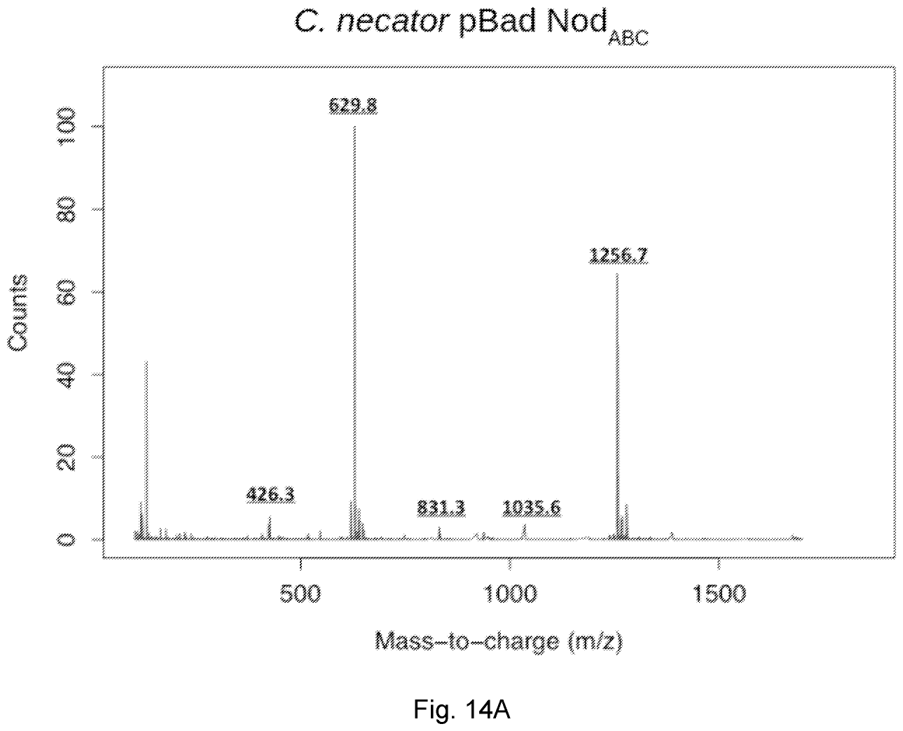

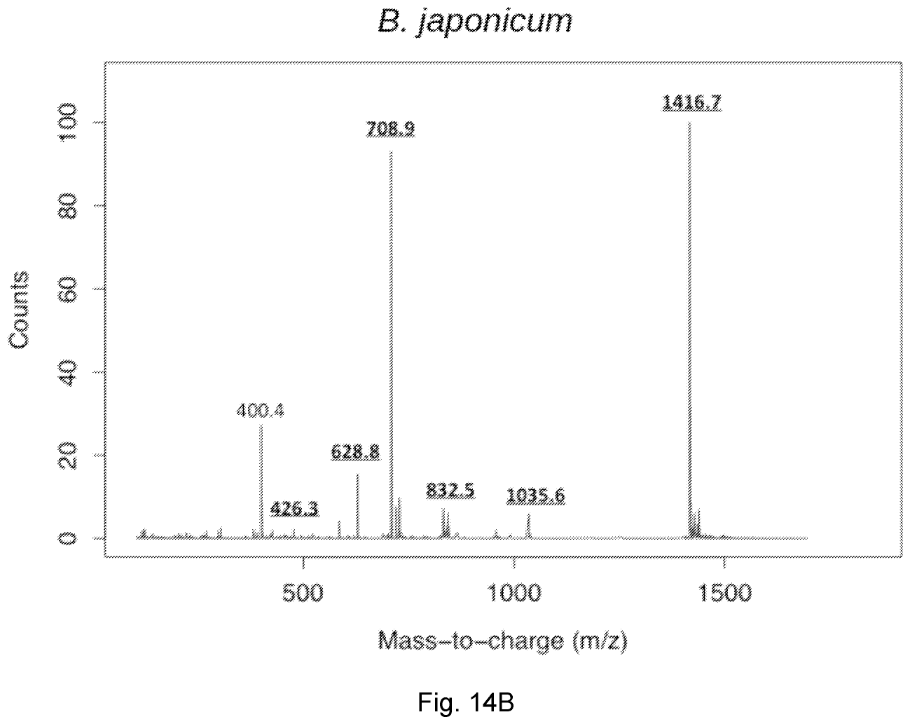

The Nod Cn-V (C_{18:1}) was initially detected from the engineered strain by HPLC and purified B. japonicum Nod Bj-V (C_{18:1}MeFuc) was used as a reference. Two peaks were observed at the expected retention times (see e.g., FIG. 13A-13B). In lithotrophic conditions, the engineered C. necator produced 1.37±0.44 SE mg/L over 72 hours (see e.g., FIG. 4A). LC-MS analysis confirmed the identity of Nod Cn-V (C_{18:1}). While all fragmentation peaks are consistent with Nod Bj-V (C_{18:1} MeFuc) (m/z=1035, 831, 629, and 426 m/z), the base peak at 1256—indicated the lack of the fucose moiety (see e.g., FIG. 4B, FIG. 14A-14B).

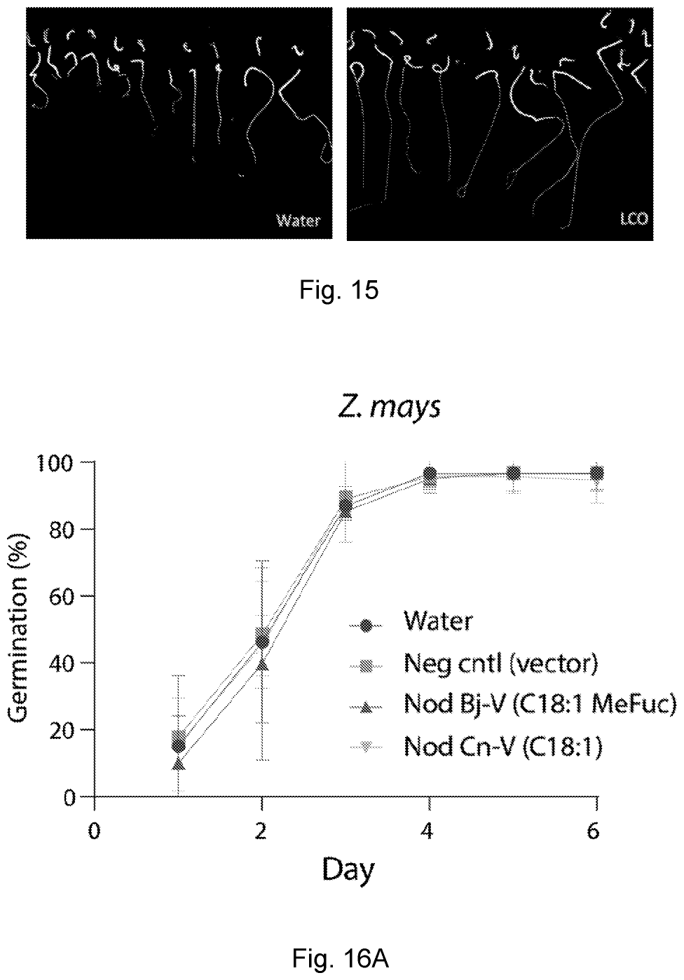

Following quantification and spectrometric analysis, purified Nod Cn-V (C₁₈: 1) was applied to the seeds of different plant species (see e.g., FIG. 6A); spinach, corn, and soybean were used for their fast growth rates. Because plant responses to LCOs are variable, a range of concentrations were tested for each species. After a germination time of 8 days, the engineered Nod Cn-V (C_{18:1}) increased the percent germination for spinach seeds by 125%, with an average of 60% seeds germinated compared to 40% for vector control and 27% for water as a control, respectively (see e.g., FIG. 4C, FIG. 15). Nod Cn-V (C_{18:1}) had no effect on corn or soybean germination (data not shown). In addition to percent germination, Nod Cn-V (C_{18:1}) increased growth parameters in spinach. Nod Cn-V (C_{18:1}) significantly increased the overall spinach sprout weight compared to the vector control (p<0.05) with a 41% increase compared with Nod Bj-V (C_{18:1}MeFuc) (see e.g., FIG. 4C).

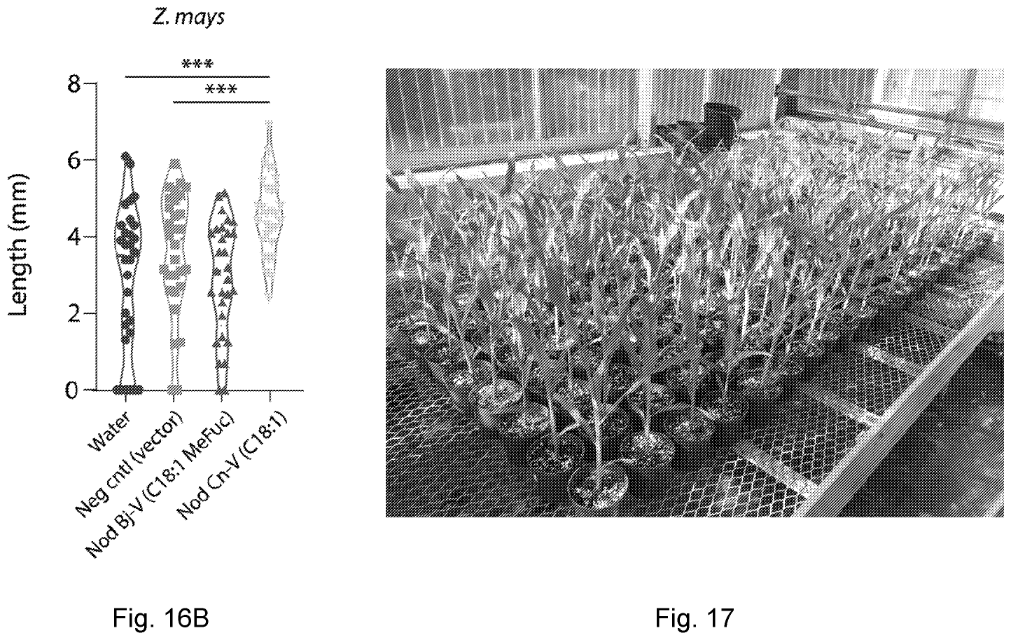

No significant difference was found for spinach length (data not shown). Germinated corn sprout weight with Nod Cn-V (C_{18:1}) was significantly increased 42% compared with water (p<0.0001), vector control (p<0.0001), and Nod Bj-V (C_{18:1}MeFuc) (p=0.0003) (see e.g., FIG. 4E). Because all of the corn germinated and sprouted during the experiment while the spinach did not, there are more samples for the corn. Nod Cn-V (C_{18:1}) significantly increased corn shoot weight 55% compared to water (p<0.0001). Corn shoot length increased significantly with Nod Cn-V (C_{18:1}) by 25% compared to water (p=0.002) (see e.g., FIG. 16). No differences were found for corn root weight or length (data not shown). No significant differences were found in soybean (data not shown).

In addition to germination experiments, the application of Cn-V (C_{18:1}) increased corn yield in greenhouse crop growth experiments (see e.g., FIG. 17). Corn wet weight increased significantly with Nod Cn-V (C_{18:1}) when compared with water by 18% (p=0.0023) (see e.g., FIG. 4F). Nod Cn-V (C_{18:1}) also significantly increased corn leaf number when compared to water (p<0.05), fertilizer (p=0.036), and Nod Bj-V (C_{18:1}MeFuc) (p=0.007) (see e.g., FIG. 4G). Thirdly, Nod Cn-V (C 18:1) significantly increased corn height compared to water (p=0.04) and was equivalent to the fertilizer condition (see e.g., FIG. 4H). Nod Cn-V (C_{18:1}) shows that it can lead to increased germination and growth in spinach, as well as increased growth of corn shoots and whole plant, generally outperforming the leguminous Nod Bj-V (C_{18:1} MeFuc) control. The engineered LCO described herein is a more generalizable plant-growth enhancer than the standard from Bradyrhizobium spp.

### DISCUSSION

The experiments described herein focus on these three products in an effort to support the use of engineered lithoautotrophs that can contribute to land use minimization and pollution caused by petrochemical and agricultural industries. As such, shown herein is the production of feedstocks for bioproduction, biodegradable bioplastics to offset petrochemical plastics production, and pollution and plant-growth enhancers to promote increased food crop yields and efficiency while offsetting the use of synthetic fertilizers. Decoupling bioproduction from plant-based feedstocks reduces the competition for crop land use, promotes commercialization by lowering costs, and supports the existing infrastructure of industrial heterotrophs.

C. necator is a versatile strain to use in lithotrophic growth. Bacteria were engineered to make sucrose and support growth of heterotrophs, to expand the scope of gas-based PHAs, and to improve the growth of plants through production of general purpose LCOs.

While a variety of co-culture systems have been developed with cyanobacteria with E. coli and S. cerevisiae and well as the acetogen M. thermoacetica with Y. lipolytica, the systems described herein combine the higher energy feedstock, sucrose, with non-photosynthetic gas fermentation in a single reactor. Engineered heterotrophs can produce complex products with growing together with C. necator without deleterious growth effects.

A spectrum of de novo PHA co-polymers were produced from CO₂ and H₂. Three primary levers were used to tailor the composition: overexpression of TEs to phaCs, the presence or absence of the endogenous phaC1_{Cn}, and the addition of acrylic acid—a β-oxidation inhibitor. Data with standard medium chain-length fatty acids demonstrated the feasibility of the approach. The composition of the co-polymers can thus be genetically controlled such that specific and industrially relevant PHAs can be produced lithotrophically.

LCOs—unlike synthetic fertilizers that are volatile and require high concentrations—act at nanomolar-micromolar concentrations and have no known potential for destructive runoff. While there have been some attempts to engineer B. japonicum, it remains challenging and still in the early stages. Herein is demonstrated that C. necator is a viable chassis for LCO production. Previous work has enhanced LCO production in native rhizobia strains, but there have been several unsuccessful attempts to produce LCOs through chemical synthesis and engineered E. coli (see e.g., Despras et al. Angew. Chem. 126, 12106-12110 (2014); Samain et al. J. Biotechnol. 72, 33-47 (1999)). LCOs can be produced using a combination of chemical and biological methods, but this is not a scalable or sustainable system. The approach described herein, inspired by mycelial (myc) LCOs with which over 60% of all plants are able to form arbuscular mycorrhiza associations and can promote growth, sought to produce a general purpose LCO to apply to a variety of non-legume plants. The design included only the genes that are necessary and sufficient for building a functional basic Nod LCO. A strength of this approach is that through genetic engineering the LCOs can also be tailored to a plant of interest for more effective growth enhancement. Use of C. necator allows for a more tractable genetic chassis that can grow to higher cell densities and faster rates than Bradyrhizobium spp.

Gas fermentation can be implemented in an industrial platform. Advances in fermenter construction have permitted the operation of large industrial bioproduction plants on site with syngas (CO, CO₂, H₂) point sources to minimize costs. In the systems described herein, CO₂ costs are negligible for gas fermentation through the use of concentrated, mostly pure CO₂ point sources such as breweries and ethanol-production plants. Pure H₂—produced by steam reforming or water electrolysis—is the primary feedstock cost driver. In addition to cost, it is important to consider the thermodynamic efficiencies of each process. While anaerobic methanogens and acetogens are extremely efficient at reducing CO/CO₂, that efficiency sharply declines once the product becomes more complex than ethanol or acetate. C. necator produces 8-fold more ATP per H₂ than methanogen or acetogens, and 4-fold more biomass per CO₂ via the Calvin-Benson-Bassham Cycle. Comparing H₂ to plant-derived carbohydrates as feedstocks, the energy-to-feedstock efficiency is approximately 0.1% for plants and 14% for solar H₂.

Given this, bioproduction can be expanded beyond sugar-based feedstocks. Despite their prevalence, plant-derived sugars have hidden environmental costs to the planet. Their low price is born, in part, from a highly developed industrial agriculture—which has significant GHG emissions, particularly due to fertilizer production. While microbes hold promise for sustainable intensification of agriculture, the reliance of biomanufacturing on plants limits the global green economy, as it also competes with food for arable cropland and fresh water supply—and as issues of food security increase, the tradeoff between food and bioproducts will become increasingly difficult to make. Using a CO₂-based gas fermentation, the scope of a lithotrophic microbial chassis can be expanded.

### Sustainability Considerations

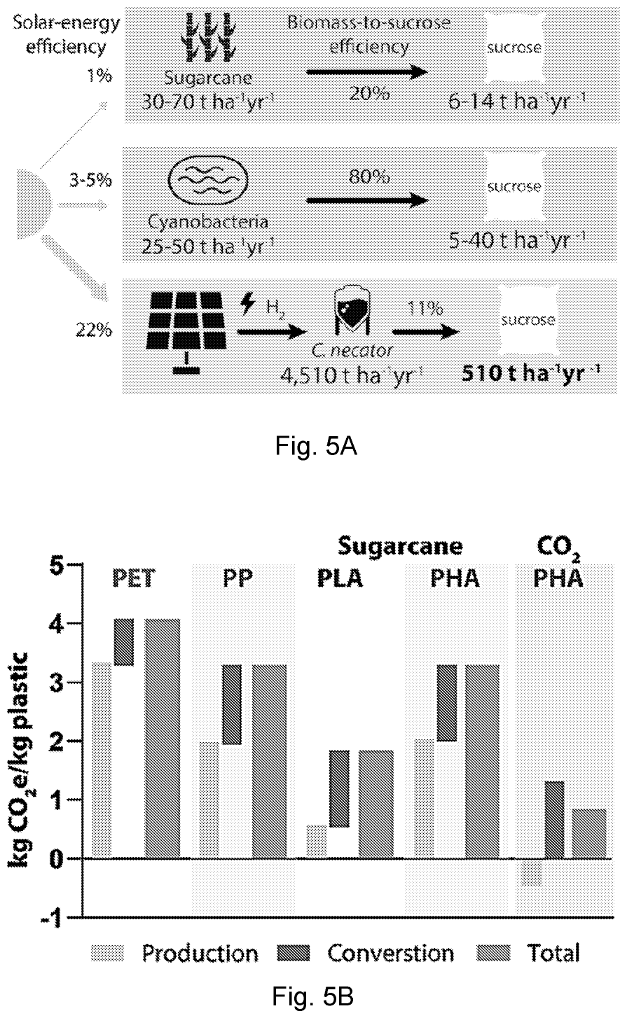

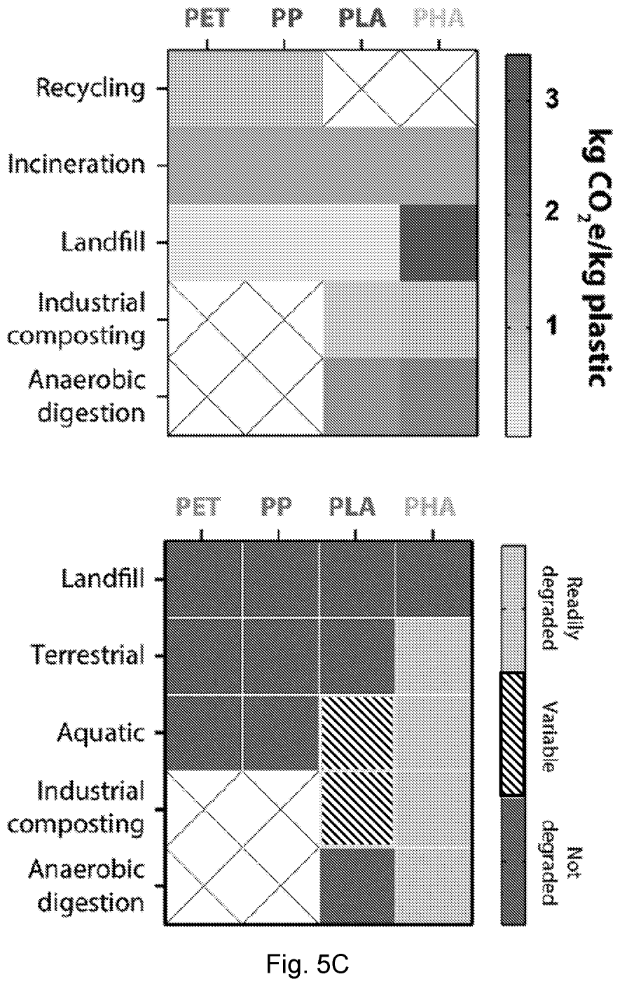

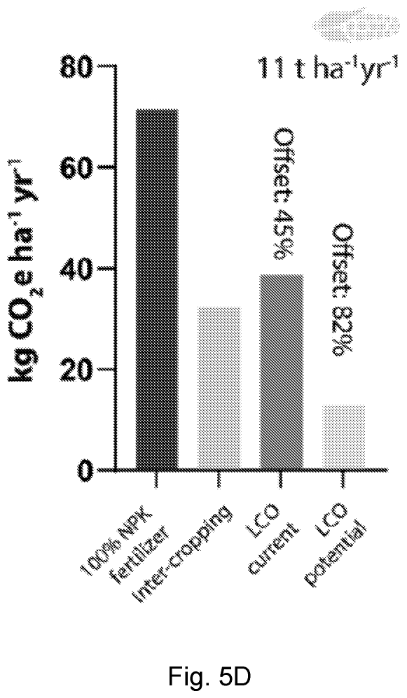

C. necator is one of the most effective microbes in converting H₂ into biomass. The solar-to-biomass efficiency for terrestrial plant photosynthesis is 1% with only 10-20% conversion efficiency of CO₂ into sucrose itself (e.g., sugar beets, sugarcane), compared to 3-5% and 80% for cyanobacteria; and 18% and 11% for C. necator (see e.g., FIG. 5A). Lithoautotrophic production is non-photosynthetic and is not limited by the thermodynamic and technological limitations of plant and cyanobacteria-based bioproduction systems. Here, based on general assumptions and using solar radiation as the common energy source, with an estimated 90 g/L biomass—typical yield for C. necator grown in a (CO₂/O₂/H₂) gas fermentation system—and with the reported production efficiency of 11.3%—510 tons of sucrose can be produced per hectare (ha) of photovoltaics per year. These estimates, based on equivalent land use, indicate that this approach is 35-fold more productive than plants and 13-fold more productive than cyanobacteria (see e.g., FIG. 5A).

When considering sustainable petrochemical plastics alternatives, several factors must be considered: material properties, cost of production, carbon footprint of production, and end-of-life. PHAs, unlike commodity bioplastics such as PLA, are polymerized biologically. Manipulation of PHA composition and, consequently, material properties can be made through genetic engineering. The importance of making industrially relevant polymers from gaseous feedstocks is to reduce costs and the carbon footprint of production. PLA, for example, is primarily derived from corn—which while it sequesters CO₂—the carbon footprint of the industrial agricultural supply chain and polymerization negates much of the benefits of CO₂ drawdown. Similarly, PHAs produced from carbohydrate feedstocks—from the perspective of carbon emissions—are less sustainable than their petrochemical analogs (see e.g., FIG. 5B).

End-of-life concerns are relevant to consolidating the GHG emissions of waste processing as well as the accumulation of pollution in the environment. Balancing the different environmental impacts of plastics will be crucial to choosing the most sustainable alternatives. Given the highly compacted and anaerobic environment of landfills, biodegradation occurs slowly. In the case of PHAs, anaerobic methanogens degrade the polymers into methane, which could ideally be captured and purified for use as a fuel source (see e.g., FIG. 5C, top panel). While dependent on a variety of factors including composition and physical dimensions, petrochemical plastics and PLA that find their way into aquatic and terrestrial environments persist on long, and in the case of nanoplastics, potentially indefinite timescales (see e.g., FIG. 5C, bottom panel). As of 2010, 4.8-12.7 Mt of plastic persist in the oceans, by 2025 estimates predict 90-250 Mt. Plastic pollution permeates the planet and is a major threat to global biodiversity. Constructing a mitigation strategy that addresses GHG emissions and pollution will be vital to a sustainable economy.

Gas fermentation is a mechanism to enhance agricultural productivity. In the context of climate change—and the ensuing reduction of arable land and crop yields—in combination with a push towards greener industries, the bioproduct-vs-fuel dilemma must be considered for commodity bioproduction (see e.g., Powell et al., Energy Environ. Sci. 5, 8116-8133 (2012)). Synthetic fertilizers currently support the production of food for half of the world's population food requirements but also use 2% of the global energy and produces 3% of the global GHG emissions. In addition to GHG emissions from its synthesis, nitrogen once in the environment leads to long-term ecological damage. In an effort to offset synthetic fertilizer production with bioproduction to minimize competition for land use, the efficiency with which agriculture can respond to increasing demand for plant-derived food, fuel, and textiles can be improved. A solution to more efficient and sustainable fertilizer use is the development of plant growth enhancers like LCOs (see e.g., FIG. 5D).

Gaseous feedstocks like CO₂ and H₂ provide inexpensive and abundant sources of energy for industrial bioproduction. The systems described herein promote the use of gas fermentation to both offset sources of GHG as well as do not compete with food for arable land.

### Methods

Strain construction: All plasmid construction used Gibson Assembly in E. coli DH5a. Expression vector pBadT (JBEI) was conjugated into C. necator (ATCC 17699) from the donor strain MFDpir (generously provided by George Church's lab). All genes, except the nodABC gene cluster, which was amplified from B. japonicum USDA6, were codon optimized and synthesized by SGI™ C. necator knock-outs were constructed using integration vector pT18mobsacB via the conjugation methods described above and sucrose counterselection. E. coli W ΔcscR strain (see e.g., Zheng et al. Nat. Clim. Change 9, 374-378 (2019)) and was transduced with the ΔaraC from the Keio collection, which conferred kanamycin resistance and removed the arabinose utilization operon through genetic linkage. S. cerevisiae W303^{clump}(see e.g., Steinbüchel, A. Polyhydroxyalkanoic acids. in Biomaterials (ed. Byrom, D.) 123-213 (Palgrave Macmillan UK, 1991)). Synthesized genes: Umbellularia californica FatB2 (12:0-ACP thioesterase); Pseudomonas aeruginosa phaC1; Pseudomonas aeruginosa phaC2; Anabaena cylindrica PCC 7122 SPS (sucrose phosphate synthase) and SPP (sucrose phosphate phosphatase); Synechocystis sp. PCC 6803 SPS (sucrose phosphate synthase) and SPP (sucrose phosphate phosphatase); Escherichia coli scrY (sucrose porin).

Cell culture: Bacterial strains and growth protocols. Growth protocols follow the procedures previously reported (see e.g., Liu et al. 2016, supra). E. coli DH5a and MFDpir were grown in LB and LB with 300 uM diaminopimelate (DAP), respectively at 37° C. C. necator was grown at 30° C., for pre-culture in rich media (17.5 g/L nutrient broth, 7.5 g/L yeast extract, 5 g/L (NH4)2S04). For lithotrophic growth, C. necator was grown in minimal medium was 3.5 g/L Na2HPO4, 1.5 g/L KH2PO4, 1.0 g/L (NH4)2S04, 80 mg/L MgSO₄.7H₂O, 1 mg/L CaSO4.2H₂O, 0.56 mg/L NiSO4.7H2O, 0.4 mg/L ferric citrate, and 200 mg/L NaHCO₃. For nitrogen-limited growth to produce PHA, the (NH4)2S04 concentration was reduced to 0.3 g/L. All solutions were filter-sterilized prior to use except the ferric citrate component, which was added after the filter sterilization step. Media was supplemented with 300 ug/mL kanamycin (PHA and LCO) or 25 ug/mL chloramphenicol (sucrose co-culture). The cultures were placed in a Vacu-Quick™ jar filled with H2 (8 inHg) and CO2 (2 inHg) with air as balance. Cultures were magnetically stirred and jars were refilled everyday with fresh gas mixture. To transfer the C. necator from heterotrophic to lithotrophic growth, an overnight culture grown in rich media was pelleted and washed twice with PBS, seeded in minimal media at OD 600=0.2 and allowed to grow for 5 days until OD=2. Cultures were then transferred into fresh minimal media and seeded at OD 600=0.2. Bradyrhizobium japonicum strain 6 was cultured in a medium with 2.6 g/L HEPES, 1 g/L yeast extract, 0.5 g/L gluconic acid, 0.5 g/L mannitol, 0.22 g/L KH₂PO₄, 0.25 g/L Na₂SO₄, 0.3 g/L NH₄Cl, 0.0112 g/L FeCl₃.6H₂O, 0.017 g/L CuCl₂.2H₂O, 0.18 g/L MgSO_{4.}7H₂O, NaMoO₄. 7H₂O, 0.0021 g/L NiCl₂.6H₂O, 0.01 g/L CaCl₂.2H₂O. Grown at a 30° C. under continuous shaking at 200 rpm.

Sucrose assay. 75 mL of sucrose-producing C. necator cultures were grown lithotrophically as described above in 1 g/L (NH₄)₂SO₄. Growth in co-cultures was monitored every 48 hours: OD600 was measured using the Ultrospec 10 Cell™ density meter (Amersham Biosciences™) and cell numbers were assayed by plating dilution series on rich media to count colony forming units (CFU). Cell numbers of W303 Clump were derived by counting CFUs, and numbers were adjusted for the ˜6.6 cells/clump. At each time point, cultures were pelleted and sucrose was measured from the supernatant and lysed pellet fractions using sucrose/D-glucose assay kits (Megazyme™).

Heterotrophic cross-feeding and co-culture with C. necator: Back-diluted C. necator from lithotrophic growth at OD 600=0.5 into lithotrophic growth and let grow for 2 days. Induced with 0.3% arabinose and added pre-condition E. coli at OD 600=0.01. Took samples and plated selectively for cfu/mL counts every other day.

Growth and induction for thioesterase. For thioesterase experiments, cells were grown in minimal media (described above) with 5% fructose. Once the cells reached mid-log, they were induced with 0.5% arabinose along with 800 μL 1-octanol as the organic phase. Aqueous and organic phases were harvested after 24, 72, and 120 h for GC-MS analysis.

Fatty acid identification and quantification using GC-MS. FFAs dissolved in the aqueous culture phase were harvested by acidifying 400 μL aqueous culture with 50 μl 10% (wt/vol) NaCl and 50 μL glacial acetic acid and extracting the FFAs into 200 μL ethyl acetate. 100 μL of the ethyl acetate phase was then esterified in 900 μL of a 30:1 mixture of ethyl alcohol (EtOH) and 37% (vol/vol) HCl by incubating at 55° C. for 1 h, and the ethyl esters were extracted into 500 μL hexanes for GCMS analysis. FFAs dissolved in the 1-octanol culture phase were esterified by acidifying 100 μL organic culture with 10 μL 37% (vol/vol) HCl then incubating at 55° C. for 1 h, and the octyl esters were extracted into 500 μL hexanes for GCMS analysis. Fatty esters were analyzed on an Agilent GC-MS 5975/7890 (Agilent Technologies™) using an DB-35MS column. Samples were heated on a gradient from 40° C. to 250° C. at 5° C./min. FFA chain lengths were identified by GC retention times and the mass spectra of the octyl esters at m/z=112 and quantified using an internal standard (800 mg/L pentadecanoate added to the culture before the extraction procedure). Known concentrations of C6, C8, C10, and C12 fatty acids were used to generate a standard curve and to quantify the production of single fatty acid species.

PHA extraction and analysis: After 48 hr of growth in the second media exchange, 100 ml lithotrophic PHA-producing C. necator cultures were induced with 0.3% arabinose where indicated along with 240 ug/mL acrylic acid where indicated and grown for 5 days at 30° C. Cultures were harvest, pelleted, and lyophilized overnight. Freeze dried pellets were weighed for dry cell weight. PHA was purified with 0.2 ml/mg DCW of 13% NaClO⁻ for 4 hr at 30° C., washed twice with dH2O, washed once with acetone, then dried at 25° C. overnight. 1:1 methanol and HCl in dioxane to a final volume of 3 ml (1% pentadecanoate as internal standard), the tubes were sealed with crimp top and incubated in oil bath at 90° C. for 20 hr. Tubes were placed on ice, once cooled, 2 ml of chloroform was added and vigorously vortexed, 3 ml of dH2O was added followed by extensive vortexing, the organic phase was separated by centrifugation (10 min, 4,000×g), organic phase removed and stored at −20° C. until GCMS analysis.

GCMS analysis: PHA samples were analyzed by GC-MS 5975/7890 (Agilent Technologies™) on a DB-35 ms column. Samples were heated on a gradient from 40 to 250° C. at 5° C./min. The co-polymer composition was determined with the mass spectra of the 3-hydroxy alkanoic acid methyl esters at m/z=103 and NIST Mass Spectral Library™.

LCO production and isolation: Overnight cultures were back-diluted into 50 mL cultures, which were also grown up to stationary phase and diluted into 200 mL cultures to an OD 600=0.2. Cultures were grown until an OD 600=1.0 (1-2 days) and induced by adding genistein (Sigma™) to B. japonicum to a final concentration of 5 mM and 0.3% arabinose to C. necator. The cultures were incubated for an additional 76 hours. These cultures were then extracted with 0.4 vol of HPLC-grade 1-butanol by shaking vigorously for 10 min. The material was then centrifuged for 10 min at 4000 rpm. The upper butanol phase was separated and dried in a rotary evaporator under vacuum at 55° C. (Yamato™, New York, USA). This product was redissolved in 2 mL of 20% acetonitrile and analyzed by HPLC. A Vydac™ C18 reverse-phase column (Vydac™; 5 mm, 250×4.6 mm) was used with a flow rate of 0.7 mL min−1. As a baseline, 30% acetonitrile was run through the system for at least 10 min prior to injection. The purification was done for 10 min in isocratic solvent A (water-acetonitrile, 70:30 [vol/vol]), followed by a linear gradient from solvent A to solvent B (water-acetonitrile, 50:50 [vol/vol]) for 25 min. The chromatographic peaks corresponding to the LCOs eluted between 24 and 28 minutes and were identified by comparison with an LCO standard, Nod Bj-V (C18:1), MeFuc, prepared from Bradyrhizobium japonicum strain USDA 523C. There were two LCO peaks that were detected using UV absorption at 206 and 214 nm.

LCO analysis: The LCO sample structures were also confirmed by positive-ion QTOF mass spectrometric analysis using a tandem mass spectrometer and Collision-Induced Dissociation in combination with HPLC on a 10 μL aliquot. The method and mobile phases were the same as those used with the HPLC alone except the mobile phases had an addition of 1% formic acid to both LCMS grade water and LCMS grade acetonitrile. LCOs from the LCO standard, induced B. japonicum, and C. necator strains were all analyzed. The energy of the Cs⁺ ions was 25 keV, and the accelerating voltage of the instrument was set to 8 kV. Results were recorded using a scanning method from m/z 1415 to 1417 over 1 second. The main compound for the standard and B. japonicum gives a peak at z=1416.7 (M₁H). The main compound for the C. necator strains give a peak at z=1256.7 (M₁H). Samples from induced and uninduced wild type C. necator as well as uninduced vector control and engineered strains were all tested for LCOs using this method and none were found.

Germination: Three species were tested; Spinacia oleracea L. (spinach) variety Regent, Glycine max L. (soybean), and Zea mays L. (corn) variety Trinity F1. Varieties were partially chosen for their relevance to agriculture in order to make this study as easily applied as possible. Seeds of all three species were surface-sterilized with 2% sodium hypochlorite (NaClO—) for 2 min, rinsed with sterile distilled water (dH2O) 10 times for spinach and 6 times for soybean and corn and then blotted dry. Each seed was soaked in the appropriate treatment solution for 30 minutes after being sterilized. A 9-cm diameter sterile petri dish was made with 1% noble agar and a 1 mm filter paper disk was placed on top of the agar. Five seeds were transferred onto the filter paper in six plates and five milliliters of distilled water (negative control), LCO standard (positive control), B. japonicum LCO extract (control), traditional N-based fertilizer (Miracle Grow™, 1 mg/mL), or solutions of purified extract from C. necator vector control strain or Nod factor producing strain at concentrations of 10⁻⁵, 10⁻⁶, 10⁻⁷, 10⁻⁸, 10⁻⁹ or 10⁻¹⁰ M were dispensed into each Petri dish. The Petri dishes containing spinach seeds were incubated at 21±2° C. and those containing corn and soybean were incubated at 25±2° C., all in the dark. The number of sprouted seeds was obtained at least every 2^{nd} day for 9 days for spinach and 6 days for both soybean and corn. At the end of these time periods, each seed and sprout were weighed. The root and shoot systems were disconnected from the rest of the sprout when applicable and weighed separately. The root length and shoot length were measured for each seed that sprouted when appropriate by identifying the longest root or shoot and measuring this against a metric ruler. The number of roots was also counted for each seed when appropriate. Since overall growth rates were relatively low for spinach, only successful, sprouted seeds were included. Corn was the only species where we were also able to measure enough shoots to determine shoot weight and length. Only successful sprouting and growth events with roots greater than 0.5 mm were considered for soybean. All tests were repeated at least twice and assays were performed blinded.

Plant Yield/Growth: A greenhouse experiment was carried out using the same Zea mays (corn) variety Trinity F1 as in the germination experiment. Seeds were surface-sterilized with 2% sodium hypochlorite for 2 min, rinsed with sterile distilled water 6 times, blotted dry, and each seed was soaked in treatment solution for 30 minutes. The growth tests were conducted in 6-inch diameter plastic pots. The pots were filled with growing media and seeds were planted just underneath the surface of the soil. Each pot received 25 ml of either distilled water, induced B. japonicum LCO, traditional N-based fertilizer (Miracle Gro™, 1 mg/mL), solutions of purified extract from C. necator Nod factor producing strain at a concentration of 10⁻⁷ M or a solution from filtered cultures of the LCO producing strain at a concentration of 10⁻⁷ M. The culture solution was first lysed through a freeze thaw cycle and sonication for 20 minutes at high intensity and then filtered through a 0.22 mm filter. The pots were placed in temperature controlled greenhouse at 25±2° C. during late June and July at atmospheric levels of CO₂. Each treatment had ten replicates. After 3 days the pots were irrigated from above every 3 days. After two weeks, the number of leaves and length of the longest shoot were measured with a metric ruler and above ground biomass was harvested. The wet weight was taken and the samples were lyophilized for 2 days until dry after which, the dry weight was taken. Assays were performed blinded.

Statistical Analyses: All statistical analyses were performed in GraphPad Prism. The multiple comparisons were done using Tukey's HSD method and differences between individual treatments were done using Mann-Whitney Test.

TABLE 2
Engineered bacteria used herein.

| Name | Species | Strain | Origin |
|---|---|---|---|
| PAS824 | E. coli | MG1655 | Church lab |
| | | RP4-2-Tc:: [ΔMu1::aac(3)IV- | |
| | | ΔaphA-Δnic35-ΔMu2::zeo] | |
| | | ΔdapA::(erm-pir) ΔrecA | |
| PAS825 | E. coli | pBADT FatB1_{Cp}, | Tanaka et al., |
| | | Fat2_{Cp} (chim4) | 1995 |
| PAS826 | C. necator | pBADT rfp | Herein |
| PAS827 | C. necator | ΔphaC_{Cn}, pBADT rfp | Herein |
| PAS828 | C. necator | pBADT FatB2_{Uc}, phaC1_{Pa} | Herein |
| PAS829 | C. necator | ΔphaC_{Cn}, pBADT FatB2_{Uc}, phaC1_{Pa} | Herein |
| PAS830 | C. necator | pBADT Chim4, phaC1_{Ps} | Herein |
| PAS831 | C. necator | ΔphaC_{Cn}, pBADT Chim4, phaC1_{Ps} | Herein |
| PAS832 | C. necator | pBADT FatB2_{Uc}, phaC2_{Pa} | Herein |
| PAS833 | C. necator | ΔphaC_{Cn}, pBADT FatB2_{Uc}, phaC2_{Pa} | Herein |
| PAS834 | C. necator | pBADT SPS_{Ac}, SPP_{Ac} | Herein |
| PAS835 | C. necator | pBADT SPS_{Ac}, SPP_{Ac}, scrY_{Ec} | Herein |
| PAS836 | C. necator | pBADT SPS_{Se}, SPP_{Se} | Herein |
| PAS837 | C. necator | pBADT SPS_{Se}, SPP_{Se}, scrY_{Ec} | Herein |
| PAS838 | C. necator | pBADT nodABC_{Bj} | Herein |
| PAS839 | B. | USDA 6 | Powell et al., |
| | japonicum | | 2012 |
| PAS840 | B. | USDA 523C | Smith lab |
| | japonicum | | |
| PAS841 | E. coli | Wild-type; csc+ | Antonio et al., |
| | | (ATCC 9637) | 2000 |
| PAS842 | E. coli | WcscR::FRT | Antonio et al., |
| | | | 2000 |
| PAS843 | S. | S. cerevisiae W303 | Tanaka et al., |
| | cerevisiae | Ancestor strain | 1995 |
| PAS844 | S. | S. cerevisiae W303 | Tanaka et al., |
| | cerevisiae | Clump Recreated02 | 1995 |
| PAS845 | E. coli | pET21b-VioABCDE | Sabri et al., |
| | | | 2013 |
| PAS846 | E. coli | pSB1C3-CrtEBIY | Arifin et al., |
| | | | 2011 |

### Comparison to Cyanobacterial Co-Culture Systems

As bioproduction technologies have expanded, co-culture and cross-feeding are a solution to lower feedstock costs while supporting the existing infrastructure of engineered heterotrophs. Efforts towards autotrophic-heterotrophic co-cultures have primarily focused on cyanobacteria as the autotroph. Cyanobacteria natively produce sucrose as an osmoprotectant—rather than a carbon source—to high concentrations without toxicity, making it an attractive feedstock-producer for heterotrophs. Engineered cyanobacterial strains able to convert and export up to 80% of their fixed carbon can successfully feed three phylogenetically distinct heterotrophic microbes (E. coli, B. subtilis, and S. cerevisiae). However, cyanobacteria produce reactive oxygen species through photosynthesis and protective cyanotoxins, which are ultimately toxic to the heterotrophs. While cyanobacteria have higher solar-to-biomass conversion efficiencies than plants, efficiency remains 5-7% and is thermodynamically limited to ˜12%—several fold lower than photovoltaics (see e.g., FIG. 5A). In addition to their biological limitations, there are a variety of implementation constraints that hinder industrial scale-up. Because cyanobacteria grown at scale require sunlight, two common culturing methods allow for optimal sunlight penetration: pools and photobioreactors. The large shallow pools can only be used in certain regions, are susceptible to environmental changes and contamination—and so it is difficult to maintain consistent batch-to-batch cultivation. In an effort to mitigate some of these issues, these pools can be modified to grow the cyanobacteria in small diameter tubing, but this kind of containment often deteriorates from radiation exposure as well as generates substantial plastic waste. Because these issues are all challenges for cyanobacteria monoculture, it is not clear how a Cyanobacteria-heterotroph co-culture system would be successfully implemented at scale. As such, the C. necator-heterotroph co-culture systems described herein are more efficient and practical than using Cyanobacteria as the autotroph.

### Sucrose Production Calculations

In comparing the different modes of sucrose production, the respective productivities were calculated per hectare of land per year. Because the footprints of fermenters are orders of magnitude smaller than the land area needed to grow cyanobacteria and crops, an equivalence was drawn to the land area needed to satisfy the H₂ demand were it to be derived from photovoltaics and water splitting. Sugarcane and engineered cyanobacteria (see e.g., Ducat et al., Appl Env. Microbiol 78, 2660-2668 (2012)) were compared with the engineered C. necator described herein. Solar-to-biomass conversion in plant crops have an annual efficiency of 1% and 3.5% (C3) or 4.3% (C4) while cyanobacteria have an efficiency of 3% in open pools and 5-7% in bubbled bioreactors. For C. necator, the solar-H₂-efficiency of photovoltaic cells coupled to water electrolysis is 14% (see e.g., Blankenship et al., Science 332, 805-809 (2011)). Land area was estimated based on an NREL case study reporting 2 kWh H₂ kg⁻¹, and with the PVWatt calculator set to the Los Angeles area, there is 2,510,000 kWh ha⁻¹ yr⁻¹. Based on a California Energy Commission report, there was assumed to 3.3-3.6 g of biomass per gram of H₂. Of the accumulated biomass, sugarcane is 20% sucrose, S. elongatus can be optimized to convert 80% of its biomass into sucrose, and this work (unoptimized) reports a biomass-to-sucrose conversion of 11.3%. Applying these values at hectare scale, it was assumed that sugarcane produces 30-70 t ha⁻¹ yr⁻¹ biomass; at a conversion of 20% that yields 14 t ha⁻¹ yr⁻¹ sucrose. Likewise, cyanobacteria in open-pond designs produce 25-50 t ha⁻¹ yr⁻¹ sucrose; at a conversion of 80% that yields 40 t ha⁻¹ yr⁻¹ sucrose. The engineered C. necator described herein, in such a system, can reach a productivity of 510 t ha⁻¹ yr⁻¹ sucrose which is a 35-fold increase over sugarcane and 13-fold increase over cyanobacterial ponds.

### Carbon Footprint of Plastics Calculations

Coupled with its production, petrochemical plastics—depending on the type—persist in the environment for long periods of time that are largely undetermined (ranges from decades to thousands of years). Unfortunately, the most commonly used bioplastics (e.g., PLA, bio-PET) do not show much improvement in this context. Microbial production of biodegradable bioplastics through gas fermentation is an alternative to reduce both GHG emissions and pollution.

The CO₂e values were used for the production, conversion, and end-of-life of PET, PP, PLA, and PHA made from sugarcane (see e.g., Zheng et al. Nat. Clim. Change 9, 374-378 (2019)). The potential CO₂e emissions were calculated from the energy input for a scaled system. The same emissions were assumed for processing for CO₂-derived PHA as sugar-derived PHA. Biodegradability of these plastics in the environment is still not well characterized and depend on a variety of physical and chemical characteristics of the product itself (e.g., dimensions, compounding, blending) Because of this a qualitative approach to how these materials biodegrade naturally was used. Depending on the context, PET and PP can degrade into nanoplastics over decades or persist in the environment indefinitely. The functional outcomes of PLA in industrial composters remains unclear as the standard cycle times tend not to be long enough to degrade most PLA products, but they will degrade if the conditions are optimized. PLA biodegradation in the environment is also unclear but appears to be limited to specific forms (e.g., agricultural mulch films). In contrast, PHA biodegradation in the environment is well established and can degrade within weeks to years depending on the product specification and environment.

### Carbon Footprint of Fertilizer Calculations

Aligned with the focus on industrial bioproduction to not compete for arable land, a plant growth enhancer was produced that could be used to offset fertilizer use. Despite their central role in supporting modern agriculture, synthetic fertilizers generate significant GHG emissions, ecological hazards, and consequent adverse health and economic effects. As such, more sustainable solutions are needed to plant growth and fertilization. The average amount of NH₃ fertilizer applied in the United States is 92 kg ha⁻¹ yr⁻¹. The average CO₂e from industrial Haber-Bosch process is 2.6 kg CO₂ kg⁻¹ of NH₃. This amount of NH₃ generates 239.2 kg CO₂ ha⁻¹ yr⁻¹. Synthetic fertilizers were compared to cover cropping, intercropping, and LCO addition. Cover cropping is a strategy used to improve the soil for the main crop by growing different plants when not growing the main crop. Intercropping is also used to improve the soil but does so by growing the main crop with others simultaneously; typically the additional crop is a legume. In the calculation, a variety of legumes and crops, locations, soil types, and application times were included. A wide variety of responses were observed with cover/intercropping and there are many management considerations that must also be included when using this method of growth enhancement.

It was assumed that the 40% increase conferred by synthetic fertilizer in productivity is 100% of possible yield. The growth that results from intercropping or LCO addition was subtracted from the equivalent amount of fertilizer. That reduction is represented in the offset CO₂e not generated from the alternative strategies. These values were applied to corn, whose production in the U.S. is approximately 11.1 tons ha⁻¹. The percent increase conferred by LCOs on different plants was then determined. Two different scenarios were considered: 1) the growth enhancements in field studies and 2) optimized conditions in greenhouses. Because multiple studies addressing the same crop were limited, different crop species were used in calculations (e.g., corn, soybean, artichoke, rice, pea, and vetch). It was assumed that there was no additional CO₂ drawdown from CO₂ fixation by C. necator to produce the LCOs.

### Example 2

A Platform for Biomanufacturing from Waste Streams

### Abstract

Described herein is a platform that uses gaseous waste streams to produce a variety of sustainable chemicals. Advances in the genetic engineering of microbial metabolisms has facilitated the further development of a bio-based economy that focuses on sustainability and independence from fossil fuels. This platform uses a chassis organism, C. necator, that fixes CO₂ as the sole carbon source and H₂ as the sole energy source. This organism was developed to produce tunable compostable plastics, feedstock for co-culturing heterotrophs and potent plant fertilizers. Herein is demonstrated product modularity of the system, creating an improved biomanufacturing system that is adaptable to multiple waste streams and product demands. Biomanufacturing can thus occupy a larger proportion of the market as well as compete with high volume low-medium margin products with the utilization of waste streams, distributed implementation, and an increased customer willingness to pay premiums for sustainable materials.

### Introduction

This system was demonstrated in the context of the bionic leaf—the cultures growing together with water-splitting electrodes (see e.g., US 2018/0265898 A1). This system has a CO₂-reduction energy efficiency of ˜50% when producing biomass and liquid fusel alcohols, scrubbing 180 grams of CO₂, equivalent of 230,000 liters of air per 1 kWh of electricity using C. necator. The engineered strains can produce isopropanol, a fusel alcohol and disinfectant, with a 32±2% electricity-to-product efficiency. Higher yields (42±2%) have been achieved for the bioplastic precursor PHB, demonstrating the versatility of the system. Solar-to-biomass yields are estimated to average 9% of the thermodynamic maximum, exceeding solar-to-biomass conversion efficiencies typically demonstrated by natural and industrially used organisms (1-5%). A 1 L reactor of C. necator can mitigate approximately 500 L of atmospheric CO₂ per day; thus a significant volumetric CO₂ turnover can occur when scaled up. This system while energy efficient is still limited by the capital costs associated with its scale-up. Described herein is an expanded product portfolio, which is a more economically viable solution.

The systems described herein can be used either directly for production or as producer of feedstock in co-culture with other heterotrophs. As a direct producer, the system is engineered to produce tunable bio-degradable plastics and carbon-based potent fertilizers for plants. As an indirect approach, C. necator was engineered to produce sucrose to cross-feed to heterotrophs that have been engineered for chemicals production (e.g., yeast, E. coli, B. subtilis). Metabolic engineering facilitated these tasks. The product portfolio of the chassis organism has been expanded, thus making it a platform technology for production of chemicals directly from hydrogen and carbon dioxide.

### Bioplastics Production

A robust genetic engineering protocol was successfully established and these bacteria were modified to create new and more industrially-relevant bioplastics than the native polymer, polyhydroxybutyrate (PHB). PHB is a homopolymer of 3-hydroxybutyrate that accumulates at times of nutrient deficiency (e.g., N, P, Fe, S, O2) and carbon excess as a means of energy and carbon storage. While PHB is brittle, stiff, and has low resistance to thermal degradation, modified co-polymers—polyhydroxyalkanoates (PHA)—have a variety of desirable properties. PHAs that contain medium chain-length fatty acids (mcl-PHA, C6-C12) have properties that are similar to petrochemical plastics polyethylene and polypropylene and are promising targets for large-scale production.

Various polymerization pathways of PHA are engineered that impact the physical properties. By engineering C. necator to enrich for C−12 fatty acid chain lengths using thioesterase UcFatB2, lauric acid production can be increased by 60-fold. C. necator can be engineered to synthesize different chain-length fatty acids for subsequent integration into the PHA polymer. Building from known modifications of fatty acid metabolism, modifications to well-established proteins from the PHA biosynthetic pathway are introduced to fine-tune the material characteristics of the polymer.

Thioesterases were inducibly expressed, which determine the chain length of substrate fatty acids, in order to modulate polymer length and ratio or components. In addition to the thioesterases, PHA synthases (PhaC) provide another major point of control in the polymer properties. As the last step in PHA synthesis, PhaCs assemble the polymer based on their intrinsic substrate specificities and enzymatic rates. Manipulation of PhaCs to change the polymer composition produces a positive correlation in malleability as the chain-length of the fatty acid increases. The properties of these bacterially-derived biopolymers are optimized, paying particular focus to thermoplasticity, durability, and strength. PHA production is an economically tenable product that can be engineered into strains of C. necator. By introducing two heterologous enzymes (i.e., thioesterase and PHA synthase) that have variable specificities for different chain-length fatty acids bacteria overproduce a variety of fatty acid chain lengths. Coupling with PHA synthases, these fatty acid groups are incorporated into the mcl-PHA polymer at different rates thereby creating bioplastics with tunable properties. Additionally, by either removing or complementing the native phaC further alterations to composition and production can be made. Through GCMS analysis, shown herein is a strain that produces C8-C12 chain-length fatty acid PHAs. Strains can be created that have different combinations of thioesterase and phaC enzymes to expand the bioplastics scope. Thioesterases can be added that either create a broad range of free fatty acids (e.g., FatB1Cp, C4-C14 FFA) and those that are highly specific for one chain length (e.g., engineered FatB2Cp-FatB1Cp chimera, C8 free fatty acids).

### Sucrose and Co-Culture

C. necator was engineered convert CO₂ into molecular substrates for media to feed other microorganisms. To accomplish this and address the project goals, a fermenter can be built that maintains the growth of a C. necator and a heterotroph (e.g., E. coli, S. cerevisiae, B. subtilis) co-culture using CO₂, H₂, and a minimal media as the only inputs. Sucrose was selected for its high energy-density and usability by a wide variety of heterotrophs except, importantly, by C. necator. Previous work has shown that engineered cyanobacteria can overproduce and export sucrose at 36.1 mg L−1 h illumination-1. Cyanobacterial sucrose phosphate synthase and sucrose phosphate phosphatase enzymes can be overexpressed in combination with E. coli sucrose transporters that export sucrose into the media in C. necator. The sucrose-utilizing Y. lipolytica can be fed with sterilized spent media from the sucrose-producing C. necator grown under CO₂. Additionally, a C. necator extract can be created from the sucrose-producing strains, akin to yeast extract, to provide richer media conditions.

The platform system described herein can be used to enhance plant growth through fertilizer production, for example carbon-based molecules, lipid-chitin oligosaccharides (LCOs), that are known to enhance plant growth. Specifically, LCOs comprise an acylated chitin oligomeric backbone with a variety of functional groups that can be attached to the terminal or non-terminal residues. Natively, LCOs are signal molecules that are produced in symbiotic bacteria on the plant in response to plant-secreted flavonoids to begin a cascade of signals that lead to the formation of root nodules—primarily on legumes. Specifically, they induce deformation and curling of root hairs and cell division to form the nodule. Nodules provide a location for diazotrophs, nitrogen-fixing bacteria, to infect legume roots and produce reactive nitrogen. However, LCOs can act separately as a fertilizer when applied externally to plant roots, specifically crops such as corn, tomato, soybean, and artichoke. Studies have found external application to lead to the accumulation of LCOs on the outside of roots. They mimic these hormones' ability to enhance cell division and indirectly affect the internal auxin/cytokinin hormone balance by preventing the transport of internal auxin away from the roots. This leads to a buildup of auxin and further enhancement of cell division. This increase in the auxin to cytokinin ratio leads to root growth and the observed fertilization effect. A benefit of using this system is that the genes controlling the production of LCOs are known. Thus the necessary components can be identified for the production and export of LCOs from a host cell. Another positive aspect in terms of production in this system is that in deep space missions that they function at micromolar concentrations. Plants are extremely sensitive to LCOs, so only a small quantity is required to significantly influence plant growth, which is beneficial for resource limited conditions. As shown herein, C. necator was engineered to produce LCOs in gas conditions. The efficiency of this fertilizer on food crops such as soybean and spinach can be tested by application of the cells from the bionic leaf directly onto the soil. Since LCOs are also the initial signaling molecules for the creation of nodules, the production of these molecules can contribute to the development of engineered relationships between bacteria and non-native hosts. For example, systems as described herein can recreate the nodule relationship between diazotrophs and legumes in non-legume crops, which provides a localized source of fertilizers such as nitrogen and limit the need for inefficient external application of fertilizers. Creating a simple associative relationship between a microbe producing a fertilizer, whether its nitrogen or a carbon based fertilizer such as LCOs, is an incredibly important tool for optimizing fertilizer use, which is also relevant for resource limited environments.

### CONCLUSION

Described herein is a variety of viable commercial products. A circular bio-based economy can thus be expanded with the use of these carbon-neutral to carbon-negative products.

What is claimed herein is:

1. An engineered Cupriavidus necator bacterium, comprising: at least one exogenous copy of at least one functional polyhydroxyalkanoate (PHA) synthase gene; and at least one exogenous copy of at least one functional thioesterase gene.

2. The engineered bacterium of claim 1, further comprising: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product.

3. The engineered bacterium of claim 1, further comprising: (i) at least one endogenous beta-oxidation gene comprising at least one engineered inactivating modification; or (ii) at least one exogenous inhibitor of an endogenous beta-oxidation gene or gene product.

4. The engineered bacterium of any one of claims 1-3, wherein said engineered bacteria is a chemoautotroph.

5. The engineered bacterium of any one of claims 1-4, wherein said engineered bacteria uses CO₂ as its sole carbon source, and/or said engineered bacteria uses H₂ as its sole energy source.

6. The engineered bacterium of claim 2, wherein the endogenous PHA synthase comprises phaC.

7. The engineered bacterium of any one of claims 1-6, wherein the functional PHA synthase gene is heterologous.

8. The engineered bacterium of claim 7, wherein the functional heterologous PHA synthase gene comprises a Pseudomonas aeruginosa phaC1, a Pseudomonas aeruginosa phaC2 gene, and/or Pseudomonas spp. 61-3 phaC1.

9. The engineered bacterium of any one of claims 1-8, wherein the functional thioesterase gene is heterologous.

10. The engineered bacterium of claim 9, wherein the functional heterologous thioesterase gene comprises a Umbellularia californica FatB2 gene, a Cuphea palustris FatB1 gene, a Cuphea palustris FatB2 gene, or a Cuphea palustris FatB2-FatB1 hybrid gene.

11. The engineered bacterium of claim 3, wherein the endogenous beta-oxidation gene is 3-hydroxyacyl-CoA dehydrogenase (fadB) or acyl-CoA ligase.

12. The engineered bacterium of any one of claims 1-11, wherein an engineered inactivating modification of a gene comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

13. The engineered bacterium of claim 3, wherein the inhibitor of an endogenous beta-oxidation enzyme is acrylic acid.

14. The engineered bacterium of any one of claims 1-13, wherein said engineered bacteria produces medium chain length PHA.

15. A method of producing medium-chain-length polyhydroxyalkanoate (MCL-PHA), comprising:
a) culturing the engineered bacterium of any of claims 1-14 in a culture medium comprising CO₂ and/or H₂; and
b) isolating, collecting, or concentrating MCL-PHA from said engineered bacterium or from the culture medium of said engineered bacterium.

16. The method of claim 15, wherein the isolated MCL-PHA comprises an R group fatty acid which is 6 to 14 carbons long (C6-C14).

17. The method of any one of claims 15-16, wherein the total PHA isolated comprises at least 50% MCL-PHA.

18. The method of any one of claims 15-17, wherein the total PHA isolated comprises at least 80% MCL-PHA.

19. The method of any one of claims 15-18, wherein the total PHA isolated comprises at least 95% MCL-PHA.

20. The method of any one of claims 15-19, wherein the total PHA isolated comprises at least 98% MCL-PHA.

21. The method of any one of claims 15-20, wherein the total PHA isolated comprises at least 95% MCL-PHA with an R group fatty acid of C10-C14.

22. The method of any one of claims 15-21, wherein the total PHA isolated comprises at least 80% MCL-PHA with an R group fatty acid of C12-C14.

23. The method of any one of claims 15-22, wherein the culture medium comprises CO₂ as the sole carbon source, and/or the culture medium comprises H₂ as the sole energy source.

24. An engineered C. necator bacterium, comprising one or more of the following:
a) at least one exogenous copy of at least one functional sugar synthesis gene; and/or
b) at least one exogenous copy of at least one functional sugar porin gene.

25. The engineered bacterium of claim 24, wherein said engineered bacteria is a chemoautotroph.

26. The engineered bacterium of any one of claims 24-25, wherein said engineered bacteria uses CO₂ as its sole carbon source, and/or said engineered bacteria uses H₂ as its sole energy source.

27. The engineered bacterium of any one of claims 24-26, wherein the at least one functional sugar synthesis gene is heterologous.

28. The engineered bacterium of any one of claims 24-27, wherein the at least one functional sugar synthesis gene comprises at least one functional sucrose synthesis gene.

29. The engineered bacterium of any one of claims 24-28, wherein the at least one functional heterologous sucrose synthesis gene comprises Synechocystis sp. PCC 6803 sucrose phosphate synthase (SPS) and/or Synechocystis sp. PCC 6803 sucrose phosphate phosphatase (SPP).

30. The engineered bacterium of any one of claims 24-29, wherein the functional sugar porin gene is heterologous.

31. The engineered bacterium of any one of claims 24-30, wherein the functional sugar porin gene is a functional sucrose porin gene.

32. The engineered bacterium of any one of claims 24-31, wherein the functional heterologous sucrose porin gene comprises E. coli sucrose porin (scrY).

33. The engineered bacterium of any one of claims 24-32, wherein said engineered bacteria produces a feedstock solution.

34. The engineered bacterium of any one of claims 24-33, wherein said bacterium is co-cultured with a second microbe that consumes the feedstock solution.

35. An engineered heterotroph, comprising one or more of the following:
a) at least one overexpressed functional sucrose catabolism gene;
b) (i) at least one endogenous sucrose catabolism repressor gene comprising at least one engineered inactivating modification; or (ii) at least one exogenous inhibitor of an endogenous sucrose catabolism repressor gene or gene product;
c) (i) at least one endogenous arabinose utilization gene comprising at least one engineered inactivating modification; or (ii) at least one exogenous inhibitor of an endogenous arabinose utilization gene or gene product; and/or
d) at least one exogenous copy of at least one functional secondary product synthesis gene.

36. The engineered heterotroph of claim 35, wherein the engineered heterotroph is E. coli.

37. The engineered heterotroph of any one of claims 35-36, wherein the at least overexpressed functional sucrose catabolism gene is endogenous.

38. The engineered heterotroph of any one of claims 35-37, wherein the at least overexpressed functional sucrose catabolism gene comprises an invertase (CscA), a sucrose permease (CscB), and/or a fructokinase (CscK).

39. The engineered heterotroph of any one of claims 35-38, wherein the endogenous sucrose catabolism repressor gene comprises the repressor (CscR).

40. The engineered heterotroph of any one of claims 35-39, wherein the endogenous arabinose utilization gene comprises araB, araA, araD, and/or araC.

41. The engineered heterotroph of any one of claims 35-40, wherein the at least one functional secondary product synthesis gene is heterologous.

42. The engineered heterotroph of any one of claims 35-41, wherein the at least one functional secondary product synthesis gene comprises a violacein synthesis gene.

43. The engineered heterotroph of any one of claims 35-42, wherein the at least one functional violacein synthesis gene comprises VioA, VioB, VioC, VioD, and/or VioE.

44. The engineered heterotroph of any one of claims 35-43, wherein the at least one functional secondary product synthesis gene comprises a β-carotene synthesis gene.

45. The engineered heterotroph of any one of claims 35-44, wherein the at least one functional β-carotene synthesis gene comprises CrtE, CrtB, CrtI, and/or CrtY.

46. The engineered heterotroph of any one of claims 35-45, wherein the engineered heterotroph has enhanced sucrose utilization as compared to the same heterotroph lacking the engineered sucrose catabolism gene(s), sucrose catabolism repressor(s), arabinose utilization gene(s), and/or secondary product synthesis gene(s).

47. A method of producing a feedstock solution, comprising:
a) culturing the engineered bacterium of any of claims 24-34 in a culture medium comprising CO₂ and/or H₂; and
b) isolating, collecting, or concentrating a feedstock solution from said engineered bacterium or from the culture medium of said engineered bacterium.

48. The method of any of claim 47, wherein the culture medium comprises CO₂ as the sole carbon source, and/or the culture medium comprises H₂ as the sole energy source.

49. The method of any one of claims 47-48, wherein the culture medium further comprises arabinose.

50. The method of any one of claims 47-49, wherein the feedstock solution comprises a sucrose concentration of at least 100 mg/mL.

51. The method of any one of claims 47-50, wherein the feedstock solution comprises a sucrose concentration of at least 150 mg/mL.

52. The method of any one of claims 47-51, wherein the feedstock solution comprises a sucrose feedstock for at least one heterotroph.

53. The method of any one of claims 47-52, wherein the at least one heterotroph comprises an organism with enhanced sucrose utilization.

54. The method of any one of claims 47-53, wherein the at least one heterotroph comprises E. coli and/or S. cerevisiae.

55. The method of any one of claims 47-54, wherein the at least one heterotroph comprises an engineered bacterium of any one of claims 35-46.

56. An engineered C. necator bacterium comprising at least one exogenous copy of at least one functional lipochitooligosaccharide synthesis gene.

57. The engineered bacterium of claim 56, wherein said engineered bacteria is a chemoautotroph.

58. The engineered bacterium of any one of claims 56-57, wherein said engineered bacteria uses CO₂ as its sole carbon source, and/or said engineered bacteria uses H₂ as its sole energy source.

59. The engineered bacterium of any one of claims 56-58, wherein the at least one functional lipochitooligosaccharide synthesis gene comprises an N-acetylglucosaminyltransferase gene, a deacetylase gene, and/or an acetyltransferase gene.

60. The engineered bacterium of any one of claims 56-59, wherein the at least one functional lipochitooligosaccharide synthesis gene is heterologous.

61. The engineered bacterium of any one of claims 56-60, wherein the at least one functional heterologous lipochitooligosaccharide synthesis gene comprises B. japonicum NodC, B. japonicum NodB, and/or B. japonicum NodA.

62. The engineered bacterium of any one of claims 56-61, wherein said engineered bacteria produces lipochitooligosaccharide.

63. A method of producing a fertilizer solution, comprising:
a) culturing the engineered bacterium of any of claims 56-62 in a culture medium comprising CO₂ and/or H₂; and
b) isolating, collecting, or concentrating a fertilizer solution from said engineered bacterium or from the culture medium of said engineered bacterium.

64. The method of any of claim 63, wherein the culture medium comprises CO₂ as the sole carbon source, and/or the culture medium comprises H₂ as the sole energy source.

65. The method of any one of claims 63-64, wherein the fertilizer comprises lipochitooligosaccharides.

66. The method of any one of claims 63-65, the fertilizer solution comprises a lipochitooligosaccharide concentration of at least 1 mg/L.

67. A system comprising:
a) a reactor chamber with a solution contained therein, wherein the solution comprises hydrogen (H₂) and carbon dioxide (CO₂); and
b) at least one of the following engineered bacteria in the solution:
i) the engineered bioplastics bacterium of any of claims 1-14;
ii) the engineered sugar feedstock bacterium of any of claims 24-34;
iii) the engineered heterotroph of any of claims 35-46; or
iv) the engineered fertilizer solution bacterium of any of claims 56-62.

68. The system of claim 67, further comprising a pair of electrodes in contact with the solution that split water to form the hydrogen.

69. The system of any one of claims 67-68, further comprising an isolated gas volume above a surface of the solution within a head space of a reactor chamber.

70. The system of any one of claims 67-69, wherein the isolated gas volume comprises primarily carbon dioxide.

71. The system of any one of claims 67-70, further comprising a power source comprising a renewable source of energy.

72. The system of any one of claims 67-71, wherein the renewable source of energy comprises a solar cell, wind turbine, generator, battery, or grid power.


SEQUENCE LISTING (100 sequences)

SEQ ID NO: 1 - DNA, 1770 bp, Cupriavidus necator
atggcgaccggcaaaggcgcggcagcttccactcaggaaggcaagtcccaaccattcaagttcacgccggggccattcgatccagccacatggctggaatggtcccgccagtggcagggcactgaaggcaacggccacgcggccgcgtccggcattccgggcctggatgcgctggcaggcgtcaagatcgagccggcgcagctgggtgatatccagcagcgttacatgaaggacttctcagccctgtggcaggccatggccgagggcaaggccgaggccaccgggccgctgcacgaccggcgcttcgccggcgacgcgtggcgcaccaacctgccataccgcttcgctgccgcgttctacctgctcaatgcgcgcgccttgaccgagctggccgatgctgttgaggccgatgccaagacgcgccagcgcatccgctttgcgatctcgcaatgggtcgatgcgatgtcgcccgccaacttcctcgccacgaatcccgaggcgcagcgcctgctgatcgagtcgggcggcgaatcgctgcgtgccggcgtgcgcaacatgatggaagacctgacgcgcggcaagatctcgcagaccgacgagagcgcgtttgaggtcggccgcaatgtcgcggtgagcgaaggcgccgtagtcttcgagaacgaatacttccagctgttgcagtacaagccgctgaccgacaaggtgcatgcgcgcccgctgctgatggtgccgccgtgcatcaacaagtactacatcctggacctgcagccggagagctcgctggtgcgtcatgtggtggagcaggggcatacggtgttcctggtgtcgtggcgcaatccggacgccagcatggctggcagcacctgggacgactacatcgagcacgcggccatccgcgccatcgaagtcgcgcgcgacatcagcggccaggacaagatcaacgtgctcggcttctgcgtgggcggcaccattgtgtcgactgcgctggcggtgatggccgcgcgcggccagcacccggctgccagcgtcacgctgctgaccacgctgctggactttgccgacaccggcatcctcgacgtctttgtcgacgagggccatgtgcagctgcgcgaggccacgctgggcggcgccgccggcgcgccgtgcgcgctgctgcgcggccttgagctggccaataccttctcgttcctgcgcccgaacgacctggtgtggaactacgtggtcgacaactacctgaagggcaacacgccggtgccgttcgacctgctgttctggaacggcgacgccaccaacctgccggggccttggtactgctggtacctgcgccacacctacctgcaggacgagctcaaggtgccgggcaagctgactgtgtgcggcgtgcccgtggacctggccagcatcgacgtgccgacctacatctacggctcgcgcgaagaccatatcgtgccatggaccgcggcctatgcctcgaccgcgctgctggcgaacaagctgcgcttcgtgctgggtgcgtcgggccatatcgccggtgtgatcaacccgccggccaagaacaagcgcagccactggaccaacgatgcgctgccggagtcgccgcagcaatggctggctggcgccaccgagcatcacggcagctggtggccggactggaccgcatggctggcaggccaggccggcgcgaaacgtgccgcgcccgccaactacggcaatgcgcgctatcccgcgatcgaacccgcgcctgggcgatacgtcaaagccaaggcatga

SEQ ID NO: 2 - protein, 589 aa, Cupriavidus necator
MATGKGAAASTQEGKSQPFKFTPGPFDPATWLEWSRQWQGTEGNGHAAASGIPGLDALAGVKIEPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVSEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVMAARGQHPAASVTLLTTLLDFADTGILDVFVDEGHVQLREATLGGAAGAPCALLRGLELANTFSFLRPNDLVWNYVVDNYLKGNTPVPFDLLFWNGDATNLPGPWYCWYLRHTYLQDELKVPGKLTVCGVPVDLASIDVPTYIYGSREDHIVPWTAAYASTALLANKLRFVLGASGHIAGVINPPAKNKRSHWTNDALPESPQQWLAGATEHHGSWWPDWTAWLAGQAGAKRAAPANYGNARYPAIEPAPGRYVKAKA

SEQ ID NO: 3 - DNA, 1680 bp, Pseudomonas aeruginosa
atgagtcagaagaacaataacgagcttcccaagcaagccgcggaaaacacgctgaacctgaatccggtgatcggcatccggggcaaggacctgctcacctccgcgcgcatggtcctgctccaggcggtgcgccagccgctgcacagcgccaggcacgtggcgcatttcagcctggagctgaagaacgtcctgctcggccagtcggagctacgcccaggcgatgacgaccgacgcttttccgatccggcctggagccagaatccactgtacaagcgctacatgcagacctacctggcctggcgcaaggagctgcacagctggatcagccacagcgacctgtcgccgcaggacatcagtcgtggccagttcgtcatcaacctgctgaccgaggcgatgtcgccgaccaacagcctgagcaacccggcggcggtcaagcgcttcttcgagaccggcggcaagagcctgctggacggcctcggccacctggccaaggacctggtgaacaacggcgggatgccgagccaggtggacatggacgccttcgaggtgggcaagaacctggccaccaccgagggcgccgtggtgttccgcaacgacgtgctggaactgatccagtaccggccgatcaccgagtcggtgcacgaacgcccgctgctggtggtgccgccgcagatcaacaagttctacgtcttcgacctgtcgccggacaagagcctggcgcgcttctgcctgcgcaacggcgtgcagaccttcatcgtcagttggcgcaacccgaccaagtcgcagcgcgaatggggcctgaccacctatatcgaggcgctcaaggaggccatcgaggtagtcctgtcgatcaccggcagcaaggacctcaacctcctcggcgcctgctccggcgggatcaccaccgcgaccctggtcggccactacgtggccagcggcgagaagaaggtcaacgccttcacccaactggtcagcgtgctcgacttcgaactgaatacccaggtcgcgctgttcgccgacgagaagactctggaggccgccaagcgtcgttcctaccagtccggcgtgctggagggcaaggacatggccaaggtgttcgcctggatgcgccccaacgacctgatctggaactactgggtcaacaactacctgctcggcaaccagccgccggcgttcgacatcctctactggaacaacgacaccacgcgcctgcccgccgcgctgcacggcgagttcgtcgaactgttcaagagcaacccgctgaaccgccccggcgccctggaggtctccggcacgcccatcgacctgaagcaggtgacttgcgacttctactgtgtcgccggtctgaacgaccacatcaccccctgggagtcgtgctacaagtcggccaggctgctgggtggcaagtgcgagttcatcctctccaacagcggtcacatccagagcatcctcaacccaccgggcaaccccaaggcacgcttcatgaccaatccggaactgcccgccgagcccaaggcctggctggaacaggccggcaagcacgccgactcgtggtggttgcactggcagcaatggctggccgaacgctccggcaagacccgcaaggcgcccgccagcctgggcaacaagacctatccggccggcgaagccgcgcccggaacctacgtgcatgaacgatga

SEQ ID NO: 4 - DNA, 1683 bp, Pseudomonas aeruginosa
atgcgagaaaagcaggaatcgggtagcgtgccggtgcccgccgagttcatgagtgcacagagcgccatcgtcggcctgcgcggcaaggacctgctgacgacggtccgcagcctggctgtccacggcctgcgccagccgctgcacagtgcgcggcacctggtcgccttcggaggccagttgggcaaggtgctgctgggcgacaccctgcaccagccgaacccacaggacgcccgcttccaggatccatcctggcgcctcaatcccttctaccggcgcaccctgcaggcctacctggcgtggcagaaacaactgctcgcctggatcgacgaaagcaacctggactgcgacgatcgcgcccgcgcccgcttcctcgtcgccttgctctccgacgccgtggcacccagcaacagcctgatcaatccactggcgttaaaggaactgttcaataccggcgggatcagcctgctcaatggcgtccgccacctgctcgaagacctggtgcacaacggcggcatgcccagccaggtgaacaagaccgccttcgagatcggtcgcaacctcgccaccacgcaaggcgcggtggtgttccgcaacgaggtgctggagctgatccagtacaagccgctgggcgagcgccagtacgccaagcccctgctgatcgtgccgccgcagatcaacaagtactacatcttcgacctgtcgccggaaaagagcttcgtccagtacgccctgaagaacaacctgcaggtcttcgtcatcagttggcgcaaccccgacgcccagcaccgcgaatggggcctgagcacctatgtcgaggccctcgaccaggccatcgaggtcagccgcgagatcaccggcagccgcagcgtgaacctggccggcgcctgcgccggcgggctcaccgtagccgccttgctcggccacctgcaggtgcgccggcaactgcgcaaggtcagtagcgtcacctacctggtcagcctgctcgacagccagatggaaagcccggcgatgctcttcgccgacgagcagaccctggagagcagcaagcgccgctcctaccagcatggcgtgctggacgggcgcgacatggccaaggtgttcgcctggatgcgccccaacgacctgatctggaactactgggtcaacaactacctgctcggcaggcagccgccggcgttcgacatcctctactggaacaacgacaacacgcggctgcccgcggcgttccacggcgaactgctcgacctgttcaagcacaacccgctgacccgcccgggcgcgctggaggtcagcgggaccgcggtggacctgggcaaggtggcgatcgacagcttccacgtcgccggcatcaccgaccacatcacgccctgggacgcggtgtatcgctcggccctcctgctgggcggccagcgccgcttcatcctgtccaacagcgggcacatccagagcatcctcaaccctcccggaaaccccaaggcctgctacttcgagaacgacaagctgagcagcgatccacgcgcctggtactacgacgccaagcgcgaagagggcagctggtggccggtctggctgggctggctgcaggagcgctcgggcgagctgggcaaccctgacttcaaccttggcagcgccgcgcatccgcccctcgaagcggccccgggcacctacgtgcatatacgctga

SEQ ID NO: 5 - DNA, 1680 bp, Pseudomonas aeruginosa
atgtcgcagaagaacaacaacgagctgccgaagcaggccgccgagaacaccctgaacctgaacccggtgatcggcattcgtggtaaagatctgctgacgtcggcccgcatggtgctgctgcaagcagtgcgtcaaccgctgcatagtgcacgtcatgttgcgcatttctcgctggagctgaagaacgtgctgctgggccagtcggaactgcgtccgggcgatgatgatcgtcgtttcagtgatccggcatggtcgcaaaatccgctgtacaagcgctacatgcagacgtacctggcctggcgcaaggaactgcactcgtggatctcgcactcggatttgtcgccgcaggatatttcgcgtggccagttcgtgatcaacctgctgacggaggccatgtcgccgaccaattcgctgtcgaatccagcagccgtgaagcgcttcttcgaaaccggcggtaagtcgctgctggacggtctgggtcatctggcaaaggacctggtgaacaatggcggtatgccgtcgcaggtggacatggacgcgttcgaagtgggcaagaatctggccaccaccgaaggtgcagtggtgttccgcaacgacgtgctggagctgatccagtatcgcccgattaccgaatcggtgcacgaacgtccgctgctggttgtgccgccgcagatcaacaagttctacgtgttcgacttgtcgccggacaagtcgctggcccgcttttgcctgcgcaacggcgtgcagacctttattgtgagttggcgtaacccgaccaagtcgcagcgcgaatggggtctgaccacctacatcgaggccctgaaggaagcgatcgaagtggtgctgtcgattaccggctcgaaggacctgaacctgctgggtgcctgcagtggtggcattacaaccgcaaccctggttggccattacgtggcatcgggcgaaaagaaggtcaacgcgttcactcagctggtgtcggtgctggacttcgagctgaacacccaggtggccctgtttgccgatgaaaagacgctggaagccgcaaagcgccgctcgtatcaatcgggtgtgctggagggcaaggacatggcaaaggtgttcgcatggatgcgcccgaacgacctgatctggaactactgggtgaacaactacctgctgggcaaccaaccgccggccttcgacatcctgtactggaacaacgacaccacccgtttgcctgcagcactgcatggcgaattcgtggagctgttcaagtcgaacccgctgaatcgtccgggcgcactggaagtgtcgggtacgccgattgacctgaagcaagtgacctgcgacttctattgcgtggccggcctgaacgaccacatcaccccgtgggaatcgtgctacaagtcggcacgtctgttaggtggtaagtgcgagttcatcctgtcgaactcgggccacatccagtcgatcctgaacccgccgggtaacccgaaagcacgcttcatgaccaacccagaactgccggcagaaccgaaagcatggctggaacaggccggcaagcatgccgatagttggtggctgcattggcagcagtggctggcagaacgttcgggtaaaacgcgcaaggcaccggcaagtctgggcaacaagacctatccggcaggcgaagcagcaccaggcacatatgtgcatgagcgctag

SEQ ID NO: 6 - DNA, 1707 bp, Pseudomonas aeruginosa
atgcgcgagaagcaggagtcgggctcagtgccagtgccggccgagttcatgtcggcccagtcggccattgtgggcctgagaggcaaggacctgctgaccaccgtgcgctcgctggcagtgcatggcctgcgccaaccactgcattcggcccgtcatctggtggcctttggcggccaactgggcaaggtgctgctgggcgacaccctgcatcagccgaacccgcaggatgcccgcttccaggatccgtcgtggcgcctgaatccgttctaccgccgtaccctgcaggcctacctggcctggcagaagcagctgctggcctggatcgacgagtcgaacctggactgcgacgatcgcgcacgtgcccgcttcctggtggcactgctgtcggatgccgtggcaccgtcgaattcgctgatcaacccgctggccctgaaggagctgttcaacaccggcggcatctcgctgctgaacggcgtgcgccacctgctggaggacctggtgcacaatggcggtatgccgtcgcaggtgaacaagaccgccttcgagatcggccgcaacctggccaccacccaaggcgccgtggtgttccgcaacgaggtgctggagctgatccagtacaagccgctgggcgagcgccagtacgccaagccgctgctgatcgtgccgccgcagatcaacaagtactacatcttcgacctgtcgccggagaagtcgttcgtgcagtacgccctgaagaacaacctgcaggtgttcgtgatctcgtggcgcaacccggacgcccagcaccgcgagtggggcctgtcgacctacgtggaagcactggatcaggccatcgaagtgtcgcgcgagatcaccggctcgcgctcggtgaacctggccggcgcatgtgcaggtggtctgactgttgcagccctgctgggtcacctgcaggtgcgccgtcaactgcgcaaggtgtcgtcggtgacctacctggtgtcgctgctggactcgcagatggagtcgccggccatgctgttcgccgacgagcagaccctggagtcgtcgaaacgccgctcgtaccagcatggcgtgctggatggccgcgacatggccaaggtgttcgcctggatgcgcccgaacgacctgatctggaactactgggtgaacaactacctgctgggccgccagccgccggccttcgacatcctgtactggaacaacgacaacacccgcctgccagccgccttccacggcgagctgctggacctgttcaagcacaacccgctgaccagaccaggcgccctggaggtgtcaggcaccgccgtggatctgggcaaggtggccattgactcgttccatgtggccggcatcaccgaccacatcaccccgtgggacgccgtgtaccgctcggcactgctgttgggtggccaacgccgcttcatcctgtcgaattcgggccacatccagtcgatcctgaacccgccgggcaacccgaaggcctgctacttcgagaacgacaagctgtcgtcggacccgcgcgcctggtactacgacgccaagcgcgaggagggctcatggtggccagtttggttgggttggctgcaggagcgctcgggcgaactgggcaacccggacttcaacctgggctcggccgcacatccaccactggaagcagcaccgggcacctacgtgcacatccgtggcggccaccaccaccatcaccattga

SEQ ID NO: 7 - protein, 559 aa, Pseudomonas aeruginosa
MSQKNNNELPKQAAENTLNLNPVIGIRGKDLLTSARMVLLQAVRQPLHSARHVAHFSLELKNVLLGQSELRPGDDDRRFSDPAWSQNPLYKRYMQTYLAWRKELHSWISHSDLSPQDISRGQFVINLLTEAMSPTNSLSNPAAVKRFFETGGKSLLDGLGHLAKDLVNNGGMPSQVDMDAFEVGKNLATTEGAVVFRNDVLELIQYRPITESVHERPLLVVPPQINKFYVFDLSPDKSLARFCLRNGVQTFIVSWRNPTKSQREWGLTTYIEALKEAIEVVLSITGSKDLNLLGACSGGITTATLVGHYVASGEKKVNAFTQLVSVLDFELNTQVALFADEKTLEAAKRRSYQSGVLEGKDMAKVFAWMRPNDLIWNYWVNNYLLGNQPPAFDILYWNNDTTRLPAALHGEFVELFKSNPLNRPGALEVSGTPIDLKQVTCDFYCVAGLNDHITPWESCYKSARLLGGKCEFILSNSGHIQSILNPPGNPKARFMTNPELPAEPKAWLEQAGKHADSWWLHWQQWLAERSGKTRKAPASLGNKTYPAGEAAPGTYVHER

SEQ ID NO: 8 - protein, 560 aa, Pseudomonas aeruginosa
MREKQESGSVPVPAEFMSAQSAIVGLRGKDLLTTVRSLAVHGLRQPLHSARHLVAFGGQLGKVLLGDTLHQPNPQDARFQDPSWRLNPFYRRTLQAYLAWQKQLLAWIDESNLDCDDRARARFLVALLSDAVAPSNSLINPLALKELFNTGGISLLNGVRHLLEDLVHNGGMPSQVNKTAFEIGRNLATTQGAVVFRNEVLELIQYKPLGERQYAKPLLIVPPQINKYYIFDLSPEKSFVQYALKNNLQVFVISWRNPDAQHREWGLSTYVEALDQAIEVSREITGSRSVNLAGACAGGLTVAALLGHLQVRRQLRKVSSVTYLVSLLDSQMESPAMLFADEQTLESSKRRSYQHGVLDGRDMAKVFAWMRPNDLIWNYWVNNYLLGRQPPAFDILYWNNDNTRLPAAFHGELLDLFKHNPLTRPGALEVSGTAVDLGKVAIDSFHVAGITDHITPWDAVYRSALLLGGQRRFILSNSGHIQSILNPPGNPKACYFENDKLSSDPRAWYYDAKREEGSWWPVWLGWLQERSGELGNPDFNLGSAAHPPLEAAPGTYVHIR

SEQ ID NO: 9 - DNA, 1426 bp, Umbellularia californica
aaaaaagtacaaactgtatggtagccatttacatataactactctataattttcaacatggtcaccacctctttagcttccgctttcttctcgatgaaagctgtaatgttggctcctgatggcagtggcataaaacccaggagcagtggtttgcaggtgagggcgggaaaggaacaaaactcttgcaagatgatcaatgggaccaaggtcaaagacacggagggcttgaaagggcgcagcacattgcatggctggagcatgccccttgaattgatcacaaccatcttttcggctgctgagaagcagtggaccaatctagttagtaagccaccgcagttgcttgatgaccatttaggtctgcatgggctagttttcaggcgcacctttgcaatcagatgcagtgaggttggacctgaccgctccacatccatagtggctgttatgaattacttgcaggaagctgcatgtaatcatgcggagagtctgggacttctaggagatggattcggtgagacactagagatgagtaggagagatctgatatgggttgtgagacgcacgcatgttgttgtggaacggtaccctgcttggggcgatactgttgaagtcgaggcctggatcggtgcagctggaaacattggcatgcgccgccattttcttgtccgcgactgcaaaactggccacattcttgcaagatgtaccagtgtttcagtgatgatgaatatgaggacaaggagattgtccaaaattccccaagaagttagaggggagattgaccctcttttcatcgaaaagtttgctgtcaaggaaggggaaattaagaaattacagaagttcaatgatagcactgcagattacattcaagggggttggactccgcgatggaatgatttggatgtcaatcagcacgtgaacaatatcaaatacgttggctggatttttaagagcgtcccagactctatctatgagaatcatcatctttctagcatcactctcgaatacaggagagagtgcacaaggggcagagcactgcagtccctgaccactgtttgtggtggctcgtccgaagctgggatcatatgtgagcacctactccagcttgaggatgggtctgaggttttgaggggaagaacagattggaggcccaagcgcaccgatagtttcgaaggcattagtgagagattcccgcagcaagaaccgcataattaatgacagaagcatcagatatagtttctcctgtgctgttcctgagaatgcatcttacaagtcgtggtttggattgcttgtgcagaatcatggtttgtgctttcagaagtatatctaaattagtccaagttatatgactccatattggaaaataactcaatgagtcgtgctcttgaaatggtcttttaagctttgaaataaagttccacttaatccatgt

SEQ ID NO: 10 - DNA, 942 bp, Umbellularia californica
atgaccaacctggagtggaagccgaagccgaagctgccgcagctgctggacgaccacttcggcctgcacggcctggtgtttcgtcgcaccttcgccattcgctcgtacgaggtgggcccggatcgttcgacctcgatcctggccgtgatgaaccacatgcaggaggccaccctgaaccacgccaagtcggtgggcatcctgggcgacggcttcggcactaccctggagatgtcgaagcgcgacctgatgtgggtggtgcgccgcacccatgtggccgtggagcgctatccgacctggggtgataccgtggaggtggaatgctggatcggcgcctcgggcaacaacggcatgcgccgcgacttcctggtgcgcgactgcaaaaccggcgagattctgacccgctgcacctcgctgtcggtgctgatgaacacccgcacccgccgcctgtcgaccatcccggatgaagtgcgcggcgaaattggcccggccttcatcgacaacgtggccgtgaaggacgacgagatcaagaagctgcagaagctgaacgactcgaccgccgactacatccagggcggcctgaccccgcgctggaacgacctggacgtgaaccagcacgtgaacaacctgaagtacgtggcctgggtgttcgagaccgtgccggactcgatcttcgagtcgcaccacatctcgtcgttcaccctggagtaccgccgcgagtgcacccgcgactcggtgctgcgttcgctgaccaccgtgtcaggtggctcatcggaagcaggcctggtgtgcgaccacctgctgcagctggaaggcggctcggaggtgctgagagcacgcactgaatggcgcccgaagctgaccgactcgtttcgcggcatctcggtgatcccggccgaaccgcgtgtgggctcatcaggcggccaccaccatcaccaccactga

SEQ ID NO: 11 - DNA, 1236 bp, Cuphea palustris
atggtggctgctgcagcaagttctgcatgcttccctgttccatccccaggagcctcccctaaacctgggaagttaggcaactggtcatcgagtttgagcccttccttgaagcccaagtcaatccccaatggcggatttcaggttaaggcaaatgccagtgcgcatcctaaggctaacggttctgcagtaactctaaagtctggcagcctcaacactcaggaggacactttgtcgtcgtcccctcctccccgggctttttttaaccagttgcctgattggagtatgcttctgactgcaatcacaaccgtcttcgtggcaccagagaagcggtggactatgtttgataggaaatctaagaggcctaacatgctcatggactcgtttgggttggagagagttgttcaggatgggctcgtgttcagacagagtttttcgattaggtcttatgaaatatgcgctgatcgaacagcctctatagagacggtgatgaaccacgtccaggaaacatcactcaatcaatgtaagagtataggtcttctcgatgacggctttggtcgtagtcctgagatgtgtaaaagggacctcatttgggtggttacaagaatgaagataatggtgaatcgctatccaacttggggcgatactatcgaggtcagtacctggctctctcaatcggggaaaatcggtatgggtcgcgattggctaataagtgattgcaacacaggagaaattcttgtaagagcaacgagtgtgtatgccatgatgaatcaaaagacgagaagattctcaaaactcccacacgaggttcgccaggaatttgcgcctcattttctggactctcctcctgccattgaagacaacgacggtaaattgcagaagtttgatgtgaagactggtgattccattcgcaagggtctaactccggggtggtatgacttggatgtcaatcagcacgtaagcaacgtgaagtacattgggtggattctcgagagtatgccaacagaagttttggagactcaggagctatgttctctcacccttgaatataggcgggaatgcggaagggacagtgtgctggagtccgtgacctctatggatccctcaaaagttggagaccggtttcagtaccggcaccttctgcggcttgaggatggggctgatatcatgaagggaagaactgagtggcggccgaagaatgcaggaactaacggggcgatatcaacaggaaagacttga

SEQ ID NO: 12 - DNA, 1408 bp, Cuphea palustris
ccacgcgtccgctgagtttgctggttaccattttccctgcgaacaaacatggtggctgccgcagcaagtgctgcattcttctccgtcgcaaccccgcgaacaaacatttcgccatcgagcttgagcgtccccttcaagcccaaatcaaaccacaatggtggctttcaggttaaggcaaacgccagtgcccatcctaaggctaacggttctgcagtaagtctaaagtctggcagcctcgagactcaggaggacaaaacttcatcgtcgtcccctcctcctcggactttcattaaccagttgcccgtctggagtatgcttctgtctgcagtcacgactgtcttcggggtggctgagaagcagtggccaatgcttgaccggaaatctaagaggcccgacatgcttgtggaaccgcttggggttgacaggattgtttatgatggggttagtttcagacagagtttttcgattagatcttacgaaataggcgctgatcgaacagcctcgatagagaccctgatgaacatgttccaggaaacatctcttaatcattgtaagattatcggtcttctcaatgacggctttggtcgaactcctgagatgtgtaagagggacctcatttgggtggtcacgaaaatgcagatcgaggtgaatcgctatcctacttggggtgatactatagaggtcaatacttgggtctcagcgtcggggaaacacggtatgggtcgagattggctgataagtgattgccatacaggagaaattcttataagagcaacgagcgtgtgggctatgatgaatcaaaagacgagaagattgtcgaaaattccatatgaggttcgacaggagatagagcctcagtttgtggactctgctcctgtcattgtagacgatcgaaaatttcacaagcttgatttgaagaccggtgattccatttgcaatggtctaactccaaggtggactgacttggatgtcaatcagcacgttaacaatgtgaaatacatcgggtggattctccagagtgttcccacagaagttttcgagacgcaggagctatgtggcctcacccttgagtataggcgagaatgcggaagggacagtgtgctggagtccgtgaccgctatggatccatcaaaagagggagaccggtctctttaccagcaccttctccgactcgaggacggggctgatatcgtcaaggggagaaccgagtggcggccgaagaatgcaggagccaagggagcaatattaaccggaaagacctcaaatggaaactctatatcttagaaggaggaagggacctttccgagttgtgtgtttatttgctttgctttgattcactccattgtataataatactacggtcagccgtctttgtatttgctaagacaaatagcacagtcattaagtt

SEQ ID NO: 13 - DNA, 981 bp, Artificial Sequence
  synthetic polynucleotide
atgctgctgaccgccatcacgaccgtgttcgtggccccggagaagcgctggaccatgttcgaccgcaagtcgaagcgcccgaacatgctgatggactcgttcggcctggagcgcgtggtgcaggacggcctggtgttccgccagtcgttctcgatccgctcgtacgagatctgcgccgaccgcaccgcctcgatcgagaccgtgatgaaccacgtgcaggagacctcgctgaaccagtgcaagtcgatcggcctgctggacgacggcttcggccgttcgccggagatgtgcaagcgcgacctgatctgggtggtgacccgcatgaagatcatggtgaaccgctacccgacctggggcgacaccatcgaggtgagcacctggctgtcgcagtcgggcaagatcggcatgggccgcgattggctgatctcggactgcaacaccggcgagatcctggtgcgcgccacctcggtgtacgccatgatgaaccagaagacccgccgcttctcgaagctgccgcacgaggtgcgccaggagttcgccccgcacttcctggattcaccaccggccatcgaggacaatgacggcaagctgcagaagttcgacgtgaagaccggcgactcgatccgcaagggcctgaccccgggctggtacgacctggacgtgaaccagcacgtgaacaacgtgaagtacatcggctggatcctgcagtcggtgccgaccgaggtgttcgagacccaggagctgtgcggcctgaccctggagtatcgccgcgaatgcggccgcgattcggtgctggaatcggtgaccgccatggacccgtcgaaggagggcgatcgctcgctgtaccagcacctgctgcgcctggaagatggcgccgacatcgtgaagggccgcaccgaatggcgcccgaagaatgcaggcgcaaagggcgccattctgaccggcaagacctcaggcggccaccaccaccaccatcattga

SEQ ID NO: 14 - protein, 383 aa, Umbellularia californica
MVTTSLASAFFSMKAVMLAPDGSGIKPRSSGLQVRAGKEQNSCKMINGTKVKDTEGLKGRSTLHGWSMPLELITTIFSAAEKQWTNLVSKPPQLLDDHLGLHGLVFRRTFAIRCSEVGPDRSTSIVAVMNYLQEAACNHAESLGLLGDGFGETLEMSRRDLIWVVRRTHVVVERYPAWGDTVEVEAWIGAAGNIGMRRHFLVRDCKTGHILARCTSVSVMMNMRTRRLSKIPQEVRGEIDPLFIEKFAVKEGEIKKLQKFNDSTADYIQGGWTPRWNDLDVNQHVNNIKYVGWIFKSVPDSIYENHHLSSITLEYRRECTRGRALQSLTTVCGGSSEAGIICEHLLQLEDGSEVLRGRTDWRPKRTDSFEGISERFPQQEPHN

SEQ ID NO: 15 - protein, 313 aa, Umbellularia californica
MTNLEWKPKPKLPQLLDDHFGLHGLVFRRTFAIRSYEVGPDRSTSILAVMNHMQEATLNHAKSVGILGDGFGTTLEMSKRDLMWVVRRTHVAVERYPTWGDTVEVECWIGASGNNGMRRDFLVRDCKTGEILTRCTSLSVLMNTRTRRLSTIPDEVRGEIGPAFIDNVAVKDDEIKKLQKLNDSTADYIQGGLTPRWNDLDVNQHVNNLKYVAWVFETVPDSIFESHHISSFTLEYRRECTRDSVLRSLTTVSGGSSEAGLVCDHLLQLEGGSEVLRARTEWRPKLTDSFRGISVIPAEPRVGSSGGHHHHHH

SEQ ID NO: 16 - protein, 326 aa, Artificial Sequence
  synthetic polypeptide
MLLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSGGHHHHHH

SEQ ID NO: 17 - protein, 411 aa, Cuphea palustris
MVAAAASSACFPVPSPGASPKPGKLGNWSSSLSPSLKPKSIPNGGFQVKANASAHPKANGSAVTLKSGSLNTQEDTLSSSPPPRAFFNQLPDWSMLLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVSNVKYIGWILESMPTEVLETQELCSLTLEYRRECGRDSVLESVTSMDPSKVGDRFQYRHLLRLEDGADIMKGRTEWRPKNAGTNGAISTGKT

SEQ ID NO: 18 - protein, 316 aa, Artificial Sequence
  synthetic polypeptide
LLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVSNVKYIGWILESMPTEVLETQELCSLTLEYRRECGRDSVLESVTSMDPSKVGDRFQYRHLLRLEDGADIMKGRTEWRPKNAGTNGAISTGKT

SEQ ID NO: 19 - protein, 411 aa, Cuphea palustris
MVAAAASAAFFSVATPRTNISPSSLSVPFKPKSNHNGGFQVKANASAHPKANGSAVSLKSGSLETQEDKTSSSSPPPRTFINQLPVWSMLLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSNGNSIS

SEQ ID NO: 20 - protein, 315 aa, Artificial Sequence
  synthetic polypeptide
LLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKT

SEQ ID NO: 21 - protein, 316 aa, Artificial Sequence
  synthetic polypeptide
LLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKT

SEQ ID NO: 22 - DNA, 2145 bp, Cupriavidus necator
atgcaagccccgattcagtaccacaagaccgacgacggcatcgtcacgctgacgttcgatgcgcctgagcaaagcgtcaataccatgaccgatgagatgcggcaatgtctggcggacatggtgagccggctggaagcggagaaggaagcggttagcggcgtcattcttacctcggccaaggagacgttctttgcgggaggcaatctcaatcgcctgtacaagctgcagccggcggatgcggctacgcagttcgatgcctcggagcgtgccaagtctgcgctgaggcggctcgaaacgctgggcaagccggtggtggcggcgctcaatggcacggcgctgggtggcggcttcgaaattgcgctggcctgccaccatcgcattgcgctggacaagcccaaagtgcaattcggcctgcccgaagcgacgctgggcctgatgccgggggcgggcggcgtcgtgcgtctgaatcggctgctggggcttgctgcgagccagccttatttgcaggacagcaagctcatgtcgccggcagaggcgaccaaggttgggctggtgcatgagcttgcggacacacccgcggcactgctggagaaggcacgagcatggatcgcggcccacccggaaagcaagcagccgtgggacaaggccggctacacgccgccgggaggctgggccgatgcgagtgaggcgcggcgctggatctccacggccgccgcgcaggtgcgcgccaagaccaaaggttgctaccctgcgccggaagccatcttgtgcgcttcggtcgaaggcatgcaggtggacttcgacaccgctagccgcattgagacgcgctacttcgtgaagcttgtgactggccaggttgcgaagaacatcatcagcaccttctggttccacgccaacggcatcaagtcaggcgcgcagcgtcctgcaggggtggccaagggcaagatcaagacggtgggcgtgctgggcgcagggatgatgggcaaggggattgcgtatgtggcggcctcgcgtggtatcgaggtgtgggtcaaggatgccacccttgcgcaggccgaaggggcacgtgccaatgcggaccaactgctggccaagcgtgaggagaagggggaaattgatgccgcgacccgccgacagattgtcgagcgcattcacgcgactgaccgctatgaggactttgcccatgtcgacctggtggtggaagccatcccagagaaccctgcgcttaaggcggagatcacccggcaggccgagcccgtgctcggagatggggcgatctgggcctccaacacctcgacgctgcccatcaccggcctggccaaggcatcgagccggcccgagcgcttcgtcgggctgcacttcttctcgccggtgcaccgcatgcagttggtggaagtgattaagggccagcagacctcgccggagaccctggcccatgcgctggacttcgtgatgcagcttggcaagacgccgatcgtcgtcaacgacaaccgcggcttctttaccagccgggtattcagtactttcacacgcgaagcagtggcgatgctgggtgaggggcaggacccggccgccatcgaggcggcggccatcctgtcagggttccctgccgggccgctggcggtgctggacgaggtcagcttgagcttgaactacaacaaccggctcgagacgctcagggcgcatgcggaggagggtcgtccgctgccgccacatccggccgacgcagtgatggagcgcatgctcaatgaattcggccgcaaggggcgtgccgcgggtggcggcttctacgattatccggccgacggcaagaaggtgttctggagcggtctggctaagcacttcctgcgcccggccgaacagattccacagcgtgacaaacaggatcggttgttgttctgcatggccctggagtcggtgcgtgtactgcaggatggcgtgctggacagcgcgggggacggcaacattggctcggtactggggattggcttcccgcgctggagcggcggcgtgttccagttcctgaaccagtatgggctggaaaaggccgtggcacgtgcggagtacctggccgagcattatggcgaacggttcacgccaccgcaattgctacgggaaaaggccaaacgagccgagccattctga

SEQ ID NO: 23 - protein, 714 aa, Cupriavidus necator
MQAPIQYHKTDDGIVTLTFDAPEQSVNTMTDEMRQCLADMVSRLEAEKEAVSGVILTSAKETFFAGGNLNRLYKLQPADAATQFDASERAKSALRRLETLGKPVVAALNGTALGGGFEIALACHHRIALDKPKVQFGLPEATLGLMPGAGGVVRLNRLLGLAASQPYLQDSKLMSPAEATKVGLVHELADTPAALLEKARAWIAAHPESKQPWDKAGYTPPGGWADASEARRWISTAAAQVRAKTKGCYPAPEAILCASVEGMQVDFDTASRIETRYFVKLVTGQVAKNIISTFWFHANGIKSGAQRPAGVAKGKIKTVGVLGAGMMGKGIAYVAASRGIEVWVKDATLAQAEGARANADQLLAKREEKGEIDAATRRQIVERIHATDRYEDFAHVDLVVEAIPENPALKAEITRQAEPVLGDGAIWASNTSTLPITGLAKASSRPERFVGLHFFSPVHRMQLVEVIKGQQTSPETLAHALDFVMQLGKTPIVVNDNRGFFTSRVFSTFTREAVAMLGEGQDPAAIEAAAILSGFPAGPLAVLDEVSLSLNYNNRLETLRAHAEEGRPLPPHPADAVMERMLNEFGRKGRAAGGGFYDYPADGKKVFWSGLAKHFLRPAEQIPQRDKQDRLLFCMALESVRVLQDGVLDSAGDGNIGSVLGIGFPRWSGGVFQFLNQYGLEKAVARAEYLAEHYGERFTPPQLLREKAKRAEPF

SEQ ID NO: 24 - DNA, 1179 bp, Cupriavidus necator
atgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaa

SEQ ID NO: 25 - protein, 393 aa, Cupriavidus necator
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 26 - DNA, 741 bp, Cupriavidus necator
atgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 27 - protein, 246 aa, Cupriavidus necator
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 28 - DNA, 2163 bp, Synechocystis PCC6803
atgagctattcatcaaaatacattttactaattagtgtccatggtttaattcggggagaaaaccttgagttgggcagagatgccgacaccggcgggcaaaccaaatatgtgctggaactggcccgggccttggtaaaaaatccccaggtggccagggtggatttgctgacccgtttaattaaagatcccaaagtagatgcagattatgcccagcctagagaactcattggcgatcgggcccagattgttcgcattgagtgcggcccggaggaatatattgccaaggaaatgctctgggactatttggataattttgctgaccatgccctggactatctcaaagaacagcccgaactgcccgatgtcatccatagccattacgccgatgcgggttacgtgggcaccagactttctcaccaattgggtattcctttggtgcacaccggacattccctgggtcgtagtaagcgcacccgtctcctgctcagtgggattaaagccgacgaaattgaaagccgttacaatatggcccgccggattaacgcggaggaagaaaccctaggatcagcggcgagggtgattaccagtacccatcaggaaatcgcagaacagtacgcccaatacgactattaccagccagaccagatgttggttattccccccggcactgatttagaaaagttttatccccccaaagggaacgagtgggaaacgcccattgttcaagagttgcaacgatttctacggcatccccgtaagcctattatcctcgctttgtcccgaccggatccccgcaaaaatatccataaattaattgcagcctatggccagtccccgcagttacaggcccaggccaatttggtcattgtggcgggcaatcgggatgacatcacggatctagaccaggggccgagggaagtactgacggatttactgttgaccattgaccgttacgatctctacggcaaagtggcttaccccaaacagaatcaggcggaggatgtgtatgctttgtttcgcctcactgctttatcccagggagtatttatcaatccggctttgacggaaccctttggtttaactttgattgaagcggcggcctgtggtgtgcccattgtggccacggaggatgggggcccggtggatattatcaaaaattgtcagaatggctatctaattaatcccctcgatgaagtggatattgcggataaattgctcaaagtactaaacgacaaacaacaatggcaattcctttctgaaagtggtctagagggagttaagcgccattattcttggccttcccacgttgaaagttatttagaagccatcaacgctctgacccaacagacttcagtgctgaaacgtagtgatttaaagcggcggcggactttgtactataacggtgccctggttactagtttggaccaaaatttactgggggcattacaggggggattaccgggcgatcgccagacgttggacgaattactggaagtgctgtatcaacatcgaaaaaatgtcggcttttgcattgccactgggagaagattggattcggtgctgaaaattttgcgggagtatcgcattccccaaccggatatgttgatcaccagcatgggcacggaaatttattcttccccggatttgatccccgaccagagttggcgcaatcacattgattatttgtggaaccgtaacgccattgtgcgtattttgggggaattacccggtttagccctccaacccaaggaagaactgagcgcctataaaattagctatttctacgatgcggcgatcgcccctaacctagaagaaattcggcaactgttgcataaaggggaacaaaccgtaaataccatcatttcctttggtcaatttttggatattctgcccatccgagcttccaaaggctatgctgtgcgttggttgagccaacagtggaatattcccctggagcacgttttcaccgccggaggatcgggagccgacgaagatatgatgcggggtaacaccctttccgtcgtcgtggctaaccgtcaccatgaggaactttctaatctaggggagatcgaaccgatttatttttccgaaaaacgttacgccgccggtattctggacggtctggcccattaccgcttctttgagttgttagaccccgtttaa

SEQ ID NO: 29 - DNA, 2193 bp, Synechocystis PCC6803
atgcaccaccaccaccatcacggaggtggctcgtcatattcgtcgaagtacatcctgctgatcagcgtgcacggcctgattcgcggcgagaacctggagctgggtagagatgcagatactggtggtcaaaccaagtacgtgctggaactggcccgcgcactggtgaagaacccgcaagtggcaagagtggatctgctgacacgtctgatcaaagacccgaaggtggacgccgactatgcccaaccgcgcgaactgattggcgatcgtgcacagattgtgcgcatcgaatgtggcccggaggaatacatcgccaaggagatgctgtgggactacctggacaacttcgccgaccatgcactggactacctgaaggaacagccggaactgccggacgtgatccactcgcattatgccgatgccggctatgtgggtaccagactgtcgcatcaactgggcatcccgctggtgcataccggtcattcactgggcagatcgaaacgtacccgtctgctgttgtcgggcatcaaggccgacgagatcgaatcgcgctacaacatggcacgccgcatcaatgccgaggaagaaaccctgggttcggcagcacgcgttattacctcgacccatcaggagatcgccgaacagtacgcccagtacgactactaccagccggaccagatgctggtgattccaccaggcaccgacctggagaagttctatccgccgaagggcaatgagtgggaaaccccgatcgtgcaagaactgcagcgcttcctgcgtcatccgagaaagccgatcatcctggcactgtcacgcccagatccgcgtaagaacatccacaagctgatcgccgcctatggccaatcaccgcaactgcaagcacaggccaacctggtgatcgttgccggcaatcgtgacgacatcaccgacctggatcaaggcccaagagaagtgctgaccgacctgctgctgaccattgaccgctacgacctgtacggcaaagtggcctacccgaagcagaatcaggccgaagacgtgtacgccctgtttcgtctgaccgcactgtcacaaggcgtgttcatcaacccagcactgaccgagccgtttggcctgaccctgatcgaagcagcagcatgtggtgtgccgattgttgcaaccgaagatggtggcccagtggacatcatcaagaactgccagaacggctacctgatcaacccgctggacgaggtggatatcgccgacaagctgctgaaggtgctgaacgacaagcagcagtggcagttcctgtcggaatcgggcttggaaggcgtgaagcgccattactcatggccgtcacacgtggagtcgtacctggaagccatcaacgcactgacccagcaaaccagcgtgctgaagcgctcagacctgaaaagacgtcgcaccctgtactacaatggcgccctggtgacctcgctggaccagaacctgttgggtgcattacaaggcggtctgccaggtgatagacagaccctggacgagctgctggaggtgctgtaccaacaccgcaaaaatgtgggcttctgcatcgcaaccggccgtcgtctggattcggtgctgaagatcctgcgcgagtatagaatcccgcaaccggatatgctgatcacctcgatgggcaccgagatctactcgtcgccggacctgattccggatcaatcgtggcgcaaccatatcgactacctgtggaaccgcaacgccattgtgcgcatcctgggcgaattgcctggtctggcactgcaaccgaaggaagaactgagcgcctacaagatctcgtacttctacgacgccgccatcgccccgaacctggaggaaatccgtcaactgctgcacaagggcgaacaaaccgttaacaccatcatctcgttcggccagttcctggacatcctgccgatccgcgcctcgaagggctatgcagttagatggctgtcgcaacagtggaatatcccgctggaacacgtgttcaccgccggcggtagtggcgcagatgaagacatgatgcgcggcaatactctgtcagttgtggtggcaaaccgccaccacgaggagctgtcgaatctgggcgaaatcgagccgatctacttctcggaaaagcgctatgcagccggcatcctggacggcttggcccattaccgcttcttcgagctgttggatccggtttga

SEQ ID NO: 30 - protein, 720 aa, Synechocystis PCC6803
MSYSSKYILLISVHGLIRGENLELGRDADTGGQTKYVLELARALVKNPQVARVDLLTRLIKDPKVDADYAQPRELIGDRAQIVRIECGPEEYIAKEMLWDYLDNFADHALDYLKEQPELPDVIHSHYADAGYVGTRLSHQLGIPLVHTGHSLGRSKRTRLLLSGIKADEIESRYNMARRINAEEETLGSAARVITSTHQEIAEQYAQYDYYQPDQMLVIPPGTDLEKFYPPKGNEWETPIVQELQRFLRHPRKPIILALSRPDPRKNIHKLIAAYGQSPQLQAQANLVIVAGNRDDITDLDQGPREVLTDLLLTIDRYDLYGKVAYPKQNQAEDVYALFRLTALSQGVFINPALTEPFGLTLIEAAACGVPIVATEDGGPVDIIKNCQNGYLINPLDEVDIADKLLKVLNDKQQWQFLSESGLEGVKRHYSWPSHVESYLEAINALTQQTSVLKRSDLKRRRTLYYNGALVTSLDQNLLGALQGGLPGDRQTLDELLEVLYQHRKNVGFCIATGRRLDSVLKILREYRIPQPDMLITSMGTEIYSSPDLIPDQSWRNHIDYLWNRNAIVRILGELPGLALQPKEELSAYKISYFYDAAIAPNLEEIRQLLHKGEQTVNTIISFGQFLDILPIRASKGYAVRWLSQQWNIPLEHVFTAGGSGADEDMMRGNTLSVVVANRHHEELSNLGEIEPIYFSEKRYAAGILDGLAHYRFFELLDPV

SEQ ID NO: 31 - DNA, 735 bp, Synechocystis PCC6803
atgcgacagttattgctaatttctgacctggacaatacctgggtcggagatcaacaagccctggaacatttgcaagaatatctaggcgatcgccggggaaatttttatttggcctatgccacggggcgttcctaccattccgcgagggagttgcaaaaacaggtgggactcatggaaccggactattggctcaccgcggtggggagtgaaatttaccatccagaaggcctggaccaacattgggctgattacctctctgagcattggcaacgggatatcctccaggcgatcgccgatggttttgaggccttaaaaccccaatctcccttggaacaaaacccatggaaaattagctatcatctcgatccccaggcttgccccaccgtcatcgaccaattaacggagatgttgaaggaaaccggcatcccggtgcaggtgattttcagcagtggcaaagatgtggatttattgccccaacggagtaacaaaggtaacgccacccaatatctgcaacaacatttagccatggagccgtctcaaaccctggtgtgtggggactccggcaatgatattggcttatttgaaacttccgctcggggtgtcattgtccgtaatgcccagccggaattattgcactggtatgaccaatggggggattctcgtcattatcgggcccaatcgagccatgctggcgctatcctagaggcgatcgcccatttcgattttttgagctga

SEQ ID NO: 32 - DNA, 765 bp, Synechocystis PCC6803
atgcaccaccaccaccatcacggaggtggctcgagacaactgctgctgatcagcgacctggataacacttgggtgggcgatcaacaggccctggagcacctgcaggaatatctgggcgatagacgcggcaacttctatctggcctatgcaaccggccgctcgtaccattcggcaagagaactgcagaagcaggtgggcctgatggaaccggattattggctgaccgccgtgggctcggagatctatcatccggaaggtctggaccagcattgggccgactatctgtcggaacactggcagcgcgacatcttgcaagcaatcgcagacggcttcgaagccctgaagccgcaatcaccactggagcagaacccgtggaagatctcgtaccatctggacccgcaagcatgcccaaccgtgatcgatcagctgaccgagatgctgaaggaaaccggcattccggtgcaggtgatcttctcgtcgggcaaggatgtggacctgctgccgcaacgttcgaataagggcaatgccacccagtacctgcagcagcatctggccatggaaccgtcgcagaccctggtgtgtggtgattcaggcaacgatattggcctgttcgagacgtcggcaagaggcgtgatcgtgcgcaacgcacagccagaactgctgcattggtatgaccaatggggcgatagccgccattatcgcgcacagtcgtcgcatgcaggcgcaatcctggaagcaatcgcacatttcgacttcctgtcgtag

SEQ ID NO: 33 - protein, 244 aa, Synechocystis PCC6803
MRQLLLISDLDNTWVGDQQALEHLQEYLGDRRGNFYLAYATGRSYHSARELQKQVGLMEPDYWLTAVGSEIYHPEGLDQHWADYLSEHWQRDILQAIADGFEALKPQSPLEQNPWKISYHLDPQACPTVIDQLTEMLKETGIPVQVIFSSGKDVDLLPQRSNKGNATQYLQQHLAMEPSQTLVCGDSGNDIGLFETSARGVIVRNAQPELLHWYDQWGDSRHYRAQSSHAGAILEAIAHFDFLS

SEQ ID NO: 34 - DNA, 1518 bp, Escherichia coli
atgtataaaaaaacaactttggcagtgttaattgctttgctgaccggtgctacaacggtacatgcgcaaacggatattagcagtatcgaatctcgactggcggcattggaacaacgtttaaaaaatgcggaatcccgcgcccaggcggcagaagcaagggccaaaacagctgaattacaggttcagaaactggctgaaacacaacaacaaaatcagctaacaactcaagaagtagcacagagaacagttcagctcgaacagaaatccgcagaaaacagtggttttgagtttcatggctatgcccgttccgggttactgatgaatgatgccggttccagtagcaaaagtgggccgtatctgactcccgcaggtgaaactggtggagctgttggccgtctgggaaaagaagccgatacctatgtcgagttaaatgtagaacataaacaaacactggataacggtgcgaccacacgctttaaagcaatgttggctgacggacaaagagattacaacgactggactggcggctccagtaacctgaatatccgacaggcttttgccgaactgggcgcattaccaagttttaccggagcattccaagacagtactgtctgggctggaaaacgctttgatcgcgacaattttgatattcactggttagactccgatgtcgtatttttagcgggaacgggcggcggtatctatgacgtaaaatggaacgatacattccgcagtaacttttctctctacggacgtaatttcggcgatcttgatgatatcgacaataacgttcagaactacatcctcaccatgaatcattatgcaggccccttccagttgatggttagcggattaggggcaaaagataatgatgatcgaaaagatggcaatggtgatctcattcaaactgatgctgcaaatactggcgtacatgcgttagttggtctgcacaatgacactttctatggcctgcgtgaagggacggcaaaaacagcactgctatatggccatggcctgggtgcggaagtcaaagggattggctccgatggcgctctgctgtctgaggcgaatacctggcgcttcgcatcttacggcacaacacctctgggaagcggttggtatgttgcgccagcaattctcgcacaaagcagtaaagatcgttacgtcaaaggcgatagctacgaatgggtgaccttcaatacacgtctgatcaaagaggtaacacagaattttgctctggcctttgagggtagctatcaatatatggatctgaagccaaaggggtatcaaaaccacaacgccgtaaacggcagcttctataaactcacctttgctccaactctaaaagctaacgatatcaataatttctttagccgtccggagcttcgcctgtttgccacctggatggactggagcagcaaacttgatgattttgccagcaatgacgctttcggcagcagtggtttcaatactggcggagagtggaattttggtgtccaaatggaaacctggttttaa

SEQ ID NO: 35 - DNA, 1518 bp, Escherichia coli
atgtacaggaaatctacgctagcaatgctaattgcactacttacgtctgcggcaagcgcgcacgcccaaacagatatttcgacaatagaggccaggcttaatgcactggagaagcgattgcaagaagcagaaaaccgtgcccagacagctgaaaacagggcaggcgcagctgaaaagaaagttcagcaacttacggcacaacagcaaaaaaatcaaaactcaacgcaagaagtcgcgcaacggactgccaggcttgagaagaaagccgatgacaagtccggttttgaatttcacggctacgctagaagtggcgttattatgaatgattccggcgcctcaaccaaatccggggcatatatcacgcctgccggcgagacagggggagctattggacgattaggaaatcaagcagacacgtacgtagaaatgaatctagaacacaagcaaactcttgacaatggcgccacaactcggtttaaagtaatggtcgcggacggtcagaccagttataacgattggacggcatcgacgtccgatctgaacgtccgccaggccttcgtggaattagggaacctcccgaccttcgctggtccgttcaagggatcgaccctatgggccgggaagcgctttgaccgagataacttcgatattcattggatagattcagacgtggtattcctcgcggggaccggaggtggcatatatgacgtgaaatggaacgacgggttgagaagtaatttctcgttgtatggccgcaattttggggacatagatgactcttccaactccgttcagaactatattcttacaatgaatcacttcgctggacccttacagatgatggtatctggtttaagggcaaaagataacgacgaacgtaaagatagcaatggaaatctagttaagggggacgctgccaataccggcgttcatgcgttgttaggcctccataatgactcattctatggccttagagatggcagtagcaagaccgcccttctctatggacacggcttgggtgctgaagttaagggaattggttccgacggagccttacggcccggagcggatacctggagaatagcgtcgtacggtacgacacctctctcagaaaactggagtgtcgcgccggcaatgctcgctcagcgcagcaaggaccggtatgctgacggcgactcctaccaatgggcaacctttaatttgcgcctgattcaggctatcaatcagaattttgcgctagcctacgaagggtcataccaatatatggatcttaaacctgaagggtacaatgaccgccaggcagtcaatgggtcattctataaactcacttttgcaccgacgttcaaggtgggatccattggggattttttctcccggcctgaaatccgattttatacttcctggatggattggagcaagaaactaaataactatgcttctgatgacgcgttgggctcagatgggtttaactcaggtggcgagtggagttttggtgttcagatggaggcctggttctag

SEQ ID NO: 36 - protein, 505 aa, Escherichia coli
MYKKTTLAVLIALLTGATTVHAQTDISSIESRLAALEQRLKNAESRAQAAEARAKTAELQVQKLAETQQQNQLTTQEVAQRTVQLEQKSAENSGFEFHGYARSGLLMNDAGSSSKSGPYLTPAGETGGAVGRLGKEADTYVELNVEHKQTLDNGATTRFKAMLADGQRDYNDWTGGSSNLNIRQAFAELGALPSFTGAFQDSTVWAGKRFDRDNFDIHWLDSDVVFLAGTGGGIYDVKWNDTFRSNFSLYGRNFGDLDDIDNNVQNYILTMNHYAGPFQLMVSGLGAKDNDDRKDGNGDLIQTDAANTGVHALVGLHNDTFYGLREGTAKTALLYGHGLGAEVKGIGSDGALLSEANTWRFASYGTTPLGSGWYVAPAILAQSSKDRYVKGDSYEWVTFNTRLIKEVTQNFALAFEGSYQYMDLKPKGYQNHNAVNGSFYKLTFAPTLKANDINNFFSRPELRLFATWMDWSSKLDDFASNDAFGSSGFNTGGEWNFGVQMETWF

SEQ ID NO: 37 - DNA, 1458 bp, Bradyrhizobium japonicum
atggacctgctcgcgacgaccagtgctgccgccgtttcatcttatgcgctcctatcgacgatctataagagcgtgcaagcgctttatgctcagccggcgatcaactcatcgctggacaaccttggacaagccgaggtggtcgttcctgctgtggacgtgatcgtgccgtgcttcaacgagaatccgaacacactcgccgaatgtctggagtcgattgccagtcaagactacgccggaaagatgcaggtatatgtggtcgatgacggatcggcaaaccgcgacgttgtcgcgcctgtacaccggatatatgcgagcgatccgagattcagttttatcttgttggcgaacaatgtgggaaagcgcaaggcgcagatcgcagcgatacgcagctcatccggtgatctggttctcaacgtcgattccgatacgatacttgctgccgacgtcgtcacgaagcttgtattgaagatgcatgacccgggaatcggtgcggccatgggtcagctgatcgcgagcaatcgcaaccagacctggctgaccaggctgatcgacatggaatattggctcgcgtgcaacgaagagcgcgcggcacaggcgcgcttcggtgccgtcatgtgttgctgcggcccatgtgccatgtatcggcgttccgcgctcgccttgcttcttgatcaatatgaagcccaattctttcgtgggaagccgagcgatttcggcgaggaccgccacctaacgatactcatgctcaaggcggggtttcgaaccgaatacgttccggacgccatagcagccacagtcgtcccgcacagtcttcggccatatctacgacagcaactccgctgggcgcgaagtacctttcgagatacgtttcttgcttggcgcctgctgccagagctcgatggttatttgacgctagacgttatcgggcaaaatctcggcccattgctcctcgccatttcatcacttgctgcgctcgcacagctcctgatcgatggctctataccctggtggacgggattgacgattgctgcaatgactacggtccggtgctgtgtggcagcgcttcgtgcccgcgagctgcggtttatcggcttctcgctccacacgccgatcaatatctgtctcttactgcctttgaaggcctatgcgctttgtacattgagcaatagcgattggctatctcggaaagtcaccgatatgccgacggaagaggggaaacagcctgtcatcctgcacccgaatgccggacgaagtcctgctggtgtaggggggcgcctgctcctattcgtaaggcggcgttatcgcagcctccatcgagcctggcggcgacggagagtgtttccggtcgcgatcgttcgactgtctacaaataagtggtcggctgatgactcaggacgaaaaccatcagttattagagcgagagttggctgtcgacgacccgtggcgcctcgacactag

SEQ ID NO: 38 - protein, 485 aa, Bradyrhizobium japonicum
MDLLATTSAAAVSSYALLSTIYKSVQALYAQPAINSSLDNLGQAEVVVPAVDVIVPCFNENPNTLAECLESIASQDYAGKMQVYVVDDGSANRDVVAPVHRIYASDPRFSFILLANNVGKRKAQIAAIRSSSGDLVLNVDSDTILAADVVTKLVLKMHDPGIGAAMGQLIASNRNQTWLTRLIDMEYWLACNEERAAQARFGAVMCCCGPCAMYRRSALALLLDQYEAQFFRGKPSDFGEDRHLTILMLKAGFRTEYVPDAIAATVVPHSLRPYLRQQLRWARSTFRDTFLAWRLLPELDGYLTLDVIGQNLGPLLLAISSLAALAQLLIDGSIPWWTGLTIAAMTTVRCCVAALRARELRFIGFSLHTPINICLLLPLKAYALCTLSNSDWLSRKVTDMPTEEGKQPVILHPNAGRSPAGVGGRLLLFVRRRYRSLHRAWRRRRVFPVAIVRLSTNKWSADDSGRKPSVIRARVGCRRPVAPRH

SEQ ID NO: 39 - DNA, 660 bp, Bradyrhizobium japonicum
gtgacagagcgttccaccctatctactgtccgctgcgactacgctgacgcgggcggaagtcgagctgtccatttgacctttgacgatgggccaaatccattttgtacgccagaggtgctcgatgtcctggcgcaacatcgggtccccgcgacattcttcgtcatcgggacgtacgcgacggagcatcctgaactcatccgacgaatgattgcggaagggcatgaggttgcgaaccatacgatgacccatcctgatctatccagatgcggacctacggagctacacgacgaggtgctgacggcgagcgaagccatccgtctggcgtgcccgctggcctcgcccaggcatatgcgagcgccttacggcatatggacgcgagatgtgctcgcagtggcggcgagcgctggtctcacggctctgcactggtcggtcgaccctagagattggtcccgccccggggttgatgcaattgtgaattccgtactggcgaacgtacgcccgggtgcaattgtgctcctgcacgacggatatcctcccgatgaggagggattgcccagtggctctacgctgcgcgatcagaccaggacggcgctggcatatctcattccagcactacaacggcgcgggtttgtaatccgtccactccctcaactccactga

SEQ ID NO: 40 - protein, 219 aa, Bradyrhizobium japonicum
MTERSTLSTVRCDYADAGGSRAVHLTFDDGPNPFCTPEVLDVLAQHRVPATFFVIGTYATEHPELIRRMIAEGHEVANHTMTHPDLSRCGPTELHDEVLTASEAIRLACPLASPRHMRAPYGIWTRDVLAVAASAGLTALHWSVDPRDWSRPGVDAIVNSVLANVRPGAIVLLHDGYPPDEEGLPSGSTLRDQTRTALAYLIPALQRRGFVIRPLPQLH

SEQ ID NO: 41 - DNA, 633 bp, Bradyrhizobium japonicum
atgaacattgccgtctccccgactgcggaaggatcttctgggcgcgctcaagtgcagtggagccttcgttgggaaagtgaactgcagctcgccgatcatgccgagctcgcggagttcttccgcaagagttacggaccgacgggtgcgttcaatgcgcagccgtttgagcggagccgaagttgggctggagcaagacccgagctccgcgtaattggttacgacgcgcgcggggtagcggctcacatcgggctactgcgccgcttcatcaaagttggtgaagtcgatctccctgtggccgaactggggttgtatgcggtgcgccccgatctcgaggggcacgggataggccacgcaatgcgcgtgatgtatcccgcactccaggagctcggcgttccattcggatttggcgcggttcgctcggccctcgaaaaacatttgacccgactggtcgaaaggcaggggctcgccaccctgatgcgtggcattcgcgtccgctccaccttgccggatgtctatccaaatttatcgccgacgcgcatcgaagacgtgatcgtcgtggtgtttccggtcggacgctcgataagcgaatggccggccgggactgtcatcgatcgtaacgggcctgagttgtga

SEQ ID NO: 42 - protein, 210 aa, Bradyrhizobium japonicum
MNIAVSPTAEGSSGRAQVQWSLRWESELQLADHAELAEFFRKSYGPTGAFNAQPFERSRSWAGARPELRVIGYDARGVAAHIGLLRRFIKVGEVDLPVAELGLYAVRPDLEGHGIGHAMRVMYPALQELGVPFGFGAVRSALEKHLTRLVERQGLATLMRGIRVRSTLPDVYPNLSPTRIEDVIVVVFPVGRSISEWPAGTVIDRNGPEL

SEQ ID NO: 43 - DNA, 1434 bp, Escherichia coli
atgacgcaatctcgattgcatgcggcgcaaaacgcactagcaaaacttcacgagcgccgaggtaacactttctatccccattttcacctcgcgcctcctgccgggtggatgaacgatccaaacggcctgatctggtttaacgatcgttatcacgcgttttatcaacatcacccgatgagcgaacactgggggccaatgcactggggacatgccaccagcgacgatatgatccactggcagcatgagcctattgcgctagcgccaggagacgagaatgacaaagacggatgtttttcaggtagtgctgtcgatgacaatggtgtcctctcacttatctacaccggacacgtctggctcgatagtgaaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagacccaggcaacacggggcagatcctgctttatcgcggcagttcattgcgtgaatggactttcgatcgcgtactggcccacgctgatgcgggtgaaagctatatgtgggaatgtccggactttttcagccttggcgatcagcattatctgatgttttccccgcagggaatgaatgccgagggatacagttatcgaaatcgctttcaaagtggcgtaatacccggaatgtggtcgccaggacgactttttgcacaatccgggcattttactgaacttgataacgggcatgacttttatgcaccacaaagctttgtagcgaaggatggtcggcgtattgttatcggctggatggatatgtgggaatcgccaatgccctcaaaacgtgaaggctgggcaggctgcatgacgctggcgcgcgagctatcagagagcaatggcaaactcctacaacgcccggtacacgaagctgagtcgttacgccagcagcatcaatctatctctccccgcacaatcagcaataaatatgttttgcaggaaaacgcgcaagcagttgagattcagttgcagtgggagctgaagaacagtgatgccgaacattacggattacaactcggcacaggaatgcggctgtatattgataaccaatctgagcgacttgttttgtggcgatattacccacacgagaatttagacggctaccgtagtattcccctcccgcagggtgacacgctcgccctaaggatatttatcgatacatcatccgtggaagtatttattaacgacggggaaacggtgatgagtagccgaatctatccgcagccagaagaacgggaactgtcgctctatgcctcccacggagtggctgtgctgcaacatggagcactctggcaactgggttaa

SEQ ID NO: 44 - protein, 477 aa, Escherichia coli
MTQSRLHAAQNALAKLHERRGNTFYPHFHLAPPAGWMNDPNGLIWFNDRYHAFYQHHPMSEHWGPMHWGHATSDDMIHWQHEPIALAPGDENDKDGCFSGSAVDDNGVLSLIYTGHVWLDSEGNDDAIREVQCLATSRDGIHFEKQGVILTPPEGIMHFRDPKVWREADTWWMVVGAKDPGNTGQILLYRGSSLREWTFDRVLAHADAGESYMWECPDFFSLGDQHYLMFSPQGMNAEGYSYRNRFQSGVIPGMWSPGRLFAQSGHFTELDNGHDFYAPQSFVAKDGRRIVIGWMDMWESPMPSKREGWAGCMTLARELSESNGKLLQRPVHEAESLRQQHQSISPRTISNKYVLQENAQAVEIQLQWELKNSDAEHYGLQLGTGMRLYIDNQSERLVLWRYYPHENLDGYRSIPLPQGDTLALRIFIDTSSVEVFINDGETVMSSRIYPQPEERELSLYASHGVAVLQHGALWQLG

SEQ ID NO: 45 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctaggattaacagggacggaattaggtacactttattcggtcaaccagtttaccagcattctatttatgatgttctacggcatcgttcaggataaactcggtctgaagaaaccgctcatctggtgtatgagtttcattctggtcttgaccggaccgtttatgatttacgtttatgaaccgttactgcaaagcaatttttctgtaggtctaattctgggggcgctcttttttggcctggggtatctggcgggatgtggtttgcttgacagcttcactgaaaaaatggcgcgaaattttcatttcgaatatggaacagcgcgcgcctggggatcttttggctatgctattggcgcgttctttgccggcatattttttagtatcagtccccatatcaacttctggctggtctcgctatttggcgctgtatttatgatgatcaacatgcgttttaaagataagggtcaccagtgtgtagcggcggatgcgggaggggtaaaaaaagaggattttatcgcagttttcaaggatcgaaacttctgggtttttgtcatatttattgtggggacgtggtctttctataacatttttgatcaacaactctttcctgtcttttatgcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggtgttgtgattatggcgttgcgtatcctttcctgcgcgttgttcgttaacccctggattatttcattagtgaagctgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcctgagtaaaaaacgcgagcaaatagttatggaaacgcctgtaccttcagcaatatag

SEQ ID NO: 46 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKGHQCVAADAGGVKKEDFIAVFKDRNFWVFVIFIVGTWSFYNIFDQQLFPVFYAGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 47 - DNA, 924 bp, Escherichia coli
atgtcagccaaagtatgggttttaggggatgcggtcgtagatctcttgccagaatcagacgggcggctactgccttgtcctggcggcgcgccagctaacgttgcggtgggaatcgccagattaggcggaacaagtgggtttataggtcgggtcggtgatgatccttttggtgcgttaatgcaaagaacgctgctaactgagggtgtcgatatcacgtatctgaagcaagatgaatggcaccggacatccacggtgcttgtcgatctgaacgatcaaggagaacgttcatttacgtttatggtccgccccagtgccgatctttttttagagacgacagacttgccctgctggcgacatggcgaatggttacatctctgttcaattgcgttgtctgccgagccttcgcgtaccagcgcatttactgcgatgacggcgatccggcatgccggaggttttgtcagcttcgatcccaatattcgtgaagatctatggcaagacgagcatttgctccgcttgtgtttgcggcaggcgctacaactggcggatgtcgtcaagctctcggaagaagaatggcgacttatcagtggaaaaacacagaacgatcgggatatatgcgccctggcaaaagattatgagatcgccatgctgttggtgactaaaggtgcagaaggggtggtggtctgttatcgaggacaagtccaccattttgctggaatgtctgtgaattgtgtcgatagcactggggcgggagatgcgttcgttgccgggttactcacaggtctgtcctcttcgggattatctacagatgagagagaaatgcgacgaattatcgatctcgctcaacgttgcggagcgcttgcagtaacagcgaaaggggcaatgacagcgctgccatgtcgacaagaactggaaagtgagaagtaa

SEQ ID NO: 48 - protein, 307 aa, Escherichia coli
MSAKVWVLGDAVVDLLPESDGRLLPCPGGAPANVAVGIARLGGTSGFIGRVGDDPFGALMQRTLLTEGVDITYLKQDEWHRTSTVLVDLNDQGERSFTFMVRPSADLFLETTDLPCWRHGEWLHLCSIALSAEPSRTSAFTAMTAIRHAGGFVSFDPNIREDLWQDEHLLRLCLRQALQLADVVKLSEEEWRLISGKTQNDRDICALAKDYEIAMLLVTKGAEGVVVCYRGQVHHFAGMSVNCVDSTGAGDAFVAGLLTGLSSSGLSTDEREMRRIIDLAQRCGALAVTAKGAMTALPCRQELESEK

SEQ ID NO: 49 - DNA, 996 bp, Escherichia coli
atggcttcattaaaggatgtcgcacgcctggcgggagtgtcgatgatgacagtctcccgggtgatgcataatgcagaatctgtgcgtcctgcaacgcgtaaccgcgtattgcaggcaatccagaccctgaattatgttcctgatctttccgcccgtaagatgcgcgctcaaggacgtaagccgtcgactctcgccgtgctggcgcaggacacggctaccactcctttctctgttgatattctgcttgccattgagcaaaccgccagcgagttcggctggaatagttttttaatcaatattttttctgaagatgacgctgcccgcgcggcacgtcagctgcttgcccaccgtccggatggcattatctatactacaatggggctgcgacatatcacgctgcctgagtctctgtatggtgaaaatattgtattggcgaactgtgttgcggatgacccagcgttacccagttatatccctgatgattacactgcacaatatgaatcaacacagcatttgctcgcggcgggctatcgtcaaccgttatgcttctggctaccggaaagtgcgttggcaacagggtatcgtcggcagggatttgagcaggcctggcgtgatgctggacgagatctggctgaggtgaaacaatttcacatggcaacaggtgatgatcactacaccgatctcgcaagtttactcaatgaccacttcaaatctggcaaaccagattttgatgttctgatatgtggtaacgatcgcgcagcctttgtcgcttatcaggttctcctggcgaagggggtacgtatcccgcaggatgtcgccgtaatgggctttgataatctggttggcgtcgggcatctgtttttaccgccgctgaccacaattcagcttccacatgacattatcgggcgggaagctgcattgcatattattgaaggtcgtgaagggggaagagtgacgcggatcccttgcccgctgttgatccgttgttccacctga

SEQ ID NO: 50 - protein, 331 aa, Escherichia coli
MASLKDVARLAGVSMMTVSRVMHNAESVRPATRNRVLQAIQTLNYVPDLSARKMRAQGRKPSTLAVLAQDTATTPFSVDILLAIEQTASEFGWNSFLINIFSEDDAARAARQLLAHRPDGIIYTTMGLRHITLPESLYGENIVLANCVADDPALPSYIPDDYTAQYESTQHLLAAGYRQPLCFWLPESALATGYRRQGFEQAWRDAGRDLAEVKQFHMATGDDHYTDLASLLNDHFKSGKPDFDVLICGNDRAAFVAYQVLLAKGVRIPQDVAVMGFDNLVGVGHLFLPPLTTIQLPHDIIGREAALHIIEGREGGRVTRIPCPLLIRCST

SEQ ID NO: 51 - DNA, 1257 bp, Chromobacterium violaceum
atgaagcattcttccgatatctgcattgtcggcgccggcatcagcggcctgacctgcgccagccatctgctcgactcgcccgcttgccgcggcctgtcgctgcgcatcttcgacatgcagcaggaggcgggcggccgcatccgctcgaagatgctggatggcaaggcgtcgatagagctgggcgcggggcgatactccccgcagctgcacccgcatttccagagcgcgatgcagcattacagccagaagagcgaggtgtatccgttcacccagctgaaattcaagagccatgtccagcagaagctgaagcgggcgatgaacgagttgtcgcccaggctgaaagagcatggcaaggaatcctttctccagttcgtcagccgctaccagggccatgacagcgcggtgggcatgatccgctccatgggctacgacgcgctgttcctgcccgacatctcggccgagatggcctacgacatcgtcggcaagcacccggaaatccagagcgtgaccgataacgacgccaaccagtggttcgcggcggaaacgggctttgcgggcctgatccagggcatcaaggccaaggtcaaggctgccggcgcgcgcttcagcctgggttaccggctgctgtcggtgaggacggacggcgacggctacctgctgcaactggccggcgacgacggctggaagctggaacaccggacccgccatctgatcctggccattcctccgtcggcgatggccgggctcaatgtcgacttccccgaggcgtggagcggcgcgcgctacggctcgctgccgctgttcaagggtttcctcacctacggcgagccatggtggctggactacaagctggacgaccaggtgctgatcgtcgacaacccgctgcgcaagatctacttcaagggcgacaagtacctgttcttctacaccgacagcgagatggccaattactggcgcggctgcgtggccgaaggagaggacggctacctggagcagatccgcacccatctggccagcgcgctgggcatcgttcgcgagcgcattccccagcccctcgcccatgtgcacaagtattgggcgcatggcgtggagttctgccgcgacagcgatatcgaccatccgtccgcgctcagccaccgcgacagcggcatcatcgcctgttcggacgcctacaccgagcactgcggctggatggagggcggcctgctcagcgcccgcgaagccagccgtctgctgctgcagcgcatcgccgcgtga

SEQ ID NO: 52 - protein, 418 aa, Chromobacterium violaceum
MKHSSDICIVGAGISGLTCASHLLDSPACRGLSLRIFDMQQEAGGRIRSKMLDGKASIELGAGRYSPQLHPHFQSAMQHYSQKSEVYPFTQLKFKSHVQQKLKRAMNELSPRLKEHGKESFLQFVSRYQGHDSAVGMIRSMGYDALFLPDISAEMAYDIVGKHPEIQSVTDNDANQWFAAETGFAGLIQGIKAKVKAAGARFSLGYRLLSVRTDGDGYLLQLAGDDGWKLEHRTRHLILAIPPSAMAGLNVDFPEAWSGARYGSLPLFKGFLTYGEPWWLDYKLDDQVLIVDNPLRKIYFKGDKYLFFYTDSEMANYWRGCVAEGEDGYLEQIRTHLASALGIVRERIPQPLAHVHKYWAHGVEFCRDSDIDHPSALSHRDSGIIACSDAYTEHCGWMEGGLLSAREASRLLLQRIAA

SEQ ID NO: 53 - DNA, 2997 bp, Chromobacterium violaceum
atgagcattctggattttccacgcatccatttccgcggctgggcgcgggtcaacgcgcccaccgccaaccgcgatccgcacggccacatcgacatggccagcaatacggtggccatggcaggcgaaccgttcgacctcgcgcgccatccgaccgagttccaccgccacctgcggtcgctggggccgcgtttcggcctggacggccgggctgacccggaagggccgttcagcctggccgagggctacaacgcggccggcaacaaccatttctcctgggagagcgccaccgtcagccacgtgcagtgggatggcggcgaagcggaccgcggcgacggcctggtcggcgccaggctggcgctgtgggggcattacaacgattacctgcgcaccaccttcaaccgcgcgcgctgggtggacagcgaccccacccgccgcgacgcggcgcagatctacgccgggcagttcacgatcagcccggccggcgccggaccgggcacgccctggctgttcaccgccgacatcgacgacagccacggcgcgcgctggacgcgcggcggccacatcgccgagcgcggcggccatttcctggacgaggagttcggcctggcgcggctgttccagttctcggtgcccaaagaccatccgcacttcctgttccacccggggccattcgattccgaagcctggcgcaggctgcagctggcgctggaggacgacgacgtgctcggcctgacggtgcagtacgcgctgttcaatatgtcgacgccgccgcaacccaactcgccggtgttccacgacatggtcggcgtggtcggcctgtggcggcgcggcgaactggccagctacccggccggccggctgctgcgtccgcgccagcccgggctgggcgatctgacgctgcgcgtaagcggcggccgcgtggcgctgaatctggcctgcgccattccgttctccacccgggcggcgcagccgtccgcgccggacaggctgacgcccgatctcggggccaagctgccgttgggcgacctgctgctgcgcgacgaggacggcgcgttgctggcgcgggtgccgcaggcgctttaccaggattactggacgaaccacggcatcgtcgacctgccgctgctgcgcgagcccaggggctcgctgacgctgtccagcgagctggccgaatggcgcgagcaggactgggtcacgcagtccgacgcctccaatctttatttggaagcgccggaccgccgccacggccgtttctttccggaaagcatcgcgctgcgcagctatttccgcggcgaggcccgcgcgcgcccggacattccccaccggatcgaggggatgggtctggtcggcgtggagtcgcgccaggacggcgatgccgccgaatggcggctgaccggcctgcggcccggcccggcgcgcatcgtgctcgacgacggcgcggaggcgatcccgctgcgggtgctgccggacgactgggcgttggacgacgcgacggtggaggaggtcgattacgccttcctgtaccggcacgtgatggcctattacgagctggtctacccgttcatgtccgacaaggtgttcagcctggccgaccgctgcaagtgcgagacctacgccaggctgatgtggcagatgtgcgatccgcagaaccggaacaagagctactacatgcccagcacccgcgagctgtcggcgcccaaggccaggctgttcctcaaatacctggcccatgtcgagggccaggccaggctgcaggcgccgccgccggccgggccggcgcgcatcgagagcaaggcccagctggcggccgagctgcgcaaggcggtggatctggagttgtcggtgatgctgcagtacctgtacgccgcctattccattcccaattacgcccagggccagcagcgggtgcgcgacggcgcgtggacggcggagcagctgcagctggcctgcggcagcggcgaccggcgccgcgacggcggcatccgcgccgcgctgctggagatcgcccacgaggagatgatccattacctggtggtcaacaacctgctgatggcgctgggcgagccgttctacgccggcgtgccgctgatgggcgaggcggcgcggcaggcgttcggcctggacaccgaattcgcgctggagccgttctccgagtcgacgctggcgcgcttcgtccggctggaatggccgcacttcatccctgcgccgggcaaatccatcgccgactgctacgccgccatccgccaggcctttctcgatctgcccgacctgttcggcggcgaggccggcaagcgcggcggcgagcaccacttgttcctcaacgagctgaccaaccgcgcccatcccggctaccagctggaggtgttcgatcgcgacagcgcgctgttcggcatcgccttcgtcaccgaccagggcgagggcggggcgctggactcgccgcattacgagcattcgcatttccagcggctgcgggagatgtcggccaggatcatggcgcagtccgcgccgttcgagccggcgttgccggcgctgcgcaacccggtgctggacgagtcgccgggctgccagcgcgtggcggacggacgggcgcgcgcgctgatggcgctgtaccagggcgtgtacgagctgatgttcgcgatgatggcgcagcacttcgcggtcaagccgctgggcagcctcaggcgctcgcggctgatgaacgcggcgatcgacctgatgaccggcctgctcaggccgctgtcctgcgcgctgatgaacctgccgtcgggcatcgccggacgcaccgccgggccgccgctgccggggccggtggatacccgcagctacgacgactacgcgctgggctgccggatgctggcgcggcgctgcgagcgcctgctggagcaggcgtcgatgctggagccgggctggctgcccgacgcgcaaatggaactgctggatttctaccgccggcagatgctggatttggcttgtggaaagctttctagagaggcctga

SEQ ID NO: 54 - protein, 998 aa, Chromobacterium violaceum
MSILDFPRIHFRGWARVNAPTANRDPHGHIDMASNTVAMAGEPFDLARHPTEFHRHLRSLGPRFGLDGRADPEGPFSLAEGYNAAGNNHFSWESATVSHVQWDGGEADRGDGLVGARLALWGHYNDYLRTTFNRARWVDSDPTRRDAAQIYAGQFTISPAGAGPGTPWLFTADIDDSHGARWTRGGHIAERGGHFLDEEFGLARLFQFSVPKDHPHFLFHPGPFDSEAWRRLQLALEDDDVLGLTVQYALFNMSTPPQPNSPVFHDMVGVVGLWRRGELASYPAGRLLRPRQPGLGDLTLRVSGGRVALNLACAIPFSTRAAQPSAPDRLTPDLGAKLPLGDLLLRDEDGALLARVPQALYQDYWTNHGIVDLPLLREPRGSLTLSSELAEWREQDWVTQSDASNLYLEAPDRRHGRFFPESIALRSYFRGEARARPDIPHRIEGMGLVGVESRQDGDAAEWRLTGLRPGPARIVLDDGAEAIPLRVLPDDWALDDATVEEVDYAFLYRHVMAYYELVYPFMSDKVFSLADRCKCETYARLMWQMCDPQNRNKSYYMPSTRELSAPKARLFLKYLAHVEGQARLQAPPPAGPARIESKAQLAAELRKAVDLELSVMLQYLYAAYSIPNYAQGQQRVRDGAWTAEQLQLACGSGDRRRDGGIRAALLEIAHEEMIHYLVVNNLLMALGEPFYAGVPLMGEAARQAFGLDTEFALEPFSESTLARFVRLEWPHFIPAPGKSIADCYAAIRQAFLDLPDLFGGEAGKRGGEHHLFLNELTNRAHPGYQLEVFDRDSALFGIAFVTDQGEGGALDSPHYEHSHFQRLREMSARIMAQSAPFEPALPALRNPVLDESPGCQRVADGRARALMALYQGVYELMFAMMAQHFAVKPLGSLRRSRLMNAAIDLMTGLLRPLSCALMNLPSGIAGRTAGPPLPGPVDTRSYDDYALGCRMLARRCERLLEQASMLEPGWLPDAQMELLDFYRRQMLDLACGKLSREA

SEQ ID NO: 55 - DNA, 1290 bp, Chromobacterium violaceum
atgaaaagagcaatcatagtcggaggcgggctcgccggcgggctgaccgccatctacctggcgaagcgcggctacgaggtccacgtggtggaaaagcgcggcgacccgctgcgggacctgtcttcctacgtggatgtggtcagctcgcgggcgataggcgtcagcatgaccgtgcgcggcatcaagtcggtgctggcggccggcattccgcgcgcggagctggacgcctgcggcgaacccatcgtggcgatggcgttttccgtcggcggccagtaccggatgcgggagctcaagccgctggaggatttccgcccgctgtcgctgaaccgcgcggcgtttcagaagctgctgaacaagtacgccaacctggccggcgtccgctactacttcgagcacaagtgcctggacgtggatctggacggcaagtcggtgctgatccagggcaaggacggccagccgcagcgcttgcagggcgatatgatcatcggcgccgacggcgcgcactcggcggtgcggcaggcgatgcagagcgggttgcgccgcttcgaattccagcagactttcttccgccacggctacaagacgctggtgctgccggacgcgcaggcgctgggctaccgcaaggacacgctgtatttcttcggcatggactccggcggcctgttcgccggccgcgccgccaccatcccggacggcagcgtcagcatcgcggtctgcctgccgtacagcggcagccccagcctgaccaccaccgacgagccgacgatgcgcgcctttttcgaccgttacttcggcggcctgccgcgggacgcgcgcgacgagatgctgcgccagttcctggccaagcccagcaacgacctgatcaacgtccgttccagcaccttccactacaagggcaatgtgctgctgctgggcgacgccgcccacgccaccgcgcctttcctcggccagggcatgaacatggcgctggaggacgcgcgcaccttcgtcgagctgctggaccgccaccagggcgaccaggacaaggcctttcccgagttcaccgagctgcgcaaggtgcaggccgacgcgatgcaggacatggcgcgcgccaactacgacgtgctcagctgctccaatcccatcttcttcatgcgggcccgctacacccgctacatgcatagcaagtttcccggcctttacccgccggacatggcggagaagctgtacttcacgtccgagccgtacgacagactgcagcagatccagagaaaacagaacgtttggtacaagatagggagggtcaactga

SEQ ID NO: 56 - protein, 429 aa, Chromobacterium violaceum
MKRAIIVGGGLAGGLTAIYLAKRGYEVHVVEKRGDPLRDLSSYVDVVSSRAIGVSMTVRGIKSVLAAGIPRAELDACGEPIVAMAFSVGGQYRMRELKPLEDFRPLSLNRAAFQKLLNKYANLAGVRYYFEHKCLDVDLDGKSVLIQGKDGQPQRLQGDMIIGADGAHSAVRQAMQSGLRRFEFQQTFFRHGYKTLVLPDAQALGYRKDTLYFFGMDSGGLFAGRAATIPDGSVSIAVCLPYSGSPSLTTTDEPTMRAFFDRYFGGLPRDARDEMLRQFLAKPSNDLINVRSSTFHYKGNVLLLGDAAHATAPFLGQGMNMALEDARTFVELLDRHQGDQDKAFPEFTELRKVQADAMQDMARANYDVLSCSNPIFFMRARYTRYMHSKFPGLYPPDMAEKLYFTSEPYDRLQQIQRKQNVWYKIGRVN

SEQ ID NO: 57 - DNA, 1122 bp, Chromobacterium violaceum
atgaagattctggtcatcggcgcggggccggccggcctggtgttcgccagccaactgaaacaggcgcgtccgctgtgggcgatagacatcgtcgaaaagaacgacgagcaggaagtgctgggctggggcgtggtgctgcccggccggcccggccagcatccggccaatccgctgtcctacctggacgcgccggagaggctgaatccgcagttcctggaagacttcaagctggtccaccacaacgagcccagcctgatgagcaccggcgtgctgctgtgcggcgtggagcgccgcggcctggtgcacgccttgcgcgacaagtgccgctcgcagggcatcgccatccgcttcgaatcgccgctgctggagcatggcgagctgccgctggccgactacgacctggtggtgctggccaacggcgtcaatcacaagaccgcccacttcaccgaggcgctggtgccgcaggtggactacggccgcaacaagtacatctggtacggcaccagccagctgttcgaccagatgaacctggtgttccgcacccacggcaaggacattttcatcgcccacgcctacaagtactcggacacgatgagcaccttcatcgtcgagtgcagcgaggagacctatgcccgcgcccgcctgggcgagatgtcggaagaggcgtcggccgaatacgtcgccaaggtgttccaggccgagctgggcggccacggcctggtgagccagcccggcctcggctggcgcaacttcatgaccctgagccacgaccgctgccacgacggcaagctggtgctgctgggcgacgcgctgcagtccggccacttctccatcggccacggcaccacgatggcggtggtggtggcgcagctgctggtgaaggcgctgtgcaccgaggacggcgtgccggccgcgctgaagcgcttcgaggagcgcgcgctgccgctggtccagctgttccgcggccatgccgacaacagccgggtctggttcgagacggtggaggagcgcatgcacctgtccagcgccgagttcgtgcagagcttcgacgcgcgccgcaagtcgctgccgccgatgccggaagcgctggcgcagaacctgcgctacgcgctgcaacgctga

SEQ ID NO: 58 - protein, 373 aa, Chromobacterium violaceum
MKILVIGAGPAGLVFASQLKQARPLWAIDIVEKNDEQEVLGWGVVLPGRPGQHPANPLSYLDAPERLNPQFLEDFKLVHHNEPSLMSTGVLLCGVERRGLVHALRDKCRSQGIAIRFESPLLEHGELPLADYDLVVLANGVNHKTAHFTEALVPQVDYGRNKYIWYGTSQLFDQMNLVFRTHGKDIFIAHAYKYSDTMSTFIVECSEETYARARLGEMSEEASAEYVAKVFQAELGGHGLVSQPGLGWRNFMTLSHDRCHDGKLVLLGDALQSGHFSIGHGTTMAVVVAQLLVKALCTEDGVPAALKRFEERALPLVQLFRGHADNSRVWFETVEERMHLSSAEFVQSFDARRKSLPPMPEALAQNLRYALQR

SEQ ID NO: 59 - DNA, 576 bp, Chromobacterium violaceum
atggaaaaccgggaaccgccgctgctgccggcgcgctggagcagcgcctatgtgtcgtactggagtccgatgctgccggatgaccagctgacgtccggctactgctggttcgactacgagcgcgacatctgtcggatagacggcctgttcaatccctggtcggagcgcgacaccggctaccggctgtggatgtccgaggtcggcaacgccgccagcggccgcacctggaagcagaaggtggcctatggccgcgagcggaccgccctgggcgagcagctgtgcgagcggccgctggacgacgagaccggcccgttcgccgagctgttcctgccgcgcgacgtgctgcgccggctgggcgcccgccatatcggccgccgcgtggtgctgggcagggaagccgacggctggcgctaccagcgtccgggcaaggggccgtccacgttgtacctggacgccgccagcggtacgccgctgaggatggtgaccggggacgaggcgtcgcgcgcgtcgctgcgcgatttccccaacgtcagcgaggccgagattcccgacgccgtcttcgccgccaagcgctag

SEQ ID NO: 60 - protein, 191 aa, Chromobacterium violaceum
MENREPPLLPARWSSAYVSYWSPMLPDDQLTSGYCWFDYERDICRIDGLFNPWSERDTGYRLWMSEVGNAASGRTWKQKVAYGRERTALGEQLCERPLDDETGPFAELFLPRDVLRRLGARHIGRRVVLGREADGWRYQRPGKGPSTLYLDAASGTPLRMVTGDEASRASLRDFPNVSEAEIPDAVFAAKR

SEQ ID NO: 61 - DNA, 909 bp, Pantoea ananatis
atgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggccctagggcggttgaagaacgtctgagacaacatcttcatcttgccagtgagcatctctctgcggcctgccaacacgggcacgccactcaacattttattcaggcctggtttgacaaaaaactcgctgccgtcagttaa

SEQ ID NO: 62 - protein, 302 aa, Pantoea ananatis
MTVCAKKHVHLTRDAAEQLLADIDRRLDQLLPVEGERDVVGAAMREGALAPGKRIRPMLLLLTARDLGCAVSHDGLLDLACAVEMVHAASLILDDMPCMDDAKLRRGRPTIHSHYGEHVAILAAVALLSKAFGVIADADGLTPLAKNRAVSELSNAIGMQGLVQGQFKDLSEGDKPRSAEAILMTNHFKTSTLFCASMQMASIVANASSEARDCLHRFSLDLGQAFQLLDDLTDGMTDTGKDSNQDAGKSTLVNLLGPRAVEERLRQHLHLASEHLSAACQHGHATQHFIQAWFDKKLAAVS

SEQ ID NO: 63 - DNA, 930 bp, Pantoea ananatis
ttgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgaccagacgctgggcttccaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggacgatacgctgcgctattgctatcacgttgcaggcgttgtcggcttgatgatggcgcaaatcatgggcgtacgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagcgggcctggctgggttgcccctgcgttcggcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctag

SEQ ID NO: 64 - protein, 309 aa, Pantoea ananatis
MNNPSLLNHAVETMAVGSKSFATASKLFDAKTRRSVLMLYAWCRHCDDVIDDQTLGFQARQPALQTPEQRLMQLEMKTRQAYAGSQMHEPAFAAFQEVAMAHDIAPAYAFDHLEGFAMDVREAQYSQLDDTLRYCYHVAGVVGLMMAQIMGVRDNATLDRACDLGLAFQLTNIARDIVDDAHAGRCYLPASWLEHEGLNKENYAAPENRQALSRIARRLVQEAEPYYLSATAGLAGLPLRSAWAIATAKQVYRKIGVKVEQAGQQAWDQRQSTTTPEKLTLLLAASGQALTSRMRAHPPRPAHLWQRPL

SEQ ID NO: 65 - DNA, 1479 bp, Pantoea ananatis
atgaaaccaactacggtaattggtgcaggcttcggtggcctggcactggcaattcgtctacaggctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggtgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgtatgagtaactctctgtttgtgctctattttggtttgaatcaccatcatgatcagctcgcgcatcacacggtttgtttcggcccgcgttaccgcgaactgattgacgagatttttaatcatgatggcctcgcagaagacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgagtaccttgagcagcattacatgcctggcttacggagtcagctggtcacgcaccagatgtttacgccgtttgattttcgcgaccagcttaatgcctatcagggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttga

SEQ ID NO: 66 - protein, 492 aa, Pantoea ananatis
MKPTTVIGAGFGGLALAIRLQAAGIPVLLLEQRDKPGGRAYVYEDQGFTFDAGPTVITDPSAIEELFALAGKQLKEYVELLPVTPFYRLCWESGKVFNYDNDQTRLEAQIQQFNPRDVEGYRQFLDYSRAVFKEGYLKLGTVPFLSFRDMLRAAPQLAKLQAWRSVYSKVASYIEDEHLRQAFSFHSLLVGGNPFATSSIYTLIHALEREWGVWFPRGGTGALVQGMIKLFQDLGGEVVLNARVSHMETTGNKIEAVHLEDGRRFLTQAVASNADVVHTYRDLLSQHPAAVKQSNKLQTKRMSNSLFVLYFGLNHHHDQLAHHTVCFGPRYRELIDEIFNHDGLAEDFSLYLHAPCVTDSSLAPEGCGSYYVLAPVPHLGTANLDWTVEGPKLRDRIFEYLEQHYMPGLRSQLVTHQMFTPFDFRDQLNAYQGSAFSVEPVLTQSAWFRPHNRDKTITNLYLVGAGTHPGAGIPGVIGSAKATAGLMLEDLI

SEQ ID NO: 67 - DNA, 1149 bp, Pantoea ananatis
atgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagcttcgctggtggttcatcactggcccgactatcaggtacgctttcccacacgccgtcgtaagctgaacagcggctacttctgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaactcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatatcgataatgcgacattagatcctgaacgcgcgcggcaaaatatttgcgactatgccgcgcaacagggttggcagcttcagacattgctgcgtgaagaacagggcgccttacccatcaccctgtcgggcaatgccgaggcattctggcagcagcgccccctggcctgtagtggattacgtgccggtctgttccatcctaccaccggctattcactgccgctggcggttgccgtggccgaccgcctgagcgcacttgatgtctttacgtcggcctcaattcaccaggctattaggcattttgcccgcgagcgctggcagcagcagcgctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaa

SEQ ID NO: 68 - protein, 382 aa, Pantoea ananatis
MQPHYDLILVGAGLANGLIALRLQQQQPDMRILLIDAAPQAGGNHTWSFHHDDLTESQHRWIASLVVHHWPDYQVRFPTRRRKLNSGYFCITSQRFAEVLQRQFGPHLWMDTAVAEVNAESVRLKKGQVIGARAVIDGRGYAANSALSVGFQAFIGQEWRLSHPHGLSSPIIMDATVDQQNGYRFVYSLPLSPTRLLIEDTHYIDNATLDPERARQNICDYAAQQGWQLQTLLREEQGALPITLSGNAEAFWQQRPLACSGLRAGLFHPTTGYSLPLAVAVADRLSALDVFTSASIHQAIRHFARERWQQQRFFRMLNRMLFLAGPADSRWRVMQRFYGLPEDLIARFYAGKLTLTDRLRILSGKPPVPVLAALQAIMTTHR

SEQ ID NO: 69 - DNA, 209 bp, Cupriavidus necator
gcctcgctgctttccacctggcgccgcacgcggccccagacgtcgatttcccaggttgcgcccagggtcgcgctctgcccgttgagcgtgctgccgctggcgccgcgcgcgcgcgaggcgccggcctgtgcgtcgacggtcgggaagaagccggcgcgcgcggcctgcagcgacgccaccgcctggcggtactgcgcctcggcggcctt

SEQ ID NO: 70 - DNA, 1495 bp, Cupriavidus necator
aggcgccggcctgtgcgtcgacggtcgggaagaagccggcgcgcgcggcctgcagcgacgccaccgcctggcggtactgcgcctcggcggccttgatgttctggttcgagatctgcacctcggacatcagcgcgtcgagctgcgcatcgccgaacacggtccaccagtcggcgcgtgccagcgcatcctgcggctcggcgggcttccagtcgccggtccaggcgggggtggcggcatcggcttccttgaaggatgcggaaaccggcgcgtcggggcgctggtagtcggggccgacggcgcagccggccagcagcagcgcgcaggccagcgacaccggcagggcatgggtcaggaggcgggaaagaactgtcatgtcgagtcttcgcaaatctagacggcggccggctggtcaggcgtgccggcaccacggcggcgctggcgccaggccttgaccttcaggcgccagcggtccagcgtcaggtagaccaccggcgtggtgtacagcgtcagcagctggcttaccaccagtccgccgacaatggagatgcccagcggcgcgcgcagttcggcgccgtcgccgcggccgattgccagcggcaccgcgcccagcagcgcggccatggtggtcatcaggatcgggcggaagcgcagcaggcaggcgcggtagatcgcgtcgcgcggcgacaggccatcgcgccgttcggcatcgatggcgaagtcgatcatcatgatcgcgttctttttcacgatgccgatcagcaggatcacgccgatcagcgcgatgatgctgaagtcggtcttcgatgccagcagcgccagcagcgcgcccacgccggcggagggcagcgtcgacaggatcgtcagcggatgcacatagctttcatacagcacgcccagcacgatgtagatcgtgatcagcgccgccaggatcaggatcggctgactcttgagcgaatcctggaacgccttggcgccgccctggaagttggcgcgcagcgtctccggcacgccgatgcgcgccatctcgcgcgtgatcgcgtcggtcgcctgcgacagcgaagtgccctcggccaggttgaacgagatcgtcgaggccgcgaactggccctggtggttcacgcccagcggcgtgctggacggggtcacgcgcgcgaacgccgccagcggcacgcggttgccgttgccggtgaccacgtagatgtccttgagcgcatcgggcccttgcaggtattcctggctcagctccatcaccacgcggtactggttcagcggatggtagatggtggacaccagccgctggccgaaggcatcgttgagcaccgcatccacctgctgcgcggtcacgcccaggcgcgaggccgcgtcgcggtcgatgatcaccgaggtctgcaggcccttgtcgttggtatcggtgtcgatatcctccagccccttcaggttcgacaacgcggcgcgcaccttgggctcccacgcgcgcagcacttccaggtcgtcc

SEQ ID NO: 71 - DNA, 201 bp, Cupriavidus necator
gcagcttgatgccattgacgaggtagatggaaaccggcacgtgctctttgcgcagcgcgttcaggaacgggccttgtagcagttgccctttgttgctcatggcacactccaaatttataggtttagtggtgaatgatggggatggaaatccccggttcaagtcaggcggcgcaaaaacgcgccagaaaaaagatcaaaaac

SEQ ID NO: 72 - DNA, 479 bp, Cupriavidus necator
gaggatgccatgtccgaagcgcctgtccttgccccctcgacctcaacccagccgcccgccgccggccagctcaacctgatccgcccgcagccatatgccgactgggcgccgcaggtcacggccgaagaacgcgccacgctgcgccgcgagctggagcagggcgccgtgctgtacttcccgaacctgaatttccgcttccagccgggcgaagagcgcttccttgacagccgctattccgacggcaagtccaagaacatcaacctgcgcgccgacgacaccgcggtgcgcggcgcccagggcagtccgcaggacctggcggacctgtacacgctgatccgccgctacgccgacaacagcgaattgctggtgcgcacgctgttccctgaatacatcccgcacatgacgcgcgccggcacctcgctgcggcccagcgagatcgccgggcgcccggtcagctggcgcaaggacgacacccgcct

SEQ ID NO: 73 - DNA, 2202 bp, Anabaena cylindrica
atgtcaaacagccaagggttgtacattttgctggttagtgttcacggtttaattagaggtcataatctagaattaggaagagatgccgacactggtggacaaacaaaatatgcagtcgaacttgccaccacattagccaaaaatcctcaagtagaaagagtggatttagttactcggttggtgaatgatccaaaagttagtcctgactatgctcaaccaatagaaattctctcagataaagctcagatcattcgtcttgcttgcgggccacgtcgctatctccgcaaagaagttctctggcagcatttagatacctttgcagatgaattgctcagacacattcgtaaagttggcagaataccaaatgtaattcatacacactacgctgatgcaggatatgttggcagtcgggttgcaggttggttaggaacacctcttgtacatactggtcactccctgggacgggttaaacagcaaaaattattggaacagggaactaaacaggaagtgattgaagatcattttcatattagtacaagaattgaagcagaagaaattacacttggtggtgcagctttagttatagccagcactaatcaagaagttgagcagcagtatagtgtgtacgatcgctatcaaccagaaagaatggtggtgattcctcctggtgtagacttggatcgattttacctacctggagatgattggcacaatccaccgattcaaaaagaattggatcgatttctcaaagatccgcaaaagccaatcatcatggcaatttcccgtccagctattcgtaaaaacgtcagtagtctgattaaggcttatggtgaagatcctgagttgcggaaactggcaaacctagtcatagtcctgggcaagcgggacgacatcatgacgatggaatcggggccacgtcaggtatttatagagatattgcaattaatagatcgctacgacctctacggtcacattgcttatcctaaacaccataatgctgacgatgtgccagatttatatcggctgacagctagaacacaaggggtattcattaatcctgccttgacagaaccatttggacttaccttaattgaagcgagtgcttgcggtgtacctattattgccactgctgacggtggcccacgggatattttggcagcttgtgagaatggattgctaattgatcccttaaatattcaagaaatacaaaatgctctccgcaaagccttaacagataaggaacaatggcaaaactggtctagcaatggtttagttaatgttcgcaaatatttctcctggaatagtcatgtagagaaatatttagagaaaatacacctatttccccaacgacgaattcaatctctacttagtcctttgccggcatctcctgcgactgatcatccagagtggaatgtgccagataccaaccgtttacctaccgctgatcgtttcctagtttgtgaaattgataatacgctgctgggtgacaaagaagccctagaaaaattgattcagcgcattcgtaacgaaggacatacaactggggttggtattgccacaggtcgcactctagaaagtactttgagtatgttggaagaatggcgattcccgatgccagatttgttaattacatcagcaggtagtgaaatttactacgggccgcagatagttacagatacaagttggcaaaaacacattggttaccaatggcaagctgaagcaattcgggcagcaatgaagaatattcccggtgtagaattgcaaccagaagaagctcaacgcaagtttaaggttagctattttgttgatgaagctaaagcgcctaacttccgagaaattatccgccatttgcgtcgtcatcaactacctgtaaaagggatttacagccacaatatgtatttggatttagttcctatccgggcttctaagggcgatgcaattcgctatgtggctttgaaatggggtttacctgttcaacgtttcctggtagcaggggcatcaggtaatgatgaaaccatgcttggtggtaacactttaggggttgtggtggggaattacagccaagaaattgaaaagctgcggggttatccacagatttattttgctcaaggtaattatgcctggggtattttggaagccttggattattacgacttttttggtaatctgtctcaaaaacagccagaaatggtgacggtttag

SEQ ID NO: 74 - protein, 733 aa, Anabaena cylindrica
MSNSQGLYILLVSVHGLIRGHNLELGRDADTGGQTKYAVELATTLAKNPQVERVDLVTRLVNDPKVSPDYAQPIEILSDKAQIIRLACGPRRYLRKEVLWQHLDTFADELLRHIRKVGRIPNVIHTHYADAGYVGSRVAGWLGTPLVHTGHSLGRVKQQKLLEQGTKQEVIEDHFHISTRIEAEEITLGGAALVIASTNQEVEQQYSVYDRYQPERMVVIPPGVDLDRFYLPGDDWHNPPIQKELDRFLKDPQKPIIMAISRPAIRKNVSSLIKAYGEDPELRKLANLVIVLGKRDDIMTMESGPRQVFIEILQLIDRYDLYGHIAYPKHHNADDVPDLYRLTARTQGVFINPALTEPFGLTLIEASACGVPIIATADGGPRDILAACENGLLIDPLNIQEIQNALRKALTDKEQWQNWSSNGLVNVRKYFSWNSHVEKYLEKIHLFPQRRIQSLLSPLPASPATDHPEWNVPDTNRLPTADRFLVCEIDNTLLGDKEALEKLIQRIRNEGHTTGVGIATGRTLESTLSMLEEWRFPMPDLLITSAGSEIYYGPQIVTDTSWQKHIGYQWQAEAIRAAMKNIPGVELQPEEAQRKFKVSYFVDEAKAPNFREIIRHLRRHQLPVKGIYSHNMYLDLVPIRASKGDAIRYVALKWGLPVQRFLVAGASGNDETMLGGNTLGVVVGNYSQEIEKLRGYPQIYFAQGNYAWGILEALDYYDFFGNLSQKQPEMVTV

SEQ ID NO: 75 - DNA, 786 bp, Anabaena cylindrica
atgaaagcatttctcttcgttactgatttagatgacacgctagtaggtgataaaaaatctttaaactttttagagcaattgaacgaagaattaataaatcatcgcaagacatatggaactaaaattgtttatgccacggggcgatctctaactttataccagcaactgataaacacacaagaacttttagaacctgatgctttaattactgctgtaggaactgaaatatacttcaactctaattacaaaactcccgacttaaaatggtctagtaagttatcaattggctggaatcgtgatttggtagagaaaatagccgcaaattttgaagacttagttcttcaaaaagcatctgaacaacttccttttaaagttagttatcttgttaaggaagaagctgctaaaaaagtcataccccaactcgataatttattaaaaaaggaaaacattaaatttaatttaatctacagtggaaactttagtggcagcgaaaacctggaaagcaaaaacctggatattttgccttttgataccgataaaggtttagcaatgaagtatttacaaaatgaatggggacattctgctcacgaaacagttgtttgtggtgattcaggtaatgatattgccttattcagcagaggagaagaaagaggtattattgtaggaaatgcgctttctgaattacgggaatggtatacagcaaacaaaaccgactatcgctatttagcaaaatcattttatgctgaaggtattttagaaggtttgcgtcattttcagtttatttaa

SEQ ID NO: 76 - protein, 261 aa, Anabaena cylindrica
MKAFLFVTDLDDTLVGDKKSLNFLEQLNEELINHRKTYGTKIVYATGRSLTLYQQLINTQELLEPDALITAVGTEIYFNSNYKTPDLKWSSKLSIGWNRDLVEKIAANFEDLVLQKASEQLPFKVSYLVKEEAAKKVIPQLDNLLKKENIKFNLIYSGNFSGSENLESKNLDILPFDTDKGLAMKYLQNEWGHSAHETVVCGDSGNDIALFSRGEERGIIVGNALSELREWYTANKTDYRYLAKSFYAEGILEGLRHFQFI

SEQ ID NO: 77 - DNA, 2130 bp, Synechococcus elongatus
gtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaa

SEQ ID NO: 78 - protein, 709 aa, Synechococcus elongatus
MAAQNLYILHIQTHGLLRGQNLELGRDADTGGQTKYVLELAQAQAKSPQVQQVDIITRQITDPRVSVGYSQAIEPFAPKGRIVRLPFGPKRYLRKELLWPHLYTFADAILQYLAQQKRTPTWIQAHYADAGQVGSLLSRWLNVPLIFTGHSLGRIKLKKLLEQDWPLEEIEAQFNIQQRIDAEEMTLTHADWIVASTQQEVEEQYRVYDRYNPERKLVIPPGVDTDRFRFQPLGDRGVVLQQELSRFLRDPEKPQILCLCRPAPRKNVPALVRAFGEHPWLRKKANLVLVLGSRQDINQMDRGSRQVFQEIFHLVDRYDLYGSVAYPKQHQADDVPEFYRLAAHSGGVFVNPALTEPFGLTILEAGSCGVPVVATHDGGPQEILKHCDFGTLVDVSRPANIATALATLLSDRDLWQCYHRNGIEKVPAHYSWDQHVNTLFERMETVALPRRRAVSFVRSRKRLIDAKRLVVSDIDNTLLGDRQGLENLMTYLDQYRDHFAFGIATGRRLDSAQEVLKEWGVPSPNFWVTSVGSEIHYGTDAEPDISWEKHINRNWNPQRIRAVMAQLPFLELQPEEDQTPFKVSFFVRDRHETVLREVRQHLRRHRLRLKSIYSHQEFLDILPLAASKGDAIRHLSLRWRIPLENILVAGDSGNDEEMLKGHNLGVVVGNYSPELEPLRSYERVYFAEGHYANGILEALKHYRFFEAIA

SEQ ID NO: 79 - DNA, 1356 bp, Cupriavidus necator
ttagattgaacgctggcggcatgccttacacatgcaagtcgaacggcagcacgggcttcggcctggtggcgagtggcgaacgggtgagtaatacatcggaacgtgccctgtagtgggggataactagtcgaaagattagctaataccgcatacgacctgagggtgaaagcgggggaccgcaaggcctcgcgctacaggagcggccgatgtctgattagctagttggtggggtaaaagcctaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatggctctggttaatacccggggtcgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaatggcgcttgtgactgcaaggctagagtatgtcagaggggggtagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgcccgaaagggaaagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccggtct

SEQ ID NO: 80 - DNA, 1541 bp, Escherichia coli
aaattgaagagtttgatcatggctcagattgaacgctggcggcaggcctaacacatgcaagtcgaacggtaacaggaagaagcttgctctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 81 - DNA, 1680 bp, Pseudomonas 61-3
atgagtaacaagaatagcgatgacttgaatcgtcaagcctcggaaaacaccttggggcttaaccctgtcatcggcctgcgtggaaaagatctgctgacttctgcccgaatggttttaacccaagccatcaaacaacccattcacagcgtcaagcacgtcgcgcattttggcatcgagctgaagaacgtgatgtttggcaaatcgaagctgcaaccggaaagcgatgaccgtcgtttcaacgaccccgcctggagtcagaacccactctacaaacgttatctacaaacctacctggcgtggcgcaaggaactccacgactggatcggcaacagcaaactgtccgaacaggacatcaatcgcgctcacttcgtgatcaccctgatgaccgaagccatggccccgaccaacagtgcggccaatccggcggcggtcaaacgcttcttcgaaaccggcggtaaaagcctgctcgacggcctcacacatctggccaaggacctggtaaacaacggcggcatgccgagccaggtggacatgggcgctttcgaagtcggcaagagtctggggacgactgaaggtgcagtggttttccgcaacgacgtcctcgaattgatccagtaccggccgaccaccgaacaggtgcatgagcgaccgctgctggtggtcccaccgcagatcaacaagttttatgtgtttgacctgagcccggataaaagcctggcgcgcttctgcctgagcaacaaccagcaaacctttatcgtcagctggcgcaacccgaccaaggcccagcgtgagtggggtctgtcgacttacatcgatgcgctcaaagaagccgtcgacgtagtttccgccatcaccggcagcaaagacatcaacatgctcggcgcctgctccggtggcattacctgcaccgcgctgctgggtcactacgccgctctcggcgagaagaaggtcaatgccctgacccttttggtcagcgtgctcgacaccaccctcgactcccaggttgcactgttcgtcgatgagaaaaccctggaagctgccaagcgtcactcgtatcaggccggcgtgctggaaggccgcgacatggccaaagtcttcgcctggatgcgccctaacgacctgatctggaactactgggtcaacaactacctgctgggtaacgagccaccggtcttcgacattcttttctggaacaacgacaccacccggttgcctgctgcgttccacggcgatctgatcgaaatgttcaaaaataacccactggtgcgcgccaatgcactcgaagtgagcggcacgccgatcgacctcaaacaggtcactgccgacatctactccctggccggcaccaacgatcacatcacgccctggaagtcttgctacaagtcggcgcaactgttcggtggcaaggtcgaattcgtgctgtccagcagtgggcatatccagagcattctgaacccgccgggcaatccgaaatcacgttacatgaccagcaccgacatgccagccaccgccaacgagtggcaagaaaactcaaccaagcacaccgactcctggtggctgcactggcaggcctggcaggccgagcgctcgggcaaactgaaaaagtccccgaccagcctgggcaacaaggcctatccgtcaggagaagccgcgccgggcacgtatgtgcatgaacgttaa

SEQ ID NO: 82 - protein, 568 aa, Pseudomonas aeruginosa
MREKQESGSVPVPAEFMSAQSAIVGLRGKDLLTTVRSLAVHGLRQPLHSARHLVAFGGQLGKVLLGDTLHQPNPQDARFQDPSWRLNPFYRRTLQAYLAWQKQLLAWIDESNLDCDDRARARFLVALLSDAVAPSNSLINPLALKELFNTGGISLLNGVRHLLEDLVHNGGMPSQVNKTAFEIGRNLATTQGAVVFRNEVLELIQYKPLGERQYAKPLLIVPPQINKYYIFDLSPEKSFVQYALKNNLQVFVISWRNPDAQHREWGLSTYVEALDQAIEVSREITGSRSVNLAGACAGGLTVAALLGHLQVRRQLRKVSSVTYLVSLLDSQMESPAMLFADEQTLESSKRRSYQHGVLDGRDMAKVFAWMRPNDLIWNYWVNNYLLGRQPPAFDILYWNNDNTRLPAAFHGELLDLFKHNPLTRPGALEVSGTAVDLGKVAIDSFHVAGITDHITPWDAVYRSALLLGGQRRFILSNSGHIQSILNPPGNPKACYFENDKLSSDPRAWYYDAKREEGSWWPVWLGWLQERSGELGNPDFNLGSAAHPPLEAAPGTYVHIRGGHHHHHH

SEQ ID NO: 83 - protein, 559 aa, Pseudomonas 61-3
MSNKNSDDLNRQASENTLGLNPVIGLRGKDLLTSARMVLTQAIKQPIHSVKHVAHFGIELKNVMFGKSKLQPESDDRRFNDPAWSQNPLYKRYLQTYLAWRKELHDWIGNSKLSEQDINRAHFVITLMTEAMAPTNSAANPAAVKRFFETGGKSLLDGLTHLAKDLVNNGGMPSQVDMGAFEVGKSLGTTEGAVVFRNDVLELIQYRPTTEQVHERPLLVVPPQINKFYVFDLSPDKSLARFCLSNNQQTFIVSWRNPTKAQREWGLSTYIDALKEAVDVVSAITGSKDINMLGACSGGITCTALLGHYAALGEKKVNALTLLVSVLDTTLDSQVALFVDEKTLEAAKRHSYQAGVLEGRDMAKVFAWMRPNDLIWNYWVNNYLLGNEPPVFDILFWNNDTTRLPAAFHGDLIEMFKNNPLVRANALEVSGTPIDLKQVTADIYSLAGTNDHITPWKSCYKSAQLFGGKVEFVLSSSGHIQSILNPPGNPKSRYMTSTDMPATANEWQENSTKHTDSWWLHWQAWQAERSGKLKKSPTSLGNKAYPSGEAAPGTYVHER

SEQ ID NO: 84 - DNA, 2761 bp, Bradyrhizobium japonicum
atgaacatcgccgtctcccctacagcagaaggctcatcgggcagagcacaagtccagtggtcgctgagatgggaatcggaactgcagctggccgatcatgcagaactggccgagttcttccgcaagtcgtatggccctacaggcgccttcaatgcacagccgtttgaaagatcgcgctcgtgggcaggcgcaagacctgaactgagagtcatcggctatgatgcccgtggtgttgcagcacatatcggcctgctgcgccgcttcatcaaggttggcgaagtcgatttaccggtcgccgaactgggcctttatgccgtcagaccggacctggaaggtcatggtatcggccatgcaatgcgtgtcatgtatccggccctgcaggaattaggcgtcccgttcggtttcggtgcagtcagatcggcactggagaagcatctgacccgcctggtcgaaagacaaggccttgcaacccttatgcgcggcatcagagtccgctcgacacttcctgacgtctacccgaatctttcgccgacccgcatcgaagacgtcatcgtcgtcgtcttccctgtcggtcgctcaatctcggaatggcctgcaggtaccgtcatcgatcgcaatggcccggagctgtgaccgaacgttcaaccctctcaaccgtccgctgcgattatgccgatgcaggtggctcaagagccgtccatctgaccttcgatgatggcccgaatccgttctgtaccccggaagtcctggacgtccttgcccaacatagagtccctgcaaccttcttcgtcatcggcacctatgccaccgagcatccggagcttatccgccgcatgatcgcagaaggccatgaagtcgccaaccacacaatgacccatccggacctttcgagatgtggcccgaccgagctgcatgacgaagtcctgaccgcatcagaagccatcagacttgcatgtccgttagcatcgccgcgccacatgagagccccgtatggcatctggacccgtgatgtcttagcagttgcagcatcagcaggcctgaccgccttacactggtcggtcgatcctcgcgattggtcgagacctggtgttgatgcaatcgtcaactcggtcctggcgaatgtcagacctggcgccatcgtcctgctgcatgacggctatcctcctgatgaagaaggccttccttcgggttcgacccttcgcgatcaaacccgcaccgcactggcatacctgattcctgcacttcaaagacgcggcttcgtcatccgccctttaccgcagctgcactgaaaaaacgacaccccatggatctgcttgctaccacctctgcagcagcagtttcgtcatatgcactgttatcgaccatctacaagtcagtccaagccctgtatgcacaaccggccatcaactcgtcgctggataatttaggccaagcagaagttgttgttcctgccgttgatgtcatcgtcccgtgcttcaacgagaaccctaatacccttgcagaatgcttagaatcgatcgcctcgcaggattatgccggcaagatgcaggtctatgtcgtcgatgatggttcggcaaatagagatgtcgttgcacctgttcatcgcatctatgcctcggatccgagattctcgttcatcctgcttgccaataatgtcggcaaaagaaaggcccagattgcagcaatccgctcatcgtcaggcgatttagtcctgaacgttgattcggataccatcttagccgcagatgttgtcaccaagctggtcctgaagatgcatgatcctggtattggtgcagcaatgggtcagcttatcgcatcaaaccgcaatcagacctggcttaccagactgatcgatatggagtactggcttgcctgcaacgaagaaagagcagcacaagcgagatttggcgcagtcatgtgttgttgtggtccttgtgccatgtatagaagatcggccttagcactgctgttagaccagtacgaagcccagttcttcagaggtaagccgtcagattttggcgaagatcgccatctgaccatcctgatgttaaaggccggctttagaaccgagtatgtccctgatgcaattgcagcaaccgtcgtccctcattcgttaagaccgtatctgagacagcagttaagatgggcaagatcaaccttccgcgacaccttccttgcctggagacttttaccggaattagacggctatttaaccctggatgtcatcggccagaatcttggtccgctgctgcttgcaatctcatcattagccgcattagcacaactgctgattgatggctcaattccttggtggaccggtctgaccattgcagcaatgaccactgtcagatgttgtgttgcagcattaagagccagagaactgcgcttcatcggtttctcgctgcataccccgatcaatatctgtcttcttcttccgcttaaggcatatgccctgtgtaccctgtcgaactcggattggctttcacgcaaagttaccgatatgcctaccgaagaaggcaagcaacctgtcatccttcacccgaatgctggtagatcacctgcaggtgttggtggtagacttcttcttttcgtcagacgcagatatcgctcgctgcatagagcatggcgtagaagacgcgtttttcctgttgcaatcgtcagactttcgaccaacaagtggtcggcagatgattcgggcagaaagccttcagtcattagagcaagagtcggttgcagaagacctgttgcaccgcgtcattaa

SEQ ID NO: 85 - DNA, 1458 bp, Bradyrhizobium japonicum
atggatctgcttgctaccacctctgcagcagcagtttcgtcatatgcactgttatcgaccatctacaagtcagtccaagccctgtatgcacaaccggccatcaactcgtcgctggataatttaggccaagcagaagttgttgttcctgccgttgatgtcatcgtcccgtgcttcaacgagaaccctaatacccttgcagaatgcttagaatcgatcgcctcgcaggattatgccggcaagatgcaggtctatgtcgtcgatgatggttcggcaaatagagatgtcgttgcacctgttcatcgcatctatgcctcggatccgagattctcgttcatcctgcttgccaataatgtcggcaaaagaaaggcccagattgcagcaatccgctcatcgtcaggcgatttagtcctgaacgttgattcggataccatcttagccgcagatgttgtcaccaagctggtcctgaagatgcatgatcctggtattggtgcagcaatgggtcagcttatcgcatcaaaccgcaatcagacctggcttaccagactgatcgatatggagtactggcttgcctgcaacgaagaaagagcagcacaagcgagatttggcgcagtcatgtgttgttgtggtccttgtgccatgtatagaagatcggccttagcactgctgttagaccagtacgaagcccagttcttcagaggtaagccgtcagattttggcgaagatcgccatctgaccatcctgatgttaaaggccggctttagaaccgagtatgtccctgatgcaattgcagcaaccgtcgtccctcattcgttaagaccgtatctgagacagcagttaagatgggcaagatcaaccttccgcgacaccttccttgcctggagacttttaccggaattagacggctatttaaccctggatgtcatcggccagaatcttggtccgctgctgcttgcaatctcatcattagccgcattagcacaactgctgattgatggctcaattccttggtggaccggtctgaccattgcagcaatgaccactgtcagatgttgtgttgcagcattaagagccagagaactgcgcttcatcggtttctcgctgcataccccgatcaatatctgtcttcttcttccgcttaaggcatatgccctgtgtaccctgtcgaactcggattggctttcacgcaaagttaccgatatgcctaccgaagaaggcaagcaacctgtcatccttcacccgaatgctggtagatcacctgcaggtgttggtggtagacttcttcttttcgtcagacgcagatatcgctcgctgcatagagcatggcgtagaagacgcgtttttcctgttgcaatcgtcagactttcgaccaacaagtggtcggcagatgattcgggcagaaagccttcagtcattagagcaagagtcggttgcagaagacctgttgcaccgcgtcattaa

SEQ ID NO: 86 - DNA, 660 bp, Bradyrhizobium japonicum
gtgaccgaacgttcaaccctctcaaccgtccgctgcgattatgccgatgcaggtggctcaagagccgtccatctgaccttcgatgatggcccgaatccgttctgtaccccggaagtcctggacgtccttgcccaacatagagtccctgcaaccttcttcgtcatcggcacctatgccaccgagcatccggagcttatccgccgcatgatcgcagaaggccatgaagtcgccaaccacacaatgacccatccggacctttcgagatgtggcccgaccgagctgcatgacgaagtcctgaccgcatcagaagccatcagacttgcatgtccgttagcatcgccgcgccacatgagagccccgtatggcatctggacccgtgatgtcttagcagttgcagcatcagcaggcctgaccgccttacactggtcggtcgatcctcgcgattggtcgagacctggtgttgatgcaatcgtcaactcggtcctggcgaatgtcagacctggcgccatcgtcctgctgcatgacggctatcctcctgatgaagaaggccttccttcgggttcgacccttcgcgatcaaacccgcaccgcactggcatacctgattcctgcacttcaaagacgcggcttcgtcatccgccctttaccgcagctgcactga

SEQ ID NO: 87 - DNA, 633 bp, Bradyrhizobium japonicum
atgaacatcgccgtctcccctacagcagaaggctcatcgggcagagcacaagtccagtggtcgctgagatgggaatcggaactgcagctggccgatcatgcagaactggccgagttcttccgcaagtcgtatggccctacaggcgccttcaatgcacagccgtttgaaagatcgcgctcgtgggcaggcgcaagacctgaactgagagtcatcggctatgatgcccgtggtgttgcagcacatatcggcctgctgcgccgcttcatcaaggttggcgaagtcgatttaccggtcgccgaactgggcctttatgccgtcagaccggacctggaaggtcatggtatcggccatgcaatgcgtgtcatgtatccggccctgcaggaattaggcgtcccgttcggtttcggtgcagtcagatcggcactggagaagcatctgacccgcctggtcgaaagacaaggccttgcaacccttatgcgcggcatcagagtccgctcgacacttcctgacgtctacccgaatctttcgccgacccgcatcgaagacgtcatcgtcgtcgtcttccctgtcggtcgctcaatctcggaatggcctgcaggtaccgtcatcgatcgcaatggcccggagctgtga

SEQ ID NO: 88 - protein, 730 aa, Synechocystis PCC6803
MHHHHHHGGGSSYSSKYILLISVHGLIRGENLELGRDADTGGQTKYVLELARALVKNPQVARVDLLTRLIKDPKVDADYAQPRELIGDRAQIVRIECGPEEYIAKEMLWDYLDNFADHALDYLKEQPELPDVIHSHYADAGYVGTRLSHQLGIPLVHTGHSLGRSKRTRLLLSGIKADEIESRYNMARRINAEEETLGSAARVITSTHQEIAEQYAQYDYYQPDQMLVIPPGTDLEKFYPPKGNEWETPIVQELQRFLRHPRKPIILALSRPDPRKNIHKLIAAYGQSPQLQAQANLVIVAGNRDDITDLDQGPREVLTDLLLTIDRYDLYGKVAYPKQNQAEDVYALFRLTALSQGVFINPALTEPFGLTLIEAAACGVPIVATEDGGPVDIIKNCQNGYLINPLDEVDIADKLLKVLNDKQQWQFLSESGLEGVKRHYSWPSHVESYLEAINALTQQTSVLKRSDLKRRRTLYYNGALVTSLDQNLLGALQGGLPGDRQTLDELLEVLYQHRKNVGFCIATGRRLDSVLKILREYRIPQPDMLITSMGTEIYSSPDLIPDQSWRNHIDYLWNRNAIVRILGELPGLALQPKEELSAYKISYFYDAAIAPNLEEIRQLLHKGEQTVNTIISFGQFLDILPIRASKGYAVRWLSQQWNIPLEHVFTAGGSGADEDMMRGNTLSVVVANRHHEELSNLGEIEPIYFSEKRYAAGILDGLAHYRFFELLDPV

SEQ ID NO: 89 - protein, 254 aa, Synechocystis PCC6803
MHHHHHHGGGSRQLLLISDLDNTWVGDQQALEHLQEYLGDRRGNFYLAYATGRSYHSARELQKQVGLMEPDYWLTAVGSEIYHPEGLDQHWADYLSEHWQRDILQAIADGFEALKPQSPLEQNPWKISYHLDPQACPTVIDQLTEMLKETGIPVQVIFSSGKDVDLLPQRSNKGNATQYLQQHLAMEPSQTLVCGDSGNDIGLFETSARGVIVRNAQPELLHWYDQWGDSRHYRAQSSHAGAILEAIAHFDFLS

SEQ ID NO: 90 - protein, 505 aa, Escherichia coli
MYRKSTLAMLIALLTSAASAHAQTDISTIEARLNALEKRLQEAENRAQTAENRAGAAEKKVQQLTAQQQKNQNSTQEVAQRTARLEKKADDKSGFEFHGYARSGVIMNDSGASTKSGAYITPAGETGGAIGRLGNQADTYVEMNLEHKQTLDNGATTRFKVMVADGQTSYNDWTASTSDLNVRQAFVELGNLPTFAGPFKGSTLWAGKRFDRDNFDIHWIDSDVVFLAGTGGGIYDVKWNDGLRSNFSLYGRNFGDIDDSSNSVQNYILTMNHFAGPLQMMVSGLRAKDNDERKDSNGNLVKGDAANTGVHALLGLHNDSFYGLRDGSSKTALLYGHGLGAEVKGIGSDGALRPGADTWRIASYGTTPLSENWSVAPAMLAQRSKDRYADGDSYQWATFNLRLIQAINQNFALAYEGSYQYMDLKPEGYNDRQAVNGSFYKLTFAPTFKVGSIGDFFSRPEIRFYTSWMDWSKKLNNYASDDALGSDGFNSGGEWSFGVQMEAWF

SEQ ID NO: 91 - DNA, 1537 bp, Cupriavidus necator
agattgaactgaagagtttgatcctggctcagattgaacgctggcggcatgccttacacatgcaagtcgaacggcagcacgggcttcggcctggtggcgagtggcgaacgggtgagtaatacatcggaacgtgccctgtagtgggggataactagtcgaaagattagctaataccgcatacgacctgagggtgaaagcgggggaccgcaaggcctcgcgctacaggagcggccgatgtctgattagctagttggtggggtaaaagcctaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatggctctggttaatacccggggtcgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaatggcgcttgtgactgcaaggctagagtatgtcagagggggaagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgcccgaaagggaaagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacggcagggttcatgactggggtgaagtcgtaacaaggtagccgtatcggaaggtgcggctggatcacctcctttc

SEQ ID NO: 92 - DNA, 1554 bp, Escherichia coli
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagc

SEQ ID NO: 93 - DNA, 1701 bp, Escherichia coli
atggcgattgcaattggcctcgattttggcagtgattctgtgcgagctttggcggtggactgcgctaccggtgaagagatcgccaccagcgtagagtggtatccccgttggcagaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggaaacgtgctggcgctgcgcccggagtttgccgaaaacccgaacgcgatgttcgtattgtggaaagaccacactgcggttgaagaagcggaagagattacccgtttgtgccacgcgccgggcaacgttgactactcccgctacattggtggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcctgccgccagccagtttctttgatgagctggacccgatcctcaatcgccatttgccttccccgctgttcactgacacttggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggtttggtcgcgtactcggctggccgctggaacagcttgccgcccagcatccggaactgaaaacgcaaatcaacgccagccagaaacaactgcttccggcgctgaccgaagcatgggccaaaaatccgtctctggatcacctgccggtggtgctcgactggtttaacggccgccgcacaccgaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgttaataacgtgatggcactgggcggcatcgcgcggaaaaaccaggtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagctcagcaaaaaatggccagtgcggtagagaaaaccctgcaaccgtgcagcgagcaggcacaacgctttgaacagctttatcgccgctatcagcaatgggcgatgagcgccgaacaacactatcttccaacttccgccccggcacaggctgcccaggccgttgcgactctataa

SEQ ID NO: 94 - DNA, 1503 bp, Escherichia coli
atgacgatttttgataattatgaagtgtggtttgtcattggcagccagcatctgtatggcccggaaaccctgcgtcaggtcacccaacatgccgagcacgtcgttaatgcgctgaatacggaagcgaaactgccctgcaaactggtgttgaaaccgctgggcaccacgccggatgaaatcaccgctatttgccgcgacgcgaattacgacgatcgttgcgctggtctggtggtgtggctgcacaccttctccccggccaaaatgtggatcaacggcctgaccatgctcaacaaaccgttgctgcaattccacacccagttcaacgcggcgctgccgtgggacagtatcgatatggactttatgaacctgaaccagactgcacatggcggtcgcgagttcggcttcattggcgcgcgtatgcgtcagcaacatgccgtggttaccggtcactggcaggataaacaagcccatgagcgtatcggctcctggatgcgtcaggcggtctctaaacaggatacccgtcatctgaaagtctgccgatttggcgataacatgcgtgaagtggcggtcaccgatggcgataaagttgccgcacagatcaagttcggtttctccgtcaatacctgggcggttggcgatctggtgcaggtggtgaactccatcagcgacggcgatgttaacgcgctggtcgatgagtacgaaagctgctacaccatgacgcctgccacacaaatccacggcaaaaaacgacagaacgtgctggaagcggcgcgtattgagctggggatgaagcgtttcctggaacaaggtggcttccacgcgttcaccaccacctttgaagatttgcacggtctgaaacagcttcctggtctggccgtacagcgtctgatgcagcagggttacggctttgcgggcgaaggcgactggaaaactgccgccctgcttcgcatcatgaaggtgatgtcaaccggtctgcagggcggcacctcctttatggaggactacacctatcacttcgagaaaggtaatgacctggtgctcggctcccatatgctggaagtctgcccgtcgatcgccgcagaagagaaaccgatcctcgacgttcagcatctcggtattggtggtaaggacgatcctgcccgcctgatcttcaatacccaaaccggcccagcgattgtcgccagcttgattgatctcggcgatcgttaccgtctactggttaactgcatcgacacggtgaaaacaccgcactccctgccgaaactgccggtggcgaatgcgctgtggaaagcgcaaccggatctgccaactgcttccgaagcgtggatcctcgctggtggcgcgcaccataccgtcttcagccatgcactgaacctcaacgatatgcgccaattcgccgagatgcacgacattgaaatcacggtgattgataacgacacacgcctgccagcgtttaaagacgcgctgcgctggaacgaagtgtattacgggtttcgtcgctaa

SEQ ID NO: 95 - DNA, 696 bp, Escherichia coli
atgttagaagatctcaaacgccaggtattagaagccaacctggcgctgccaaaacacaacctggtcacgctcacatggggcaacgtcagcgccgttgatcgcgagcgcggcgtctttgtgatcaaaccttccggcgtcgattacagcgtcatgaccgctgacgatatggtcgtggttagcatcgaaaccggtgaagtggttgaaggtacgaaaaagccctcctccgacacgccaactcaccggctgctctatcaggcattcccctccattggcggcattgtgcatacgcactcgcgccacgccaccatctgggcgcaggcgggtcagtcgattccagcaaccggcaccacccacgccgactatttctacggcaccattccctgcacccgcaaaatgaccgacgcagaaatcaacggcgaatatgagtgggaaaccggtaacgtcatcgtagaaacctttgaaaaacagggtatcgatgcagcgcaaatgcccggcgttctggtccattcccacggcccgtttgcatggggcaaaaatgccgaagatgcggtgcataacgccatcgtgctggaagaggtcgcttatatggggatattctgccgtcagttagcgccgcagttaccggatatgcagcaaacgctgctggataaacactatctgcgtaagcatggcgcgaaggcatattacgggcagtaa

SEQ ID NO: 96 - DNA, 879 bp, Escherichia coli
atggctgaagcgcaaaatgatcccctgctgccgggatactcgtttaacgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgcattagtcaggcgaagctgcttttgagcactacccggatgcctatcgccaccgtcggtcgcaatgttggttttgacgatcaactctatttctcgcgagtatttaaaaaatgcaccggggccagcccgagcgagtttcgtgccggttgtgaagaaaaagtgaatgatgtagccgtcaagttgtcataa

SEQ ID NO: 97 - protein, 566 aa, Escherichia coli
MAIAIGLDFGSDSVRALAVDCATGEEIATSVEWYPRWQKGQFCDAPNNQFRHHPRDYIESMEAALKTVLAELSVEQRAAVVGIGVDSTGSTPAPIDADGNVLALRPEFAENPNAMFVLWKDHTAVEEAEEITRLCHAPGNVDYSRYIGGIYSSEWFWAKILHVTRQDSAVAQSAASWIELCDWVPALLSGTTRPQDIRRGRCSAGHKSLWHESWGGLPPASFFDELDPILNRHLPSPLFTDTWTADIPVGTLCPEWAQRLGLPESVVISGGAFDCHMGAVGAGAQPNALVKVIGTSTCDILIADKQSVGERAVKGICGQVDGSVVPGFIGLEAGQSAFGDIYAWFGRVLGWPLEQLAAQHPELKTQINASQKQLLPALTEAWAKNPSLDHLPVVLDWFNGRRTPNANQRLKGVITDLNLATDAPLLFGGLIAATAFGARAIMECFTDQGIAVNNVMALGGIARKNQVIMQACCDVLNRPLQIVASDQCCALGAAIFAAVAAKVHADIPSAQQKMASAVEKTLQPCSEQAQRFEQLYRRYQQWAMSAEQHYLPTSAPAQAAQAVATL

SEQ ID NO: 98 - protein, 500 aa, Escherichia coli
MTIFDNYEVWFVIGSQHLYGPETLRQVTQHAEHVVNALNTEAKLPCKLVLKPLGTTPDEITAICRDANYDDRCAGLVVWLHTFSPAKMWINGLTMLNKPLLQFHTQFNAALPWDSIDMDFMNLNQTAHGGREFGFIGARMRQQHAVVTGHWQDKQAHERIGSWMRQAVSKQDTRHLKVCRFGDNMREVAVTDGDKVAAQIKFGFSVNTWAVGDLVQVVNSISDGDVNALVDEYESCYTMTPATQIHGKKRQNVLEAARIELGMKRFLEQGGFHAFTTTFEDLHGLKQLPGLAVQRLMQQGYGFAGEGDWKTAALLRIMKVMSTGLQGGTSFMEDYTYHFEKGNDLVLGSHMLEVCPSIAAEEKPILDVQHLGIGGKDDPARLIFNTQTGPAIVASLIDLGDRYRLLVNCIDTVKTPHSLPKLPVANALWKAQPDLPTASEAWILAGGAHHTVFSHALNLNDMRQFAEMHDIEITVIDNDTRLPAFKDALRWNEVYYGFRR

SEQ ID NO: 99 - protein, 231 aa, Escherichia coli
MLEDLKRQVLEANLALPKHNLVTLTWGNVSAVDRERGVFVIKPSGVDYSVMTADDMVVVSIETGEVVEGTKKPSSDTPTHRLLYQAFPSIGGIVHTHSRHATIWAQAGQSIPATGTTHADYFYGTIPCTRKMTDAEINGEYEWETGNVIVETFEKQGIDAAQMPGVLVHSHGPFAWGKNAEDAVHNAIVLEEVAYMGIFCRQLAPQLPDMQQTLLDKHYLRKHGAKAYYGQ

SEQ ID NO: 100 - protein, 292 aa, Escherichia coli
MAEAQNDPLLPGYSFNAHLVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCEEKVNDVAVKLS